(19) United States
(12) Patent Application Publication
(10) Pub. No.: US 2026/0008770 A1
(43) Pub. Date: Jan. 8, 2026

(54) CBL-B INHIBITORS AND METHODS OF USES THEREOF

(71) Applicant: Insilico Medicine IP Limited, Hong Kong (HK)

(72) Inventors: Xiao DING, Shanghai (CN); Xiaosong LIU, Shanghai (CN); Yingtao LIU, Shanghai (CN); Fanye MENG, Shanghai (CN); Feng REN, Shanghai (CN); Yazhou WANG, Shanghai (CN); Hongbin WANG, Shanghai (CN)

(21) Appl. No.: 18/992,290

(22) PCT Filed: Jul. 17, 2023
(86) PCT No.: PCT/CN2023/107736

§ 371 Date: Jan. 8, 2025

(30) Foreign Application Priority Data
Jul. 18, 2022 (WO) PCT/CN2022/106243
Jan. 19, 2023 (WO) PCT/CN2023/073202
Jun. 7, 2023 (WO) PCT/CN2023/098931

(51) Int. Cl. C07D 401/14; A61K 31/454; A61K 31/4545; A61K 31/4725; A61K 31/498; A61K 31/4985; A61K 31/502; A61K 31/5025; A61K 31/517; A61K 31/519; A61K 31/5377; A61K 31/5386; C07B 59/00; C07D 401/12; C07D 403/10; C07D 405/14; C07D 413/14; C07D 417/14; C07D 471/04; C07D 487/04; C07D 491/107; C07D 495/04; C07D 513/04

(52) U.S. Cl. CPC C07D 401/14; A61K 31/454; A61K 31/4545; A61K 31/4725; A61K 31/498; A61K 31/4985; A61K 31/502; A61K 31/5025; A61K 31/517; A61K 31/519; A61K 31/5377; A61K 31/5386; C07B 59/002; C07D 401/12; C07D 403/10; C07D 405/14; C07D 413/14; C07D 417/14; C07D 471/04; C07D 487/04; C07D 491/107; C07D 495/04; C07D 513/04

(57) ABSTRACT

Provided for compounds and methods for modulating or inhibiting CBL-B.

### CROSS-REFERENCE

This patent application claims the benefit of International Application No. PCT/CN2022/106243, filed Jul. 18, 2022; International Application No. PCT/CN2023/073202, filed Jan. 19, 2023; and International Application No. PCT/CN2023/098931, filed Jun. 7, 2023; which is incorporated herein by reference in its entirety.

### BACKGROUND

Dysregulated signaling is a prominent feature in cellular transformation and tumorigenesis. The proto-oncogene Casitas B-lineage lymphoma (Cbl or c-Cbl), encodes an E3 ubiquitin ligase that downregulates PTK-directed cell signaling through ubiquitination, thereby targeting these kinases for lysosomal or proteasomal degradation. Cbl is a member of the Cbl family of proteins, so characterized based on a highly conserved N-terminal region that contains the structural components required for ubiquitin ligase activity. In simpler eukaryotic organisms, such as Caenorhabditis elegans and Dictyostelium discoideum, only one Cbl protein is present, but in mammals there are three, including Cbl, Cbl-b and Cbl-c.

The conserved N-terminus of Cbl family proteins contains a substrate tyrosine kinase-binding domain (TKBD), a linker helix region (LHR) and a RING domain. The TKBD confers specificity to Cbl's ligase activity based on the selective recruitment of phosphorylated substrates containing an (N/D)XpY(S/T)XXP, DpYR or RA(V/I)XNQpY(S/T) motif. The RING domain mediates the transfer of ubiquitin (Ub) from an E2 Ub-conjugating enzyme to the substrate. Within the LHR is a conserved tyrosine (Tyr371 in Cbl) that is crucial for regulating ligase activity. Phosphorylation of this tyrosine enhances ligase activity and is essential for ubiquitination of receptor PTKs. In addition to the highly conserved N-terminus, Cbl and Cbl-b also have extensive C-termini that confer adaptor-like functions to these proteins based on the ability to mediate multiple protein-protein interactions. These include a proline rich region that mediates interactions with SH3 domain-containing proteins and a tyrosine rich region that, upon phosphorylation, becomes a binding motif for other SH2 domain-containing proteins. Cbl and Cbl-b terminate with an ubiquitin-associated domain, which is crucial for homo- and heterodimerization of these two Cbl proteins.

Cbl-b is an important T cell immune response braker. In contrast to Cbl, which mainly regulates thymocyte development, Cbl-b mainly regulates peripheral T-cell activation through negative regulation of TCR (T-cell receptor) signal transduction pathways. Specifically, Cbl-b inhibits VAV1 activation upon TCR engagement and imposes a requirement for CD28 costimulation for proliferation and IL-2 production in naïve T cells. Cbl-b also ubiquitylates PIK3R1/p85, which inhibits its recruitment to CD28 and TCRζ, therefore suppressing the activation of PI3K, an important kinase involved in T cell activation and differentiation. Moreover, in activated T-cells, Cbl-b inhibits PLCG1 activation and calcium mobilization upon restimulation and thus promotes T cell anergy. Therefore, Cbl-b restricts unnecessary T cell overactivation under physiological condition. Indeed, Cbl-b KO mice had increased T cell proliferation, spontaneous autoimmunity characterized by auto-antibody production, infiltration of activated T and B lymphocytes into multiple organs. On the other hand, in tumor microenvironment, T cells anergy, exhaustion and exclusion are widely present. Thus, to unleash T cell's full power through release of the Cbl-b brake as an immune-oncological anti-cancer therapy holds great promise.

There is a need for new cancer therapies, specifically using Cbl-b inhibitors.

### SUMMARY

Disclosed herein are compounds, or a pharmaceutically acceptable salt thereof, that are Cbl-b inhibitors.

Disclosed herein is a compound of Formula (I), or a pharmaceutically acceptable salt thereof:

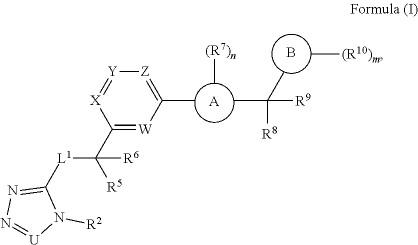

as described herein;
provided that

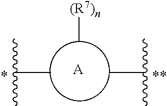

is not

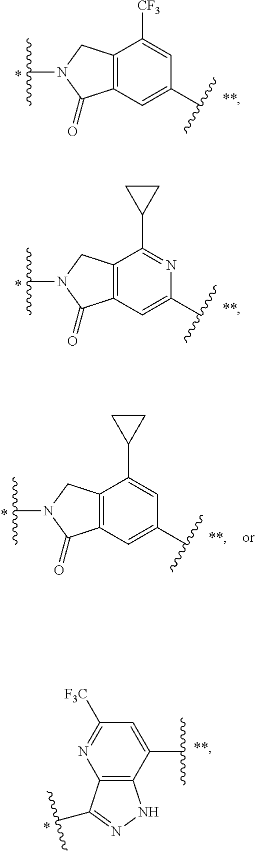 [SMILES: *c1cc2c(c(C(F)(F)F)c1)CN(*)C2=O ; *c1cc2c(c(C3CC3)n1)CN(*)C2=O ; *c1cc2c(c(C3CC3)c1)CN(*)C2=O ; *c1n[nH]c2c(*)cc(C(F)(F)F)nc12]

wherein * represents the attachment point to the ring containing X, Y, Z and W and ** represents the attachment point to —CR⁸R⁹—.

Also provided herein is a compound of Formula (III), or a pharmaceutically acceptable salt thereof:

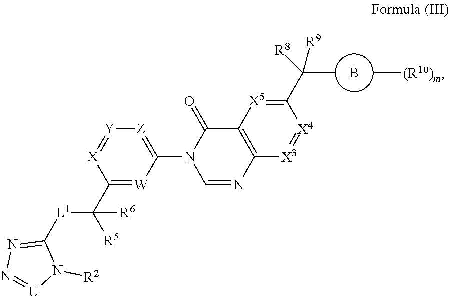

as described herein.

Also provided herein is a compound of Formula (II), or a pharmaceutically acceptable salt thereof:

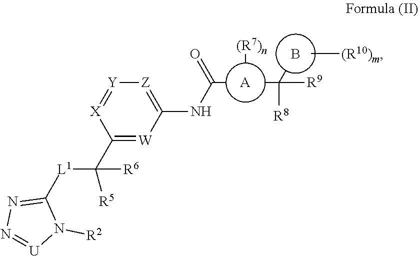

as described herein;
provided that

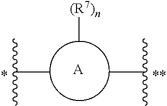

is not

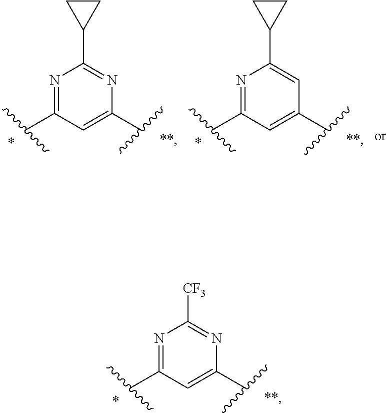 [SMILES: *c1cc(*)nc(C2CC2)n1 ; *c1cc(*)nc(C2CC2)c1 ; *c1cc(*)nc(C(F)(F)F)n1]

wherein * represents the attachment point to —C(═O)—NH— group and ** represents the attachment point to —CR⁸R⁹— and provided that the compound of Formula (II) is not

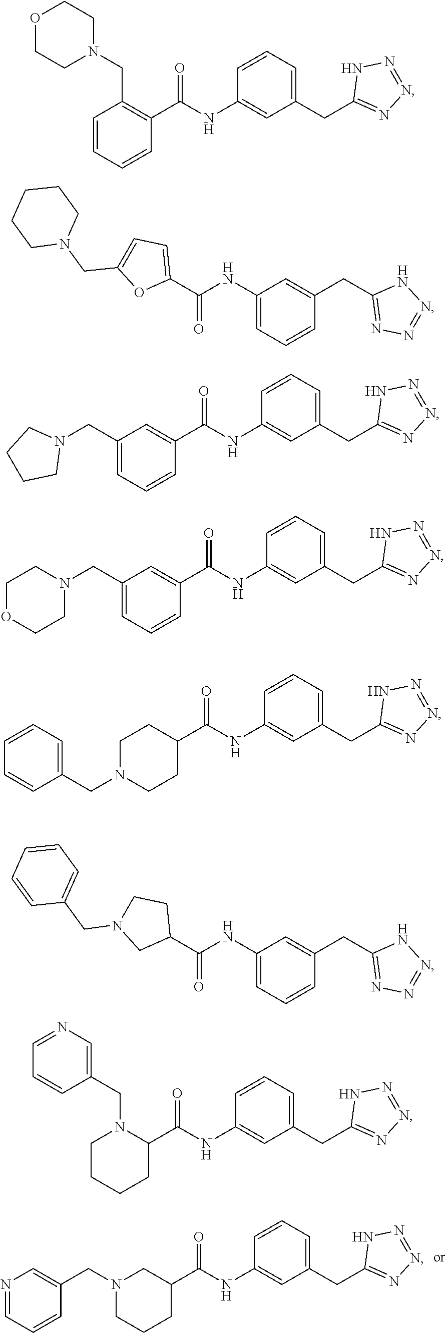

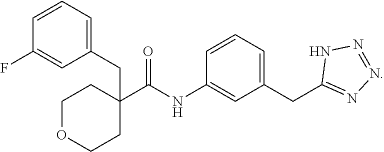 [SMILES: O=C(Nc1cccc(Cc2nnn[nH]2)c1)C1(Cc2cccc(F)c2)CCOCC1]

Also disclosed herein is a pharmaceutical composition comprising a compound disclosed herein, or a pharmaceutically acceptable salt thereof, and a pharmaceutically acceptable excipient.

Also disclosed herein is a method of modulating activity of an immune cell, the method comprising contacting the immune cell with an effective amount of a compound disclosed herein, or a pharmaceutically acceptable salt thereof, to the subject in need thereof.

Also disclosed herein is a method of treating a cancer, the method comprising administering an effective amount of a compound disclosed herein, or a pharmaceutically acceptable salt thereof, to the subject in need thereof.

Also disclosed herein is a method of treating a cancer responsive to inhibition of Cbl-b activity, the method comprising administering an effective amount of a compound disclosed herein, or a pharmaceutically acceptable salt thereof, to the subject in need thereof.

In some embodiments, the cancer is a hematologic cancer. In some embodiments, the cancer is a lymphoma, a leukemia, or a myeloma. In some embodiments, the cancer is a non-hematologic cancer. In some embodiments, the cancer is a sarcoma, a carcinoma, or a melanoma. In some embodiments, the cancer is solid tumor cancer

Also disclosed herein is a method of inhibiting abnormal cell proliferation, the method comprising administering an effective amount of a compound disclosed herein, or a pharmaceutically acceptable salt thereof, to the subject in need thereof.

Also disclosed herein is a method of modulating the immune response, the method comprising administering an effective amount of a compound disclosed herein, or a pharmaceutically acceptable salt thereof, to the subject in need thereof.

Also disclosed herein is a method of inhibiting Cbl-b activity, the method comprising administering an effective amount of a compound disclosed herein, or a pharmaceutically acceptable salt thereof, to the subject in need thereof.

Also disclosed herein is a method for treating a disease or condition associated with Cbl-b activity, the method comprising administering an effective amount of a compound disclosed herein, or a pharmaceutically acceptable salt thereof, to the subject in need thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

In the following description, certain specific details are set forth to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₆alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)=CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —Oalkyl where alkyl is as defined above. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁₀ fully saturated cycloalkyl or C₃-C₁₀ cycloalkenyl), from three to eight carbon atoms (e.g., C₃-C₅ fully saturated cycloalkyl or C₃-C₅ cycloalkenyl), from three to six carbon atoms (e.g., C₃-C₆ fully saturated cycloalkyl or C₃-C₆ cycloalkenyl), from three to five carbon atoms (e.g., C₃-C₅ fully saturated cycloalkyl or C₃-C₅ cycloalkenyl), or three to four carbon atoms (e.g., C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Haloalkoxy” refers to a radical of the formula —Ohaloalkyl where haloalkyl is as defined above. In some embodiments, the haloalkoxy comprises 1 to 3 halogens. In some embodiments, the haloalkoxy comprises 1 to 3 fluoros. In some embodiments, the haloalkoxy is —OCF₃, —OCHF₂, —OCH₂F, —OCH₂CF₃, —OCH₂CHF₂, or —OCH₂CH₂F.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₆ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₇ heterocycloalkenyl), from two to five carbon atoms (e.g., C₂-C₅ fully saturated heterocycloalkyl or C₂-C₆ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns that are sterically impractical and/or synthetically non-feasible. Thus, any substituents described should generally be understood as having a maximum molecular weight of about 1,000 daltons, and more typically, up to about 500 daltons.

The term “one or more” when referring to an optional substituent means that the subject group is optionally substituted with one, two, three, four, or more substituents. In some embodiments, the subject group is optionally substituted with one, two, three or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or “therapeutically effective amount” refers to an amount of a compound administered to a mammalian subject, either as a single dose or as part of a series of doses, which is effective to produce a desired therapeutic effect.

The terms “treat,” “treating” or “treatment,” as used herein, include alleviating, abating, or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

As used herein, a “disease or disorder associated with cbl-b” or, alternatively, “a cbl-b-mediated disease or disorder” means any disease or other deleterious condition in which cbl-b, or a mutant thereof, is known or suspected to play a role.

### Compounds

Described herein are compounds, or a pharmaceutically acceptable salt thereof useful in the treatment of a disease or disorder associated with cbl-b.

Disclosed herein is a compound of Formula (I), or a pharmaceutically acceptable salt thereof:

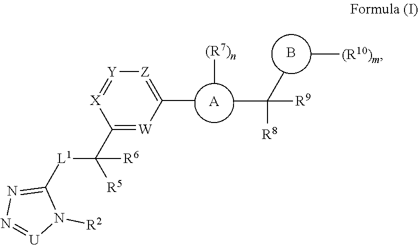

wherein:
U is —N— or —CR¹—;
R¹ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R² is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
L¹ is absent or —CR³R⁴—;
R³ and R⁴ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or R³ and R⁴ are taken together to form a cycloalkyl or heterocycloalkyl; each optionally substituted with one or more R;
or R³ and R⁴ are taken together to form an oxo;
R⁵ and R⁶ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{5a};
or R⁵ and R⁶ are taken together to form a cycloalkyl or heterocycloalkyl; each optionally substituted with one or more R^{5a};
each R^{5a} is independently halogen, —CN, —NO₂, —OH, —OR^{a}, —OC(═O)R^{b}, —OC(═O)OR^{b}, —OC(═O)NR^{c}R^{d}, —SF₅, —SH, —SR^{b}, —S(═O)R^{a}, —S(═O)₂R^{b}, —S(═O)₂NR^{c}R^{d}, —S(═O)(=NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{5a} on the same atom are taken together to form an oxo;
X is —N— or —CR^{X}—;
R^{X} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Y is —N— or —CR^{Y}—;
R^{Y} is hydrogen, halogen, —CN, —OH, —OR^{a}, —NR^{c}R^{d}, —S(═O)R^{a}, —S(═O)₂R^{a}, —S(═O)₂NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{b}, —C(═O)OR^{b}, —C(═O)NR^{c}R^{d}, C₁-C₆alkyl, C₁-C₆haloalkyl, C₁-C₆alkoxy, C₁-C₆haloalkoxy, C₁-C₆hydroxyalkyl, C₁-C₆aminoalkyl, C₁-C₆heteroalkyl, C₂-C₆alkenyl, C₂-C₆haloalkenyl C₂-C₆alkynyl, cycloalkyl optionally substituted with one or more halogens, or heterocycloalkyl optionally substituted with one or more halogens;
Z is —N— or —CR^{Z}—.
R^{Z} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
or R^{X} and R^{Y} are taken together to form a cycloalkyl, heterocycloalkyl, aryl, or heteroaryl; each optionally substituted with one or more R;
or R^{Y} and R^{Z} are taken together to form a cycloalkyl, heterocycloalkyl, aryl, or heteroaryl; each optionally substituted with one or more R;
W is —N— or —CR^{W};
R^{W} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Ring A is cycloalkyl, heterocycloalkyl, aryl, or heteroaryl;
each R⁷ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(=NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R⁷ on the same atom are taken together to form an oxo;
n is 0, 1, 2, 3, 4, 5, or 6;
R⁸ and R⁹ are each independently hydrogen, halogen, —CN, —OH, —OW, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R; or R⁸ and R⁹ are taken together to form a cycloalkyl or heterocycloalkyl; each optionally substituted with one or more R;
or R⁸ and R⁹ are taken together to form an oxo;
Ring B is cycloalkyl, heterocycloalkyl, aryl, or heteroaryl;
each R¹⁰ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(=NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or two R¹⁰ on the same atom are taken together to form an oxo;
m is 0, 1, 2, 3, 4, 5, or 6;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(=NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, or C₃-C₆cycloalkyl;
or two R on the same atom form an oxo;
provided that

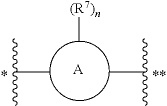

is not

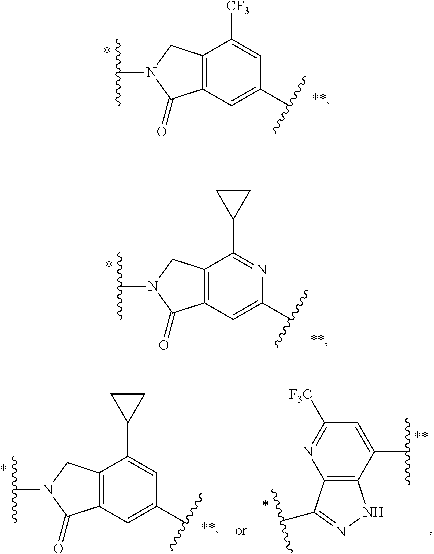 [SMILES: *c1cc2c(c(C(F)(F)F)c1)CN(*)C2=O ; *c1cc2c(c(C3CC3)n1)CN(*)C2=O ; *c1cc2c(c(C3CC3)c1)CN(*)C2=O ; *c1n[nH]c2c(*)cc(C(F)(F)F)nc12]

wherein * represents the attachment point to the ring containing X, Y, Z and W and ** represents the attachment point to —CR⁸R⁹—.

Disclosed herein is a compound of Formula (I), or a pharmaceutically acceptable salt thereof:

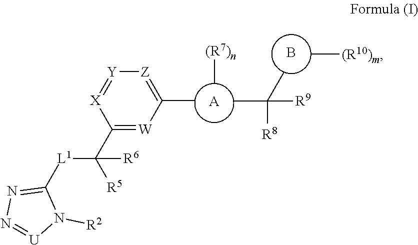

wherein:
U is —N— or —CR¹—;
R¹ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R² is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
L¹ is absent or —CR³R⁴—;
R³ and R⁴ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or R³ and R⁴ are taken together to form a cycloalkyl or heterocycloalkyl; each optionally substituted with one or more R;
or R³ and R⁴ are taken together to form an oxo;
R⁵ and R⁶ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{5a};
or R⁵ and R⁶ are taken together to form a cycloalkyl or heterocycloalkyl; each optionally substituted with one or more R^{5a};
each R^{5a} is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{b}, —S(═O)₂NR^{c}R^{d}, —S(═O)(=NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
X is —N— or —CR^{X}—;
R^{X} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; Y is —N— or —CR^{Y}—;
R^{Y} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆haloalkenyl, C₂-C₆alkynyl, cycloalkyl optionally substituted with one or more halogens, or heterocycloalkyl optionally substituted with one or more halogens;
Z is —N— or —CR^{Z}—;
R^{Z} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
W is —N— or —CR^{W}—;
R^{W} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Ring A is cycloalkyl, heterocycloalkyl, aryl, or heteroaryl;
each R⁷ is independently halogen, —CN, —NO₂, —OH, —OR^{a}, —OC(═O)R^{a}, —OC(O)OR^{b}, —OC(═O)NR^{c}R^{d}, —SF₅, —SH, —SR^{a}, —S(═O)R^{a}, —S(═O)₂R^{b}, —S(═O)₂NR^{c}R^{d}, —S(═O)(=NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{b}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R⁷ on the same atom are taken together to form an oxo;
n is 0, 1, 2, 3, 4, 5, or 6;
R⁸ and R⁹ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or R⁸ and R⁹ are taken together to form a cycloalkyl or heterocycloalkyl; each optionally substituted with one or more R;
or R⁸ and R⁹ are taken together to form an oxo;
Ring B is cycloalkyl, heterocycloalkyl, aryl, or heteroaryl;
each R¹⁰ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{b}, —S(═O)₂NR^{c}R^{d}, —S(═O)(=NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or two R⁰ on the same atom are taken together to form an oxo;
m is 0, 1, 2, 3, 4, 5, or 6;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C_{c}heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(=NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, or C₃-C₆cycloalkyl;
or two R on the same atom form an oxo;
provided that

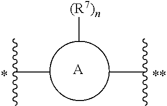

is not

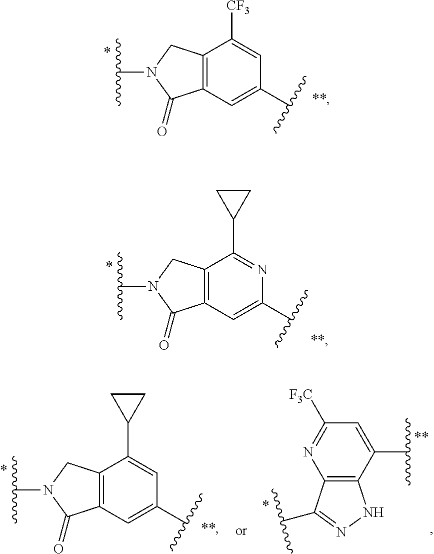 [SMILES: *c1cc2c(c(C(F)(F)F)c1)CN(*)C2=O ; *c1cc2c(c(C3CC3)n1)CN(*)C2=O ; *c1cc2c(c(C3CC3)c1)CN(*)C2=O ; *c1n[nH]c2c(*)cc(C(F)(F)F)nc12]

wherein * represents the attachment point to the ring containing X, Y, Z and W and ** represents the attachment point to —CR⁸R⁹—.

In some embodiments of a compound of Formula (I), X is —N—. In some embodiments of a compound of Formula (I), X is —CR^{X}—.

In some embodiments of a compound of Formula (I), Y is —N—. In some embodiments of a compound of Formula (I), Y is —CR^{Y}—.

In some embodiments of a compound of Formula (I), Z is —N—. In some embodiments of a compound of Formula (I), Z is —CR^{Z}—.

In some embodiments of a compound of Formula (I), W is —N—. In some embodiments of a compound of Formula (I), W is —CR^{W}—.

In some embodiments of a compound of Formula (I), the compound is of Formula (Ia):

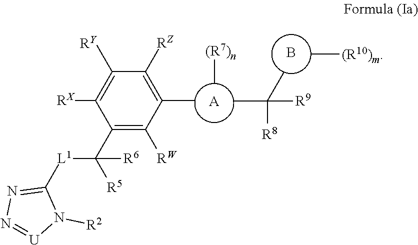

In some embodiments of a compound of Formula (I), the compound is of Formula (Ib):

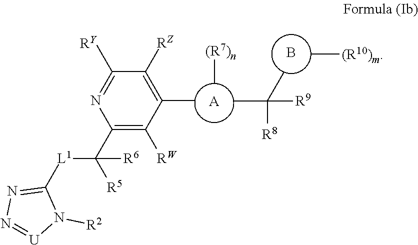

In some embodiments of a compound of Formula (I), the compound is of Formula (Ic):

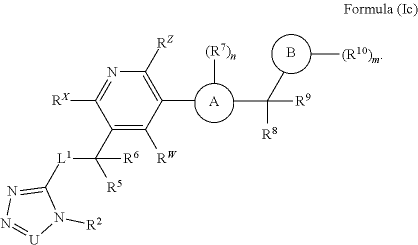

In some embodiments of a compound of Formula (I), the compound is of Formula (Id):

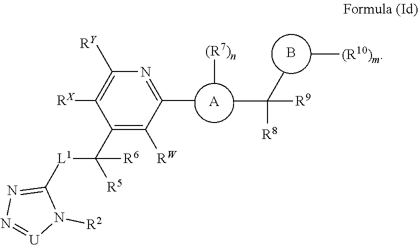

In some embodiments of a compound of Formula (I), (Ia), (Ic), or (Id), RX is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (I), (Ia), (Ic), or (Id), R^{X} is hydrogen, halogen, C₁-C₆alkyl, C₁-C₆haloalkyl, cycloalkyl, or heterocycloalkyl. In some embodiments of a compound of Formula (I), (Ia), (Ic), or (Id), R^{X} is hydrogen, halogen, C₁-C₆alkyl, or C₁-C₆haloalkyl. In some embodiments of a compound of Formula (I), (Ia), (Ic), or (Id), R^{X} is hydrogen or C₁-C₆alkyl. In some embodiments of a compound of Formula (I), (Ia), (Ic), or (Id), R^{X} is hydrogen.

In some embodiments of a compound of Formula (I), (Ia), (Ib), or (Id), R^{X} is hydrogen, halogen, —CN, —OH, —OR^{a}, —NR^{c}R^{d}, —S(═O)R^{a}, —S(═O)₂R^{b}, —S(═O)₂NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆haloalkenyl C₂-C₆alkynyl, cycloalkyl optionally substituted with one or more halogens, or heterocycloalkyl optionally substituted with one or more halogens.

In some embodiments of a compound of Formula (I), (Ia), (Ib), or (Id), R^{Y} is hydrogen, halogen, —CN, —OR^{a}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆haloalkenyl, C₂-C₆alkynyl, cycloalkyl optionally substituted with one or more halogens, or heterocycloalkyl optionally substituted with one or more halogens.

In some embodiments of a compound of Formula (I), (Ia), (Ib), or (Id), R^{Y} is hydrogen, halogen, —CN, —OR^{a}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl optionally substituted with one or more halogens, or heterocycloalkyl optionally substituted with one or more halogens.

In some embodiments of a compound of Formula (I), (Ia), (Ib), or (Id), R^{Y} is hydrogen, halogen, —CN, —OR^{a}, —C(═O)NR^{c}R^{d}, C₁-C₆alkyl, C₁-C₆haloalkyl, C₁-C₆hydroxyalkyl, C₂-C₆alkenyl, C₂-C₆haloalkenyl, C₂-C₆alkynyl, or cycloalkyl optionally substituted with one or more halogens.

In some embodiments of a compound of Formula (I), (Ia), (Ib), or (Id), R^{Y} is hydrogen, C₁-C₆alkyl, C₁-C₆haloalkyl, C₃-C₆cycloalkyl, or C₁-C₆haloalkoxy.

In some embodiments of a compound of Formula (I), (Ia), (Ib), or (Id), R^{Y} is hydrogen, C₃-C₆cycloalkyl, or C₁-C₆haloalkoxy.

In some embodiments of a compound of Formula (I), (Ia), (Ib), or (Id), R^{Y} is hydrogen, —OR^{a}, C₁-C₆alkyl, C₁-C₆haloalkyl, or C₃-C₆cycloalkyl; and R^{a} is C₁-C₆haloalkyl.

In some embodiments of a compound of Formula (I), (Ia), (Ib), or (Id), R^{Y} is hydrogen, —OR^{a}, or C₃-C₆cycloalkyl; and R^{a} is C₁-C₆haloalkyl.

In some embodiments of a compound of Formula (I), (Ia), (Ib), or (Id), R^{Y} is hydrogen, halogen, —CN, —OR^{a}, —C(═O)NR^{c}R^{d}, C₁-C₆alkyl, C₁-C₆haloalkyl, C₁-C₆hydroxyalkyl, C₂-C₆haloalkenyl, C₂-C₆alkynyl, or cycloalkyl optionally substituted with one or more halogens.

In some embodiments of a compound of Formula (I), (Ia), (Ib), or (Id), R^{Y} is hydrogen, halogen, —CN, —OR^{a}, —C(═O)NR^{c}R^{d}, C₁-C₆alkyl, C₁-C₆hydroxyalkyl, C₂-C₆alkenyl, C₂-C₆haloalkenyl, C₂C₂-C₆alkynyl, or cycloalkyl optionally substituted with one or more halogens.

In some embodiments of a compound of Formula (I), (Ia), (Ib), or (Id), R^{Y} is hydrogen, halogen, —CN, —OR^{a}, —C(═O)NR^{c}R^{d}, C₁-C₆alkyl, C₁-C₆hydroxyalkyl, C₂-C₆alkenyl, C₂-C₆alkynyl, or cycloalkyl optionally substituted with one or more halogens.

In some embodiments of a compound of Formula (I), (Ia), (Ib), or (Id), R^{Y} is hydrogen, —OW, or cycloalkyl optionally substituted with one or more halogens.

In some embodiments of a compound of Formula (I), (Ia), (Ib), or (Id), R^{Y} is hydrogen, —OR^{a}, or cycloalkyl.

In some embodiments of a compound of Formula (I), (Ia), (Ib), or (Id), R^{Y} is —OR. In some embodiments of a compound of Formula (I), (Ia), (Ib), or (Id), R^{Y} is C₁-C₆haloalkoxy. In some embodiments of a compound of Formula (I), (Ia), (Ib), or (Id), R^{Y} is C₁-C₃haloalkoxy. In some embodiments of a compound of Formula (I), (Ia), (Ib), or (Id), R^{Y} is —OCH₂F.

In some embodiments of a compound of Formula (I), (Ia), (Ib), or (Id), R^{Y} is cycloalkyl. In some embodiments of a compound of Formula (I), (Ia), (Ib), or (Id), R^{Y} is C₃-C₆cycloalkyl. In some embodiments of a compound of Formula (I), (Ia), (Ib), or (Id), R^{Y} is cyclopropyl.

In some embodiments of a compound of Formula (I), (Ia), (Ib), or (Id), R^{Y} is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl optionally substituted with one or more halogens, or heterocycloalkyl optionally substituted with one or more halogens.

In some embodiments of a compound of Formula (I), (Ia), (Ib), or (Id), R^{Y} is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl.

In some embodiments of a compound of Formula (I), (Ia), (Ib), or (Id), R^{Y} is hydrogen, halogen, C₁-C₆alkyl, C₁-C₆haloalkyl, C₃-C₆cycloalkyl optionally substituted with one or more halogens, or 3- to 6-membered heterocycloalkyl optionally substituted with one or more halogens.

In some embodiments of a compound of Formula (I), (Ia), (Tb), or (Id), R^{Y} is hydrogen, halogen, C₁-C₆alkyl, C₁-C₆haloalkyl, C₃-C₆cycloalkyl, or 3- to 6-membered heterocycloalkyl.

In some embodiments of a compound of Formula (I), (Ia), (Tb), or (Id), R^{Y} is hydrogen, methyl, ethyl, —OCH₃, —OCH₂F, Cl, F, —CH═CH₂, —CF=CH₂,

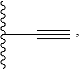 [SMILES: *C#C]

CN, —C(═O)NH₂, C(═O)NHCH₃, or —CH₂OH.

In some embodiments of a compound of Formula (I), (Ia), (Ib), or (Ic), R^{Z} is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (I), (Ia), (Tb), or (Ic), R^{Z} is hydrogen, halogen, C₁-C₆alkyl, C₁-C₆haloalkyl, cycloalkyl, or heterocycloalkyl. In some embodiments of a compound of Formula (I), (Ia), (Tb), or (Ic), R^{Z} is hydrogen, halogen, C₁-C₆alkyl, or C₁-C₆haloalkyl. In some embodiments of a compound of Formula (I), (Ia), (Tb), or (Ic), R^{Z} is hydrogen or C₁-C₆alkyl. In some embodiments of a compound of Formula (I), (Ia), (Tb), or (Ic), R^{Z} is hydrogen. In some embodiments of a compound of Formula (I), (Ia), (Tb), or (Ic), R^{Z} is hydrogen or halogen. In some embodiments of a compound of Formula (I), (Ia), (Tb), or (Ic), R^{Z} is halogen.

In some embodiments of a compound of Formula (I) or (Ia), R^{X} and R^{Y} are taken together to form a cycloalkyl, heterocycloalkyl, aryl, or heteroaryl; each optionally substituted with one or more R. In some embodiments of a compound of Formula (I) or (Ia), R^{X} and R^{Y} are taken together to form a cycloalkyl or heterocycloalkyl; each optionally substituted with one or more R. In some embodiments of a compound of Formula (I) or (Ia), R^{X} and R^{Y} are taken together to form a cycloalkyl optionally substituted with one or more R. In some embodiments of a compound of Formula (I) or (Ia), R^{X} and R^{Y} are taken together to form a heterocycloalkyl optionally substituted with one or more R.

In some embodiments of a compound of Formula (I) or (Ia), R^{Y} and R^{Z} are taken together to form a cycloalkyl, heterocycloalkyl, aryl, or heteroaryl; each optionally substituted with one or more R. In some embodiments of a compound of Formula (I) or (Ia), R^{Y} and R^{Z} are taken together to form a cycloalkyl or heterocycloalkyl; each optionally substituted with one or more R. In some embodiments of a compound of Formula (I) or (Ia), R^{Y} and R^{Z} are taken together to form a C₅-C₆cycloalkyl optionally substituted with one or more R. In some embodiments of a compound of Formula (I) or (Ia), R^{Y} and R^{Z} are taken together to form a 5- or 6-membered heterocycloalkyl optionally substituted with one or more R.

In some embodiments of a compound of Formula (I) or (Ia)-(Id), R^{W} is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (I) or (Ia)-(Id), R^{W} is hydrogen, halogen, C₁-C₆alkyl, C₁-C₆haloalkyl, cycloalkyl, or heterocycloalkyl. In some embodiments of a compound of Formula (I) or (Ia)-(Id), R^{W} is hydrogen, halogen, C₁-C₆alkyl, or C₁-C₆haloalkyl. In some embodiments of a compound of Formula (I) or (Ia)-(Id), R^{W} is hydrogen or C₁-C₆alkyl. In some embodiments of a compound of Formula (I) or (Ia)-(Id), R^{W} is hydrogen.

In some embodiments of a compound of Formula (I),

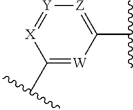

is

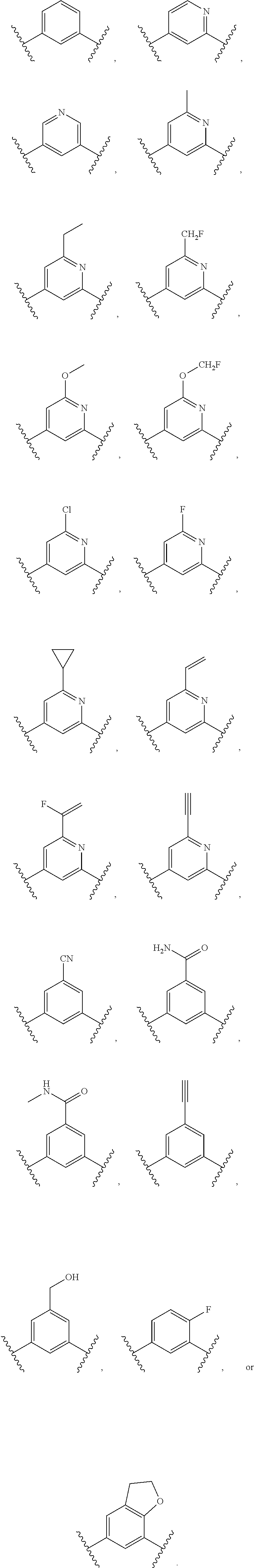

In some embodiments of a compound of Formula (I) or (Ia)-(Id), R⁸ and R⁹ are each independently hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound of Formula (I) or (Ia)-(Id), R⁸ and R⁹ are each independently hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (I) or (Ia)-(Id), R⁸ and R⁹ are each independently hydrogen, halogen, C₁-C₆alkyl, or C₁-C₆haloalkyl. In some embodiments of a compound of Formula (I) or (Ia)-(Id), R⁸ and R⁹ are each independently hydrogen or C₁-C₆alkyl. In some embodiments of a compound of Formula (I) or (Ia)-(Id), R⁸ and R⁹ are each hydrogen. In some embodiments of a compound of Formula (I) or (Ia)-(Id), R⁸ and R⁹ are taken together to form a cycloalkyl or heterocycloalkyl; each optionally substituted with one or more R. In some embodiments of a compound of Formula (I) or (Ia)-(Id), R⁸ and R⁹ are taken together to form an oxo.

In some embodiments of a compound of Formula (I) or (Ia)-(Id), Ring B is cycloalkyl or heterocycloalkyl. In some embodiments of a compound of Formula (I) or (Ia)-(Id), Ring B is cycloalkyl. In some embodiments of a compound of Formula (I) or (Ia)-(Id), Ring B is heterocycloalkyl. In some embodiments of a compound of Formula (I) or (Ia)-(Id), Ring B is 5- or 6-membered heterocycloalkyl.

In some embodiments of a compound of Formula (I) or (Ia)-(Id), Ring B is azetidinyl, pyrrolidinyl, piperidinyl, or morpholinyl. In some embodiments of a compound of Formula (I) or (Ia)-(Id), Ring B is piperidinyl.

In some embodiments of a compound of Formula (I) or (Ia)-(Id), each R¹⁰ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, C₁-C₆alkylene(cycloalkyl), or C₁-C₆alkylene(heterocycloalkyl); wherein each alkyl, alkylene, alkenyl, alkynyl, cycloalkyl, and heterocycloalkyl is independently optionally substituted with one or more R.

In some embodiments of a compound of Formula (I) or (Ia)-(Id), each R¹⁰ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, C₁-C₆alkylene(cycloalkyl), or C₁-C₆alkylene(heterocycloalkyl).

In some embodiments of a compound of Formula (I) or (Ia)-(Id), each R¹⁰ is independently halogen, —CN, —OH, —OR^{a}, —NR^{c}R^{d}, C₁-C₆alkyl, C₁-C₆haloalkyl, C₂-C₆alkenyl, C₂-C₆alkynyl, cycloalkyl, heterocycloalkyl, C₁-C₆alkylene(cycloalkyl), or C₁-C₆alkylene(heterocycloalkyl).

In some embodiments of a compound of Formula (I) or (Ia)-(Id), each R¹⁰ is independently halogen, —OH, —OR^{a}, C₁-C₆alkyl, C₁-C₆haloalkyl, C₂-C₆alkenyl, C₂-C₆alkynyl, cycloalkyl, or C₁-C₆alkylene(cycloalkyl).

In some embodiments of a compound of Formula (I) or (Ia)-(Id), each R¹⁰ is independently halogen, —OH, —OR^{a}, C₁-C₆alkyl, C₂-C₆alkenyl, C₂-C₆alkynyl, cycloalkyl, or C₁-C₆alkylene(cycloalkyl).

In some embodiments of a compound of Formula (I) or (Ia)-(Id), each R¹⁰ is independently halogen, —CN, —OH, —OR^{a}, —NR^{c}R^{d}, C₁-C₆alkyl, C₁-C₆haloalkyl, C₂-C₆alkynyl, cycloalkyl, heterocycloalkyl, C₁-C₆alkylene(cycloalkyl), or C₁-C₆alkylene(heterocycloalkyl); wherein each alkyl, alkylene, alkynyl, cycloalkyl, and heterocycloalkyl is independently optionally substituted with one or more R.

In some embodiments of a compound of Formula (I) or (Ia)-(Id), each R¹⁰ is independently halogen, C₁-C₆alkyl, C₁-C₆haloalkyl, C₂-C₆alkynyl, C₁-C₆alkylene(C₃-C₆cycloalkyl), or C₁-C₆alkylene(heterocycloalkyl); wherein each alkyl, alkylene, alkynyl, cycloalkyl, and heterocycloalkyl is independently optionally substituted with one or more R. In some embodiments of a compound of Formula (I) or (Ia)-(Id), each R¹⁰ is independently halogen or C₁-C₆alkyl. In some embodiments of a compound of Formula (I) or (Ia)-(Id), each R¹⁰ is independently C₁-C₆alkyl. In some embodiments of a compound of Formula (I) or (Ia)-(Id), each R¹⁰ is independently F, methyl, OH,

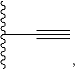 [SMILES: *C#C]

### —CH═CH₂,

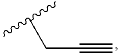 [SMILES: *CC#C]

cyclopropyl, —OCHF₂, or —CH₂F.

In some embodiments of a compound of Formula (I) or (Ia)-(Id), two R¹⁰ are taken together to form a cycloalkyl, heterocycloalkyl, aryl, or heteroaryl; each optionally substituted with one or more R. In some embodiments of a compound of Formula (I) or (Ia)-(Id), two R¹⁰ are taken together to form a C₃-C₆cycloalkyl, or 3- to 6-membered heterocycloalkyl; each optionally substituted with one or more R.

In some embodiments of a compound of Formula (I) or (Ia)-(Id), m is 0, 1, 2, or 3. In some embodiments of a compound of Formula (I) or (Ia)-(Id), m is 1 or 2. In some embodiments of a compound of Formula (I) or (Ia)-(Id), m is 1, 2, or 3. In some embodiments of a compound of Formula (I) or (Ia)-(Id), m is 0, 1, or 2. In some embodiments of a compound of Formula (I) or (Ia)-(Id), m is 1 or 2. In some embodiments of a compound of Formula (I) or (Ia)-(Id), m is 0. In some embodiments of a compound of Formula (I) or (Ia)-(Id), m is 1. In some embodiments of a compound of Formula (I) or (Ia)-(Id), m is 2. In some embodiments of a compound of Formula (I) or (Ia)-(Id), m is 3.

In some embodiments of a compound of Formula (I) or (Ia)-(Id),

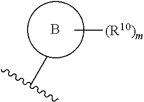

is

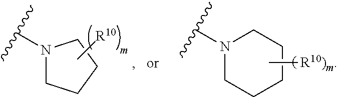

In some embodiments of a compound of Formula (I) or (Ia)-(Id),

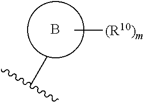

is

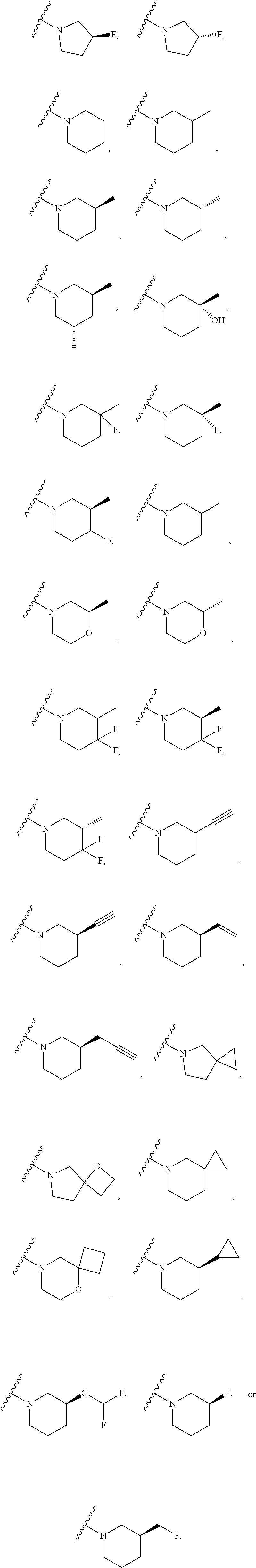

In some embodiments of a compound of Formula (I) or (Ia)-(Id),

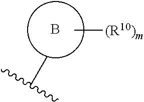

is

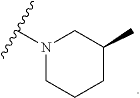 [SMILES: *N1CCC[C@H](C)C1]

In some embodiments of a compound of Formula (I) or (Ia)-(Id), L¹ is absent. In some embodiments of a compound of Formula (I) or (Ia)-(Id), L is —CR³R⁴—.

In some embodiments of a compound of Formula (I) or (Ia)-(Id), R³ and R⁴ are each independently hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound of Formula (I) or (Ia)-(Id), R³ and R⁴ are each independently hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (I) or (Ia)-(Id), R³ and R⁴ are each independently hydrogen, halogen, C₁-C₆alkyl, C₁-C₆haloalkylcycloalkyl, or heterocycloalkyl. In some embodiments of a compound of Formula (I) or (Ia)-(Id), R³ and R⁴ are each independently hydrogen, halogen, C₁-C₆alkyl, or C₁-C₆haloalkyl. In some embodiments of a compound of Formula (I) or (Ia)-(Id), R³ and R⁴ are each hydrogen. In some embodiments of a compound of Formula (I) or (Ia)-(Id), R³ and R⁴ are taken together to form a cycloalkyl or heterocycloalkyl; each optionally substituted with one or more R. In some embodiments of a compound of Formula (I) or (Ia)-(Id), R³ and R⁴ are taken together to form an oxo.

In some embodiments of a compound of Formula (I) or (Ia)-(Id), R⁶ is hydrogen, halogen, —CN, —OH, —OR^{a}, —NR^{c}R^{d}, C₁-C₆alkyl, or C₁-C₆haloalkyl. In some embodiments of a compound of Formula (I) or (Ia)-(Id), R⁶ is hydrogen, C₁-C₆alkyl, or C₁-C₆haloalkyl. In some embodiments of a compound of Formula (I) or (Ia)-(Id), R⁶ is hydrogen or C₁-C₆alkyl. In some embodiments of a compound of Formula (I) or (Ia)-(Id), R⁶ is hydrogen.

In some embodiments of a compound of Formula (I) or (Ia)-(Id), R⁵ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R⁵.

In some embodiments of a compound of Formula (I) or (Ia)-(Id), R⁵ is cycloalkyl, heterocycloalkyl, aryl, or heteroaryl; wherein each cycloalkyl, heterocycloalkyl, aryl, and heteroaryl is independently optionally substituted with one or more R^{5a}. In some embodiments of a compound of Formula (I) or (Ia)-(Id), R⁵ is cycloalkyl or heterocycloalkyl; wherein each cycloalkyl and heterocycloalkyl is independently optionally substituted with one or more R^{5a}. In some embodiments of a compound of Formula (I) or (Ia)-(Id), R⁵ is C₃-C₆cycloalkyl optionally substituted with one or more R^{5a}. In some embodiments of a compound of Formula (I) or (Ia)-(Id), R⁵ is 3- to 6-membered heterocycloalkyl optionally substituted with one or more R^{5a}.

In some embodiments of a compound of Formula (I) or (Ia)-(Id), R⁵ and R⁶ are taken together to form a cycloalkyl optionally substituted with one or more R^{5a}. In some embodiments of a compound of Formula (I) or (Ia)-(Id), R⁵ and R⁶ are taken together to form a C₃-C₆cycloalkyl optionally substituted with one or more R^{5a}.

In some embodiments of a compound of Formula (I) or (Ia)-(Id), R⁵ and R⁶ are taken together to form a heterocycloalkyl optionally substituted with one or more R^{5a}. In some embodiments of a compound of Formula (I) or (Ia)-(Id), R⁵ and R⁶ are taken together to form a 5- to 6-membered heterocycloalkyl optionally substituted with one or more R^{5a}.

In some embodiments of a compound of Formula (I) or (Ia)-(Id), R⁵ and R⁶ are taken together to form a cyclobutyl optionally substituted with one or more R^{5a}.

In some embodiments of a compound of Formula (I) or (Ia)-(Id), each R^{5a} is independently halogen, —CN, —OH, —OR^{a}, —NR^{c}R^{d}, C₁-C₆alkyl, C₁-C₆haloalkyl, C₂-C₆alkynyl, cycloalkyl, or heterocycloalkyl; wherein each alkyl, alkynyl, cycloalkyl, and heterocycloalkyl is independently optionally substituted with one or more R. In some embodiments of a compound of Formula (I) or (Ia)-(Id), each R^{5a} is independently halogen, —CN, C₁-C₆alkyl, C₁-C₆haloalkyl, or C₂-C₆alkynyl; wherein each alkyl and alkynyl is independently optionally substituted with one or more R.

In some embodiments of a compound of Formula (I) or (Ia)-(Id),

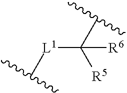

is

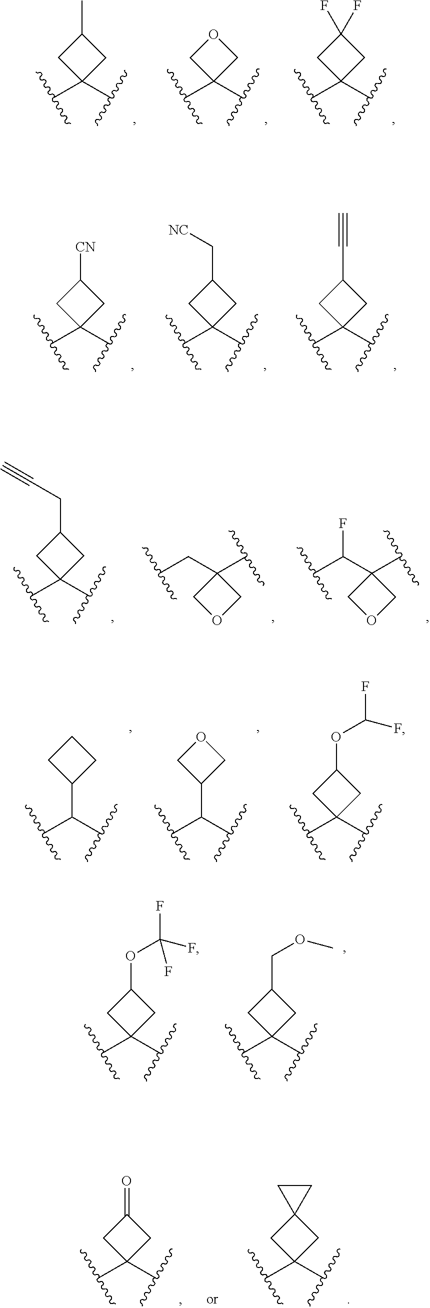

In some embodiments of a compound of Formula (I) or (Ia)-(Id),

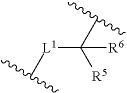

is or

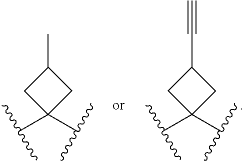

In some embodiments of a compound of Formula (I) or (Ia)-(Id),

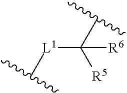

is

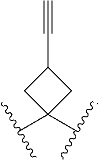 [SMILES: *C1(*)CC(C#C)C1]

In some embodiments of a compound of Formula (I) or (Ia)-(Id),

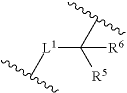

is

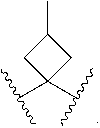 [SMILES: *C1(*)CC(C)C1]

In some embodiments of a compound of Formula (I) or (Ia)-(Id),

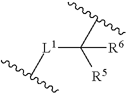

is

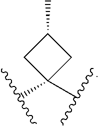

In some embodiments of a compound of Formula (I) or (Ia)-(Id),

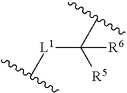

is

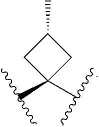

In some embodiments of a compound of Formula (I) or (Ia)-(Id), U is —N—. In some embodiments of a compound of Formula (I) or (Ia)-(Id), U is —CR¹—.

In some embodiments of a compound of Formula (I) or (Ia)-(Id), R¹ is hydrogen or C₁-C₆alkyl. In some embodiments of a compound of Formula (I) or (Ia)-(Id), R¹ is C₁-C₆alkyl. In some embodiments of a compound of Formula (I) or (Ia)-(Id), R¹ is hydrogen.

In some embodiments of a compound of Formula (I) or (Ia)-(Id), R² is hydrogen, C₁-C₆alkyl, or C₁-C₆haloalkyl. In some embodiments of a compound of Formula (I) or (Ia)-(Id), R² is hydrogen or C₁-C₆alkyl. In some embodiments of a compound of Formula (I) or (Ia)-(Id), R² is C₁-C₆alkyl. In some embodiments of a compound of Formula (I) or (Ia)-(Id), R² is hydrogen. In some embodiments of a compound of Formula (I) or (Ia)-(Id), R² is C₁-C₆alkyl or C₁-C₆haloalkyl. In some embodiments of a compound of Formula (I) or (Ia)-(Id), R² is C₁-C₆haloalkyl.

In some embodiments of a compound of Formula (I) or (Ia)-(Id),

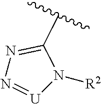

is

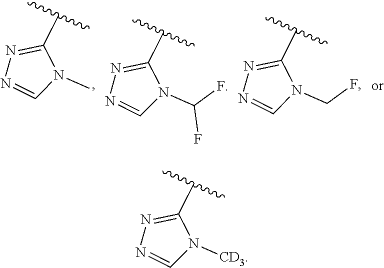

In some embodiments of a compound of Formula (I) or (Ia)-(Id),

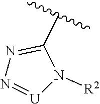

is

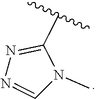 [SMILES: *c1nncn1C]

In some embodiments of a compound of Formula (I) or (Ia)-(Id),

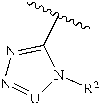

is

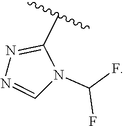 [SMILES: *c1nncn1C(F)F]

In some embodiments of a compound of Formula (I) or (Ia)-(Id),

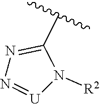

is

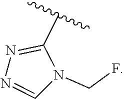 [SMILES: *c1nncn1CF]

In some embodiments of a compound of Formula (I) or (Ia)-(Id),

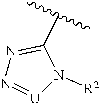

is

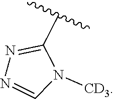 [SMILES: *c1nncn1C([2H])([2H])[2H]]

In some embodiments of a compound of Formula (I) or (Ia)-(Id), Ring A is cycloalkyl or heterocycloalkyl. In some embodiments of a compound of Formula (I) or (Ia)-(Id), Ring A is aryl or heteroaryl. In some embodiments of a compound of Formula (I) or (Ia)-(Id), Ring A is aryl. In some embodiments of a compound of Formula (I) or (Ia)-(Id), Ring A is phenyl. In some embodiments of a compound of Formula (I) or (Ia)-(Id), Ring A is heteroaryl.

In some embodiments of a compound of Formula (I) or (Ia)-(Id), Ring A is a bicyclic cycloalkyl, bicyclic heterocycloalkyl, bicyclic aryl, or bicyclic heteroaryl. In some embodiments of a compound of Formula (I) or (Ia)-(Id), Ring A is a bicyclic cycloalkyl or bicyclic heterocycloalkyl. In some embodiments of a compound of Formula (I) or (Ia)-(Id), Ring A is a bicyclic aryl or bicyclic heteroaryl.

In some embodiments of a compound of Formula (I) or (Ia)-(Id), Ring A is a bicyclic heteroaryl. In some embodiments of a compound of Formula (I) or (Ia)-(Id), wherein Ring A is a 6-6 fused ring. In some embodiments of a compound of Formula (I) or (Ia)-(Id), wherein Ring A is a 6-6 fused ring containing 1-4 ring nitrogen atoms. In some embodiments of a compound of Formula (I) or (Ia)-(Id), wherein Ring A is a 6-6 fused ring containing 2 ring nitrogen atoms. In some embodiments of a compound of Formula (I) or (Ia)-(Id), wherein Ring A is a 6-6 fused ring containing 3 ring nitrogen atoms.

In some embodiments of a compound of Formula (T) or (Ia)-(Id), Ring A is benzothiazolyl, benzimidazolyl, indazolyl, pyrazolopyridinyl, thiazolopyridinyl, quinoxalinyl, or pyridopyrazinyl.

In some embodiments of a compound of Formula (I) or (Ia)-(Id), Ring A is indazolyl or pyrazolopyridinyl. In some embodiments of a compound of Formula (I) or (Ia)-(Id), Ring A is indazolyl. In some embodiments of a compound of Formula (I) or (Ia)-(Id), Ring A is pyrazolopyridinyl.

In some embodiments of a compound of Formula (I) or (Ia)-(Id), Ring A is tetrahydroisoquinolinyl, dihydrophthalazinyl, dihydroisoquinolinyl, dihydroquinazolinyl, dihydropyridopyrimidinyl, dihydropyrroloimidazolyl, dihydroimidazopyridinyl, dihydrothienopyridazinyl, dihydroindazolyl, tetrahydropyrrolopyridinyl, or dihydropyrrolopyridine.

In some embodiments of a compound of Formula (I) or (Ia)-(Id), Ring A is tetrahydroisoquinolinyl, dihydrophthalazinyl, dihydroisoquinolinyl, dihydroquinazolinyl, dihydropyrroloimidazolyl, dihydroimidazopyridinyl, dihydrothienopyridazinyl, dihydroindazolyl, tetrahydropyrrolopyridinyl, or dihydropyrrolopyridine.

In some embodiments of a compound of Formula (I) or (Ia)-(Id), Ring A is dihydroquinazolinyl or dihydropyridopyrimidinyl. In some embodiments of a compound of Formula (I) or (Ia)-(Id), Ring A is dihydroquinazolinyl. In some embodiments of a compound of Formula (I) or (Ia)-(Id), Ring A is dihydropyridopyrimidinyl.

In some embodiments of a compound of Formula (I) or (Ia)-(Id),

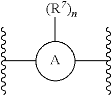

is

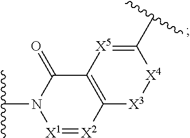

wherein
X¹, X², X³, X⁴, and X⁵ are independently N or CR^{7′}; and R^{7′} is hydrogen or R⁷.

In some embodiments, X¹ is CR^{7′}. In some embodiments, X¹ is N.

In some embodiments, X² is CR^{7′}. In some embodiments, X² is N.

In some embodiments, X³ is CR^{7′}. In some embodiments, X³ is N.

In some embodiments, X⁴ is CR^{7′}. In some embodiments, X⁴ is N.

In some embodiments, X⁵ is CR^{7′}. In some embodiments, X⁵ is N.

In some embodiments of a compound of Formula (I) or (Ia)-(Id),

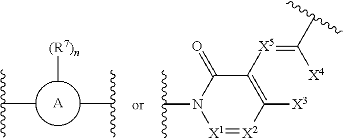

is

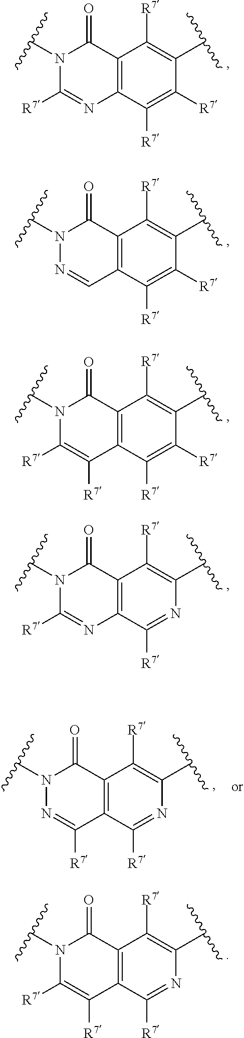

In some embodiments of a compound of Formula (I) or (Ia)-(Id),

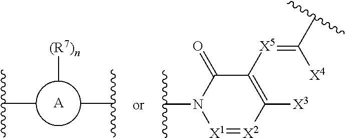

is

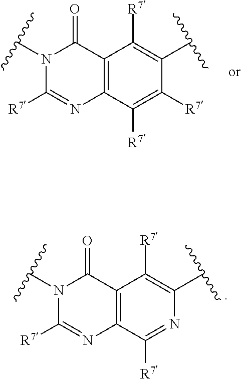

In some embodiments of a compound of Formula (I) or (Ia)-(Id),

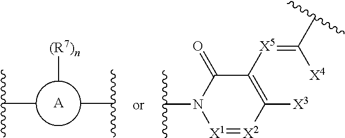

is

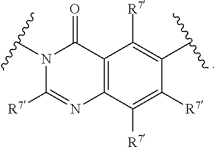

In some embodiments of a compound of Formula (I) or (Ia)-(Id),

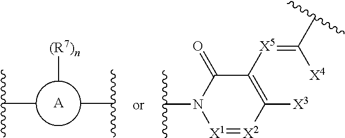

is

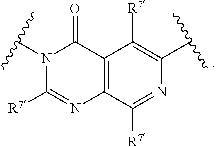

In some embodiments of a compound of Formula (I) or (Ia)-(Id),

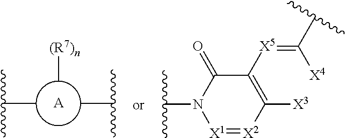

is

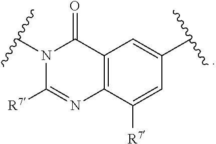

In some embodiments of a compound of Formula (I) or (Ia)-(Id),

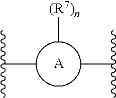

is

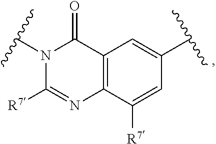

R^{7′} is hydrogen or R⁷. In some embodiments of a compound of Formula (I) or (Ia)-(Id),

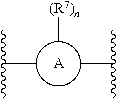

is

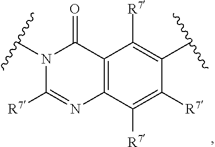

R^{7′} is hydrogen or R⁷.

In some embodiments of a compound of Formula (I) or (Ia)-(Id), each R⁷ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl; wherein each alkyl is independently optionally substituted with one or more R; or two R⁷ on the same atom are taken together to form an oxo. In some embodiments of a compound of Formula (I) or (Ia)-(Id), each R⁷ is independently halogen, —CN, —OH, —OR^{a}, —NR^{c}R^{d}, C₁-C₆alkyl, or C₁-C₆haloalkyl; wherein each alkyl is independently optionally substituted with one or more R; or two R⁷ on the same atom are taken together to form an oxo. In some embodiments of a compound of Formula (I) or (Ia)-(Id), each R⁷ is independently —OH, C₁-C₆alkyl, or C₁-C₆haloalkyl; or two R⁷ on the same atom are taken together to form an oxo. In some embodiments of a compound of Formula (I) or (Ia)-(Id), each R⁷ is independently C₁-C₆haloalkyl; or two R⁷ on the same atom are taken together to form an oxo.

In some embodiments of a compound of Formula (I) or (Ia)-(Id), each R^{7′} is independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl; wherein each alkyl is independently optionally substituted with one or more R. In some embodiments of a compound of Formula (I) or (Ia)-(Id), each R^{7′} is independently hydrogen, halogen, —CN, —OH, —OR^{a}, —NR^{c}R^{d}, C₁-C₆alkyl, or C₁-C₆haloalkyl; wherein each alkyl is independently optionally substituted with one or more R. In some embodiments of a compound of Formula (I) or (Ia)-(Id), each R^{7′} is independently hydrogen, —OH, C₁-C₆alkyl, or C₁-C₆haloalkyl. In some embodiments of a compound of Formula (I) or (Ia)-(Id), each R^{7′} is independently hydrogen or C₁-C₆haloalkyl.

In some embodiments of a compound of Formula (I) or (Ia)-(Id), n is 0, 1, 2, 3, or 4. In some embodiments of a compound of Formula (I) or (Ia)-(Id), n is 0, 1, 2, or 3. In some embodiments of a compound of Formula (I) or (Ia)-(Id), n is 1, 2, or 3. In some embodiments of a compound of Formula (I) or (Ia)-(Id), n is 1, 2, 3, or 4. In some embodiments of a compound of Formula (I) or (Ia)-(Id), n is 2, 3, or 4. In some embodiments of a compound of Formula (I) or (La)—(Id), n is 2 or 3. In some embodiments of a compound of Formula (I) or (Ia)-(Id), n is 0. In some embodiments of a compound of Formula (I) or (Ia)-(Id), n is 1. In some embodiments of a compound of Formula (I) or (Ia)-(Id), n is 2. In some embodiments of a compound of Formula (I) or (Ia)-(Id), n is 3. In some embodiments of a compound of Formula (I) or (Ia)-(Id), n is 4.

In some embodiments of a compound of Formula (I) or (Ia)-(Id),

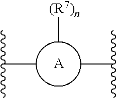

is

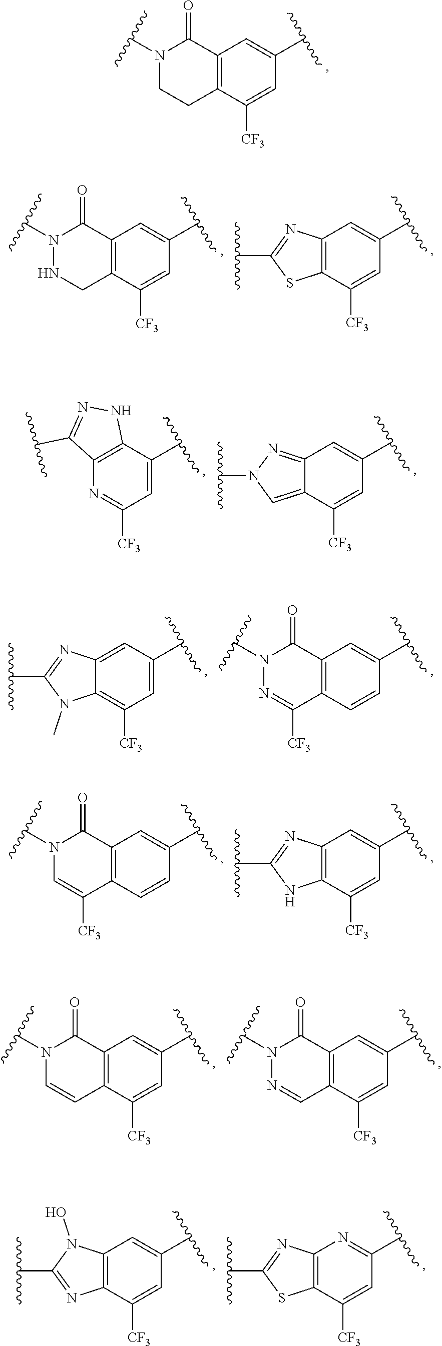

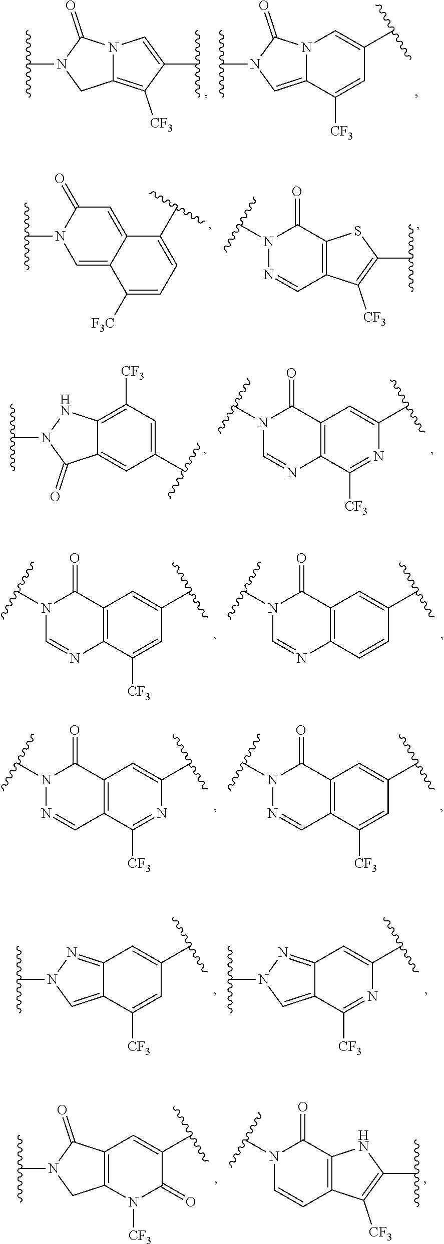

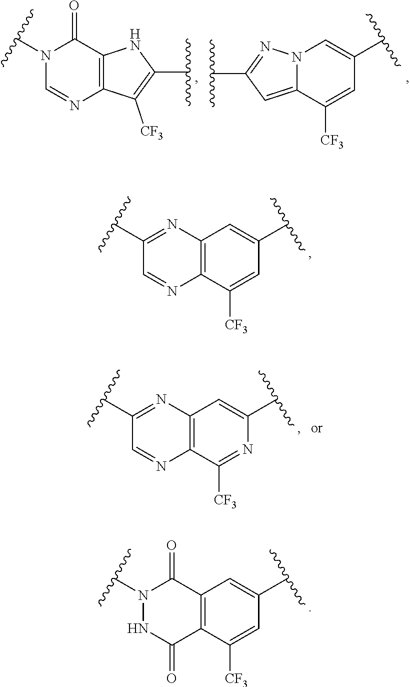

In some embodiments of a compound of Formula (I) or (Ia)-(Id),

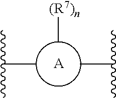

is

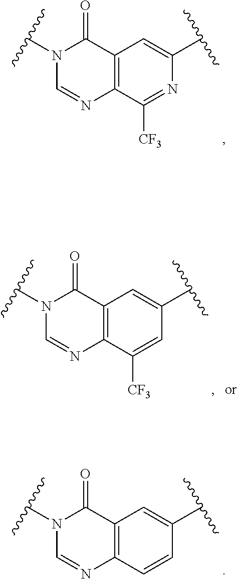 [SMILES: *c1cc2c(=O)n(*)cnc2c(C(F)(F)F)n1 ; *c1cc(C(F)(F)F)c2ncn(*)c(=O)c2c1 ; *c1ccc2ncn(*)c(=O)c2c1]

In some embodiments of a compound of Formula (I) or (Ia)-(Id),

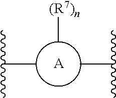

is

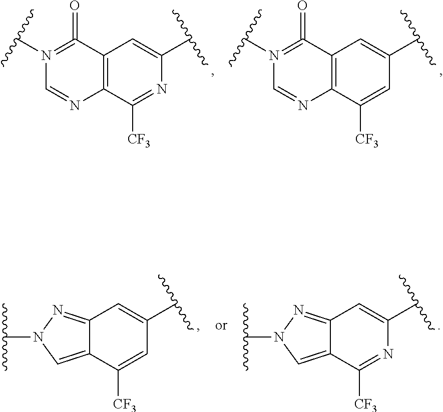

In some embodiments of a compound of Formula (I) or (Ia)-(Id),

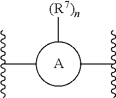

is

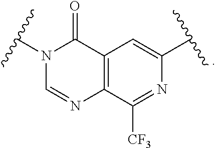 [SMILES: *c1cc2c(=O)n(*)cnc2c(C(F)(F)F)n1]

In some embodiments of a compound of Formula (I) or (Ia)-(Id

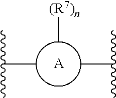

is

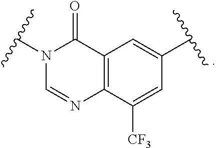 [SMILES: *c1cc(C(F)(F)F)c2ncn(*)c(=O)c2c1]

In some embodiments of a compound of Formula (I) or (Ia)-(Id),

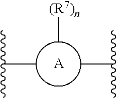

is

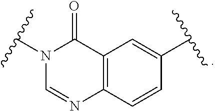 [SMILES: *c1ccc2ncn(*)c(=O)c2c1]

In some embodiments of a compound of Formula (I) or (Ia)-(Id),

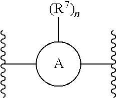

is

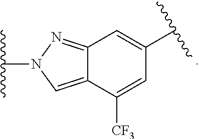 [SMILES: *c1cc(C(F)(F)F)c2cn(*)nc2c1]

In some embodiments of a compound of Formula (I) or (Ia)-(Id),

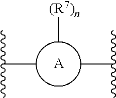

is

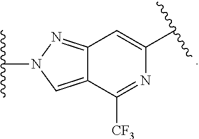 [SMILES: *c1cc2nn(*)cc2c(C(F)(F)F)n1]

Disclosed herein is a compound of Formula (III), or a pharmaceutically acceptable salt thereof:

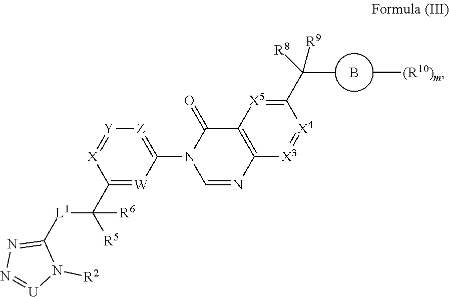

wherein:
U is —N— or —CR¹—;
R¹ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R² is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
L¹ is absent or —CR³R⁴—;
R³ and R⁴ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or R³ and R⁴ are taken together to form a cycloalkyl or heterocycloalkyl; each optionally substituted with one or more R;
or R³ and R⁴ are taken together to form an oxo;
R⁵ and R⁶ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{5a};
or R⁵ and R⁶ are taken together to form a cycloalkyl or heterocycloalkyl; each optionally substituted with one or more R^{5a}
each R^{5a} is independently halogen, —CN, —NO₂, —OH, —OR^{a}, —OC(═O)R^{a}, —OC(═O)OR^{a}, —OC(═O)NR^{c}R^{d}, —SF₅, —SH, —SR^{b}, —S(═O)R^{a}, —S(═O)₂R^{a}, —S(═O)₂NR^{c}R^{d}, —S(═O)(=NR₆)R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{b}, —N═S(═O)(R⁶)₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{5a} on the same atom are taken together to form an oxo;
X is —N— or —CR^{X}—;
R^{X} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Y is —N— or —CR^{Y}—;
R^{Y} is hydrogen, halogen, —CN, —OH, —OR^{a}, —NR^{c}R^{d}, —S(═O)R^{a}, —S(═O)₂R^{a}, —S(═O)₂NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{b}, —C(═O)OR^{b}, —C(═O)NR^{c}R^{d}, C₁-C₆alkyl, C₁-C₆haloalkyl, C₁-C₆alkoxy, C₁-C₆haloalkoxy, C₁-C₆hydroxyalkyl, C₁-C₆aminoalkyl, C₁-C₆heteroalkyl, C₂-C₆alkenyl, C₂-C₆haloalkenyl C₂-C₆alkynyl, cycloalkyl optionally substituted with one or more halogens, or heterocycloalkyl optionally substituted with one or more halogens;
Z is —N— or —CR^{Z}—.
R^{Z} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
or R^{X} and R^{Y} are taken together to form a cycloalkyl, heterocycloalkyl, aryl, or heteroaryl; each optionally substituted with one or more R;
or R^{Y} and R^{Z} are taken together to form a cycloalkyl, heterocycloalkyl, aryl, or heteroaryl; each optionally substituted with one or more R; W is —N— or —CR^{W}—;
R^{W} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
X³, X⁴, and X⁵ are independently N or CR^{7′};
each R^{7′} is independently hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(=NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
R⁸ and R⁹ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or R⁸ and R⁹ are taken together to form a cycloalkyl or heterocycloalkyl; each optionally substituted with one or more R;
or R⁸ and R⁹ are taken together to form an oxo;
Ring B is cycloalkyl, heterocycloalkyl, aryl, or heteroaryl;
each R¹⁰ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(=NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or two R¹⁰ on the same atom are taken together to form an oxo;
m is 0, 1, 2, 3, 4, 5, or 6;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C⁶aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(=NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —C(═O)C₁-C₃alkyl, —C(═O)OH, —C(═O)OCl—C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, or C₃-C₆cycloalkyl;
or two R on the same atom form an oxo.

In some embodiments of a compound of Formula (III), Y is —N—. In some embodiments of a compound of Formula (III), Y is —CR^{Y}—.

In some embodiments of a compound of Formula (III), Z is —N—. In some embodiments of a compound of Formula (III), Z is —CR^{Z}—.

In some embodiments of a compound of Formula (III), W is —N—. In some embodiments of a compound of Formula (III), W is —CR^{W}—.

In some embodiments of a compound of Formula (III), the compound is of Formula (IIIa):

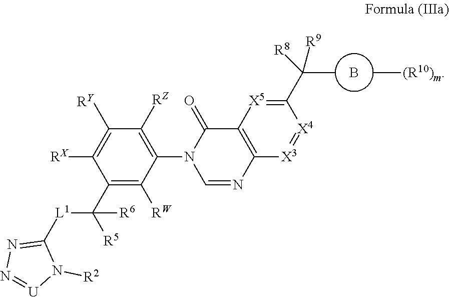

In some embodiments of a compound of Formula (III), the compound is of Formula (IIIb):

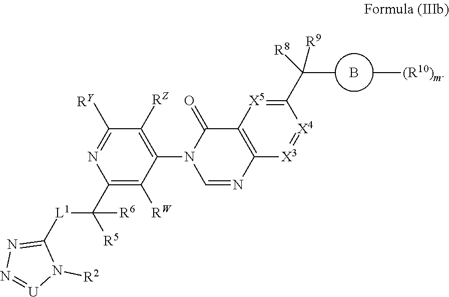

In some embodiments of a compound of Formula (III), the compound is of Formula (IIIc):

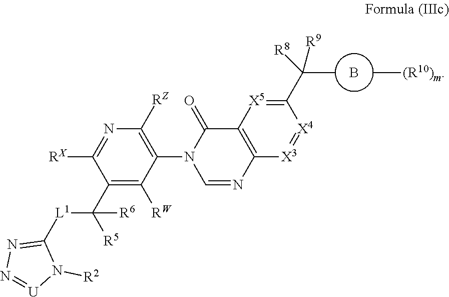

In some embodiments of a compound of Formula (III), the compound is of Formula (IIId):

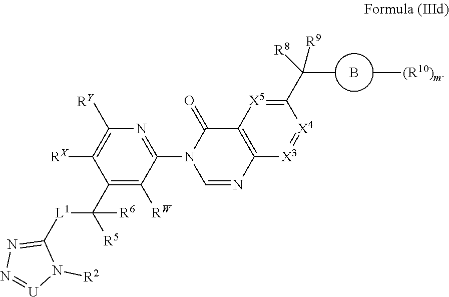

In some embodiments of a compound of Formula (III), (IIIa), (IIIc), or (IIId), R^{X} is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (III), (IIIa), (IIIc), or (IIId), R^{X} is hydrogen, halogen, C₁-C₆alkyl, C₁-C₆haloalkyl, cycloalkyl, or heterocycloalkyl. In some embodiments of a compound of Formula (III), (IIIa), (IIIc), or (IIId), R^{X} is hydrogen, halogen, C₁-C₆alkyl, or C₁-C₆haloalkyl. In some embodiments of a compound of Formula (III), (IIIa), (IIIc), or (IIId), R^{X} is hydrogen or C₁-C₆alkyl. In some embodiments of a compound of Formula (III), (IIIa), (IIIc), or (IIId), R^{X} is hydrogen.

In some embodiments of a compound of Formula (III), (IIIa), (IIIb), or (IIId), R^{Y} is hydrogen, halogen, —CN, —OR^{a}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆haloalkenyl, C₂-C₆alkynyl, cycloalkyl optionally substituted with one or more halogens, or heterocycloalkyl optionally substituted with one or more halogens.

In some embodiments of a compound of Formula (III), (IIIa), (IIIb), or (IIId), R^{Y} is hydrogen, halogen, —CN, —OR^{a}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl optionally substituted with one or more halogens, or heterocycloalkyl optionally substituted with one or more halogens.

In some embodiments of a compound of Formula (III), (IIIa), (11b), or (IIId), R^{Y} is hydrogen, halogen, —CN, —OR^{a}, —C(═O)NR^{c}R^{d}, C₁-C₆alkyl, C₁-C₆haloalkyl, C₁-C₆hydroxyalkyl, C₂-C₆alkenyl, C₂-C₆haloalkenyl, C₂-C₆alkynyl, or cycloalkyl optionally substituted with one or more halogens.

In some embodiments of a compound of Formula (III), (IIIa), (IIIb), or (IIId), R^{Y} is hydrogen, C₁-C₆alkyl, C₁-C₆haloalkyl, C₃-C₆cycloalkyl, or C₁-C₆haloalkoxy.

In some embodiments of a compound of Formula (III), (IIIa), (IIIb), or (IIId), R^{Y} is hydrogen, —OR^{a}, C₁-C₆alkyl, C₁-C₆haloalkyl, or C₃-C₆cycloalkyl; and R^{a} is C₁-C₆haloalkyl.

In some embodiments of a compound of Formula (II), (IIIa), (IIIb), or (IIId), R^{Y} is hydrogen, halogen, —CN, —OR^{a}, —C(═O)NR^{c}R^{d}, C₁-C₆alkyl, C₁-C₆haloalkyl, C₁-C₆hydroxyalkyl, C₂-C₆haloalkenyl, C₂-C₆alkynyl, or cycloalkyl optionally substituted with one or more halogens.

In some embodiments of a compound of Formula (III), (IIIa), (IIIb), or (IIId), R^{Y} is hydrogen, halogen, —CN, —OR^{a}, —C(═O)NR^{c}R^{d}, C₁-C₆alkyl, C₁-C₆hydroxyalkyl, C₁-C₆alkenyl, C₂-C₆haloalkenyl, C₂C₂-C₆alkynyl, or cycloalkyl optionally substituted with one or more halogens.

In some embodiments of a compound of Formula (III), (IIIa), (IIIb), or (IIId), R^{Y} is hydrogen, halogen, —CN, —OR^{a}, —C(═O)NR^{c}R^{d}, C₁-C₆alkyl, C₁-C₆hydroxyalkyl, C₂-C₆alkenyl, C₂C₂-C₆alkynyl, or cycloalkyl optionally substituted with one or more halogens.

In some embodiments of a compound of Formula (III), (IIIa), (IIIb), or (IIId), R^{Y} is hydrogen, —OR^{a}, or cycloalkyl optionally substituted with one or more halogens.

In some embodiments of a compound of Formula (III), (IIIa), (IIIb), or (IIId), R^{Y} is hydrogen, —OR^{a}, or cycloalkyl.

In some embodiments of a compound of Formula (III), (IIIa), (IIIb), or (IIId), R^{Y} is C₁-C₆haloalkoxy. In some embodiments of a compound of Formula (III), (IIIa), (IIIb), or (IIId), R^{Y} is C₁-C₃haloalkoxy.

In some embodiments of a compound of Formula (III), (IIIa), (IIIb), or (IIId), R^{Y} is —OR^{a}. In some embodiments of a compound of Formula (III), (IIIa), (IIIb), or (IIId), R^{Y} is —OR^{a} and R^{a} is C₁-C₆haloalkyl. In some embodiments of a compound of Formula (III), (IIIa), (IIIb), or (IIId), R^{Y} is —OCH₂F.

In some embodiments of a compound of Formula (III), (IIIa), (IIIb), or (IIId), R^{Y} is cycloalkyl. In some embodiments of a compound of Formula (III), (IIIa), (IIIb), or (IIId), R^{Y} is cyclopropyl.

In some embodiments of a compound of Formula (III), (IIIa), (IIIb), or (IIId), R^{Y} is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl optionally substituted with one or more halogens, or heterocycloalkyl optionally substituted with one or more halogens.

In some embodiments of a compound of Formula (III), (IIIa), (IIIb), or (IIId), R^{Y} is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl.

In some embodiments of a compound of Formula (III), (IIIa), (IIIb), or (IIId), R^{Y} is hydrogen, halogen, C₁-C₆alkyl, C₁-C₆haloalkyl, cycloalkyl optionally substituted with one or more halogens, or heterocycloalkyl optionally substituted with one or more halogens.

In some embodiments of a compound of Formula (III), (IIIa), (IIIb), or (IIId), R^{Y} is hydrogen, halogen, C₁-C₆alkyl, C₁-C₆haloalkyl, cycloalkyl, or heterocycloalkyl.

In some embodiments of a compound of Formula (III), (IIIa), (IIIb), or (IIId), R^{Y} is hydrogen, halogen, C₁-C₆alkyl, or C₁-C₆haloalkyl.

In some embodiments of a compound of Formula (III), (IIIa), (IIIb), or (IIId), R^{Y} is hydrogen or C₁-C₆alkyl.

In some embodiments of a compound of Formula (III), (IIIa), (IIIb), or (IIId), R^{Y} is hydrogen.

In some embodiments of a compound of Formula (III), (IIIa), (IIIb), or (IIIc), R^{Z} is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (III), (IIIa), (IIIb), or (IIIc), R^{Z} is hydrogen, halogen, C₁-C₆alkyl, C₁-C₆haloalkyl, cycloalkyl, or heterocycloalkyl. In some embodiments of a compound of Formula (III), (IIIa), (IIIb), or (Ilic), R^{Z} is hydrogen, halogen, C₁-C₆alkyl, or C₁-C₆haloalkyl. In some embodiments of a compound of Formula (III), (IIIa), (IIIb), or (IIIc), R^{Z} is hydrogen or C₁-C₆alkyl. In some embodiments of a compound of Formula (III), (IIIa), (IIIb), or (IIIc), R^{Z} is hydrogen. In some embodiments of a compound of Formula (III), (IIIa), (IIIb), or (IIIc), R^{Z} is hydrogen or halogen. In some embodiments of a compound of Formula (III), (IIIa), (IIIb), or (IIIc), R^{Z} is halogen.

In some embodiments of a compound of Formula (III) or (IIIa), R^{X} and R^{Y} are taken together to form a cycloalkyl, heterocycloalkyl, aryl, or heteroaryl; each optionally substituted with one or more R.

In some embodiments of a compound of Formula (III) or (IIIa), R^{X} and R^{Y} are taken together to form a cycloalkyl or heterocycloalkyl; each optionally substituted with one or more R. In some embodiments of a compound of Formula (III) or (IIIa), R^{X} and R^{Y} are taken together to form a cycloalkyl optionally substituted with one or more R. In some embodiments of a compound of Formula (III) or (IIIa), R^{X} and R^{Y} are taken together to form a heterocycloalkyl optionally substituted with one or more R.

In some embodiments of a compound of Formula (III) or (IIIa), R^{Y} and R^{Z} are taken together to form a cycloalkyl, heterocycloalkyl, aryl, or heteroaryl; each optionally substituted with one or more R. In some embodiments of a compound of Formula (III) or (IIIa), R^{Y} and R^{Z} are taken together to form a cycloalkyl or heterocycloalkyl; each optionally substituted with one or more R. In some embodiments of a compound of Formula (III) or (IIIa), R^{Y} and R^{Z} are taken together to form a cycloalkyl optionally substituted with one or more R. In some embodiments of a compound of Formula (III) or (IIIa), R^{Y} and R^{Z} are taken together to form a heterocycloalkyl optionally substituted with one or more R.

In some embodiments of a compound of Formula (III) or (IIIa)-(IIId), R^{W} is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (III) or (IIIa)-(IIId), R^{W} is hydrogen, halogen, C₁-C₆alkyl, C₁-C₆haloalkyl, cycloalkyl, or heterocycloalkyl. In some embodiments of a compound of Formula (III) or (IIIa)-(IIId), R^{W} is hydrogen, halogen, C₁-C₆alkyl, or C₁-C₆haloalkyl. In some embodiments of a compound of Formula (III) or (IIIa)-(IIId), R^{W} is hydrogen or C₁-C₆alkyl. In some embodiments of a compound of Formula (III) or (IIIa)-(IIId), R^{W} is hydrogen.

In some embodiments of a compound of Formula (III),

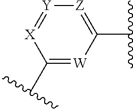

is

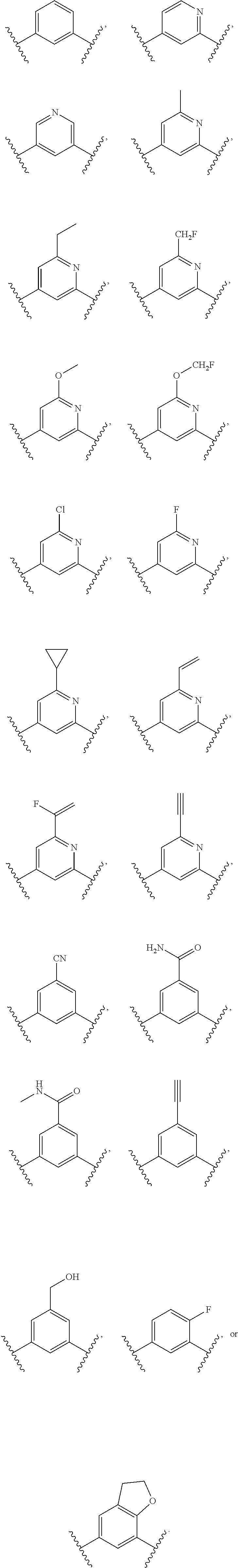

In some embodiments of a compound of Formula (III) or (IIIa)-(IIId), R⁸ and R⁹ are each independently hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound of Formula (III) or (IIIa)-(IIId), R⁸ and R⁹ are each independently hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (III) or (IIIa)-(IIId), R⁸ and R⁹ are each independently hydrogen, halogen, C₁-C₆alkyl, or C₁-C₆haloalkyl. In some embodiments of a compound of Formula (III) or (IIIa)-(IIId), R⁸ and R⁹ are each independently hydrogen or C₁-C₆alkyl. In some embodiments of a compound of Formula (III) or (IIIa)-(IIId), R⁸ and R⁹ are each hydrogen. In some embodiments of a compound of Formula (III) or (IIIa)-(IIId), R⁸ and R⁹ are taken together to form a cycloalkyl or heterocycloalkyl; each optionally substituted with one or more R. In some embodiments of a compound of Formula (III) or (IIIa)-(IIId), R⁸ and R⁹ are taken together to form an oxo.

In some embodiments of a compound of Formula (III) or (IIIa)-(IIId), Ring B is cycloalkyl or heterocycloalkyl. In some embodiments of a compound of Formula (III) or (IIIa)-(IIId), Ring B is cycloalkyl. In some embodiments of a compound of Formula (III) or (IIIa)-(IIId), Ring B is heterocycloalkyl. In some embodiments of a compound of Formula (III) or (IIIa)-(IIId), Ring B is 5- or 6-membered heterocycloalkyl.

In some embodiments of a compound of Formula (III) or (IIIa)-(IIId), Ring B is azetidinyl, pyrrolidinyl, piperidinyl, or morpholinyl. In some embodiments of a compound of Formula (III) or (IIIa)-(IIId), Ring B is piperidinyl.

In some embodiments of a compound of Formula (III) or (IIIa)-(IIId), each R¹⁰ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, C₁-C₆alkylene(cycloalkyl), or C₁-C₆alkylene(heterocycloalkyl); wherein each alkyl, alkylene, alkenyl, alkynyl, cycloalkyl, and heterocycloalkyl is independently optionally substituted with one or more R.

In some embodiments of a compound of Formula (III) or (IIIa)-(IIId), each R¹⁰ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, C₁-C₆alkylene(cycloalkyl), or C₁-C₆alkylene(heterocycloalkyl).

In some embodiments of a compound of Formula (III) or (IIIa)-(IIId), each R¹⁰ is independently halogen, —CN, —OH, —OR^{a}, —NR^{c}R^{d}, C₁-C₆alkyl, C₁-C₆haloalkyl, C₂-C₆alkenyl, C₂-C₆alkynyl, cycloalkyl, heterocycloalkyl, C₁-C₆alkylene(cycloalkyl), or C₁-C₆alkylene(heterocycloalkyl).

In some embodiments of a compound of Formula (III) or (IIIa)-(IIId), each R¹⁰ is independently halogen, —OH, —OR^{a}, C₁-C₆alkyl, C₁-C₆haloalkyl, C₂-C₆alkenyl, C₂-C₆alkynyl, cycloalkyl, or C₁-C₆alkylene(cycloalkyl).

In some embodiments of a compound of Formula (III) or (IIIa)-(IIId), each R¹⁰ is independently halogen, —OH, —OR^{a}, C₁-C₆alkyl, C₂-C₆alkenyl, C₂-C₆alkynyl, cycloalkyl, or C₁-C₆alkylene(cycloalkyl).

In some embodiments of a compound of Formula (III) or (IIIa)-(IIId), each R¹⁰ is independently halogen, —CN, —OH, —OR^{a}, —NR^{c}R^{d}, C₁-C₆alkyl, C₁-C₆haloalkyl, C₂-C₆alkynyl, cycloalkyl, heterocycloalkyl, C₁-C₆alkylene(cycloalkyl), or C₁-C₆alkylene(heterocycloalkyl); wherein each alkyl, alkylene, alkynyl, cycloalkyl, and heterocycloalkyl is independently optionally substituted with one or more R.

In some embodiments of a compound of Formula (III) or (IIIa)-(IIId), each R¹⁰ is independently halogen, C₁-C₆alkyl, C₁-C₆haloalkyl, C₂-C₆alkynyl, C₁-C₆alkylene(cycloalkyl), or C₁-C₆alkylene(heterocycloalkyl); wherein each alkyl, alkylene, alkynyl, cycloalkyl, and heterocycloalkyl is independently optionally substituted with one or more R. In some embodiments of a compound of Formula (III) or (IIIa)-(IIId), each R¹⁰ is independently halogen or C₁-C₆alkyl. In some embodiments of a compound of Formula (III) or (IIIa)-(IIId), each R¹⁰ is independently C₁-C₆alkyl.

In some embodiments of a compound of Formula (III) or (IIIa)-(IIId), m is 0, 1, 2, or 3. In some embodiments of a compound of Formula (III) or (IIIa)-(IIId), m is 1 or 2. In some embodiments of a compound of Formula (III) or (IIIa)-(IIId), m is 1, 2, or 3. In some embodiments of a compound of Formula (III) or (IIIa)-(IIId), m is 0, 1, or 2. In some embodiments of a compound of Formula (III) or (IIIa)-(IIId), m is 1 or 2. In some embodiments of a compound of Formula (III) or (IIIa)-(IIId), m is 0. In some embodiments of a compound of Formula (III) or (IIIa)-(IIId), m is 1. In some embodiments of a compound of Formula (III) or (IIIa)-(IIId), m is 2. In some embodiments of a compound of Formula (III) or (IIIa)-(IIId), m is 3. In some embodiments of a compound of Formula (III) or (IIIa)-(IIId), m is 4.

In some embodiments of a compound of Formula (III) or (IIIa)-(IIId),

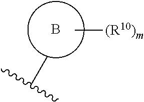

is

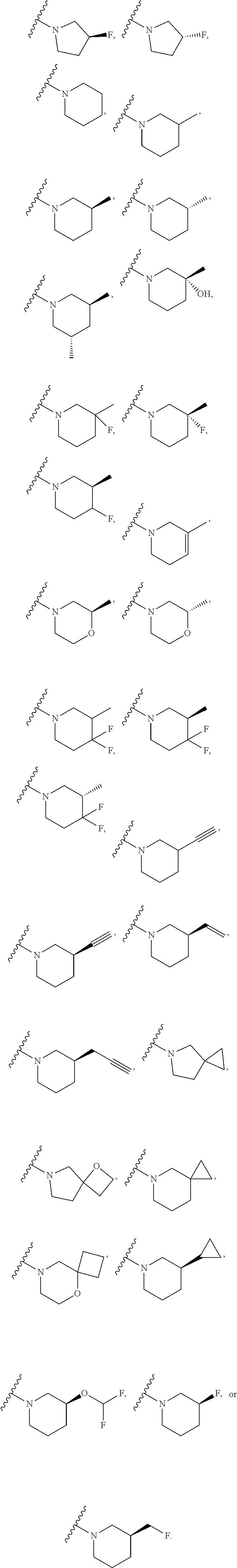

In some embodiments of a compound of Formula (III) or (IIIa)-(IIId),

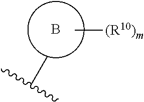

is

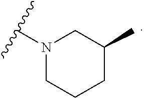 [SMILES: *N1CCC[C@H](C)C1]

In some embodiments of a compound of Formula (III) or (IIIa)-(IIId), L¹ is absent. In some embodiments of a compound of Formula (III) or (IIIa)-(IIId), L¹ is —CR³R⁴—.

In some embodiments of a compound of Formula (III) or (IIIa)-(IIId), R³ and R⁴ are each independently hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound of Formula (III) or (IIIa)-(IIId), R³ and R⁴ are each independently hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (III) or (IIIa)-(IIId), R³ and R⁴ are each independently hydrogen, halogen, C₁-C₆alkyl, C₁-C₆haloalkylcycloalkyl, or heterocycloalkyl. In some embodiments of a compound of Formula (III) or (IIIa)-(IIId), R³ and R⁴ are each independently hydrogen, halogen, C₁-C₆alkyl, or C₁-C₆haloalkyl. In some embodiments of a compound of Formula (III) or (IIIa)-(IIId), R³ and R⁴ are each hydrogen. In some embodiments of a compound of Formula (III) or (IIIa)-(IIId), R³ and R⁴ are taken together to form a cycloalkyl or heterocycloalkyl; each optionally substituted with one or more R. In some embodiments of a compound of Formula (III) or (IIIa)-(IIId), R³ and R⁴ are taken together to form an oxo.

In some embodiments of a compound of Formula (III) or (IIIa)-(IIId), R⁶ is hydrogen, halogen, —CN, —OH, —OR^{a}, —NR^{c}R^{d}, C₁-C₆alkyl, or C₁-C₆haloalkyl. In some embodiments of a compound of Formula (III) or (IIIa)-(IIId), R⁶ is hydrogen, C₁-C₆alkyl, or C₁-C₆haloalkyl. In some embodiments of a compound of Formula (III) or (IIIa)-(IIId), R⁶ is hydrogen or C₁-C₆alkyl. In some embodiments of a compound of Formula (III) or (IIIa)-(IIId), R⁶ is hydrogen.

In some embodiments of a compound of Formula (ITT) or (IIIa)-(IIId), R⁵ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{5a}.

In some embodiments of a compound of Formula (III) or (IIIa)-(IIId), R⁵ is cycloalkyl, heterocycloalkyl, aryl, or heteroaryl; wherein each cycloalkyl, heterocycloalkyl, aryl, and heteroaryl is independently optionally substituted with one or more R^{5a}. In some embodiments of a compound of Formula (III) or (IIIa)-(IIId), R⁵ is cycloalkyl or heterocycloalkyl; wherein each cycloalkyl and heterocycloalkyl is independently optionally substituted with one or more R^{5a}. In some embodiments of a compound of Formula (III) or (IIIa)-(IIId), R is cycloalkyl optionally substituted with one or more R⁵.

In some embodiments of a compound of Formula (III) or (IIIa)-(IIId), R⁵ is heterocycloalkyl optionally substituted with one or more R^{5a}.

In some embodiments of a compound of Formula (III) or (IIIa)-(IIId), R⁵ and R⁶ are taken together to form a cycloalkyl optionally substituted with one or more R^{5a}.

In some embodiments of a compound of Formula (III) or (IIIa)-(IIId), R⁵ and R⁶ are taken together to form a heterocycloalkyl optionally substituted with one or more R^{5a}.

In some embodiments of a compound of Formula (III) or (IIIa)-(IIId), R⁵ and R⁶ are taken together to form a cyclobutyl optionally substituted with one or more R^{5a}.

In some embodiments of a compound of Formula (III) or (IIIa)-(IIId), each R^{5a} is independently halogen, —CN, —OH, —O, —NR^{c}R^{d}, C₁-C₆alkyl, C₁-C₆haloalkyl, C₁-C₆heteroalkyl, C₂-C₆alkynyl, cycloalkyl, or heterocycloalkyl; wherein each alkyl, alkynyl, cycloalkyl, and heterocycloalkyl is independently optionally substituted with one or more R.

In some embodiments of a compound of Formula (III) or (IIIa)-(IIId), each R^{5a} is independently halogen, —CN, —OH, —OR^{a}, —NR^{c}R^{d}, C₁-C₆alkyl, C₁-C₆haloalkyl, C₂-C₆alkynyl, cycloalkyl, or heterocycloalkyl; wherein each alkyl, alkynyl, cycloalkyl, and heterocycloalkyl is independently optionally substituted with one or more R. In some embodiments of a compound of Formula (III) or (IIIa)-(IIId), each R⁵, is independently halogen, —CN, C₁-C₆alkyl, C₁-C₆haloalkyl, or C₂-C₆alkynyl; wherein each alkyl and alkynyl is independently optionally substituted with one or more R.

In some embodiments of a compound of Formula (III) or (IIIa)-(IIId),

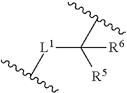

is

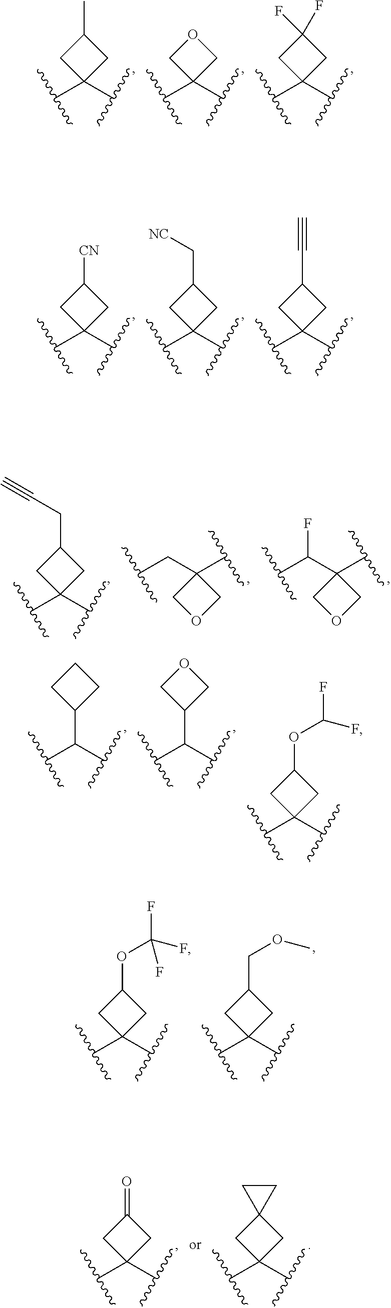

In some embodiments of a compound of Formula (III) or (IIIa)-(IIId),

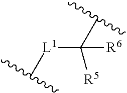

is

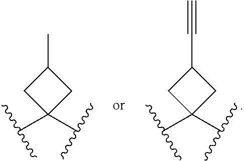

In some embodiments of a compound of Formula (III) or (IIIa)-(IIId),

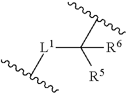

is

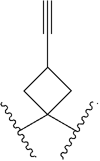 [SMILES: *C1(*)CC(C#C)C1]

In some embodiments of a compound of Formula (III) or (IIIa)-(IIId),

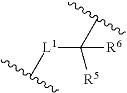

is

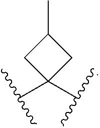 [SMILES: *C1(*)CC(C)C1]

In some embodiments of a compound of Formula (III) or (IIIa)-(IIId),

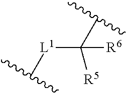

is

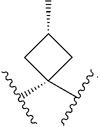

In some embodiments of a compound of Formula (III) or (IIIa)-(IIId),

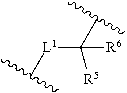

is

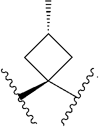

In some embodiments of a compound of Formula (III) or (IIIa)-(IIId), U is —N—. In some embodiments of a compound of Formula (III) or (IIIa)-(IIId), U is —CR¹—.

In some embodiments of a compound of Formula (III) or (IIIa)-(IIId), R¹ is hydrogen or C₁-C₆alkyl. In some embodiments of a compound of Formula (III) or (IIIa)-(IIId), R¹ is C₁-C₆alkyl. In some embodiments of a compound of Formula (III) or (IIIa)-(IIId), R¹ is hydrogen.

In some embodiments of a compound of Formula (III) or (IIIa)-(IIId), R² is hydrogen, C₁-C₆alkyl, or C₁-C₆haloalkyl. In some embodiments of a compound of Formula (III) or (IIIa)-(IIId), R² is hydrogen or C₁-C₆alkyl. In some embodiments of a compound of Formula (III) or (IIIa)-(IIId), R² is C₁-C₆alkyl. In some embodiments of a compound of Formula (III) or (IIIa)-(IIId), R² is hydrogen. In some embodiments of a compound of Formula (III) or (IIIa)-(IIId), R² is C₁-C₆alkyl or C₁-C₆haloalkyl. In some embodiments of a compound of Formula (III) or (IIIa)-(IIId), R² is C₁-C₆haloalkyl.

In some embodiments of a compound of Formula (III) or (IIIa)-(IIId),

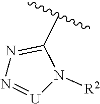

is

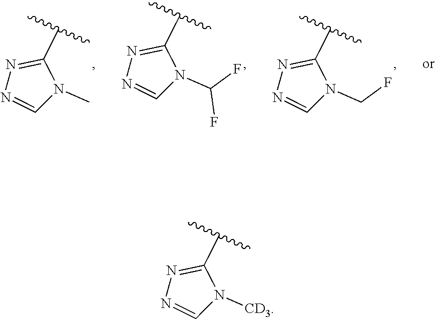

In some embodiments of a compound of Formula (III) or (IIIa)-(IIId),

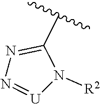

is

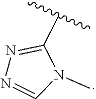 [SMILES: *c1nncn1C]

In some embodiments of a compound of Formula (III) or (IIIa)-(IIId),

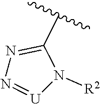

is

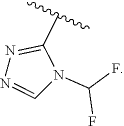 [SMILES: *c1nncn1C(F)F]

In some embodiments of a compound of Formula (III) or (IIIa)-(IIId),

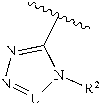

is

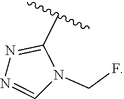 [SMILES: *c1nncn1CF]

In some embodiments of a compound of Formula (III) or (IIIa)-(IIId),

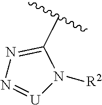

is

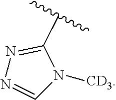 [SMILES: *c1nncn1C([2H])([2H])[2H]]

In some embodiments of a compound of Formula (III), (IIIa), (IIIb), or (IIId), X³ is CR⁷. In some embodiments of a compound of Formula (III), (IIIa), (IIIb), or (IIId), X³ is N.

In some embodiments of a compound of Formula (III), (IIIa), (IIIb), or (IIId), X⁴ is CR⁷. In some embodiments of a compound of Formula (III), (IIIa), (IIIb), or (IIId), X⁴ is N.

In some embodiments of a compound of Formula (III), (IIIa), (IIIb), or (IIId), X⁵ is CR⁷. In some embodiments of a compound of Formula (III), (IIIa), (IIIb), or (IIId), X⁵ is N.

In some embodiments of a compound of Formula (III), (IIIa), (IIIb), or (IIId),

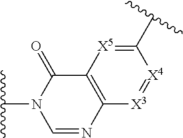

is

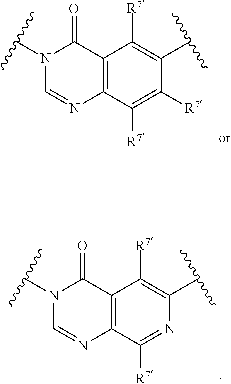

In some embodiments of a compound of Formula (III), (IIIa), (IIIb), or (IIId),

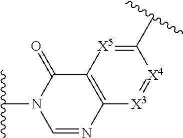

is

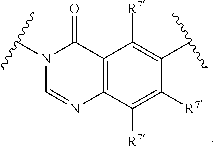

In some embodiments of a compound of Formula (III), (IIIa), (IIIb), or (IIId),

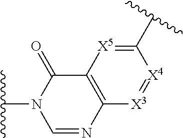

is

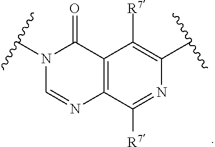

In some embodiments of a compound of Formula (III), (IIIa), (IIIb), or (IIId),

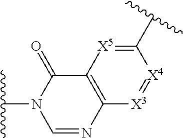

is

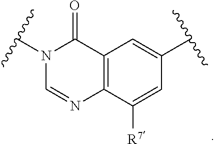

In some embodiments of a compound of Formula (III), (IIIa), (IIIb), or (IIId),

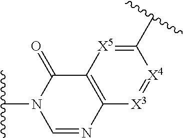

is

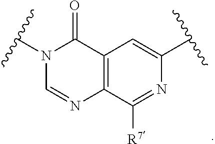

In some embodiments of a compound of Formula (III) or (IIIa)-(IIId), each R⁷ is independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl; wherein each alkyl is independently optionally substituted with one or more R. In some embodiments of a compound of Formula (III) or (IIIa)-(IIId), each R^{7′} is independently hydrogen, halogen, —CN, —OH, —OR^{a}, —NR^{c}R^{d}, C₁-C₆alkyl, or C₁-C₆haloalkyl; wherein each alkyl is independently optionally substituted with one or more R. In some embodiments of a compound of Formula (III) or (IIIa)-(IIId), each R^{7′} is independently hydrogen, —OH, C₁-C₆alkyl, or C₁-C₆haloalkyl. In some embodiments of a compound of Formula (III) or (IIIa)-(IIId), each R^{7′} is independently hydrogen or C₁-C₆haloalkyl. In some embodiments of a compound of Formula (III) or (IIIa)-(IIId), each R^{7′} is independently hydrogen or —CF₃.

In some embodiments of a compound of Formula (III) or (IIIa)-(IIId),

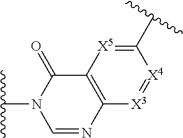

is

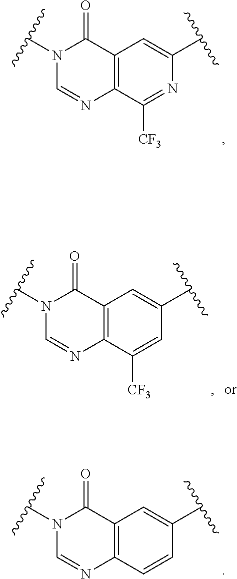 [SMILES: *c1cc2c(=O)n(*)cnc2c(C(F)(F)F)n1 ; *c1cc(C(F)(F)F)c2ncn(*)c(=O)c2c1 ; *c1ccc2ncn(*)c(=O)c2c1]

Also provided herein is a compound of Formula (II), or a pharmaceutically acceptable salt thereof:

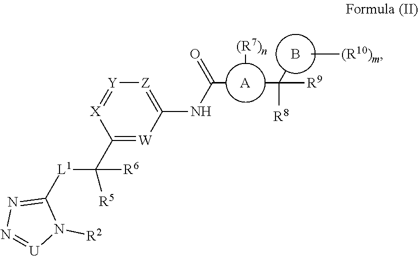

wherein:
U is —N— or —CR¹—;
R¹ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R² is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
L¹ is absent or —CR³R⁴—;
R³ and R⁴ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or R³ and R⁴ are taken together to form a cycloalkyl or heterocycloalkyl; each optionally substituted with one or more R;
R⁵ and R⁶ are each independently hydrogen, halogen, —CN, —OH, —OR^{a}, —NR^{e}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{5a};
or R⁵ and R⁶ are taken together to form a cycloalkyl or heterocycloalkyl; each optionally substituted with one or more R^{5a}
each R^{5a} is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(O)₂R^{a}, —S(═O)₂NR^{c}R^{d}, —S(═O)(=NR^{b})R^{b}, —NR^{c}R^{d}, —NR^{b}C(═O)NR^{c}R^{d}, —NR^{b}C(O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
X is —N— or —CR^{X}—;
R^{X} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Y is —N— or —CR^{Y}—;
R^{Y} is hydrogen, halogen, —CN, —OH, —OR^{a}, —NR^{c}R^{d}, —S(═O)R^{a}, —S(═O)₂R^{a}, —S(═O)₂NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆haloalkenyl C₂-C₆alkynyl, cycloalkyl optionally substituted with one or more halogens, or heterocycloalkyl optionally substituted with one or more halogens;
Z is —N— or —CR^{Z}—.

R^{Z} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
W is —N— or —CR^{W}—;
R^{W} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Ring A is cycloalkyl, heterocycloalkyl, aryl, or heteroaryl;
each R⁷ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(=NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R⁷ on the same atom are taken together to form an oxo;
n is 0, 1, 2, 3, 4, 5, or 6;
R⁸ and R⁹ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R; or R^{A} and R⁹ are taken together to form a cycloalkyl or heterocycloalkyl; each optionally substituted with one or more R;
Ring B is cycloalkyl, heterocycloalkyl, aryl, or heteroaryl;
each R¹⁰ is independently halogen, —CN, —NO₂, —OH, —OR^{a}, —OC(═O)R^{b}, —OC(═O)OR^{b}, —OC(═O)NR^{c}R^{d}, —SF₅, —SH, —SR^{a}, —S(═O)R^{a}, —S(O)₂R^{a}, —S(═O)₂NR^{c}R^{d}, —S(═O)(=NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R on the same atom are taken together to form an oxo;
m is 0, 1, 2, 3, 4, 5, or 6;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(=NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, or C₃-C₆cycloalkyl;
or two R on the same atom form an oxo;
provided that

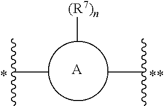

is not

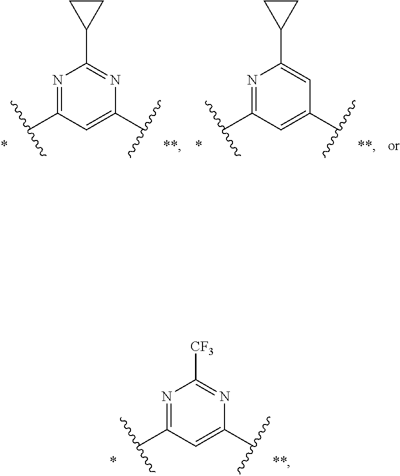 [SMILES: *c1cc(*)nc(C2CC2)n1 ; *c1cc(*)nc(C2CC2)c1 ; *c1cc(*)nc(C(F)(F)F)n1]

wherein * represents the attachment point to —C(═O)—NH— group and ** represents the attachment point to —CR³R⁹—;
and provided that the compound of Formula (II) is not

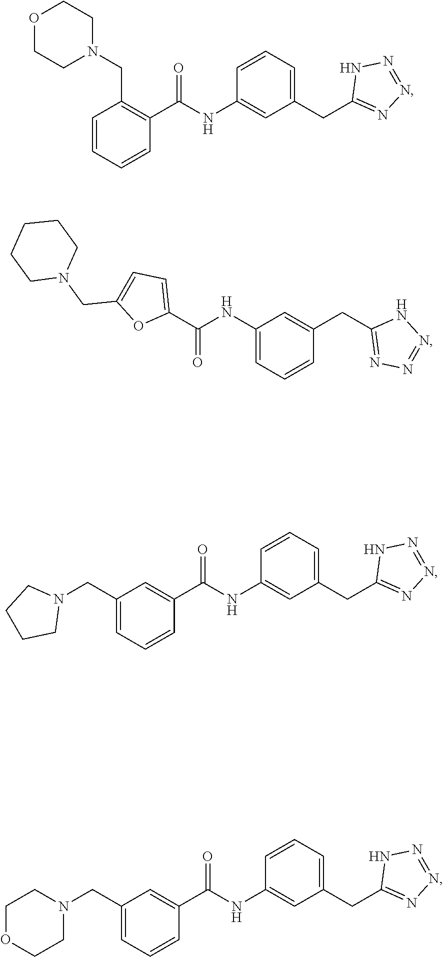 [SMILES: O=C(Nc1cccc(Cc2nnn[nH]2)c1)c1ccccc1CN1CCOCC1 ; O=C(Nc1cccc(Cc2nnn[nH]2)c1)c1ccc(CN2CCCCC2)o1 ; O=C(Nc1cccc(Cc2nnn[nH]2)c1)c1cccc(CN2CCCC2)c1 ; O=C(Nc1cccc(Cc2nnn[nH]2)c1)c1cccc(CN2CCOCC2)c1]

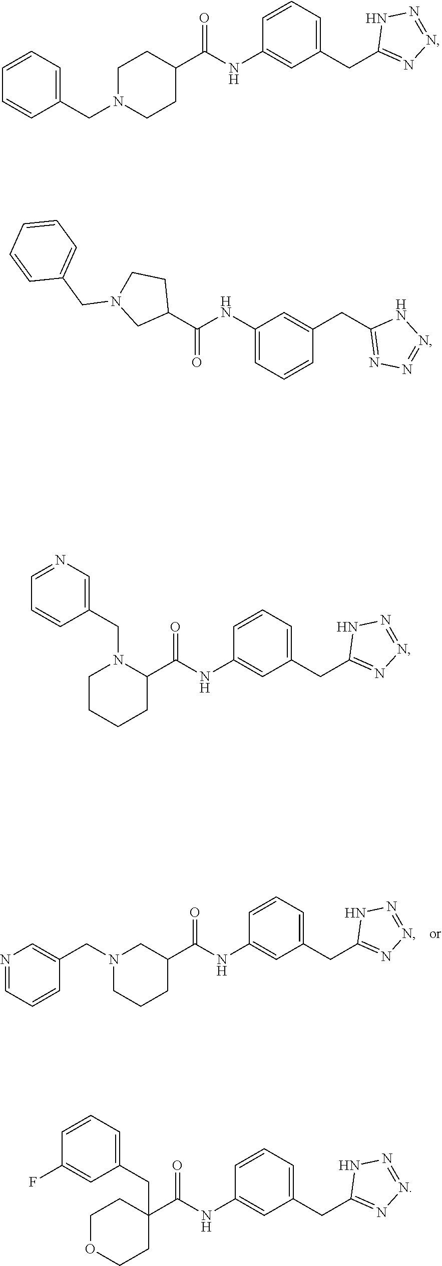 [SMILES: O=C(Nc1cccc(Cc2nnn[nH]2)c1)C1CCN(Cc2ccccc2)CC1 ; O=C(Nc1cccc(Cc2nnn[nH]2)c1)C1CCN(Cc2ccccc2)C1 ; O=C(Nc1cccc(Cc2nnn[nH]2)c1)C1CCCCN1Cc1cccnc1 ; O=C(Nc1cccc(Cc2nnn[nH]2)c1)C1CCCN(Cc2cccnc2)C1 ; O=C(Nc1cccc(Cc2nnn[nH]2)c1)C1(Cc2cccc(F)c2)CCOCC1]

Also provided herein is a compound of Formula (II), or a pharmaceutically acceptable salt thereof:

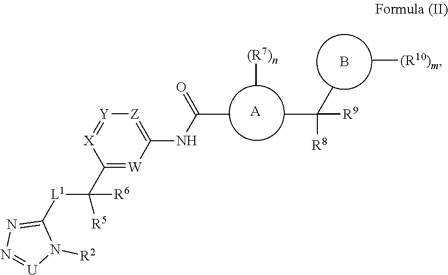

wherein:
U is —CR¹—; and
R², L¹, R⁵, R⁶, X, Y, Z, W, Ring A, R⁷, n, R⁸, R⁹, Ring B, R¹⁰, and m have the meaning as defined herein.

In some embodiments of a compound of Formula (II), R^{Y} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;

In some embodiments of a compound of Formula (II), X is —N—. In some embodiments of a compound of Formula (II), X is —CR^{X}—.

In some embodiments of a compound of Formula (II), Y is —N—. In some embodiments of a compound of Formula (II), Y is —CR^{Y}—.

In some embodiments of a compound of Formula (II), Z is —N—. In some embodiments of a compound of Formula (II), Z is —CR^{Z}—.

In some embodiments of a compound of Formula (II), W is —N—. In some embodiments of a compound of Formula (II), W is —CR^{W}—.

In some embodiments of a compound of Formula (II), the compound is of Formula (IIa):

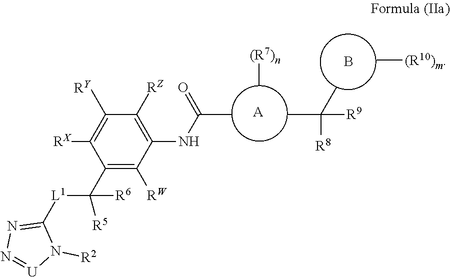

In some embodiments of a compound of Formula (II) or (IIa), R^{X} is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (II) or (IIa), R^{X} is hydrogen, halogen, C₁-C₆alkyl, C₁-C₆haloalkyl, cycloalkyl, or heterocycloalkyl. In some embodiments of a compound of Formula (II) or (IIa), R^{X} is hydrogen, halogen, C₁-C₆alkyl, or C₁-C₆haloalkyl. In some embodiments of a compound of Formula (II) or (IIa), R^{X} is hydrogen or C₁-C₆alkyl. In some embodiments of a compound of Formula (II) or (IIa), R^{X} is hydrogen.

In some embodiments of a compound of Formula (II) or (IIa), R^{Y} is hydrogen, halogen, —CN, —OR^{a}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆haloalkenyl, C₂-C₆alkynyl, cycloalkyl optionally substituted with one or more halogens, or heterocycloalkyl optionally substituted with one or more halogens.

In some embodiments of a compound of Formula (II) or (IIa), R^{Y} is hydrogen, halogen, —CN, —OR^{a}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl optionally substituted with one or more halogens, or heterocycloalkyl optionally substituted with one or more halogens.

In some embodiments of a compound of Formula (II) or (IIa), R^{Y} is hydrogen, halogen, —CN, —OR^{a}, —C(═O)NR^{c}R^{d}, C₁-C₆alkyl, C₁-C₆haloalkyl, C₁-C₆hydroxyalkyl, C₂-C₆alkenyl, C₂-C₆haloalkenyl, C₂-C₆alkynyl, or cycloalkyl optionally substituted with one or more halogens.

In some embodiments of a compound of Formula (II) or (IIa), R^{Y} is hydrogen, C₁-C₆alkyl, C₁-C₆haloalkyl, C₃-C₆cycloalkyl, or C₁-C₆alkoxy, wherein the alkoxy is optionally substituted with one or more halogen.

In some embodiments of a compound of Formula (II) or (IIa), R^{Y} is hydrogen, —OR^{a}, C₁-C₆alkyl, C₁-C₆haloalkyl, or C₃-C₆cycloalkyl; and R^{a} is C₁-C₆haloalkyl.

In some embodiments of a compound of Formula (II) or (IIa), R^{Y} is hydrogen, halogen, —CN, —OR^{a}, —C(═O)NR^{c}R^{d}, C₁-C₆alkyl, C₁-C₆haloalkyl, C₁-C₆hydroxyalkyl, C₂-C₆haloalkenyl, C₂-C₆alkynyl, or cycloalkyl optionally substituted with one or more halogens.

In some embodiments of a compound of Formula (II) or (IIa), R^{Y} is hydrogen, halogen, —CN, —OR^{a}, —C(═O)NR^{c}R^{d}, C₁-C₆alkyl, C₁-C₆hydroxyalkyl, C₂-C₆alkenyl, C₂-C₆haloalkenyl, C₂C₂-C₆alkynyl, or cycloalkyl optionally substituted with one or more halogens.

In some embodiments of a compound of Formula (II) or (IIa), R^{Y} is hydrogen, halogen, —CN, —OR^{a}, —C(═O)NR^{c}R^{d}, C₁-C₆alkyl, C₁-C₆hydroxyalkyl, C₂-C₆alkenyl, C₂C₂-C₆alkynyl, or cycloalkyl optionally substituted with one or more halogens.

In some embodiments of a compound of Formula (II) or (IIa), R^{Y} is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl optionally substituted with one or more halogens, or heterocycloalkyl optionally substituted with one or more halogens.

In some embodiments of a compound of Formula (II) or (IIa), R^{Y} is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl.

In some embodiments of a compound of Formula (II) or (IIa), R^{Y} is hydrogen, halogen, C₁-C₆alkyl, C₁-C₆haloalkyl, cycloalkyl optionally substituted with one or more halogens, or heterocycloalkyl optionally substituted with one or more halogens.

In some embodiments of a compound of Formula (II) or (IIa), R^{Y} is hydrogen, halogen, C₁-C₆alkyl, C₁-C₆haloalkyl, cycloalkyl, or heterocycloalkyl.

In some embodiments of a compound of Formula (II) or (IIa), R^{Y} is hydrogen, halogen, C₁-C₆alkyl, or C₁-C₆haloalkyl.

In some embodiments of a compound of Formula (II) or (IIa), R^{Y} is hydrogen or C₁-C₆alkyl. In some embodiments of a compound of Formula (II) or (IIa), R^{Y} is hydrogen.

In some embodiments of a compound of Formula (II) or (IIa), R^{Z} is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (II) or (IIa), R^{Z} is hydrogen, halogen, C₁-C₆alkyl, C₁-C₆haloalkyl, cycloalkyl, or heterocycloalkyl. In some embodiments of a compound of Formula (II) or (IIa), R^{Z} is hydrogen, halogen, C₁-C₆alkyl, or C₁-C₆haloalkyl. In some embodiments of a compound of Formula (II) or (IIa), R^{Z} is hydrogen or C₁-C₆alkyl. In some embodiments of a compound of Formula (II) or (IIa), R^{Z} is hydrogen.

In some embodiments of a compound of Formula (II) or (IIa), R^{W} is hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (II) or (IIa), R^{W} is hydrogen, halogen, C₁-C₆alkyl, C₁-C₆haloalkyl, cycloalkyl, or heterocycloalkyl. In some embodiments of a compound of Formula (II) or (IIa), R^{W} is hydrogen, halogen, C₁-C₆alkyl, or C₁-C₆haloalkyl. In some embodiments of a compound of Formula (II) or (IIa), R^{W} is hydrogen or C₁-C₆alkyl. In some embodiments of a compound of Formula (II) or (IIa), R^{W} is hydrogen.

In some embodiments of a compound of Formula (II) or (IIa), R⁸ and R⁹ are each independently hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound of Formula (II) or (IIa), R⁸ and R⁹ are each independently hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (II) or (IIa), R⁸ and R⁹ are each independently hydrogen, halogen, C₁-C₆alkyl, or C₁-C₆haloalkyl. In some embodiments of a compound of Formula (II) or (IIa), R⁸ and R⁹ are each independently hydrogen or C₁-C₆alkyl. In some embodiments of a compound of Formula (II) or (IIa), R⁸ and R⁹ are each hydrogen. In some embodiments of a compound of Formula (II) or (IIa), R⁸ and R⁹ are taken together to form a cycloalkyl or heterocycloalkyl; each optionally substituted with one or more R. In some embodiments of a compound of Formula (II) or (IIa), R⁸ and R⁹ are taken together to form an oxo.

In some embodiments of a compound of Formula (II) or (IIa), Ring B is cycloalkyl or heterocycloalkyl. In some embodiments of a compound of Formula (II) or (IIa), Ring B is cycloalkyl. In some embodiments of a compound of Formula (II) or (IIa), Ring B is heterocycloalkyl. In some embodiments of a compound of Formula (II) or (IIa), Ring B is 5- or 6-membered heterocycloalkyl.

In some embodiments of a compound of Formula (II) or (IIa), Ring B is azetidinyl, pyrrolidinyl, piperidinyl, or morpholinyl. In some embodiments of a compound of Formula (II) or (IIa), Ring B is piperidinyl.

In some embodiments of a compound of Formula (II) or (IIa), each R¹⁰ is independently halogen, —CN, —OH, —OR^{a}, —NR^{c}R^{d}, C₁-C₆alkyl, C₁-C₆haloalkyl, C₂-C₆alkynyl, cycloalkyl, heterocycloalkyl, C₁-C₆alkylene(cycloalkyl), or C₁-C₆alkylene(heterocycloalkyl); wherein each alkyl, alkylene, alkynyl, cycloalkyl, and heterocycloalkyl is independently optionally substituted with one or more R.

In some embodiments of a compound of Formula (II) or (IIa), each R¹⁰ is independently halogen, C₁-C₆alkyl, C₁-C₆haloalkyl, C₂-C₆alkynyl, C₁-C₆alkylene(cycloalkyl), or C₁-C₆alkylene(heterocycloalkyl); wherein each alkyl, alkylene, alkynyl, cycloalkyl, and heterocycloalkyl is independently optionally substituted with one or more R. In some embodiments of a compound of Formula (II) or (IIa), each R¹⁰ is independently halogen or C₁-C₆alkyl. In some embodiments of a compound of Formula (II) or (IIa), each R¹⁰ is independently C₁-C₆alkyl.

In some embodiments of a compound of Formula (II) or (IIa), m is 0, 1, 2, or 3. In some embodiments of a compound of Formula (II) or (IIa), m is 1 or 2. In some embodiments of a compound of Formula (II) or (IIa), m is 1, 2, or 3. In some embodiments of a compound of Formula (II) or (IIa), m is 0, 1, or 2. In some embodiments of a compound of Formula (II) or (IIa), m is 1 or 2. In some embodiments of a compound of Formula (II) or (IIa), m is 0. In some embodiments of a compound of Formula (II) or (IIa), m is 1. In some embodiments of a compound of Formula (II) or (IIa), m is 2. In some embodiments of a compound of Formula (II) or (IIa), m is 3. In some embodiments of a compound of Formula (II) or (IIa), m is 4.

In some embodiments of a compound of Formula (II) or (IIa),

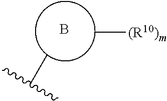

is

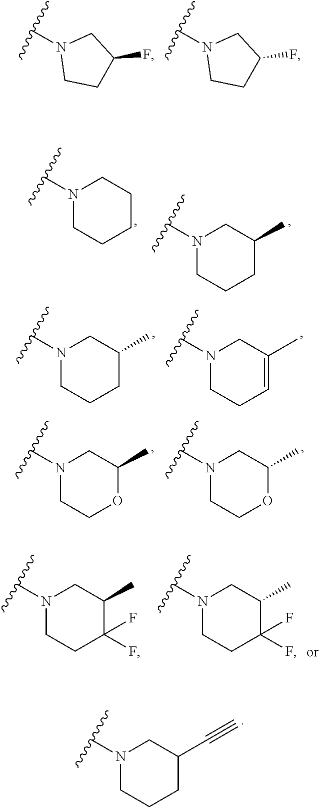

In some embodiments of a compound of Formula (II) or (IIa), is

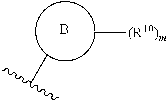

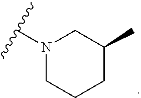 [SMILES: *N1CCC[C@H](C)C1]

In some embodiments of a compound of Formula (II) or (IIa), L¹ is absent. In some embodiments of a compound of Formula (II) or (IIa), L¹ is —CR³R⁴—.

In some embodiments of a compound of Formula (II) or (IIa), R³ and R⁴ are each independently hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound of Formula (II) or (IIa), R³ and R⁴ are each independently hydrogen, halogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments of a compound of Formula (II) or (IIa), R³ and R⁴ are each independently hydrogen, halogen, C₁-C₆alkyl, C₁-C₆haloalkylcycloalkyl, or heterocycloalkyl. In some embodiments of a compound of Formula (II) or (IIa), R³ and R⁴ are each independently hydrogen, halogen, C₁-C₆alkyl, or C₁-C₆haloalkyl. In some embodiments of a compound of Formula (II) or (IIa), R³ and R⁴ are each hydrogen. In some embodiments of a compound of Formula (II) or (IIa), R³ and R⁴ are taken together to form a cycloalkyl or heterocycloalkyl; each optionally substituted with one or more R. In some embodiments of a compound of Formula (II) or (IIa), R³ and R⁴ are taken together to form an oxo.

In some embodiments of a compound of Formula (II) or (IIa), R⁶ is hydrogen, halogen, —CN, —OH, —OR^{a}, —NR^{c}R^{d}, C₁-C₆alkyl, or C₁-C₆haloalkyl. In some embodiments of a compound of Formula (II) or (IIa), R⁶ is hydrogen, C₁-C₆alkyl, or C₁-C₆haloalkyl. In some embodiments of a compound of Formula (II) or (IIa), R⁶ is hydrogen or C₁-C₆alkyl. In some embodiments of a compound of Formula (II) or (IIa), R⁶ is hydrogen.

In some embodiments of a compound of Formula (II) or (IIa), R⁵ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{5a}.

In some embodiments of a compound of Formula (II) or (IIa), R⁵ is cycloalkyl, heterocycloalkyl, aryl, or heteroaryl; wherein each cycloalkyl, heterocycloalkyl, aryl, and heteroaryl is independently optionally substituted with one or more R^{5a}. In some embodiments of a compound of Formula (II) or (IIa), R⁵ is cycloalkyl or heterocycloalkyl; wherein each cycloalkyl and heterocycloalkyl is independently optionally substituted with one or more R^{5a}. In some embodiments of a compound of Formula (II) or (IIa), R⁵ is cycloalkyl optionally substituted with one or more R^{5a}. In some embodiments of a compound of Formula (II) or (IIa), R⁵ is heterocycloalkyl optionally substituted with one or more R^{5a}.

In some embodiments of a compound of Formula (II) or (IIa), R⁵ and R⁶ are taken together to form a cycloalkyl optionally substituted with one or more R⁵.

In some embodiments of a compound of Formula (II) or (IIa), R⁵ and R⁶ are taken together to form a heterocycloalkyl optionally substituted with one or more R^{5a}.

In some embodiments of a compound of Formula (II) or (IIa), R⁵ and R⁶ are taken together to form a cyclobutyl optionally substituted with one or more R^{5a}.

In some embodiments of a compound of Formula (II) or (IIa), each R^{5a} is independently halogen, —CN, —OH, —OR^{a}, —NR^{c}R^{d}, C₁-C₆alkyl, C₁-C₆haloalkyl, C₂-C₆alkynyl, cycloalkyl, or heterocycloalkyl; wherein each alkyl, alkynyl, cycloalkyl, and heterocycloalkyl is independently optionally substituted with one or more R. In some embodiments of a compound of Formula (II) or (IIa), each R^{5a} is independently halogen, —CN, C₁-C₆alkyl, C₁-C₆haloalkyl, or C₂-C₆alkynyl; wherein each alkyl and alkynyl is independently optionally substituted with one or more R.

In some embodiments of a compound of Formula (II) or (IIa),

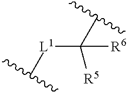

is

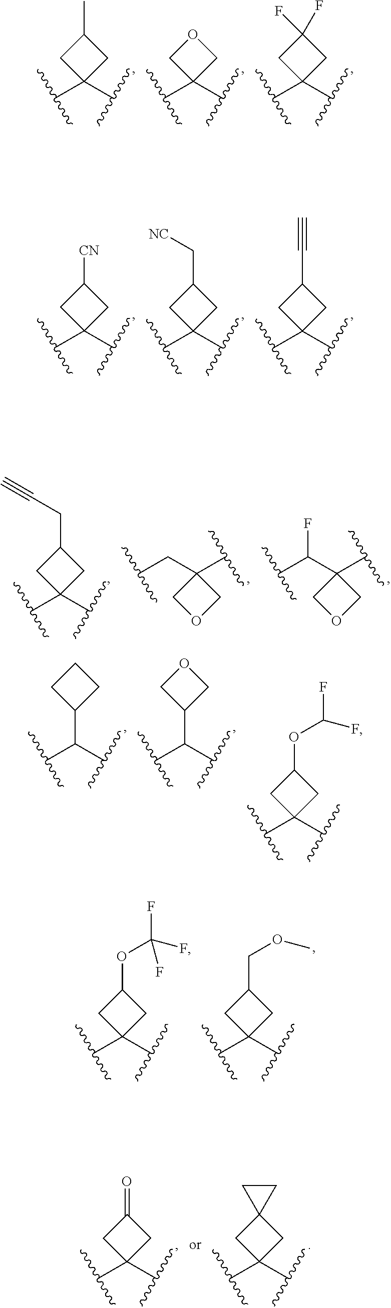

In some embodiments of a compound of Formula (II) or (IIa),

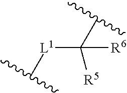

is

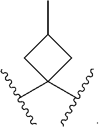 [SMILES: *C1(*)CC(C)C1]

In some embodiments of a compound of Formula (II) or (IIa), U is —N—. In some embodiments of a compound of Formula (II) or (IIa), U is —CR¹—.

In some embodiments of a compound of Formula (II) or (IIa), R¹ is hydrogen or C₁-C₆alkyl. In some embodiments of a compound of Formula (II) or (IIa), R¹ is C₁-C₆alkyl. In some embodiments of a compound of Formula (II) or (IIa), R¹ is hydrogen.

In some embodiments of a compound of Formula (II) or (IIa), R² is hydrogen or C₁-C₆alkyl. In some embodiments of a compound of Formula (II) or (IIa), R² is C₁-C₆alkyl. In some embodiments of a compound of Formula (II) or (IIa), R² is hydrogen.

In some embodiments of a compound of Formula (II) or (IIa), Ring A is cycloalkyl or heterocycloalkyl. In some embodiments of a compound of Formula (II) or (IIa), Ring A is aryl or heteroaryl. In some embodiments of a compound of Formula (II) or (IIa), Ring A is aryl. In some embodiments of a compound of Formula (II) or (IIa), Ring A is phenyl. In some embodiments of a compound of Formula (II) or (IIa), Ring A is heteroaryl.

In some embodiments of a compound of Formula (II) or (IIa), Ring A is a bicyclic cycloalkyl, bicyclic heterocycloalkyl, bicyclic aryl, or bicyclic heteroaryl. In some embodiments of a compound of Formula (II) or (IIa), Ring A is a bicyclic cycloalkyl or bicyclic heterocycloalkyl. In some embodiments of a compound of Formula (II) or (IIa), Ring A is a bicyclic aryl or bicyclic heteroaryl. In some embodiments of a compound of Formula (II) or (IIa), Ring A is a bicyclic heteroaryl.

In some embodiments of a compound of Formula (II) or (IIa), Ring A is benzothiazolyl, benzimidazolyl, indazolyl, pyrazolopyridinyl, thiazolopyridinyl, quinoxalinyl, or pyridopyrazinyl.

In some embodiments of a compound of Formula (II) or (IIa), Ring A is tetrahydroisoquinolinyl, dihydrophthalazinyl, dihydroisoquinolinyl, dihydroquinazolinyl, dihydropyrroloimidazolyl, dihydroimidazopyridinyl, dihydrothienopyridazinyl, dihydroindazolyl, tetrahydropyrrolopyridinyl, or dihydropyrrolopyridine.

In some embodiments of a compound of Formula (II) or (IIa), each R⁷ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, or C₁-C₆heteroalkyl; wherein each alkyl is independently optionally substituted with one or more R; or two R⁷ on the same atom are taken together to form an oxo. In some embodiments of a compound of Formula (II) or (IIa), each R⁷ is independently halogen, —CN, —OH, —OR^{a}, —NR^{c}R^{d}, C₁-C₆alkyl, or C₁-C₆haloalkyl; wherein each alkyl is independently optionally substituted with one or more R; or two R⁷ on the same atom are taken together to form an oxo. In some embodiments of a compound of Formula (II) or (IIa), each R⁷ is independently —OH, C₁-C₆alkyl, or C₁-C₆haloalkyl; or two R⁷ on the same atom are taken together to form an oxo. In some embodiments of a compound of Formula (II) or (IIa), each R⁷ is independently C₁-C₆haloalkyl; or two R⁷ on the same atom are taken together to form an oxo.

In some embodiments of a compound of Formula (II) or (IIa), n is 0, 1, 2, 3, or 4. In some embodiments of a compound of Formula (II) or (IIa), n is 0, 1, 2, or 3. In some embodiments of a compound of Formula (II) or (IIa), n is 1, 2, or 3. In some embodiments of a compound of Formula (II) or (IIa), n is 1, 2, 3, or 4. In some embodiments of a compound of Formula (II) or (IIa), n is 2, 3, or 4. In some embodiments of a compound of Formula (II) or (IIa), n is 2 or 3. In some embodiments of a compound of Formula (II) or (IIa), n is 0. In some embodiments of a compound of Formula (II) or (IIa), n is 1. In some embodiments of a compound of Formula (II) or (IIa), n is 2. In some embodiments of a compound of Formula (II) or (IIa), n is 3. In some embodiments of a compound of Formula (II) or (IIa), n is 4.

In some embodiments of a compound of Formula (II) or (IIa),

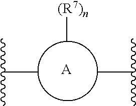

is

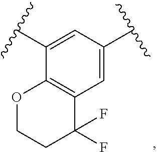 [SMILES: *c1cc(*)c2c(c1)C(F)(F)CCO2]

In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl), wherein each alkyl, alkylene, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl, wherein each alkyl, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, or C₁-C₆heteroalkyl, wherein each alkyl, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, or C₁-C₆heteroalkyl. In some embodiments of a compound disclosed herein, each R^{a} is independently C₁-C₆alkyl or C₁-C₆haloalkyl. In some embodiments of a compound disclosed herein, each R is independently C₁-C₆alkyl.

In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl), wherein each alkyl, alkylene, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl, wherein each alkyl, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, or C₁-C₆heteroalkyl, wherein each alkyl, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, or C₁-C₆heteroalkyl. In some embodiments of a compound disclosed herein, each R^{b} is independently hydrogen, C₁-C₆alkyl, or C₁-C₆haloalkyl. In some embodiments of a compound disclosed herein, each R^{b} is independently hydrogen or C₁-C₆alkyl. In some embodiments of a compound disclosed herein, each R^{b} is hydrogen. In some embodiments of a compound disclosed herein, each R^{b} is independently C₁-C₆alkyl.

In some embodiments of a compound disclosed herein, R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl), wherein each alkyl, alkylene, cycloalkyl, and heterocycloalkyl is independently optionally substituted with one or more R. In some embodiments of a compound disclosed herein, R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl, wherein each alkyl, cycloalkyl, and heterocycloalkyl is independently optionally substituted with one or more R. In some embodiments of a compound disclosed herein, R^{c} and R^{d} are each independently hydrogen, C₁-C₆alkyl, C₁-C₆haloalkyl, C₁-C₆hydroxyalkyl, C₁-C₆aminoalkyl, or C₁-C₆heteroalkyl, wherein each alkyl, cycloalkyl, and heterocycloalkyl is independently optionally substituted with one or more R. In some embodiments of a compound disclosed herein, R^{c} and R^{d} are each independently hydrogen, C₁-C₆alkyl, C₁-C₆haloalkyl, C₁-C₆hydroxyalkyl, C₁-C₆aminoalkyl, or C₁-C₆heteroalkyl. In some embodiments of a compound disclosed herein, R^{c} and R^{d} are each independently hydrogen, C₁-C₆alkyl, or C₁-C₆haloalkyl. In some embodiments of a compound disclosed herein, R and R^{d} are each independently hydrogen or C₁-C₆alkyl. In some embodiments of a compound disclosed herein, R^{c} and R^{d} are each hydrogen. In some embodiments of a compound disclosed herein, R^{c} and R^{d} are each independently C₁-C₆alkyl.

In some embodiments of a compound disclosed herein, R^{c} and R^{d} are taken together with the atom to which they are attached to form a heterocycloalkyl optionally substituted with one or more R. In some embodiments of a compound disclosed herein, R^{c} and R^{d} are taken together with the atom to which they are attached to form a heterocycloalkyl.

In some embodiments of a compound disclosed herein, each R is independently halogen, —CN, —OH, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, or C₃-C₆cycloalkyl; or two R on the same atom form an oxo. In some embodiments of a compound disclosed herein, each R is independently halogen, —CN, —OH, —NH₂, —NHC₁-C₃alkyl, —N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, or C₃-C₆cycloalkyl; or two R on the same atom form an oxo. In some embodiments of a compound disclosed herein, each R is independently halogen, —CN, —OH, —NH₂, C₁-C₃alkyl, C₁-C₃alkoxy, C₁-C₃haloalkyl, or C₁-C₃haloalkoxy; or two R on the same atom form an oxo. In some embodiments of a compound disclosed herein, each R is independently halogen, —CN, —OH, —NH₂, C₁-C₃alkyl, or C₁-C₃haloalkyl; or two R on the same atom form an oxo. In some embodiments of a compound disclosed herein, each R is independently halogen or C₁-C₃alkyl.

In some embodiments of a compound disclosed herein, one or more of R, R¹, R², R³, R⁴, R⁵, R^{5a}, R⁶, R⁷, R⁸, R⁹, R¹⁰, R^{X}, R^{Y}, R^{Z}, R^{W}, R^{a}, R^{b}, R^{c}, and R^{d} groups comprise deuterium at a percentage higher than the natural abundance of deuterium.

In some embodiments of a compound disclosed herein, one or more ¹H are replaced with one or more deuteriums in one or more of the following groups R, R¹, R², R³, R⁴, R⁵, R^{5a}, R⁶, R⁷, R⁸, R⁹, R¹⁰, R^{X}, R^{Y}, R^{Z}, R^{W}, R^{a}, R^{b}, R^{c}, and R^{d}.

In some embodiments of a compound disclosed herein, the abundance of deuterium in each of R, R¹, R², R³, R⁴, R⁵, R^{5a}, R⁶, R⁷, R⁸, R⁹, R¹⁰, R^{X}, R^{Y}, R^{Z}, R^{W}, R^{a}, R^{b}, R^{c}, and R^{d} is independently at least 1%, at least 10%, at least 20%, at least 30%, at least 40%, at least 50%, at least 60%, at least 70%, at least 80%, at least 90%, or 100% by molar.

In some embodiments of a compound disclosed herein, one or more ¹H of Ring A or Ring B are replaced with one or more deuteriums.

Any combination of the groups described above for the various variables is contemplated herein. Throughout the specification, groups and substituents thereof are chosen by one skilled in the field to provide stable moieties and compounds.

In some embodiments the compound disclosed herein, or a pharmaceutically acceptable salt thereof, is one of the compounds in Table 1.

TABLE 1

| EX | Structure |
|---|---|
| 1* | 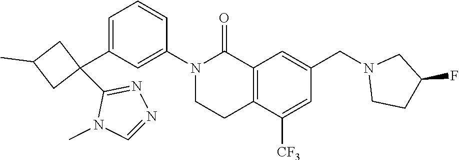 [SMILES: CC1CC(c2cccc(N3CCc4c(cc(CN5CC[C@H](F)C5)cc4C(F)(F)F)C3=O)c2)(c2nncn2C)C1] |
| 2*** | 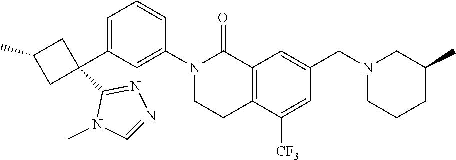 |
| 3*** | 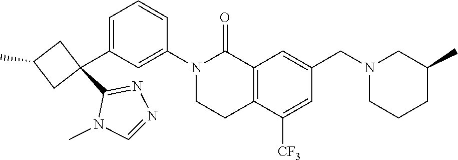 |
| 4* | 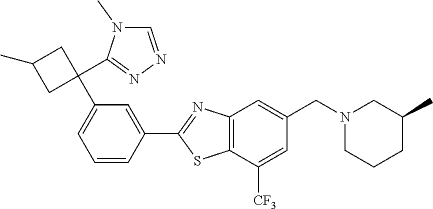 |
| 5* | 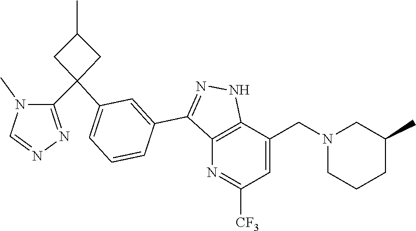 |
| 6* | 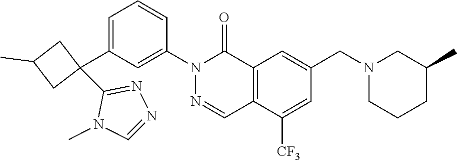 |
| 7* | 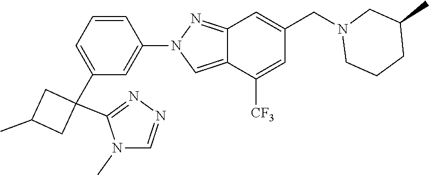 |
| 8* | 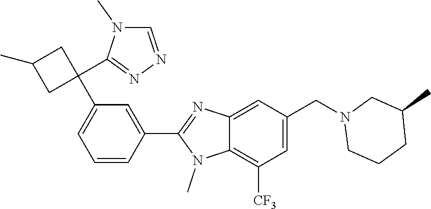 |
| 9* | 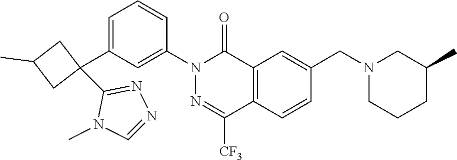 |
| 10* | 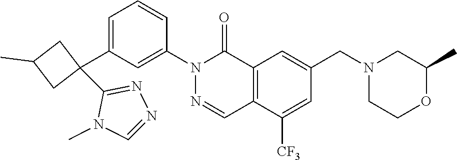 |
| 11* | 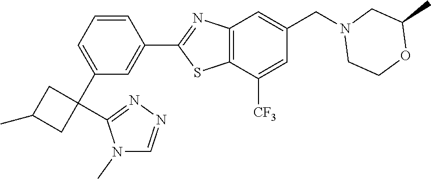 |
| 12* | 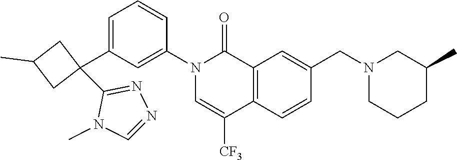 |
| 13 | 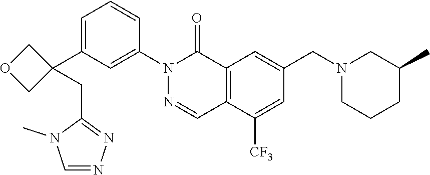 [SMILES: C[C@H]1CCCN(Cc2cc(C(F)(F)F)c3cnn(-c4cccc(C5(Cc6nncn6C)COC5)c4)c(=O)c3c2)C1] |
| 14* | 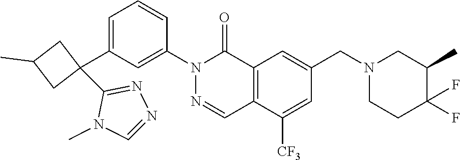 [SMILES: CC1CC(c2cccc(-n3ncc4c(C(F)(F)F)cc(CN5CCC(F)(F)[C@H](C)C5)cc4c3=O)c2)(c2nncn2C)C1] |
| 15* | 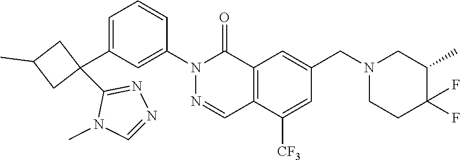 [SMILES: CC1CC(c2cccc(-n3ncc4c(C(F)(F)F)cc(CN5CCC(F)(F)[C@@H](C)C5)cc4c3=O)c2)(c2nncn2C)C1] |
| 16 | 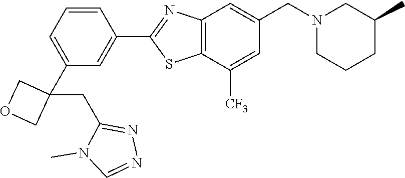 [SMILES: C[C@H]1CCCN(Cc2cc(C(F)(F)F)c3sc(-c4cccc(C5(Cc6nncn6C)COC5)c4)nc3c2)C1] |
| 17* | 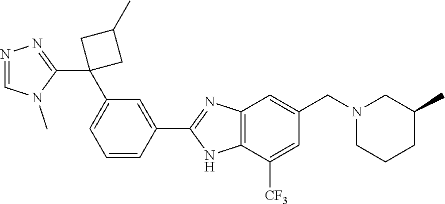 [SMILES: CC1CC(c2cccc(-c3nc4cc(CN5CCC[C@H](C)C5)cc(C(F)(F)F)c4[nH]3)c2)(c2nncn2C)C1] |
| 18 | 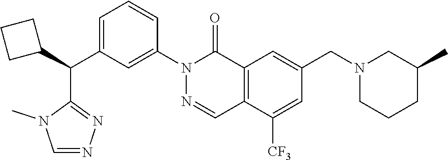 [SMILES: C[C@H]1CCCN(Cc2cc(C(F)(F)F)c3cnn(-c4cccc([C@@H](c5nncn5C)C5CCC5)c4)c(=O)c3c2)C1] |
| and | |
| 19** | 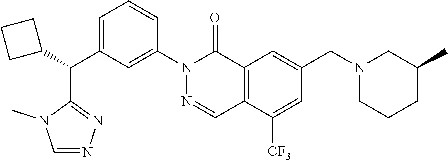 [SMILES: C[C@H]1CCCN(Cc2cc(C(F)(F)F)c3cnn(-c4cccc([C@H](c5nncn5C)C5CCC5)c4)c(=O)c3c2)C1] |
| 20* | 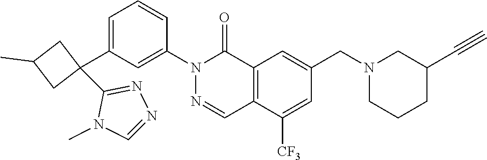 [SMILES: C#CC1CCCN(Cc2cc(C(F)(F)F)c3cnn(-c4cccc(C5(c6nncn6C)CC(C)C5)c4)c(=O)c3c2)C1] |
| 21* | 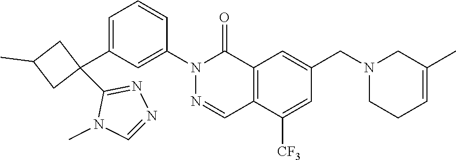 |
| 22* | 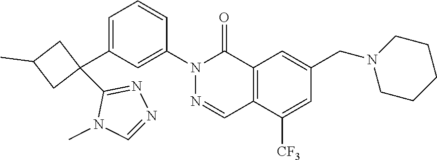 [SMILES: CC1CC(c2cccc(-n3ncc4c(C(F)(F)F)cc(CN5CCCCC5)cc4c3=O)c2)(c2nncn2C)C1] |
| 23* | 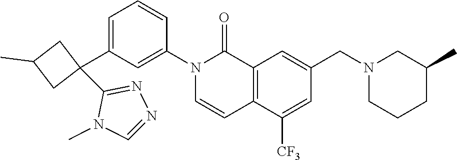 |
| 24* | 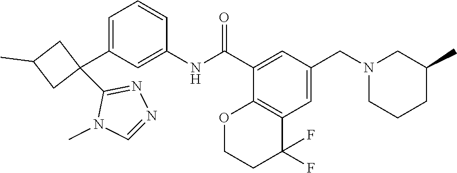 |
| 25* | 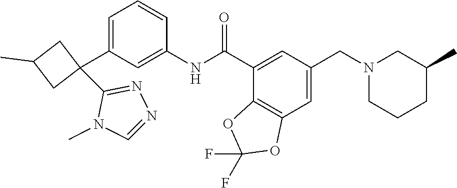 |
| 26 | 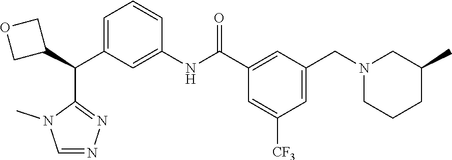 [SMILES: C[C@H]1CCCN(Cc2cc(C(=O)Nc3cccc([C@@H](c4nncn4C)C4COC4)c3)cc(C(F)(F)F)c2)C1] |
| and | |
| 27 | 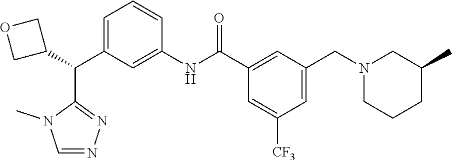 [SMILES: C[C@H]1CCCN(Cc2cc(C(=O)Nc3cccc([C@H](c4nncn4C)C4COC4)c3)cc(C(F)(F)F)c2)C1] |
| 28 | 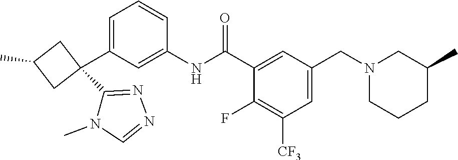 |
| and | |
| 29** | 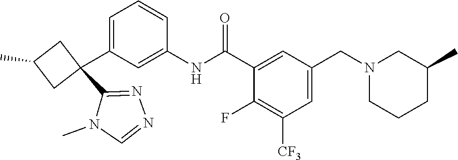 |
| 30* | 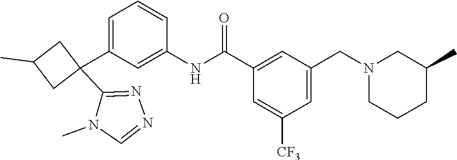 |
| 31* | 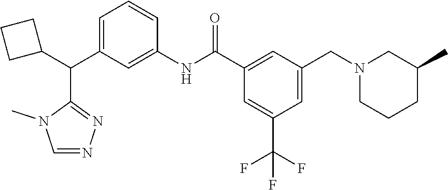 [SMILES: C[C@H]1CCCN(Cc2cc(C(=O)Nc3cccc(C(c4nncn4C)C4CCC4)c3)cc(C(F)(F)F)c2)C1] |
| 32* | 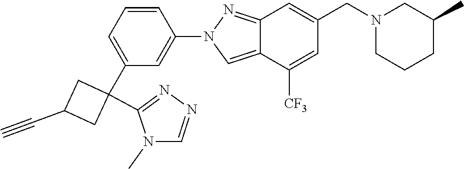 [SMILES: C#CC1CC(c2cccc(-n3cc4c(C(F)(F)F)cc(CN5CCC[C@H](C)C5)cc4n3)c2)(c2nncn2C)C1] |
| 33* | 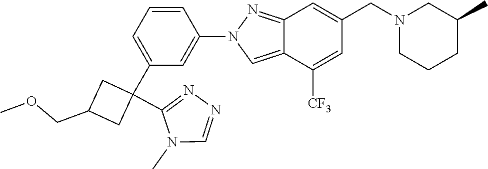 |
| 34*** | 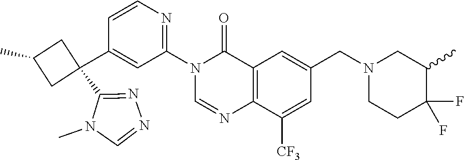 [SMILES: CC1CN(Cc2cc(C(F)(F)F)c3ncn(-c4cc([C@]5(c6nncn6C)C[C@@H](C)C5)ccn4)c(=O)c3c2)CCC1(F)F] |
| 35 | 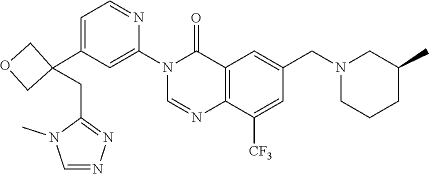 [SMILES: C[C@H]1CCCN(Cc2cc(C(F)(F)F)c3ncn(-c4cc(C5(Cc6nncn6C)COC5)ccn4)c(=O)c3c2)C1] |
| 36*** | 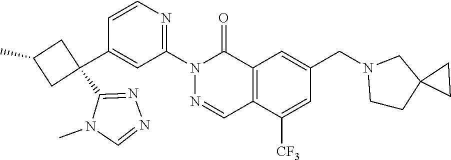 [SMILES: Cn1cnnc1[C@]1(c2ccnc(-n3ncc4c(C(F)(F)F)cc(CN5CCC6(CC6)C5)cc4c3=O)c2)C[C@H](C)C1] |
| 37*** | 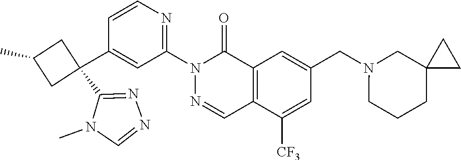 [SMILES: Cn1cnnc1[C@]1(c2ccnc(-n3ncc4c(C(F)(F)F)cc(CN5CCCC6(CC6)C5)cc4c3=O)c2)C[C@H](C)C1] |
| 38* | 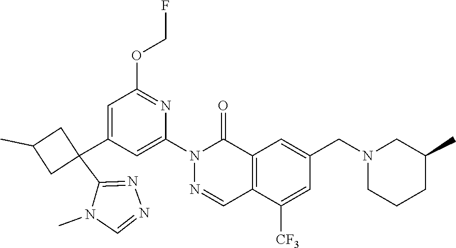 |
| 39*** | 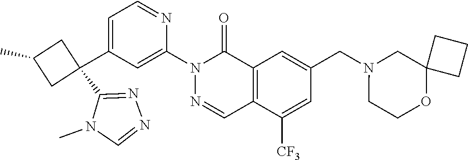 [SMILES: Cn1cnnc1[C@]1(c2ccnc(-n3ncc4c(C(F)(F)F)cc(CN5CCOC6(CCC6)C5)cc4c3=O)c2)C[C@H](C)C1] |
| 40*** | 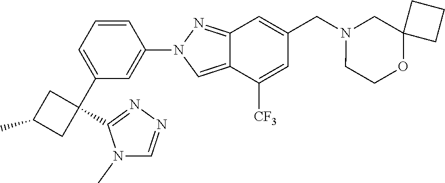 [SMILES: Cn1cnnc1[C@]1(c2cccc(-n3cc4c(C(F)(F)F)cc(CN5CCOC6(CCC6)C5)cc4n3)c2)C[C@H](C)C1] |
| 41* | 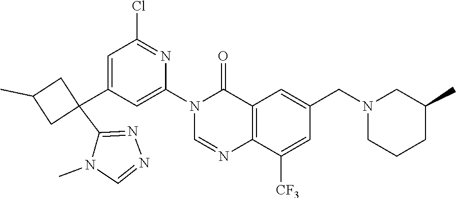 |
| 42*** | 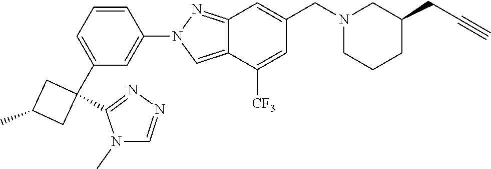 [SMILES: C#CC[C@H]1CCCN(Cc2cc(C(F)(F)F)c3cn(-c4cccc([C@]5(c6nncn6C)C[C@@H](C)C5)c4)nc3c2)C1] |
| 43* | 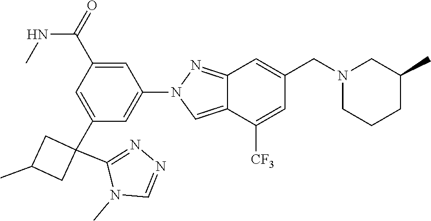 |
| 44* | 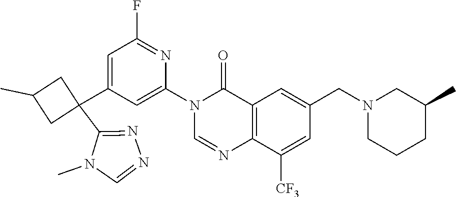 |
| 45* | 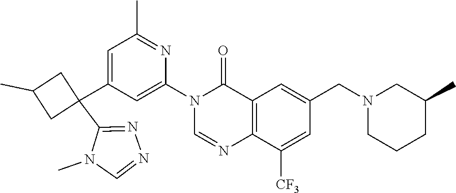 |
| 46* | 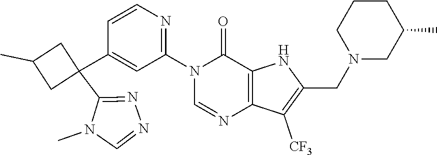 |
| 47* | 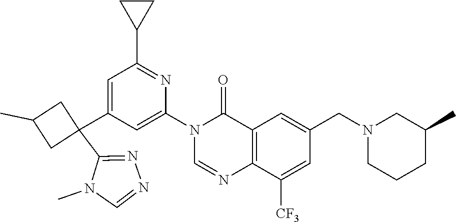 |
| 48*** | 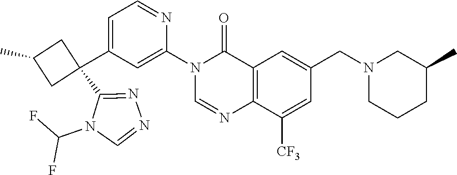 |
| 49* | 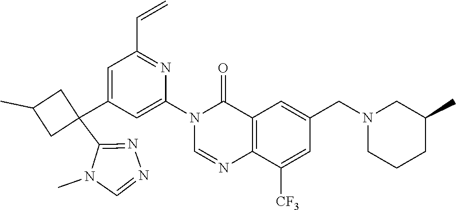 |
| 50 | 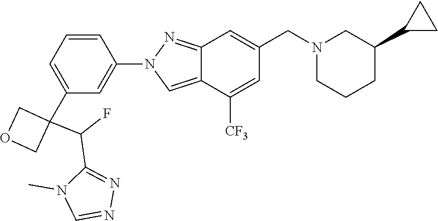 [SMILES: Cn1cnnc1C(F)C1(c2cccc(-n3cc4c(C(F)(F)F)cc(CN5CCC[C@H](C6CC6)C5)cc4n3)c2)COC1] |
| 51* | 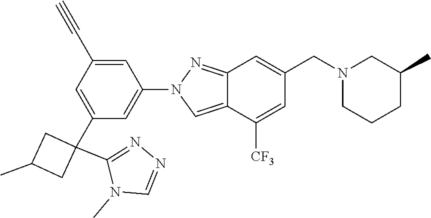 |
| 52* | 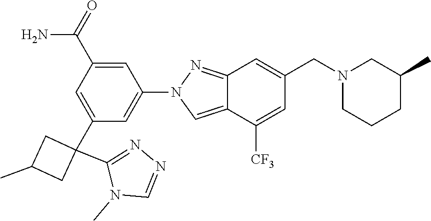 |
| 53* | 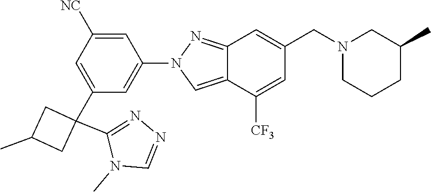 |
| 54* | 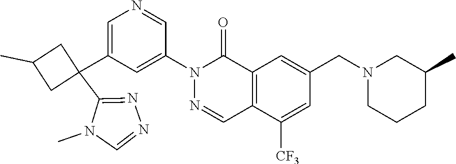 |
| 55 | 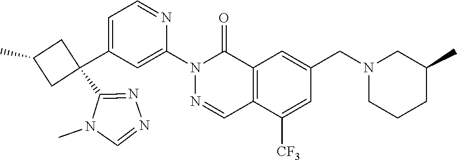 |
| and | |
| 65** | 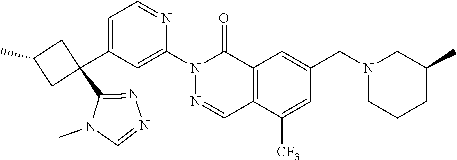 |
| 56*** | 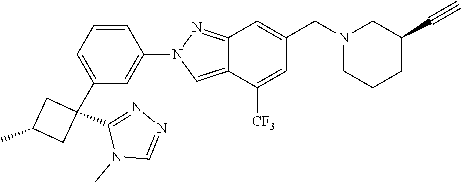 [SMILES: C#C[C@H]1CCCN(Cc2cc(C(F)(F)F)c3cn(-c4cccc([C@]5(c6nncn6C)C[C@@H](C)C5)c4)nc3c2)C1] |
| 57* | 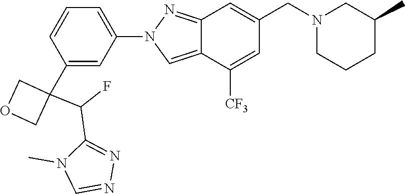 [SMILES: C[C@H]1CCCN(Cc2cc(C(F)(F)F)c3cn(-c4cccc(C5(C(F)c6nncn6C)COC5)c4)nc3c2)C1] |
| 58 | 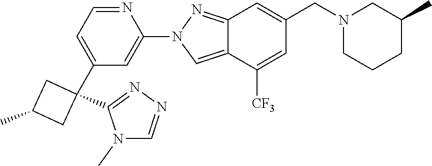 |
| and | |
| 71** | 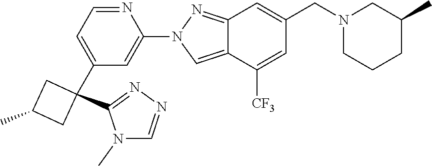 |
| 59* | 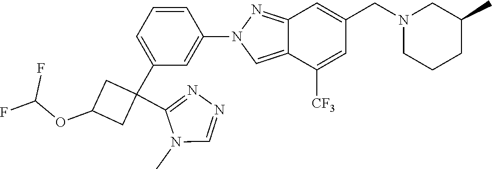 [SMILES: C[C@H]1CCCN(Cc2cc(C(F)(F)F)c3cn(-c4cccc(C5(c6nncn6C)CC(OC(F)F)C5)c4)nc3c2)C1] |
| 60* | 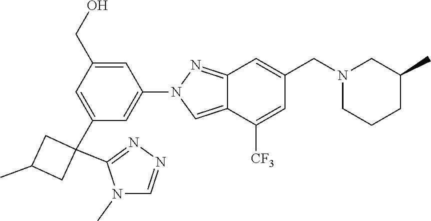 |
| 61*** | 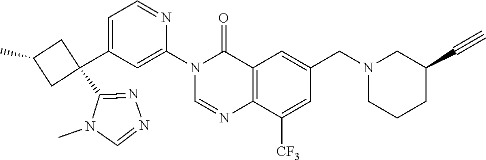 [SMILES: C#C[C@H]1CCCN(Cc2cc(C(F)(F)F)c3ncn(-c4cc([C@]5(c6nncn6C)C[C@@H](C)C5)ccn4)c(=O)c3c2)C1] |
| 62*** | 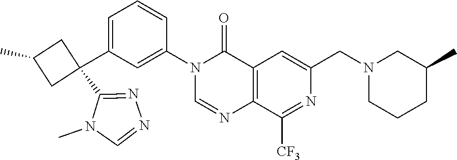 |
| 63* | 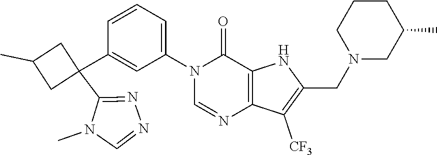 |
| 64 | 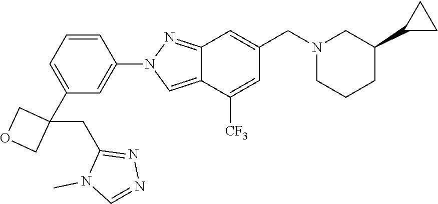 [SMILES: Cn1cnnc1CC1(c2cccc(-n3cc4c(C(F)(F)F)cc(CN5CCC[C@H](C6CC6)C5)cc4n3)c2)COC1] |
| 66* | 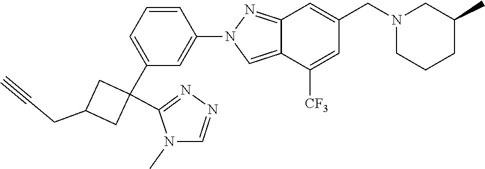 [SMILES: C#CCC1CC(c2cccc(-n3cc4c(C(F)(F)F)cc(CN5CCC[C@H](C)C5)cc4n3)c2)(c2nncn2C)C1] |
| 67 | 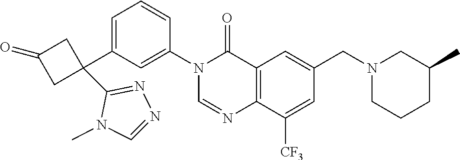 [SMILES: C[C@H]1CCCN(Cc2cc(C(F)(F)F)c3ncn(-c4cccc(C5(c6nncn6C)CC(=O)C5)c4)c(=O)c3c2)C1] |
| 68 | 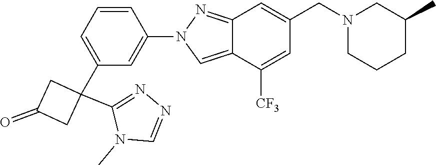 [SMILES: C[C@H]1CCCN(Cc2cc(C(F)(F)F)c3cn(-c4cccc(C5(c6nncn6C)CC(=O)C5)c4)nc3c2)C1] |
| 69 | 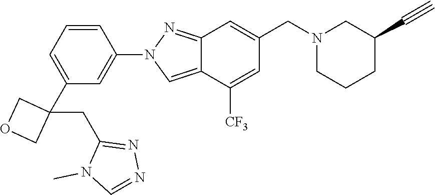 [SMILES: C#C[C@H]1CCCN(Cc2cc(C(F)(F)F)c3cn(-c4cccc(C5(Cc6nncn6C)COC5)c4)nc3c2)C1] |
| 70* | 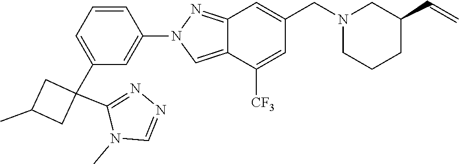 [SMILES: C=C[C@H]1CCCN(Cc2cc(C(F)(F)F)c3cn(-c4cccc(C5(c6nncn6C)CC(C)C5)c4)nc3c2)C1] |
| 72* | 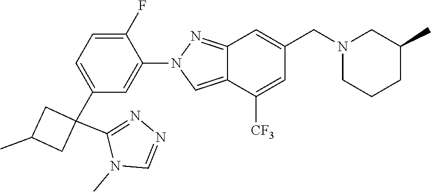 |
| 73* | 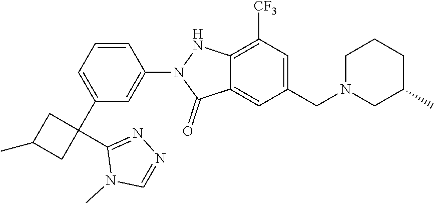 |
| 74*** | 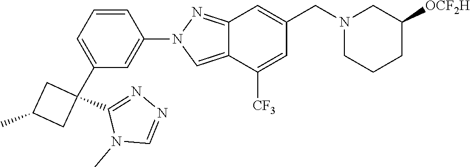 |
| 75 | 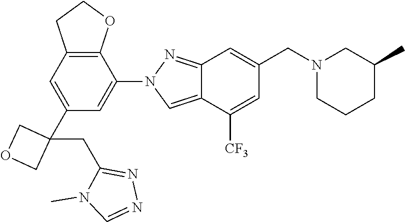 [SMILES: C[C@H]1CCCN(Cc2cc(C(F)(F)F)c3cn(-c4cc(C5(Cc6nncn6C)COC5)cc5c4OCC5)nc3c2)C1] |
| 76* | 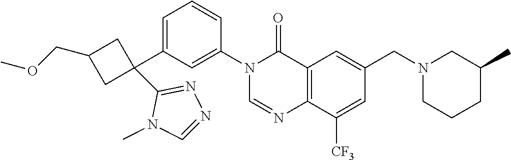 |
| 77* | 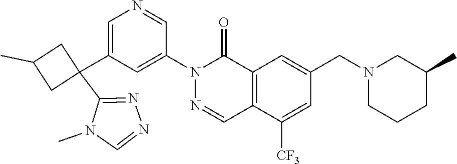 |
| 78*** | 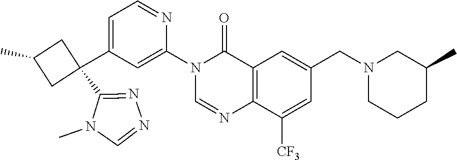 |
| 79* | 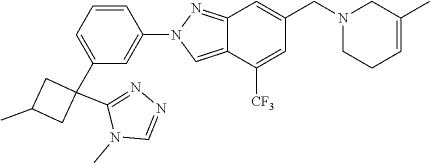 |
| 80 | 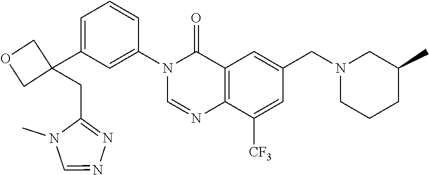 [SMILES: C[C@H]1CCCN(Cc2cc(C(F)(F)F)c3ncn(-c4cccc(C5(Cc6nncn6C)COC5)c4)c(=O)c3c2)C1] |
| 81* | 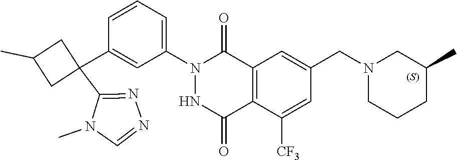 |
| 82* | 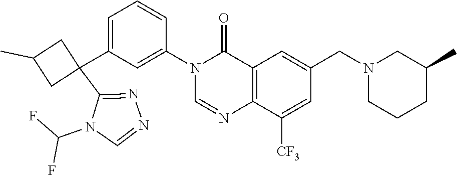 |
| 83 | 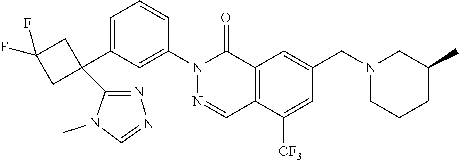 [SMILES: C[C@H]1CCCN(Cc2cc(C(F)(F)F)c3cnn(-c4cccc(C5(c6nncn6C)CC(F)(F)C5)c4)c(=O)c3c2)C1] |
| 84* | 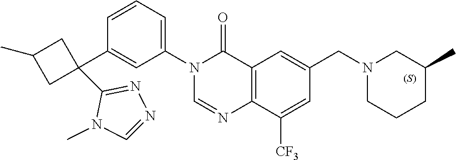 |
| 85* | 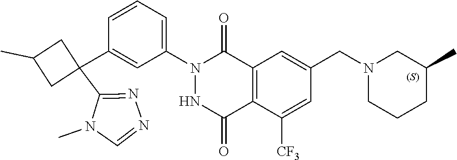 |
| 86* | 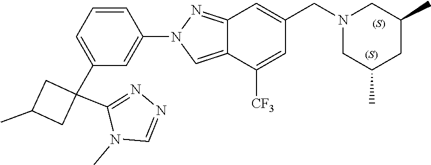 |
| 87 | 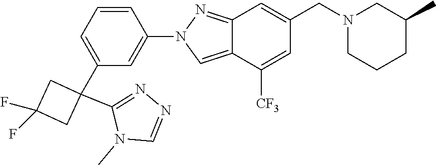 [SMILES: C[C@H]1CCCN(Cc2cc(C(F)(F)F)c3cn(-c4cccc(C5(c6nncn6C)CC(F)(F)C5)c4)nc3c2)C1] |
| 88 | 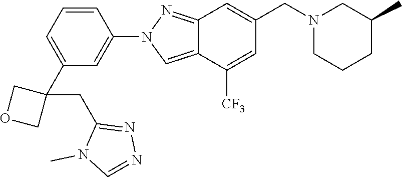 [SMILES: C[C@H]1CCCN(Cc2cc(C(F)(F)F)c3cn(-c4cccc(C5(Cc6nncn6C)COC5)c4)nc3c2)C1] |
| 89* | 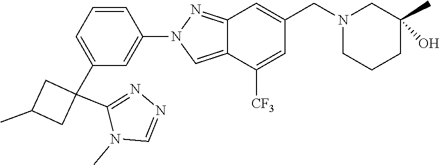 |
| 90* | 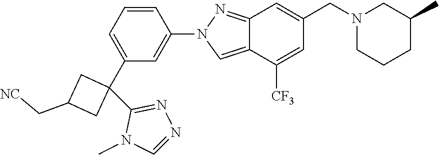 [SMILES: C[C@H]1CCCN(Cc2cc(C(F)(F)F)c3cn(-c4cccc(C5(c6nncn6C)CC(CC#N)C5)c4)nc3c2)C1] |
| 91* | 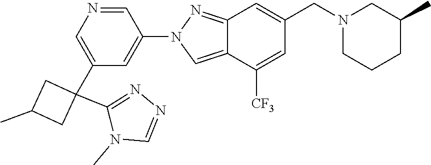 |
| 92*** | 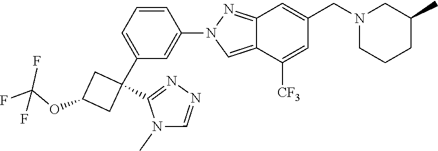 [SMILES: C[C@H]1CCCN(Cc2cc(C(F)(F)F)c3cn(-c4cccc([C@]5(c6nncn6C)C[C@@H](OC(F)(F)F)C5)c4)nc3c2)C1] |
| 93*** | 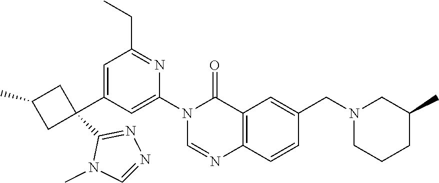 |
| 94*** | 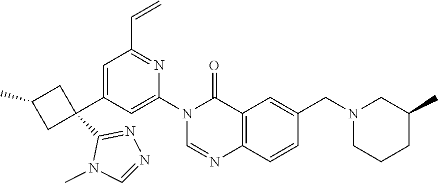 |
| 95*** | 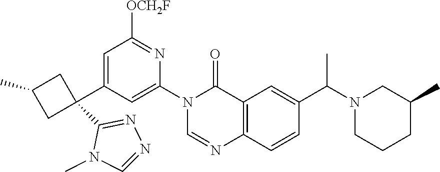 |
| 96*** | 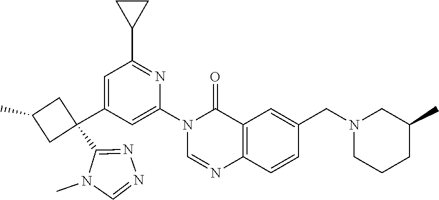 |
| 97*** | 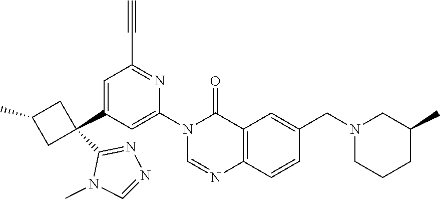 |
| 98*** | 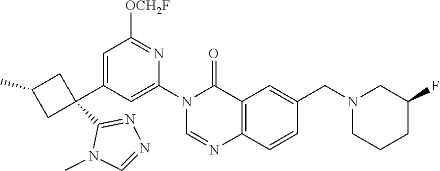 |
| 99*** | 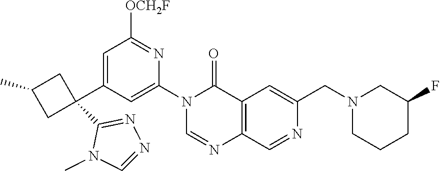 |
| 100*** | 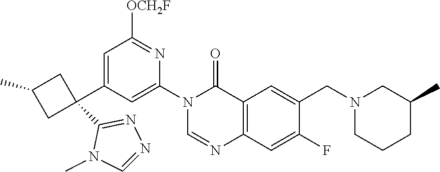 |
| 101* | 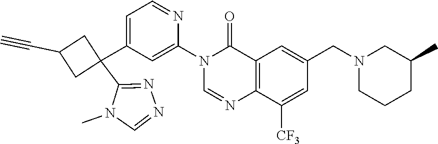 [SMILES: C#CC1CC(c2ccnc(-n3cnc4c(C(F)(F)F)cc(CN5CCC[C@H](C)C5)cc4c3=O)c2)(c2nncn2C)C1] |
| 102*** | 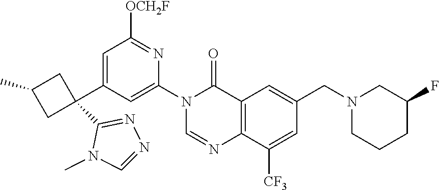 |
| 103* | 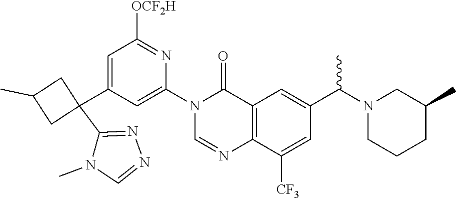 |
| 104*** | 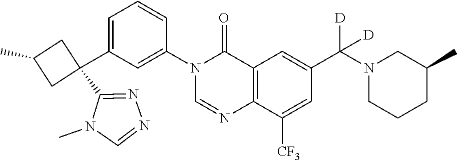 |
| 105*** | 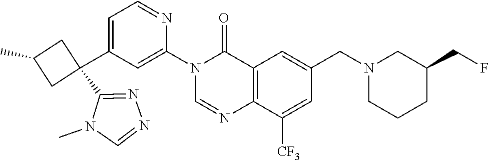 [SMILES: Cn1cnnc1[C@]1(c2ccnc(-n3cnc4c(C(F)(F)F)cc(CN5CCC[C@H](CF)C5)cc4c3=O)c2)C[C@H](C)C1] |
| 106*** | 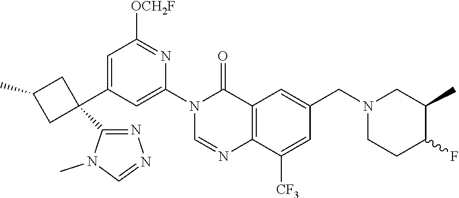 |
| 107*** | 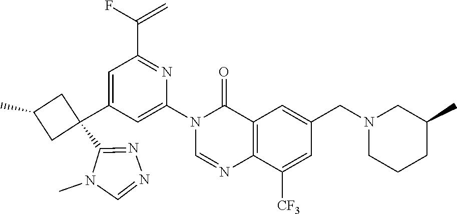 |
| 108*** | 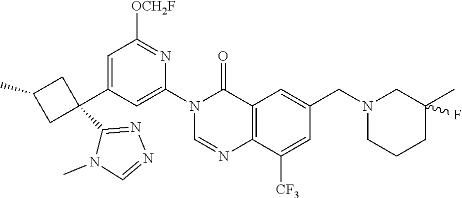 |
| 109*** | 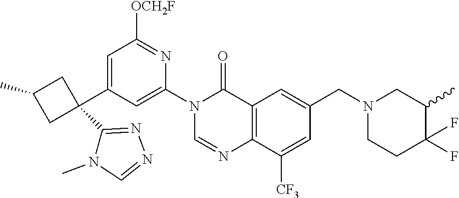 |
| 110*** | 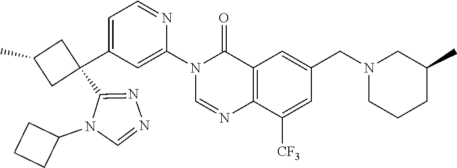 |
| 111*** | 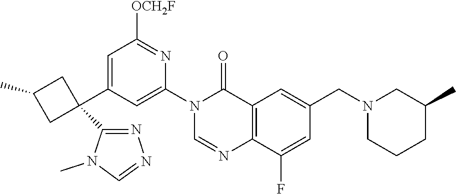 |
| 112*** | 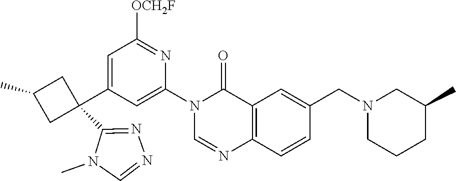 |
| 113* | 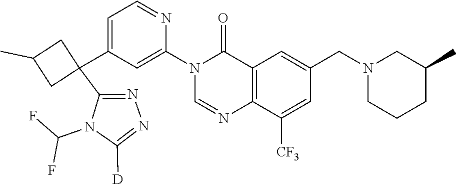 |
| 114*** | 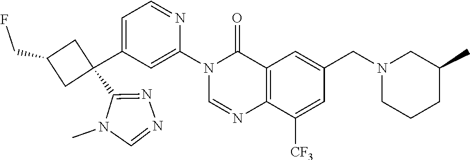 [SMILES: C[C@H]1CCCN(Cc2cc(C(F)(F)F)c3ncn(-c4cc([C@]5(c6nncn6C)C[C@@H](CF)C5)ccn4)c(=O)c3c2)C1] |
| 115*** | 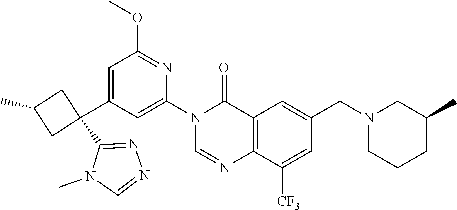 |
| 116* | 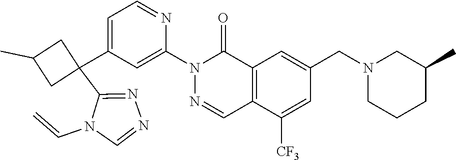 |
| 117* | 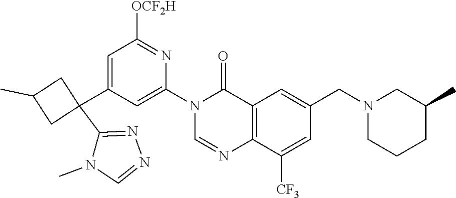 |
| 118 | 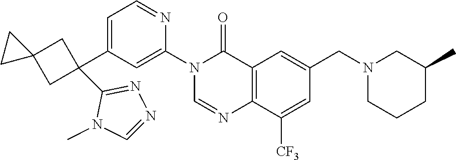 [SMILES: C[C@H]1CCCN(Cc2cc(C(F)(F)F)c3ncn(-c4cc(C5(c6nncn6C)CC6(CC6)C5)ccn4)c(=O)c3c2)C1] |
| 119*** | 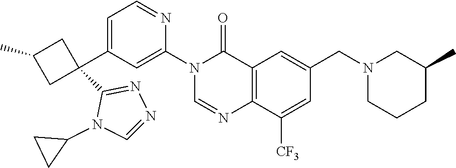 |
| 120*** | 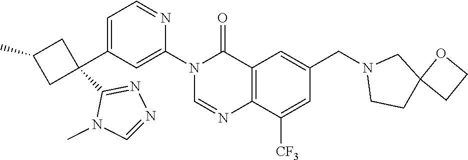 [SMILES: Cn1cnnc1[C@]1(c2ccnc(-n3cnc4c(C(F)(F)F)cc(CN5CCC6(CCO6)C5)cc4c3=O)c2)C[C@H](C)C1] |
| 121* | 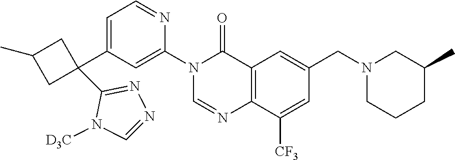 |
| 122*** | 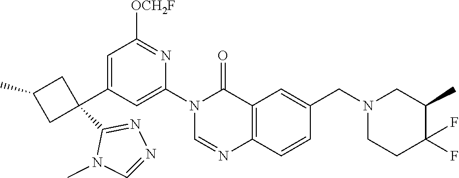 |
| 123*** | 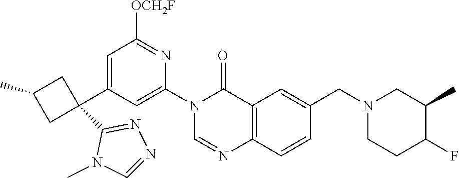 |
| 124*** | 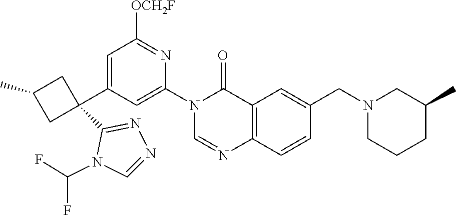 |

*trans and cis isomers were not separated.
**trans and cis isomers or diastereoisomers were separated and the absolute stereochemistry wasn't determined.
***trans and cis isomers were separated and the absolute stereochemistry at cyclobutyl position was determined.

In some embodiments the compound disclosed herein, or a pharmaceutically acceptable salt thereof, is one of the compounds in Table 2.

TABLE 2

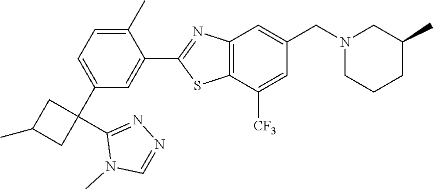

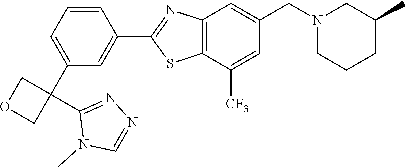 [SMILES: C[C@H]1CCCN(Cc2cc(C(F)(F)F)c3sc(-c4cccc(C5(c6nncn6C)COC5)c4)nc3c2)C1]

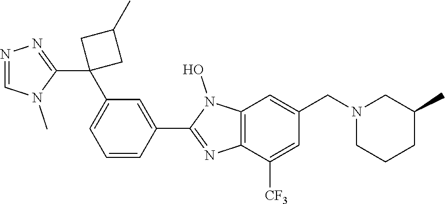 [SMILES: CC1CC(c2cccc(-c3nc4c(C(F)(F)F)cc(CN5CCC[C@H](C)C5)cc4n3O)c2)(c2nncn2C)C1]

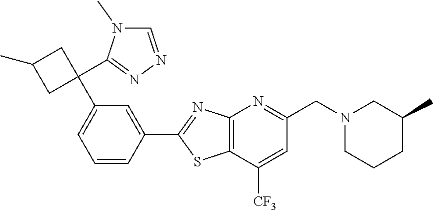

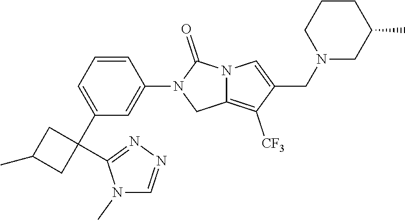

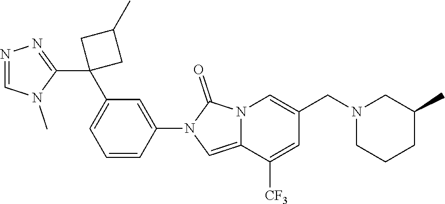 [SMILES: CC1CC(c2cccc(-n3cc4c(C(F)(F)F)cc(CN5CCC[C@H](C)C5)cn4c3=O)c2)(c2nncn2C)C1]

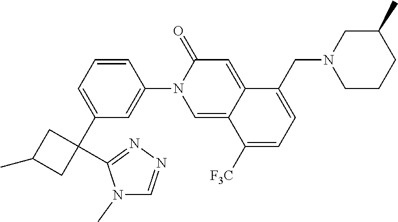

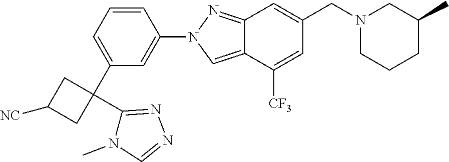 [SMILES: C[C@H]1CCCN(Cc2cc(C(F)(F)F)c3cn(-c4cccc(C5(c6nncn6C)CC(C#N)C5)c4)nc3c2)C1]

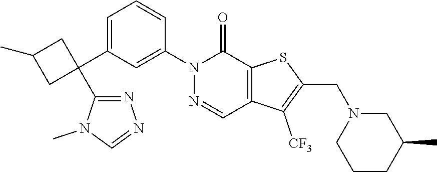

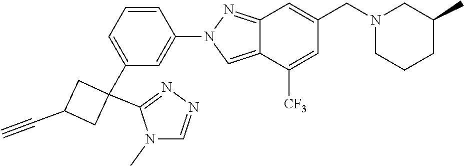 [SMILES: C#CC1CC(c2cccc(-n3cc4c(C(F)(F)F)cc(CN5CCC[C@H](C)C5)cc4n3)c2)(c2nncn2C)C1]

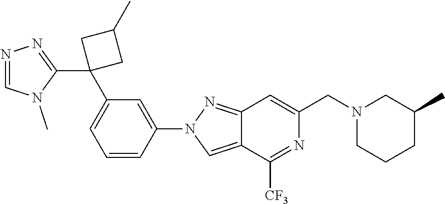 [SMILES: CC1CC(c2cccc(-n3cc4c(C(F)(F)F)nc(CN5CCC[C@H](C)C5)cc4n3)c2)(c2nncn2C)C1]

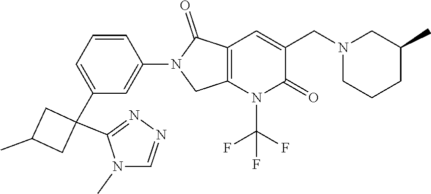

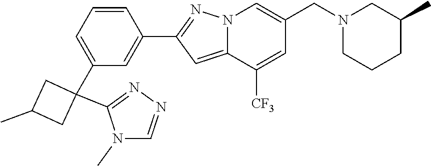

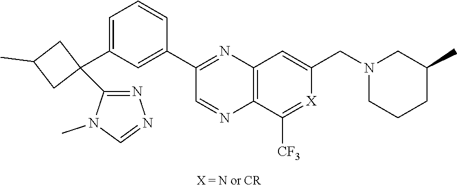

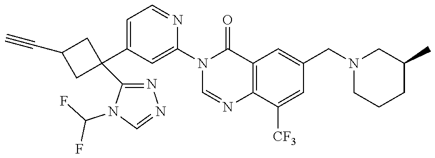 [SMILES: C#CC1CC(c2ccnc(-n3cnc4c(C(F)(F)F)cc(CN5CCC[C@H](C)C5)cc4c3=O)c2)(c2nncn2C(F)F)C1]

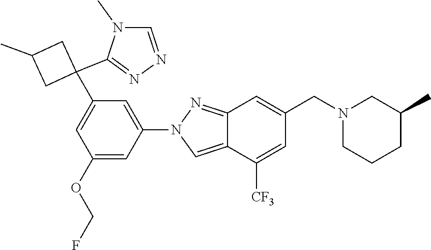

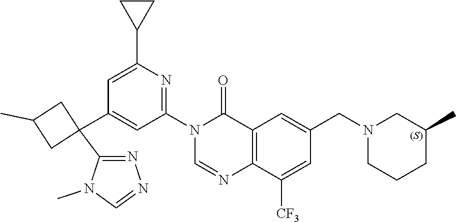

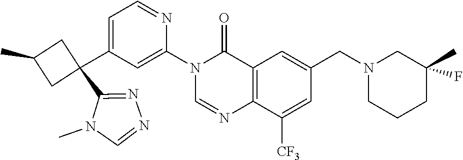 [SMILES: Cn1cnnc1[C@]1(c2ccnc(-n3cnc4c(C(F)(F)F)cc(CN5CCC[C@@](C)(F)C5)cc4c3=O)c2)C[C@H](C)C1]

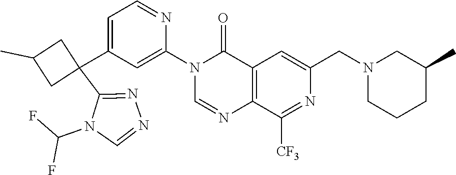

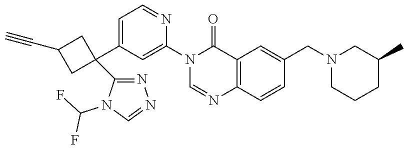 [SMILES: C#CC1CC(c2ccnc(-n3cnc4ccc(CN5CCC[C@H](C)C5)cc4c3=O)c2)(c2nncn2C(F)F)C1]

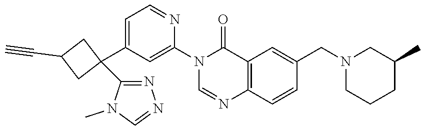 [SMILES: C#CC1CC(c2ccnc(-n3cnc4ccc(CN5CCC[C@H](C)C5)cc4c3=O)c2)(c2nncn2C)C1]

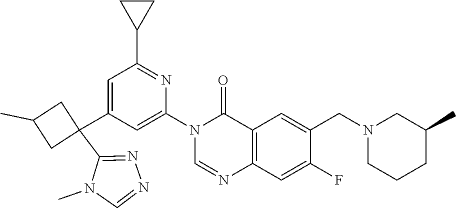

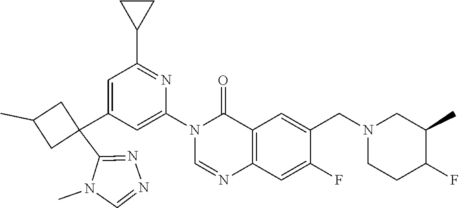 [SMILES: CC1CC(c2cc(C3CC3)nc(-n3cnc4cc(F)c(CN5CCC(F)[C@H](C)C5)cc4c3=O)c2)(c2nncn2C)C1]

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H (D), ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability.

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% by molar. In some embodiments, one or more of the substituents disclosed herein comprise deuterium at a percentage higher than the natural abundance of deuterium. In some embodiments, one or more ¹H are replaced with one or more deuteriums in one or more of the substituents disclosed herein.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein are methods of modulating the activity of an immune cell (e.g., a T-cell, a B-cell, or a NK-cell) such as by contacting the immune cell with an effective amount of a Cbl-b inhibitor described herein, or a pharmaceutically acceptable salt thereof, or a composition thereof.

Also provided are in vitro methods of producing said immune cells with modulated activity, referred to herein as “modified immune cells,” wherein said modified immune cells can be administered to an individual in need thereof (e.g., an individual having cancer) by ex vivo methods. Further provided are in vivo methods of modulating a response in an individual in need thereof (e.g., an individual with cancer), wherein the method comprises administration of an effective amount of a Cbl-b inhibitor described herein or a composition thereof. Moreover, the present disclosure provides in vitro methods of producing an expanded population of lymphocytes after in vivo lympho-conditioning in an individual, wherein the lympho-conditioning occurs as a result of administration of an effective amount of a Cbl-b inhibitor described herein or a composition thereof to the individual. The expanded population of lymphocytes can then be administered to the individual. The expanded population of lymphocytes can then be administered to the individual with cancer. In some embodiments, the modified immune cells or the expanded population of lymphocytes are produced from a biological sample comprising immune cells obtained from the individual, such as a blood sample comprising peripheral blood mononuclear cells or a tumor biopsy comprising tumor infiltrating lymphocytes (IILs).

Also disclosed herein is a method of treating a cancer, the method comprising administering an effective amount of a compound disclosed herein, or a pharmaceutically acceptable salt thereof, to the subject in need thereof.

Also disclosed herein is a method of treating a cancer responsive to inhibition of Cbl-b activity, the method comprising administering an effective amount of a compound disclosed herein, or a pharmaceutically acceptable salt thereof, to the subject in need thereof.

Also disclosed herein is a method of inhibiting abnormal cell proliferation, the method comprising administering an effective amount of a compound disclosed herein, or a pharmaceutically acceptable salt thereof, to the subject in need thereof.

Also disclosed herein is a method of modulating the immune response, the method comprising administering an effective amount of a compound disclosed herein, or a pharmaceutically acceptable salt thereof, to the subject in need thereof.

Also disclosed herein is a method of inhibiting Cbl-b activity, the method comprising administering an effective amount of a compound disclosed herein, or a pharmaceutically acceptable salt thereof, to the subject in need thereof.

Also disclosed herein is a method for treating a disease or condition associated with Cbl-b activity, the method comprising administering an effective amount of a compound disclosed herein, or a pharmaceutically acceptable salt thereof, to the subject in need thereof.

In some embodiments, the disease or condition associated with Cbl-b activity is cancer.

In some embodiments, the cancer is a hematologic cancer. In some embodiments, the cancer is a lymphoma, a leukemia, or a myeloma. In some embodiments, the cancer is a non-hematologic cancer. In some embodiments, the cancer is a sarcoma, a carcinoma, or a melanoma. In some embodiments, the cancer is solid tumor cancer.

Hematologic cancers include, but are not limited to, one or more leukemias such as B-cell acute lymphoid leukemia (“BALL”), T-cell acute lymphoid leukemia (“TALL”), acute lymphoid leukemia (ALL); one or more chronic leukemias including, but not limited to, chronic myelogenous leukemia (CML) and chronic lymphocytic leukemia (CLL); additional hematologic cancers or hematologic conditions including, but not limited to, B-cell prolymphocytic leukemia, blastic plasmacytoid dendritic cell neoplasm, Burkitt's lymphoma, diffuse large B-cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia,” which are a diverse collection of hematological conditions united by ineffective production (or dysplasia) of myeloid blood cells.

Non-hematologic cancers include but are not limited to, a neuroblastoma, renal cell carcinoma, colon cancer, colorectal cancer, breast cancer, epithelial squamous cell cancer, melanoma, stomach cancer, brain cancer, lung cancer (e.g., NSCLC), pancreatic cancer, cervical cancer, ovarian cancer, liver cancer, bladder cancer, prostate cancer, testicular cancer, thyroid cancer, uterine cancer, adrenal cancer, and head and neck cancer.

### Dosing

In certain embodiments, the compositions containing the compound(s) described herein are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In some embodiments, doses employed for adult human treatment are typically in the range of 0.01 mg-5000 mg per day. In some embodiments, the daily dosages appropriate for the compound described herein, or a pharmaceutically acceptable salt thereof, are from about 0.01 to about 50 mg/kg per body weight. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

### Combination

Disclosed herein are methods of treating a disease or disorder associated with cbl-b using a compound disclosed herein, or a pharmaceutically acceptable salt thereof, in combination with an additional therapeutic agent.

In some embodiments, the additional therapeutic agent is administered at the same time as the compound disclosed herein. In some embodiments, the additional therapeutic agent and the compound disclosed herein are administered sequentially. In some embodiments, the additional therapeutic agent is administered less frequently than the compound disclosed herein. In some embodiments, the additional therapeutic agent is administered more frequently than the compound disclosed herein. In some embodiments, the additional therapeutic agent is administered prior than the administration of the compound disclosed herein. In some embodiments, the additional therapeutic agent is administered after the administration of the compound disclosed herein.

In some embodiments, the additional therapeutic agent is an anti-cancer agent.

In some embodiments, the additional therapeutic agent is a cancer vaccines,

In some embodiments, the additional therapeutic agent is an oncolytic viruses.

### EXAMPLES

The following examples are offered to illustrate, but not to limit the claimed invention. The following examples further illustrate the invention but, of course, should not be construed as in any way limiting its scope.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### Example 1

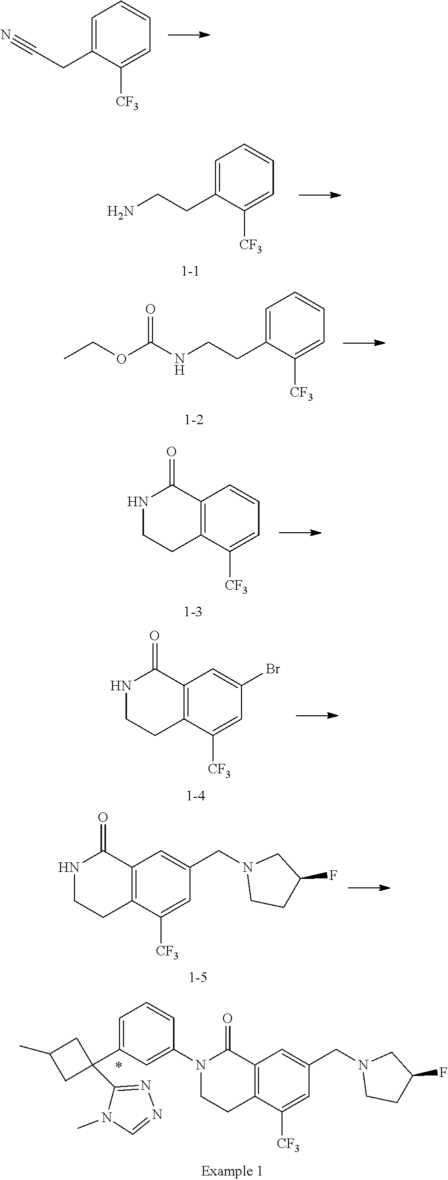

### Step 1: Preparation of Compound 1-1

To a solution of 2-(2-(trifluoromethyl)phenyl)acetonitrile (23.0 g, 124.2 mmol) in THE (200 mL) was added BH₃-Me₂S (10 M, 24.8 mL) dropwise at 0° C. over 30 mins and then the mixture was heated to reflux for 16 h. The reaction mixture was quenched by addition of MeOH (300 mL) at 25° C. and the mixture was concentrated under reduced pressure to give a residue. The residue was purified by flash silica gel chromatography to afford Compound 1-1. LCMS: 189.9 [M+H]⁺;

### Step 2: Preparation of Compound 1-2

To a solution of Compound 1-1 (16.0 g, 84.6 mmol) in CHCl₃ (200 mL) was added ethyl carbonochloridate (10.8 g, 99.9 mmol, 9.51 mL) and aq. Na₂CO₃ solution (1 M, 160.0 mL) at 0° C. The resulting mixture was stirred at 25° C. for 2 h. The reaction mixture was diluted with H₂O (200 mL) and extracted with DCM (100 mL×3). The combined organic layers were washed with brine (100 mL×2), dried over Na₂SO₄, filtered, and concentrated under reduced pressure to give a residue. The residue was purified by flash silica gel chromatography to afford Compound 1-2. ¹H NMR: (400 MHz, DMSO-d₆) δ 7.71-7.59 (m, 2H), 7.49-7.39 (m, 2H), 7.33-7.18 (m, 1H), 3.97 (q, J=7.1 Hz, 2H), 3.27-3.17 (m, 2H), 2.89 (t, J=7.3 Hz, 2H), 1.14 (t, J=7.1 Hz, 3H);

### Step 3: Preparation of Compound 1-3

To a solution of Compound 1-2 (8.00 g, 30.6 mmol) in POCl₃ (20 mL) was added P₂O₅ (8.69 g, 61.3 mmol). The resulting mixture was stirred at 115° C. for 2 h. The reaction mixture was concentrated under reduced pressure to remove POCl₃. The residue was poured into H₂O (50 mL) carefully at 25° C., and then diluted with H₂O (50 mL). The mixture was extracted with EtOAc (50 mL×3). The combined organic layers were washed with brine (50 mL×2), dried over Na₂SO₄, filtered, and concentrated under reduced pressure to give a residue. The residue was purified by flash silica gel chromatography to afford Compound 1-3. LCMS: 216.1 [M+H]⁺; ¹H NMR: (400 MHz, DMSO-d₆) δ 8.24 (br s, 1H), 8.16 (d, J=7.8 Hz, 1H), 7.88 (d, J=7.8 Hz, 1H), 7.56 (t, J=7.8 Hz, 1H), 3.41 (dt, J=2.9, 6.6 Hz, 2H), 3.03 (t, J=6.4 Hz, 2H);

### Step 4: Preparation of Compound 1-4

To a solution of Compound 1-3 (413.0 mg, 1.92 mmol) in H₂SO₄ (10 mL) was added NBS (409.9 mg, 2.30 mmol). The mixture was stirred at 25° C. for 16 h. The reaction mixture was added dropwise into ice water (20 mL) at 0° C., and the mixture was extracted with DCM (10 mL×2). The organic phase was dried over Na₂SO₄, filtered, and concentrated under reduced pressure to give a residue. Compound 1-4 was used into the next step without further purification. LCMS: 292.0/294.0 [M−H]⁻;

### Step 5: Preparation of Compound 1-5

To a solution of Compound 1-4 (150.0 mg, 0.5 mmol) in dioxane (4 mL) and H₂O (1 mL) was added trifluoro-[[(3S)-3-fluoropyrrolidin-1-yl]methyl]boranuide (213.3 mg, 1.02 mmol, K* salt), K₃PO₄ (324.8 mg, 1.53 mmol) and Ruphos-Pd-G4 (43.4 mg, 51.0 μmol). The mixture was stirred at 80° C. for 16 h under N₂. The reaction mixture was concentrated. The residue was purified by flash silica gel chromatography to afford Compound 1-5. LCMS: 317.2 [M+H]⁺;

### Step 6: Preparation of (S)-7-((3-fluoropyrrolidin-1-yl)methyl)-2-(3-(3-methyl-1-(4-methyl-4H-1,2,4-triazol-3-yl)cyclobutyl)phenyl)-5-(trifluoromethyl)-3,4-dihydroisoquinolin-1(2H)-one

A mixture of Compound 1-5 (80.0 mg, 252.9 μmol), 3-(1-(3-bromophenyl)-3-methyleyclobutyl)-4-methyl-4H-1,2,4-triazole (92.9 mg, 303.5 mol), CuI (9.63 mg, 50.6 mol), DMEDA (8.92 mg, 101.2 μmol, 10.9 VL) and K₂CO₃ (69.9 mg, 505.9 mol) in dioxane (2 mL) was degassed and purged with N₂ for 3 times, and then the mixture was stirred at 100° C. for 16 h under N₂ atmosphere. The reaction mixture was partitioned between brine (3 mL) and EtOAc (3 mL). The organic phase was separated, dried over Na₂SO₄, filtered, and concentrated under reduced pressure to give a residue. The residue was purified by Prep-HPLC to afford Example 1 as a HCl salt. LCMS: 542.4 [M+H]⁺; ¹H NMR: (400 MHz, DMSO-d⁶) δ 12.20-11.54 (m, 1H), 9.70-9.39 (m, 1H), 8.51 (br s, 1H), 8.40 (br s, 1H), 7.62-7.31 (m, 3H), 7.17 (d, J=7.5 Hz, 1H), 5.62-5.30 (m, 1H), 4.59 (br s, 2H), 4.11-3.98 (m, 2H), 3.91-3.70 (m, 1H), 3.47 (s, 4H), 3.30 (br s, 3H), 3.22-3.11 (m, 2H), 3.00-2.87 (m, 1H), 2.69-2.57 (m, 1H), 2.45-2.29 (m, 3H), 2.28-2.15 (m, 1H), 1.20-0.99 (m, 3H).

### Example 2 and 3

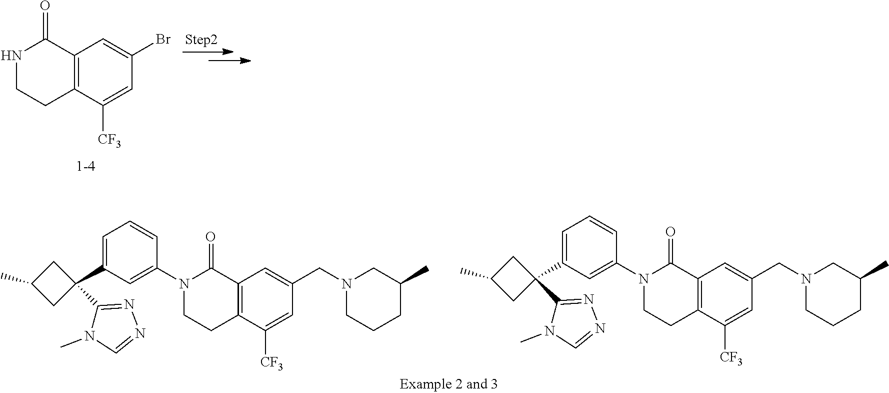

### Example 2 and 3

According to the procedures in Example 1, Example 2 and 3 was synthesized with Compound 1-4 (3.6 g, 12.242 mmol) and potassium (S)-trifluoro((3-methylpiperidin-1-yl)methyl)borate (335.28 mg, 1.530 mmol) as the substrates. Chiral SFC analysis was performed on a DAICELCHIRALPAK® AD (100×3.0 mm, 3.0 μm) column, eluted with mobile phase (A: Supercritical CO₂; B: EtOH (0.1% DEA); Gradient: 5% of B for 0.5 min, then from 5% to 40% of B in 5 min, then 40% of B for 2.5 min), flowing at 1.5 mL/min, at 35° C., with pressure set at 1800 psi.

### Example 2

LCMS: 552.2 [M+H]⁺; ¹H NMR: (400 MHz, DMSO-d₆) δ 8.30 (s, 1H), 8.17 (s, 1H), 7.85-7.80 (m, 1H), 7.49-7.39 (m, 2H), 7.33-7.27 (m, 1H), 7.23-7.15 (m, 1H), 4.01 (t, J=6.4 Hz, 2H), 3.55 (s, 2H), 3.30-3.20 (m, 7H), 2.90-2.78 (m, 2H), 2.73-2.66 (m, 2H), 2.36-2.31 (m, 1H), 1.98-1.82 (m, 1H), 1.75-1.43 (m, 5H), 1.11-1.05 (m, 3H), 0.89-0.77 (m, 4H). t_{R}=4.49 min, chiral purity: 99.6%.

### Example 3

LCMS: 552.2 [M+H]⁺; ¹H NMR: (400 MHz, DMSO-d₆) δ 8.37 (s, 1H), 8.17 (s, 1H), 7.82 (s, 1H), 7.40 (t, J=7.8 Hz, 1H), 7.30-7.24 (m, 2H), 7.04 (d, J=8.1 Hz, 1H), 3.99 (t, J=6.2 Hz, 2H), 3.55 (s, 2H), 3.29-3.05 (m, 7H), 2.78-2.64 (m, 2H), 2.37-2.18 (m, 3H), 1.94-1.86 (m, 1H), 1.68-1.49 (m, 5H), 1.10 (d, J=6.4 Hz, 3H), 0.86-0.79 (m, 4H). t_{R}=4.20 min, chiral purity: 99.5%.

### Example 4

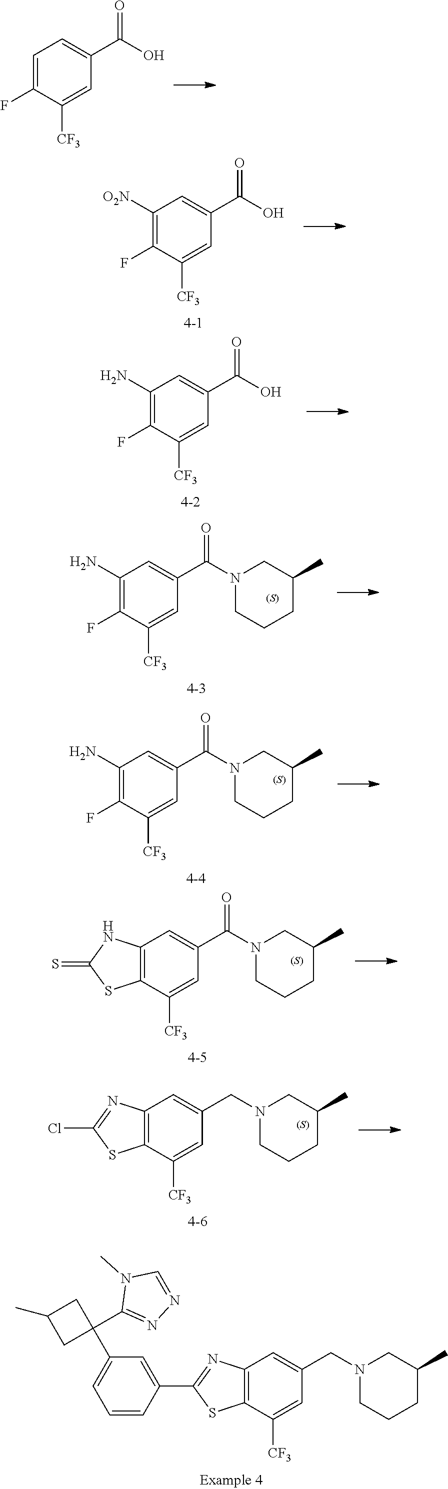

### Step 1: Preparation of Compound 4-1

To a solution of 4-fluoro-3-(trifluoromethyl)benzoic acid (6 g, 28.831 mmol) in HNO₃ (50 mL) was added sulfuric acid (7.7 mL, 144.155 mmol) at 0 T, and then the mixture was stirred at 75 T for 5 h. The reaction mixture was quenched with ice water (40 mL) and extracted with EtOAc (50 mL×2). The combined organic layers were dried, filtered and concentrated to afford Compound 4-1. LCMS: 251.9 [M−H]⁻.

### Step 2: Preparation of Compound 4-2

To a solution of Compound 4-1 (3 g, 11.853 mmol) in THF (35 mL) was added Pd/C (1.26 g, 11.853 mmol). The resulting mixture was stirred at 25° C. for 12 h under H₂ (15 psi). The suspension was filtered, and the filter cake was washed with MeOH (100 mL). The combined filtrates were concentrated to afford Compound 4-2. LCMS: 222.0 [M−H]⁻.

### Step 3: Preparation of Compound 4-3

To a solution of Compound 4-2 (2 g, 8.963 mmol) in DCM (20 mL) was added HATU (6.82 g, 17.927 mmol) and DIEA (4 mL), the mixture was stirred at room temperature for 30 min, then (3S)-3-methylhexahydropyridine hydrochloride (2.43 g, 17.927 mmol) was added to the mixture and the mixture was stirred at 25° C. for another 12 h. The reaction was quenched with water (100 mL). The resulting mixture was extracted with DCM (100 mL×2). The combined organic layers were dried with Na₂SO₄, filtered, and concentrated under reduced pressure to give a residue. The residue was purified by flash silica gel chromatography to afford Compound 4-3. LCMS: 305.2 [M+H]⁺.

### Step 4: Preparation of Compound 4-4

To a solution of Compound 4-3 (500 mg, 1.643 mmol) in THF (10 mL) was added Borane tetrahydrofuran complex solution 1.0 M in THF (8 mL, 8.216 mmol) slowly, and then the mixture was stirred at 40° C. for 12 h under N₂ atmosphere. The reaction mixture was quenched by MeOH (50 mL), and the mixture was stirred at 80° C. for 8 h, then the mixture was concentrated under vacuo to give Compound 4-4. LCMS: 291.2[M+H]*.

### Step 5: Preparation of Compound 4-5

To a solution of Compound 4-4 in DMF (10 mL) was added potassium ethoxymethanedithioate (635.15 mg, 3.962 mmol). The reaction was stirred at 120° C. for 12 h. The mixture was purified by reversed phase column chromatography directly to afford Compound 4-5. LCMS: 347.1 [M+H]⁺;

### Step 6: Preparation of Compound 4-6

A mixture of Compound 4-5 (400 mg, 1.155 mmol) in DCM (5 mL) and SOCl₂ (10 mL) was stirred at room temperature for 12 h. The reaction mixture was quenched by the addition of sat. NaHCO₃(aq) 100 mL at 0° C. The resulting mixture was extracted with EtOAc (50 mL×3). The combined organic layers were dried over anhydrous sodium sulfate, filtered, and concentrated under reduced pressure to afford Compound 4-6. LCMS: 349.1 [M+H]⁺.

### Step 7: Preparation of (S)-2-(3-(3-methyl-1-(4-methyl-4H-1,2,4-triazol-3-yl)cyclobutyl)phenyl)-5-((3-methylpiperidin-1-yl)methyl)-7-(trifluoromethyl)benzo[d]thiazole

To a solution of Compound 4-6 (100 mg, 0.287 mmol) and 4-methyl-3-(3-methyl-1-(3-(4,4,5,5-tetramethyl-1,3,2-dioxaborolan-2-yl)phenyl)cyclobutyl)-4H-1,2,4-triazole (202.56 mg, 0.573 mmol) in dioxane (3 mL) and H₂O (0.3 mL) was added K₂CO₃ (79.24 mg, 0.573 mmol) and Pd(dppf)Cl₂ (20.98 mg, 0.029 mmol). The mixture was stirred at 100° C. for 12 h under nitrogen atmosphere. The mixture was concentrated to give a residue. The residue was further purified by pre-HPLC to give Example 4. LCMS: 540.4 [M+H]⁺; ¹H NMR: (400 MHz, CDCl₃) δ 8.33-7.83 (m, 4H), 7.77 (s, 1H), 7.53-7.43 (m, 2H), 3.71 (s, 2H), 3.24 (d, J=12.7 Hz, 3H), 2.95 (s, 2H), 2.82-2.64 (m, 3H), 2.44-2.04 (m, 2H), 1.89-1.61 (m, 6H), 1.17 (t, J=6.0 Hz, 3H), 0.88 (d, J=4.5 Hz, 4H).

### Example 5: Preparation of(S)-3-(3-(3-methyl-1-(4-methyl-4H-1,2,4-triazol-3-yl)cyclobutyl)phenyl)-7-((3-methylpiperidin-1-yl)methyl)-5-(trifluoromethyl)-1H-pyrazolo[4,3-b]pyridine

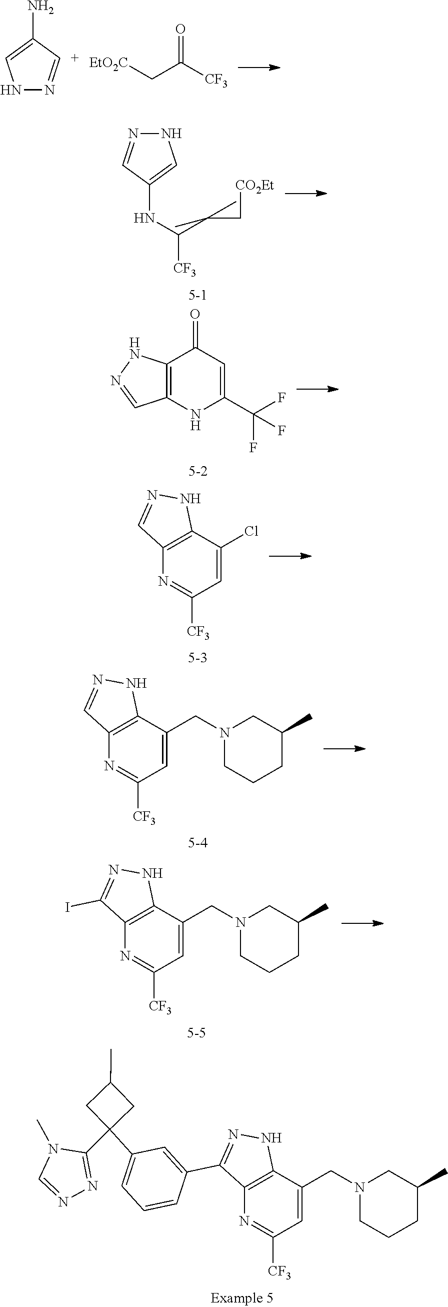

### Step 1: Preparation of Compound 5-1

A mixture of 1H-pyrazol-4-amine (10.5 g, 126.369 mmol) in toluene (50 mL) was added ethyl 4,4,4-trifluoro-3-oxobutanoate (25.59 g, 139.006 mmol), 4-methylbenzenesulfonic acid (2.18 g, 12.637 mmol). Then the mixture was stirred at 120° C. for 16 h. After cooled to room temperature, the solution was filtered. The filtrated was concentrated to afford Compound 5-1. LCMS: 250.2 [M+H]*.

### Step 2: Preparation of Compound 5-2

A mixture of Compound 5-1 (5 g, 20.065 mmol) in diphenyl ether (10 mL) was stirred at 260° C. for 1 hour. The reaction mixture was cooled to room temperature and triturated with MTBE (10 mL) at 25° C. for 2 h. The mixture was filtered. The filter cake was collected. The filtrate was concentrated to afford Compound 5-2. LCMS: 204.1 [M+H]⁺.

### Step 3: Preparation of Compound 5-3

A mixture of Compound 5-2 (1.0 g, 4.923 mmol) in POCl₃ (10 mL) was stirred at 90° C. for 1 hour. The reaction mixture was cooled and the phosphoryl chloride was removed under reduced pressure. The residue was poured onto ice water and adjusted to pH=8-9 with 10% sodium carbonate solution. The mixture was extracted with ethyl acetate (150 ml×3), the combined organic layers were dried and concentrated to afford Compound 5-3. LCMS: 222.1 [M+H]⁺.

### Step 4: Preparation of Compound 5-4

To a solution of potassium (S)-trifluoro((3-methylpiperidin-1-yl)methyl)borate (741.76 mg, 3.386 mmol) and Compound 5-3 (500 mg, 2.257 mmol) in dioxane (15 mL) and H₂O (3 mL) was added Cs₂CO₃ (2206.13 mg, 6.771 mmol), Catacxium A (80.92 mg, 0.226 mmol) and Pd(OAc)₂ (25.34 mg, 0.113 mmol). The resulting mixture was stirred for 12 h at 120° C. under N₂. The reaction mixture was concentrated to give a residue. The residue was purified by flash silica gel chromatography to afford Compound 5-4. LCMS: 299.2 [M+H]⁺.

### Step 5: Preparation of Compound 5-5

To a solution of Compound 5-4 (230 mg, 0.771 mmol) in Chloroform (5 mL) and TFA (175.82 mg, 1.542 mmol) was added NIS (173.47 mg, 0.771 mmol). The reaction solution was stirred at 20° C. for 2 h. The mixture was adjusted to pH=8 with NaHCO₃ (aq.), then sat. Na₂S₂O₃ (aq.) (20 mL) was added. After 10 min, the mixture was extracted with Ethyl acetate (50 mL×2). The combined organic layers were concentrated under reduced pressure to give a residue. The residue was purified by flash silica gel chromatography to afford Compound 5-5. LCMS: 425.1 [M+H]⁺.

### Step 6: Preparation of Example 5

To a solution of Compound 5-5 (50 mg, 0.118 mmol) and 4-methyl-3-(3-methyl-1-(3-(4,4,5,5-tetramethyl-1,3,2-dioxaborolan-2-yl)phenyl)cyclobutyl)-4H-1,2,4-triazole (83.37 mg, 0.236 mmol) in dioxane (3 mL) and H₂O (0.3 mL) was added K₂CO₃ (32.58 mg, 0.236 mmol) and Pd(dppf)Cl₂ (8.62 mg, 0.012 mmol). The mixture was stirred at 100° C. for 16 h under nitrogen atmosphere. The mixture was concentrated to give a residue. The residue was purified by pre-HPLC to afford Example 5. LCMS: 524.3 [M+H]⁺; ¹H NMR: (400 MHz, CDCl₃) δ 11.58 (br s, 1H), 8.84 (s, 1H), 8.40-8.21 (m, 1H), 7.98 (m, 1H), 7.56-7.42 (m, 2H), 7.23-7.09 (m, 1H), 3.98 (br s, 2H), 3.31 (s, 3H), 3.05-2.95 (m, 2H), 2.84-2.59 (m, 3H), 1.90-1.71 (m, 211), 1.58 (s, 6H), 1.16 (d, J=5.5 Hz, 3H), 1.01 (s, 1H), 0.90 (d, J=4.4 Hz, 3H).

### Example 6

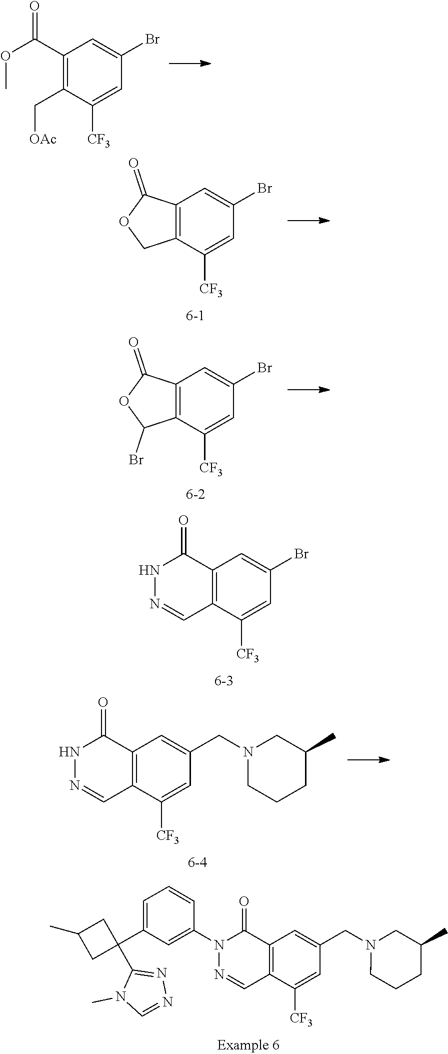

### Step 1: Preparation of Compound 6-1

To a solution of methyl 2-(acetoxymethyl)-5-bromo-3-(trifluoromethyl)benzoate (2 g, 5.632 mmol) in MeOH (6 mL) was added MeONa (0.06 g, 1.126 mmol) at room temperature. The mixture was stirred at room temperature for 2 h, then poured into 1M HCl solution (3 mL) and extracted with DCM (10 mL×3). The combined organic layers were dried, filtered, and concentrated under reduced pressure to afford Compound 6-1. ¹H NMR: (400 MHz, CDCl₃) δ 8.25 (s, 1H), 8.04 (s, 1H), 5.42 (s, 2H).

### Step 2: Preparation of Compound 6-2

To a solution of Compound 6-1 (730 mg, 2.598 mmol) in CCl₄ (7 mL) was added NBS (554.81 mg, 3.117 mmol) and BPO (62.86 mg, 0.260 mmol). The reaction mixture was stirred at 85° C. for 10 h. The reaction was quenched by the addition of H₂O (10 mL). The mixture was extracted with DCM (20 mL×3). The organic layer was washed with brine (20 mL), dried over anhydrous Na₂SO₄, filtered and concentrated to afford Compound 6-2 which was used without further purification. ¹HNMR: (400 MHz, CDCl₃) δ 8.27 (s, 1H), 8.11 (s, 1H), 7.46 (s, 1H).

### Step 3: Preparation of Compound 6-3

To a solution of Compound 6-2 (855 mg, 2.376 mmol) in EtOH (10 mL) was added hydrazine hydrate (0.577 mL, 11.878 mmol). The mixture was stirred at 80° C. for 18 h. The reaction mixture was cooled to room temperature and diluted with cold water. Then solid was precipitate out. The mixture was filtered and the filter cake was collected to afford Compound 6-3. LCMS: 293.0 [M+H]⁺.

### Step 4: Preparation of Compound 6-4

Compound 6-4 was synthesized according to step 4 in Example 5. LCMS: 326.2 [M+H]

### Step 5: Preparation of Example 6

To a solution of Compound 6-4 (150 mg, 0.461 mmol) in dioxane (2 mL) was added 2,5-diazahexane (16.26 mg, 0.184 mmol), K₂CO₃ (127.44 mg, 0.922 mmol), 3-(1-(3-bromophenyl)-3-methylcyclobutyl)-4-methyl-4H-1,2,4-triazole (141.18 mg, 0.461 mmol) and CuI (17.56 mg, 0.092 mmol). The mixture was stirred at 100° C. for 12 h under N₂ atmosphere. Then the mixture was concentrated to give a residue. The residue was purified by pre-HPLC to afford Example 6. LCMS: 551.4 [M+H]⁺; ¹H NMR: (400 MHz, DMSO-d₆) δ 8.6 (s, 2H), 8.5-8.1 (m, 2H), 7.7-7.2 (m, 4H), 3.8 (s, 2H), 3.3-3.2 (m, 3H), 3.2-3.0 (m, 1H), 3.0-2.7 (m, 3H), 2.6-2.5 (m, 2H), 2.5-2.1 (m, 2H), 1.9-1.4 (m, 5H), 1.2-1.0 (m, 3H), 0.8 (d, J=6.1 Hz, 4H).

### Example 7

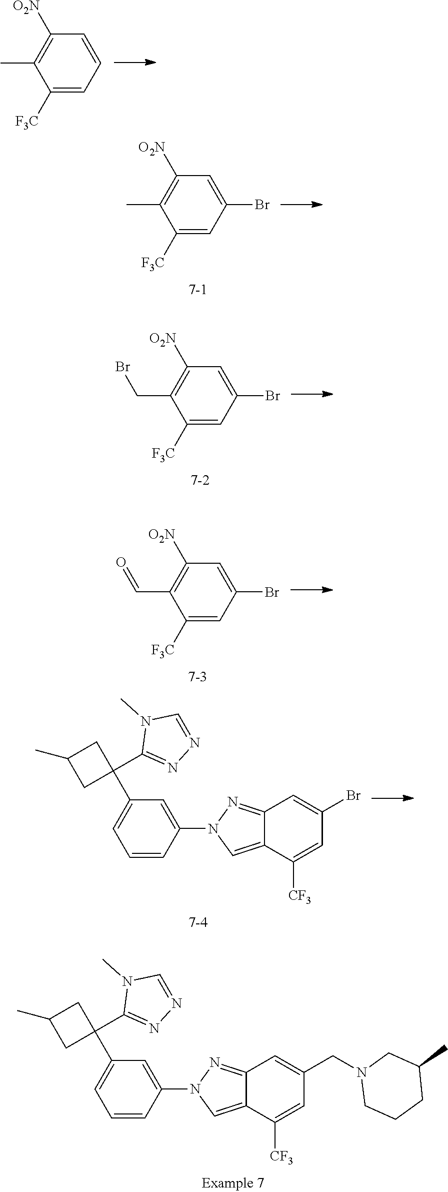

### Step 1: Preparation of Compound 7-1

To a solution of 2-methyl-1-nitro-3-(trifluoromethyl)benzene (15 g, 73.121 mmol) in concentrated H₂SO₄ (150 mL) was added 1,3-dibromo-4,4-dimethyl-2-oxotetrahydro-1H-imidazol-5-one (12.54 g, 43.872 mmol) at 0° C. Then the reaction mixture was stirred at room temperature for 3 h. The reaction mixture was poured into ice water (200 mL). After 10 min, the mixture was extracted with ethyl acetate (300 mL×3). The combined organic layers were washed with saturated NaHCO₃ (aq.), filtered, and concentrated under reduced pressure to give a residue. The residue was purified by flash silica gel chromatography to afford Compound 7-1. ¹H NMR: (400 MHz, CDCl₃) δ 8.04 (d, J=1.8 Hz, 1H), 7.99 (d, J=1.8 Hz, 1H), 2.51 (d, J=1.4 Hz, 3H).

### Step 2: Preparation of Compound 7-2

To a solution of Compound 7-1 (1.00 g, 3.521 mmol) and benzoic peroxyanhydride (0.09 g, 0.352 mmol) in tetrachloromethane (30 mL) was added NBS (0.66 g, 3.697 mmol). The mixture was stirred at 80° C. for 18 h. The reaction mixture was concentrated under reduced pressure to remove solvent. The residue was purified by flash silica gel chromatography to afford Compound 7-2. LCMS: 383.9 [M+Na]⁺.

### Step 3: Preparation of Compound 7-3

To a mixture of Compound 7-2 (1 g, 2.755 mmol) and 4 Å molecular sieves (200 mg) in MeCN (10 mL) was added 4-methyl-1,4-oxazinane 4-oxide (0.68 g, 5.841 mmol) at room temperature and the resulting mixture was stirred for 1.5 h under N₂. The mixture was diluted with ethyl acetate and filtered. The filtrate was washed with H₂O, 1N HCl. The organic layer was washed with brine 70 mL, dried over anhydrous sodium sulfate, filtered, and concentrated under reduced pressure to give a residue. The residue was purified by flash silica gel chromatography to afford Compound 7-3.

### Step 4: Preparation of Compound 7-4

To a solution of Compound 7-3 (300 mg, 0.101 mmol) in propan-2-ol (6 mL) was added 3-(3-methyl-1-(4-methyl-4H-1,2,4-triazol-3-yl)cyclobutyl)aniline (26.83 mg, 0.111 mmol), and the mixture was stirred at 80° C. for 4 h. Then tributylphosphane (93.95 mg, 0.464 mmol) was added and the resulting mixture was stirred at 80° C. for 16 h. The mixture was cooled to room temperature and diluted with EtOAc (10 mL). The organics was washed with brine (5 mL), dried over Na₂SO₄, filtered, and concentrated in vacuo. The residue was purified by flash silica gel chromatography to afford Compound 7-4. LCMS: 490.0 [M+1]⁺.

### Step 5: Preparation of Example 7

To a solution of Compound 7-4 (50 mg, 0.101 mmol), potassium (S)-trifluoro((3-methylpiperidin-1-yl)methyl)borate (44.68 mg, 0.204 mmol), Cs₂CO₃ (99.67 mg, 0.306 mmol) in dioxane (2 mL) and H₂O (1 mL) was added Ruphos Pd G₄ (9.81 mg, 0.010 mmol). And the reaction mixture was stirred at 90° C. for 16 h under N₂. The mixture was concentrated to give a residue, which was purified by pre-HPLC to afford Example 7. LCMS: 523.2 [M+H]⁺; ¹H NMR: (400 MHz, Methanol-d₄) δ 8.5-8.3 (m, 2H), 8.0-7.4 (m, 6H), 4.0 (s, 2H), 3.4-3.4 (m, 3H), 3.2-2.9 (m, 4H), 2.8-2.4 (m, 4H), 2.2-2.0 (m, 1H), 1.9-1.6 (m, 4H), 1.2 (d, J=6.1 Hz, 3H), 1.1-0.9 (m, 4H).

### Example 8

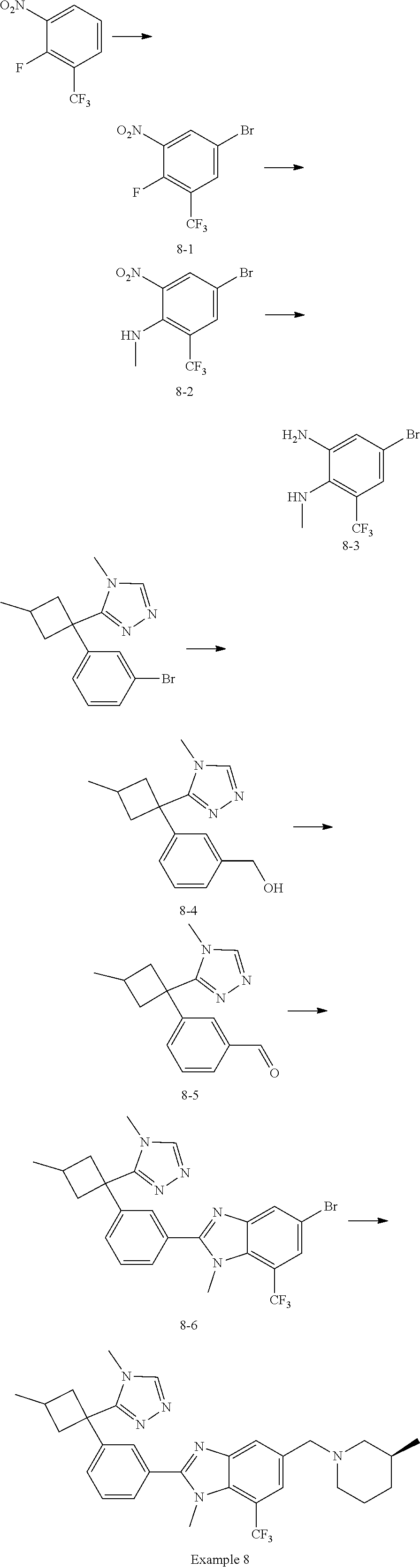

### Step 1: Preparation of Compound 8-1

To a solution of 2-fluoro-1-nitro-3-(trifluoromethyl)benzene (6.67 g, 31.899 mmol) in TFA (20 mL) and H₂SO₄ (30 mL) was added NBS (6.81 g, 38.278 mmol). The reaction mixture was stirred at 60° C. for 12 h. The mixture was cooled to room temperature and diluted with ice water (100 mL). Then the mixture was extracted with ethyl acetate (40 mL×3). the organic layer was separated and washed with a saturated sodium bicarbonate aqueous solution and brine, dried over anhydrous sodium sulfate, and concentrated under reduced pressure to afford a residue, which was purified by flash silica gel chromatography to afford Compound 8-1.

### Step 2: Preparation of Compound 8-2

To a solution of Compound 8-1 (2 g, 6.944 mmol) in MeOH (5 mL) was added MeNH₂ in MeOH (9.2 mL, 69.444 mmol) at 25° C. under N₂. The reaction was stirred at 25° C. for 18 h. The mixture was diluted with H₂O (20 mL) and extracted with EtOAc (40 mL×3). The combined organic layers were washed with brine (50 mL), dried with anhydrous sodium sulfate, and concentrated under reduced pressure. The residue was purified by flash silica gel chromatography to afford Compound 8-2. LCMS: 299.0 [M+H]⁺.

### Step 3: Preparation of Compound 8-3

To a solution of Compound 8-2 (200 mg, 0.669 mmol) in EtOH (1 mL) and EA (2 mL) was added Tin chloride dihydrate (450.90 mg, 2.004 mmol) at 25° C. under N₂. The reaction was stirred at 70° C. for 3 h. The mixture was concentrated under reduced pressure to give a residue. The residue was purified by Reversed Phase Chromatography to afford Compound 8-3. LCMS: 269.0 [M+H]⁺.

### Step 4: Preparation of Compound 8-4

To a solution of 3-(1-(3-bromophenyl)-3-methylcyclobutyl)-4-methyl-4H-1,2,4-triazole (1 g, 3.266 mmol) in 1,4-dioxane (12 mL) was added (tributylstannyl) methanol (1.58 g, 4.899 mmol), XPhos Pd G₂ (0.26 g, 0.327 mmol) at room temperature, and the reaction mixture was stirred at 90° C. for 18 h under N₂. The mixture was filtered through a celite pad, and the filtrate was concentrated to give a residue. The residue was purified by flash silica gel chromatography to afford Compound 8-4. LCMS: 258.3 [M+H]⁺.

### Step 5: Preparation of Compound 8-5

To a solution of Compound 8-4 (789 mg, 3.066 mmol) in DCM (10 mL) at 0° C., was added DMP (197.78 mg, 0.466 mmol), and the mixture was stirred at room temperature for 2 h. The reaction mixture was diluted with Na₂SO₃ (aq., 10 mL) and extracted with DCM (15 mL×2). The combined organic layers were washed with brine (20 mL), dried over anhydrous sodium sulfate, filtered, and concentrated under reduced pressure to give a residue. The residue was purified by flash silica gel chromatography to afford Compound 8-5. LCMS: 256.3 [M+H]⁺.

### Step 6: Preparation of Compound 8-6

To a stirred solution of Compound 8-5 (300 mg, 1.115 mmol) and Compound 8-3 (284.67 mg, 1.115 mmol) in DMF (5 mL) and H₂O (1 mL) at room temperature was added Oxone (411.24 mg, 0.669 mmol) in portions over 15 min. The mixture was stirred at room temperature for overnight. The reaction mixture was diluted with water (20 mL) and extracted with DCM (20 mL×2). The combined organic layers were washed with brine (30 mL), dried over anhydrous sodium sulfate, filtered, and concentrated under reduced pressure to give a residue. The residue was purified by flash silica gel chromatography to afford Compound 8-6. LCMS: 503.8 [M+H]⁺.

### Step 7: Preparation of Example 8

Example 8 was synthesized according to step 5 in Example 7. LCMS: 537.3 [M+H]⁺; ¹H NMR: (400 MHz, Chloroform-d) δ 8.1-7.7 (m, 4H), 7.6-7.4 (m, 3H), 4.2 (s, 2H), 3.9 (s, 3H), 3.5-3.1 (m, 5H), 3.0-2.6 (m, 4H), 2.5-2.2 (m, 2H), 2.1-1.7 (m, 5H), 1.2 (d, J=4.4 Hz, 3H), 1.0-0.8 (m, 4H).

### Example 9

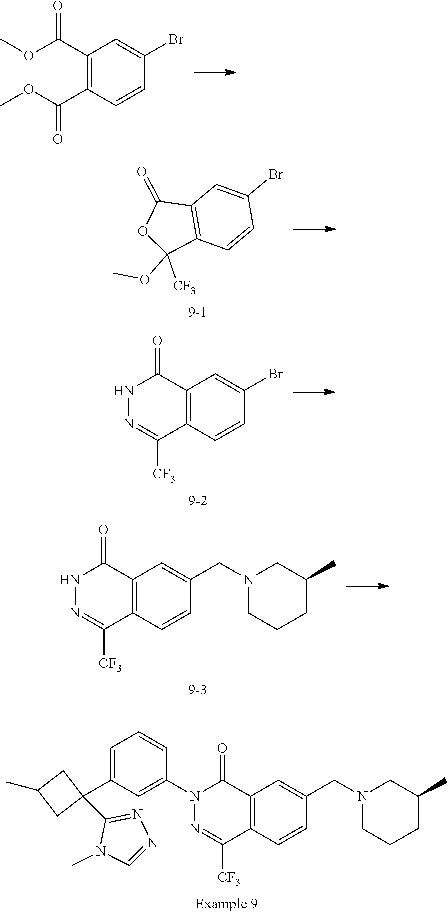

### Step 1: Preparation of Compound 9-1

To a solution of dimethyl 4-bromophthalate (5 g, 18.310 mmol) in 1,2-dichloroethane (50 mL) was added Cesium fluoride (0.56 g, 3.662 mmol), trimethyl(trifluoromethyl)silane (3.2 mL, 21.972 mmol) at 0° C., and then the mixture was stirred at rt for 1 hr. The mixture was then partitioned between ethyl acetate (100 mL) and water (15.0 mL). The organic phase was separated, washed with brine (15.0 mL×3), dried over anhydrous sodium sulfate, filtered, and concentrated under reduced pressure to give Compound 9-1.

### Step 2: Preparation of Compound 9-2

To a solution of Compound 9-1 (4 g, 12.860 mmol) in THF (5 mL) was added hydrazine hydrate (1.3 mL, 25.719 mmol) at rt, then the mixture was stirred at 75° C. for 12 h. The mixture was concentrated to give a residue. The residue was purified by flash silica gel chromatography to afford Compound 9-2. LCMS: 293.0 [M+H]⁺.

### Step 3: Preparation of Compound 9-3

To a mixture of Compound 9-2 (300 mg, 1.024 mmol), potassium (S)-trifluoro((3-methylpiperidin-1-yl)methyl)borate (224.30 mg, 1.024 mmol) in H₂O (0.5 mL) and dioxane (5 mL) was added K₃PO₄ (434.60 mg, 2.048 mmol), Ruphos Pd G₄ (435.30 mg, 0.512 mmol), the mixture was stirred at 100° C. for 12 h. The mixture was then partitioned between ethyl acetate (100 mL) and water (15.0 mL). The organic phase was separated, washed with brine (15.0 mL×3), dried over anhydrous sodium sulfate, filtered, and concentrated under reduced pressure to give a residue. The residue was purified by flash silica gel chromatography to afford Compound 9-3. LCMS: 326.2 [M+H]⁺.

### Step 4: Preparation of Example 9

To a solution of Compound 9-3 (50 mg, 0.154 mmol), 3-(1-(3-bromophenyl)-3-methylcyclobutyl)-4-methyl-4H-1,2,4-triazole (47.06 mg, 0.154 mmol) in dioxane (5 mL) was added CuI (5.85 mg, 0.031 mmol), K₂CO₃ (42.48 mg, 0.307 mmol) and 2,5-diazahexane (5.42 mg, 0.061 mmol). The mixture was stirred at 100° C. for 12 h under N₂. The mixture was concentrated under reduced pressure to give a residue, which was further purified by prep-HPLC to afford Example 9. LCMS: 551.4 [M+H]⁺; ¹H NMR: (400 MHz, Chloroform-d) b 8.7-8.0 (m, 4H), 7.9-7.3 (m, 4H), 4.4-4.0 (m, 2H), 3.6-3.1 (m, 5H), 3.0-2.3 (m, 6H), 2.2-1.6 (m, 5H), 1.3-1.1 (m, 3H), 1.1 (s, 1H), 0.9 (d, J=5.4 Hz, 3H).

### Example 10

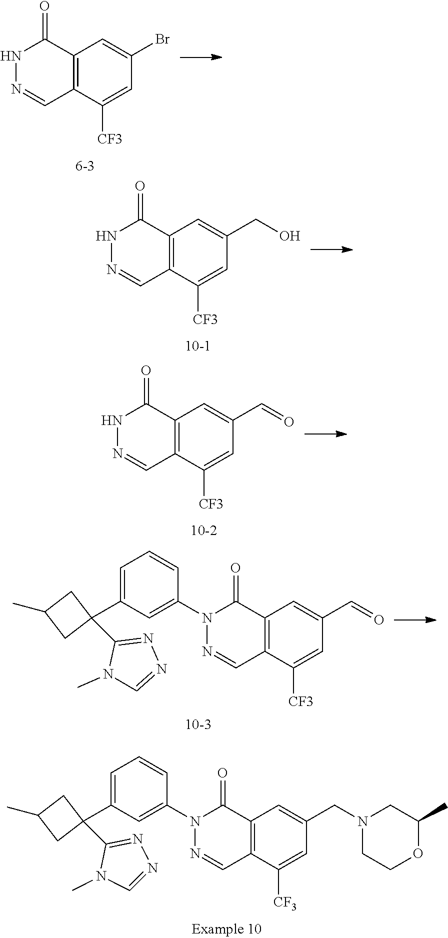

### Step 1: Preparation of Compound 10-1

To a solution of Compound 6-3 (500 mg, 1.706 mmol) in dioxane (8 mL) were added (Tributylstannyl)methanol (1095 mg, 3.412 mmol), and XPhos Pd G₂ (8 mg, 0.010 mmol). The reaction was stirred at 80° C. for overnight under N₂. The reaction mixture was concentrated under reduced pressure to give a residue. The residue was purified by flash silica gel chromatography to afford Compound 10-1. LCMS: 245.0 [M+H]⁺.

### Step 2: Preparation of Compound 10-2

To a solution of Compound 10-1 (200 mg, 0.819 mmol) in DCM (10 mL) were added DMP (521 mg, 1.229 mmol), and the reaction was stirred at room temperature for 2 h. The reaction was diluted with DCM (20 mL), then washed with saturated Na₂SO₃ (10 M), saturated aqueous NaHCO₃ (10 mL) and brine. The organic layer was purified by flash silica gel chromatography to afford Compound 10-2. LCMS: 243.0 [M+H]⁺.

### Step 3: Preparation of Compound 10-3

To a solution of Compound 10-2 (100 mg, 0.413 mmol) in dioxane (5 mL) were added 3-(1-(3-bromophenyl)-3-methylcyclobutyl)-4-methyl-4H-1,2,4-triazole (151.74 mg, 0.496 mmol), K₂CO₃ (114.14 mg, 0.826 mmol), DMEDA (14.56 mg, 0.165 mmol) and Cut (15.73 mg, 0.083 mmol). The reaction was stirred at 100° C. for 16 h under N₂. The residue was purified by flash silica gel chromatography to afford Compound 10-3. LCMS: 468.1 [M+H]⁺.

### Step 4: Preparation of Example 10

To a solution of Compound 10-3 (16 mg, 0.034 mmol) in DCE (2 mL) was added CH₃COONa (3 mg, 0.041 mmol) and AcOH (one drop). The mixture was stirred at room temperature for 30 min. Sodium Triacetoxyborohydride (21 mg, 0.103 mmol) was added to the mixture, then the reaction was stirred at room temperature for overnight. The reaction mixture was concentrated under reduced pressure to afford a residue, which was further purified by pre-HPLC to afford Example 10. LCMS: 553.2 [M+H]⁺; ¹H NMR: (400 MHz, Chloroform-d) δ 8.63 (s, 1H), 8.56 (s, 1H), 8.26 (s, 1H), 7.70 (s, 1H), 7.58-7.34 (m, 4H), 3.93-3.65 (m, 5H), 3.26 (s, 3H), 2.92-2.64 (m, 6H), 2.38-1.99 (m, 3H), 1.18-1.09 (m, 6H).

### Example 11

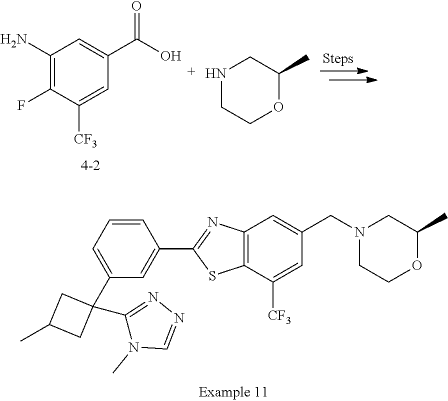

### Step 1: Preparation of Compound 11-1

According to procedures of Example 4, Example 11 was synthesized with Compound 4-2 (300 mg, 1.345 mmol) and (2R)-2-methyl-1,4-oxazinane (136.00 mg, 1.345 mmol) as the substrates. LCMS: 542.2 [M+H]⁺; ¹HNMR: (400 MHz, DMSO-d₆) δ 8.40-8.29 (m, 2H), 8.08-7.97 (m, 1H), 7.89-7.82 (m, 2H), 7.69-7.47 (m, 2H), 3.83-3.64 (m, 3H), 3.61-3.48 (m, 2H), 3.26-3.19 (m, 3H), 2.98-2.87 (m, 2H), 2.77-2.56 (m, 4H), 2.39-2.26 (m, 1H), 2.18-2.06 (m, 1H), 1.83 (t, J 10.7 Hz, 1H), 1.15-1.08 (m, 3H), 1.04 (d, J 6.2 Hz, 3H).

### Example 12

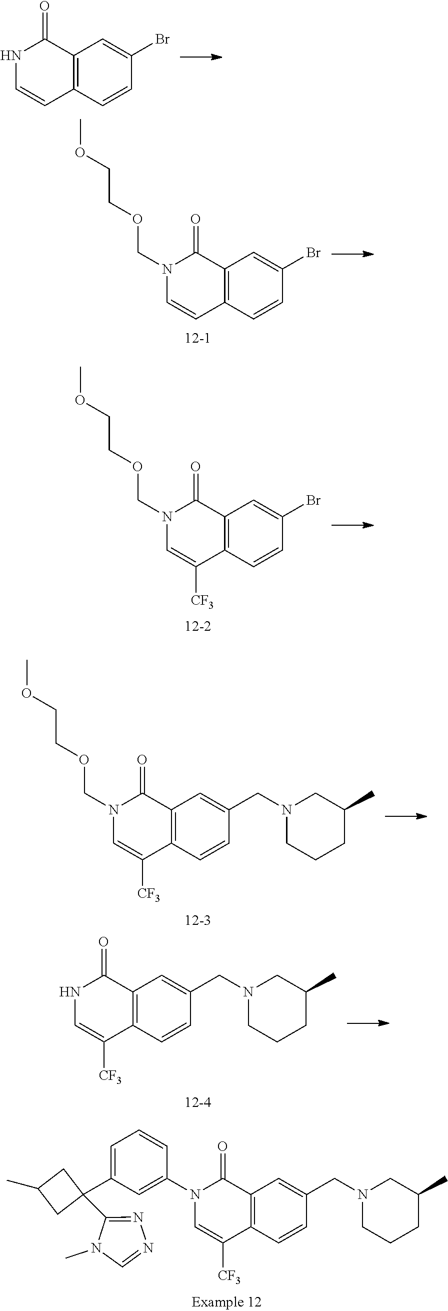

### Step 1: Preparation of Compound 12-1

To a solution of 7-bromoisoquinolin-1(2H)-one (1 g, 4.463 mmol) in THF (20 mL) was added potassium 2-methylpropan-2-olate (in THF, 4.9 mL, 4.909 mmol) at 0° C. The solution was stirred for 20 min. Then 1-chloro-2,5-dioxahexane (0.56 g, 4.463 mmol) was added to the mixture. The resulting mixture was stirred at rt for 40 min. The mixture was concentrated under vacuo to give a residue, which was further purified by flash silica gel chromatography to afford Compound 12-1.

### Step 2: Preparation of Compound 12-2

To a solution of Compound 12-1 (1.4 g, 4.485 mmol) in MeCN (10 mL) was added [bis(trifluoroacetoxy)iodo]benzene (5.81 g, 13.455 mmol), cupric acetate (0.45 g, 2.242 mmol), potassium fluoride (0.78 g, 13.455 mmol) and trimethyl(trifluoromethyl)silane (3.0 mL, 20.182 mmol). The resulting mixture was stirred at 40° C. for 12 h. The mixture was then partitioned between ethyl acetate (100 mL) and water (15.0 mL). The organic layer was washed with brine (100 mL), dried over anhydrous Na₂SO₄, filtered, and concentrated to afford a residue, which was further purified by flash silica gel chromatography to afford Compound 12-2. LCMS: 380.1 [M+H]⁺.

### Step 3: Preparation of Compound 12-3

To a solution of Compound 12-2 (200 mg, 0.526 mmol) and potassium (S)-trifluoro((3-methylpiperidin-1-yl)methyl)borate (115.27 mg, 0.526 mmol) in H₂O (0.5 mL) and dioxane (5 mL) was added Ruphos Pd G₄ (50.61 mg, 0.053 mmol) and Cs₂CO₃ (514.24 mg, 1.578 mmol). The resulting mixture was stirred at 90° C. for 18 h. The mixture was then partitioned between ethyl acetate (100 mL) and water (20.0 mL). The organic layer was washed with brine (100 mL), dried over anhydrous Na₂SO₄, filtered, and concentrated to afford a residue. The residue was purified by flash silica gel chromatography to afford Compound 12-3. LCMS: 413.3 [M+H]⁺.

### Step 4: Preparation of Compound 12-4

A mixture of Compound 12-3 (200 mg, 0.485 mmol) in H₂SO₄ (1 mL) was stirred at 90° C. for 5 h. The mixture was concentrated under vacuo to give the crude Compound 12-4, which could be used for the next step without further purification. LCMS: 325.3 [M+H]⁺.

### Step 5: Preparation of Example 12

To a solution of Compound 12-4 (30 mg, 0.092 mmol) and 3-(1-(3-bromophenyl)-3-methylcyclobutyl)-4-methyl-4H-1,2,4-triazole (28.32 mg, 0.092 mmol) in dioxane (1 mL) was added K₂CO₃ (25.43 mg, 0.184 mmol), CuI (3.50 mg, 0.018 mmol) and DMEDA (3.24 mg, 0.037 mmol). The mixture was stirred at 120° C. for 12 h under N₂. The mixture was concentrated under reduced pressure. The resulting residue was purified by prep-HPLC to afford Example 12. LCMS: 550.4 [M+H]⁺; ¹H NMR: (400 MHz, Chloroform-d) δ 8.38 (s, 1H), 8.09-7.81 (m, 2H), 7.64 (s, 1H), 7.55-7.21 (m, 5H), 3.78-3.48 (m, 2H), 3.37-3.22 (m, 3H), 2.98-2.60 (m, 6H), 2.13-1.61 (m, 7H), 1.15 (d, J=4.7 Hz, 3H), 0.96-0.89 (m, 1H), 0.85 (d, J=5.8 Hz, 3H).

### Example 13

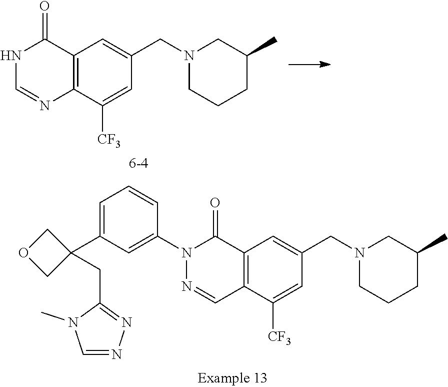

To a solution of Compound 6-4 (30 mg, 0.092 mmol) in dioxane (2 mL) were added 3-((3-(3-bromophenyl)oxetan-3-yl)methyl)-4-methyl-4H-1,2,4-triazole (28 mg, 0.092 mmol), K₂CO₃ (25.49 mg, 0.184 mmol), DMEDA (4.06 mg, 0.046 mmol) and CuI (3.51 mg, 0.018 mmol). The reaction was stirred at 100° C. for 16 h under N₂. The residue was purified by pre-HPLC to afford Example 13. LCMS: 553.2 [M+H]⁺; ¹H NMR: (400 MHz, DMSO-d₆) δ 8.54 (s, 2H), 8.33-8.26 (m, 1H), 8.22 (s, 1H), 7.55-7.38 (m, 2H), 7.22 (s, 1H), 7.01 (s, 1H), 4.95 (d, J=6.1 Hz, 2H), 4.86 (d, J=6.0 Hz, 2H), 3.51 (s, 2H), 2.95 (s, 3H), 2.79-2.43 (m, 6H), 1.76-1.53 (m, 4H), 0.97-0.76 (m, 4H).

### Example 14&15

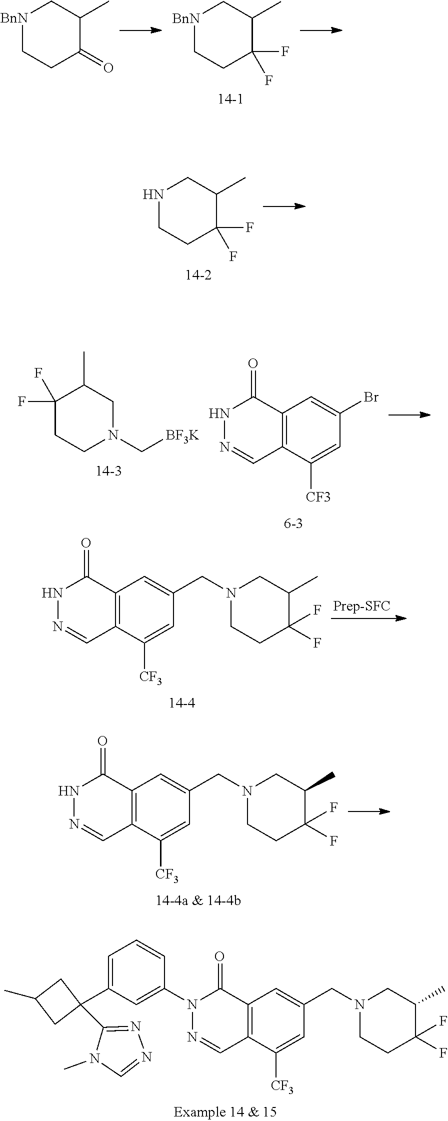

### Step 1: Preparation of Compound 14-1

To a solution of 1-benzyl-3-methylpiperidin-4-one (5 g, 24.597 mmol) in DCM (50 mL) was added DAST (16.2 mL, 122.983 mmol) at rt. The resulting solution was heated to 40° C. and stirred for 14 h. After cooling to the rt, the reaction mixture was added dropwise into ice water (100 mL), followed by the addition of saturated aqueous NaHCO₃ (20 mL). The resulting mixture was extracted with DCM (3×100 mL), and the combined organic layers were washed with brine (50 mL), dried over anhydrous Na₂SO₄, filtered, and concentrated under reduced pressure to afford a residue, which was further purified by silica gel chromatography to afford Compound 14-1. LCMS: 226.2 [M+H]⁺.

### Step 2: Preparation of Compound 14-2

To a solution of Compound 14-1 (0.9 g, 3.995 mmol) in MeOH (20 mL) was added Pd/C (1.28 g) and 1,1,2-trichloroethane (2.66 g, 19.975 mmol). The reaction was stirred at room temperature for 12 h under H₂. The reaction mixture was concentrated under reduced pressure to afford Compound 14-2. LCMS: 136.0 [M+H]⁺.

### Step 3: Preparation of Compound 14-3

To a solution of Compound 14-2 (500 mg, 3.329 mmol) in THF (20 mL) was added Potassium(bromomethyl)trifluoroborate (193.16 mg, 0.962 mmol), KHCO₃ (787.49 mg, 7.866 mmol) and KI (43.53 mg, 0.262 mmol). The reaction was stirred at 80° C. for 4 h. The reaction mixture was concentrated to afford Compound 14-3, which was used to next step directly.

### Step 4: Preparation of Compound 14-4

To a solution of Compound 6-3 (200 mg, 0.683 mmol) in dioxane/water (5 mL, 4:1) was added Cs₂CO₃ (444 mg, 1.365 mmol), Compound 14-3 (174 mg, 0.683 mmol) and Ruphos Pd G₄ (6 mg, 0.007 mmol). The resulting mixture was stirred at 100° C. for 16 h. The residue was purified by silica gel chromatography to afford Compound 14-4. LCMS: 362.0 [M+H]⁺; Step 5: Preparation of Compound 14-4a/14-4b Compound 14-4 was further separated by pre-SFC (performed on a DAICELCHIRALPAK®IG (250×25 mm, 10 m) column, eluted with mobile phase (A: Supercritical CO₂; B: MEOH (+0.1% 7.0 mol/l Ammonia in MEOH); A/B=90/10), flowing at 80 mL/min, at room temperature, with pressure set at 100 bar) to afford Compound 14-4a (6.72 min) and Compound 14-4b (7.9 min). LCMS: 362.0 [M+H]⁺.

### Step 6: Preparation of Example 14 and Example 15

A solution of Compound 14-4a (20 mg, 0.055 mmol), 3-(1-(3-bromophenyl)-3-methylcyclobutyl)-4-methyl-4H-1,2,4-triazole (16 mg, 0.055 mmol), K₂CO₃ (22 mg, 0.166 mmol), DMEDA (2 mg, 0.022 mmol) and CuI (2.5 mg, 0.011 mmol) in dioxane (3 mL) was stirred at 100° C. for 16 h under N₂. The mixture was purified by pre-HPLC to afford Example 14. LCMS: 587.2 [M+H]⁺; ¹H NMR: (400 MHz, Chloroform-d) δ 8.62 (s, 1H), 8.56 (s, 1H), 8.18 (s, 1H), 8.06-7.91 (m, 1H), 7.70 (s, 1H), 7.58-7.43 (m, 2H), 7.37 (d, J=7.6 Hz, 1H), 3.74 (s, 2H), 3.36-3.16 (m, 3H), 2.90 (s, 2H), 2.77 (s, 1H), 2.69 (d, J=5.8 Hz, 4H), 2.47-1.99 (m, 5H), 1.24-1.09 (m, 3H), 1.01 (d, J=5.8 Hz, 3H).

Example 15 was synthesized according to a similar procedure of synthesizing Example 14 with Compound 14-4b as the substrate. LCMS: 587.2 [M+H]⁺; ¹H NMR: (400 MHz, Chloroform-d) δ 8.62 (s, 1H), 8.56 (s, 1H), 8.18 (s, 11H), 8.07-7.92 (m, 11H), 7.70 (s, 1H), 7.60-7.43 (m, 2H), 7.37 (d, J=7.9 Hz, 11H), 3.74 (s, 2H), 3.33-3.14 (m, 3H), 2.96-2.82 (m, 2H), 2.82-2.74 (m, 11H), 2.74-2.63 (m, 4H), 2.46-1.94 (m, 5H), 1.25-1.08 (m, 3H), 1.01 (d, J=5.9 Hz, 3H).

### Example 16

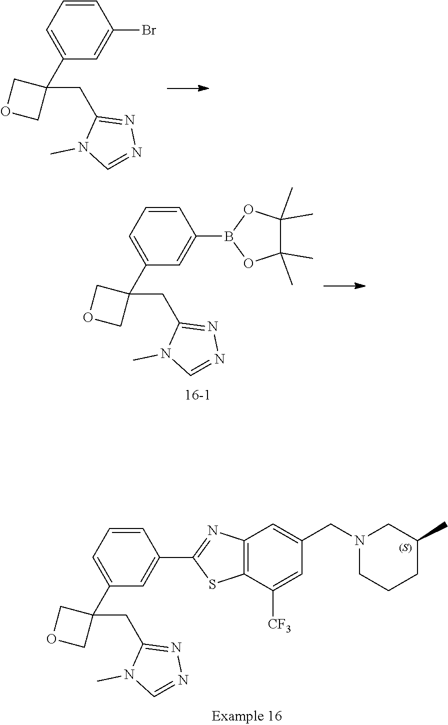

### Step 1: Preparation of Compound 16-1

To a solution of 3-((3-(3-bromophenyl)oxetan-3-yl)methyl)-4-methyl-4H-1,2,4-triazole (150 mg, 0.487 mmol), 4,4,5,5-tetramethyl-2-(4,4,5,5-tetramethyl-1,3,2-dioxaborolan-2-yl)-1,3,2-dioxaborolane (247.20 mg, 0.973 mmol), KOAc (95.54 mg, 0.973 mmol) in dioxane (3 mL) was added Pd(dppf)Cl₂ (35.61 mg, 0.049 mmol). The mixture was stirred at 80° C. for 16 h under N₂. The reaction mixture was cooled to room temperature, quenched with water (5 mL), and extracted with dichloromethane (10 mL×2). The combine organic layers were concentrated under reduced pressure to give a residue, which was purified by prep-TLC to give Compound 16-1. LCMS: 356.0 [M+H]⁺.

### Step 2: Preparation of Example 16

To a solution of Compound 4-6 (9.82 mg, 0.028 mmol) and Compound 16-1 (20 mg, 0.059 mmol) in dioxane (1 mL) and H₂O (0.2 mL) were added K₂CO₃ (7.78 mg, 0.056 mmol) and Pd(dppt)Cl₂ (2.06 mg, 0.003 mmol). The mixture was stirred at 80° C. for 12 h under nitrogen atmosphere. The reaction mixture was filtered and concentrated. The residue was purified prep-HPLC to give Example 16. LCMS: 542.2 [M+H]⁺; ¹H NMR: (400 MHz, Chloroform-d) δ 8.30 (s, 1H), 8.03-7.80 (m, 2H), 7.78-7.62 (m, 2H), 7.40 (t, J=7.5 Hz, 1H), 7.05-6.89 (m, 1H), 5.31-5.01 (m, 4H), 3.62 (s, 2H), 2.93 (s, 3H), 2.00-1.70 (m, 5H), 1.34-1.17 (m, 4H), 0.98-0.78 (m, 5H).

### Example 17

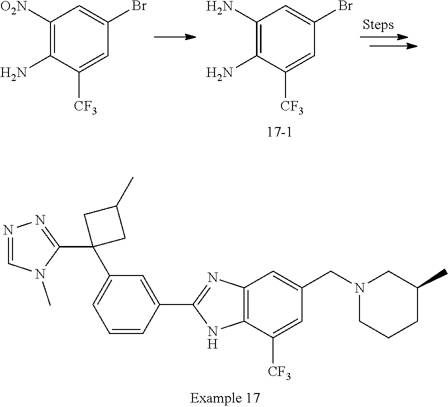 [SMILES: Nc1c([N+](=O)[O-])cc(Br)cc1C(F)(F)F ; Nc1cc(Br)cc(C(F)(F)F)c1N ; CC1CC(c2cccc(-c3nc4cc(CN5CCC[C@H](C)C5)cc(C(F)(F)F)c4[nH]3)c2)(c2nncn2C)C1]

### Step 1: Preparation of Compound 17-1

To a solution of 4-bromo-2-nitro-6-(trifluoromethyl)aniline (500 mg, 1.754 mmol) in EtOH (5 mL) and EA (2.5 mL) was added Tin chloride dihydrate (427.48 mg, 1.895 mmol) at 25° C. under N₂. The reaction was stirred at 70° C. for 3 h. The mixture was concentrated under reduced pressure to give a residue. The residue was purified by Reversed Phase Chromatography to afford Compound 17-1. LCMS: 254.0 [M+H]⁺.

### Step 2: Preparation of Example 17

According to the procedures of Example 8, Example 17 was synthesized with Compound 17-1 and Compound 8-5 as the substrates. LCMS: 523.0 [M+H]⁺; ¹H NMR: (400 MHz, DMSO-d₆) δ 8.41-8.26 (m, 1H), 8.23-7.98 (m, 2H), 7.71 (s, 1H), 7.58-7.27 (m, 3H), 3.60 (s, 2H), 3.21-3.14 (m, 4H), 2.97-2.86 (m, 2H), 2.74 (t, J=7.9 Hz, 2H), 2.62-2.53 (m, 3H), 1.90 (t, J=10.7 Hz, 1H), 1.71-1.54 (m, 4H), 1.53-1.41 (m, 1H), 1.16-1.06 (m, 3H), 0.91-0.77 (m, 4H).

### Example 18 and 19

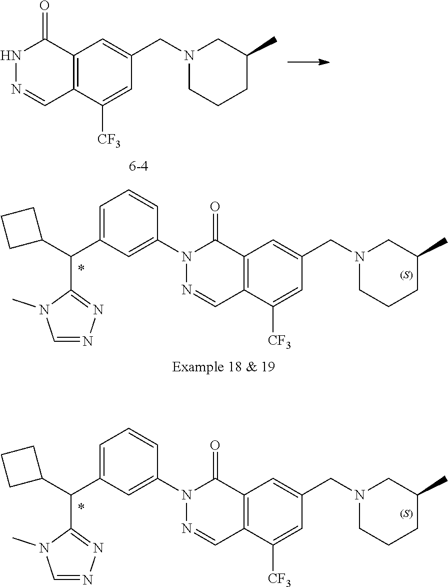

To a solution of Compound 6-4 (50 mg, 0.154 mmol), K₂CO₃ (42.48 mg, 0.307 mmol), 3-((3-bromophenyl)(cyclobutyl)methyl)-4-methyl-4H-1,2,4-triazole (70.59 mg, 0.231 mmol) in 1, 4-dioxane (2 mL) was added 2, 5-diazahexane (5.42 mg, 0.061 mmol) and CuI (5.85 mg, 0.031 mmol). The mixture was stirred at 100° C. for 16 h under N₂ atmosphere. The mixture was concentrated to give a residue. The residue was purified by pre-HPLC to give desired compound which was further separated by SFC to afford Example 18 and 19. Chiral SFC analysis was performed on a DAICELCHIRALPAK®IB (100×3.0 mm, 3.0 μm) column, eluted with mobile phase (A: Supercritical CO₂; B: MeOH (0.10% DEA); 40% of B for 8 min), flowing at 1.5 mL/min, at 35° C., with pressure set at 1800 psi.

### Example 18

LCMS: 551.6 [M+H]⁺; ¹H NMR: (400 MHz, DMSO-d₆) δ 8.54 (d, J=6.6 Hz, 2H), 8.34 (s, 1H), 8.26 (s, 1H), 7.54-7.43 (m, 3H), 7.33 (d, J=7.2 Hz, 1H), 4.29 (d, J=10.4 Hz, 1H), 3.73 (s, 2H), 3.44 (s, 3H), 3.23-3.11 (m, 1H), 2.76-2.66 (m, 2H), 2.15-1.95 (m, 2H), 1.83-1.41 (m, 6H), 1.29-1.18 (m, 4H), 0.88-0.78 (m, 4H). t_{R}=4.40 min; Chiral purity: 99.9%.

### Example 19

LCMS: 551.6 [M+H]⁺; ¹H NMR: (400 MHz, DMSO-d₆) δ 8.53 (d, J=6.3 Hz, 2H), 8.34 (s, 1H), 8.26 (s, 1H), 7.55-7.43 (m, 3H), 7.36-7.31 (m, 1H), 4.29 (d, J=10.4 Hz, 1H), 3.73 (s, 2H), 3.44 (s, 3H), 3.21-3.14 (m, 1H), 2.70 (d, J=21.7 Hz, 2H), 2.03 (d, J=46.9 Hz, 2H), 1.85-1.42 (m, 6H), 1.28-1.19 (m, 4H), 0.87-0.78 (m, 4H). t_{R}=4.65 min; Chiral purity: 99.9%.

### Example 20

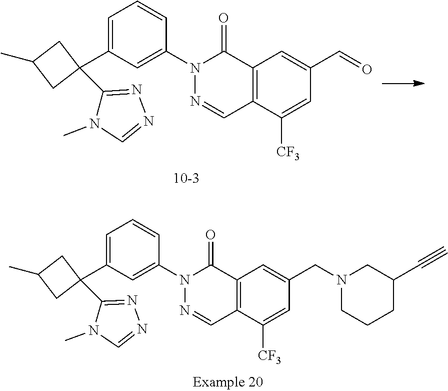 [SMILES: CC1CC(c2cccc(-n3ncc4c(C(F)(F)F)cc(C=O)cc4c3=O)c2)(c2nncn2C)C1 ; C#CC1CCCN(Cc2cc(C(F)(F)F)c3cnn(-c4cccc(C5(c6nncn6C)CC(C)C5)c4)c(=O)c3c2)C1]

To a solution of Compound 10-3 (30 mg, 0.064 mmol), 3-ethynylhexahydropyridine chlorate (11.18 mg, 0.077 mmol) in MeOH (1 mL) and DCM (1 mL) was added AcOH (1 drop). The mixture was stirred at room temperature for 0.5 h, followed by the addition of 2-methylpyridine borane (10.27 mg, 0.096 mmol). Then the reaction mixture was stirred at room temperature for 16 h. The mixture was further purified by pre-HPLC to afford Example 20. LCMS: 561.4 [M+H]⁺; ¹H NMR: (400 MHz, Chloroform-d) δ 8.73-8.45 (m, 2H), 8.14-7.92 (m, 1H), 7.73-7.26 (m, 5H), 3.97-3.39 (m, 2H), 3.36-3.07 (m, 4H), 3.01-2.55 (m, 7H), 2.44-1.67 (m, 7H), 1.18-1.11 (m, 3H).

### Example 21

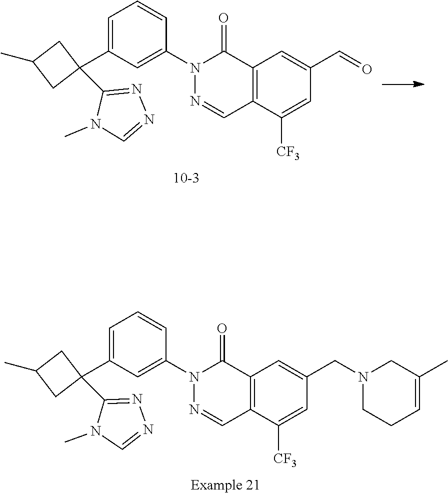

Example 21 was synthesized according a similar procedure with Example 20. LCMS: 549.2 [M+H]⁺; ¹H NMR: (400 MHz, Chloroform-d) δ 8.64 (s, 1H), 8.56 (s, 1H), 8.37 (s, 1H), 8.06-7.96 (m, 1H), 7.72-7.45 (m, 3H), 7.36 (d, J=7.8 Hz, 1H), 5.52 (s, 1H), 4.07-3.75 (m, 2H), 3.35-3.13 (m, 4H), 3.06-2.79 (m, 4H), 2.73-2.62 (m, 4H), 2.33-2.18 (m, 2H), 1.68-1.56 (m, 3H), 1.14 (t, J=5.4 Hz, 3H).

### Example 22

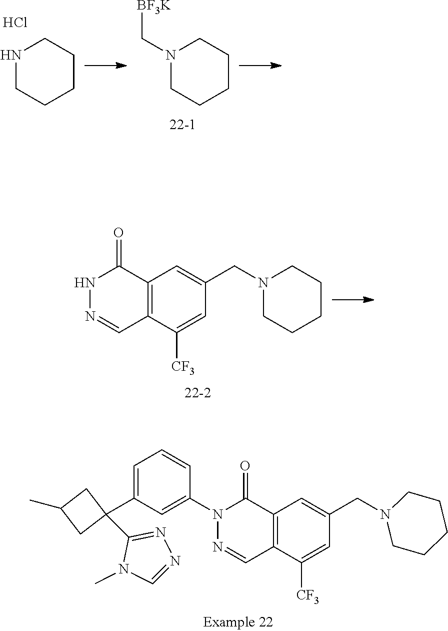

### Step 1: Preparation of Compound 22-1

To a solution of piperidine hydrochloride (714.09 mg, 5.872 mmol) in THF (10 mL) was added Potassium(bromomethyl)trifluoroborate (1179.10 mg, 5.872 mmol), K₂CO₃ (1623.02 mg, 11.744 mmol) and KI (97.48 mg, 0.587 mmol). The mixture was stirred at 80° C. for 4 h. The solid was remove by filtration and the filtrate was concentrated to afford Compound 22-1 which was used to next step directly.

### Step 2: Preparation of Compound 22-2

To a solution of Compound 22-1 (400 mg, 1.952 mmol), 7-bromo-5-(trifluoromethyl)phthalazin-1(2H)-one (160 mg, 0.546 mmol) and Cs₂CO₃ (533.69 mg, 1.638 mmol) in dioxane (5 mL) and H₂O (1 mL) was added Pd (OAc)₂ (12.26 mg, 0.055 mmol) and Butyldi-1-adamantylphosphine (19.58 mg, 0.055 mmol). The resulting mixture was stirred at 90° C. for 16 h. The mixture was concentrated to give a residue, which was purified by reversed phase chromatography to afford Compound 22-2. LCMS: 312.1[M+H]⁺.

### Step 3: Preparation of Example 22

To a solution of Compound 22-2 (50 mg, 0.161 mmol) in dioxane (2 mL) was added DMEDA (56.63 mg, 0.642 mmol), K₂CO₃ (44.39 mg, 0.321 mmol), 3-(1-(3-bromophenyl)-3-methylcyclobutyl)-4-methyl-4H-1,2,4-triazole (49.18 mg, 0.161 mmol) and CuI (30.59 mg, 0.161 mmol). The mixture was stirred at 100° C. for 16 h under N₂ atmosphere. The residue was purified by Prep-HPLC to afford Example 22. LCMS: 537.3 [M+H]⁺; ¹H NMR: (400 MHz, DMSO-d₆) δ 8.83-8.18 (m, 4H), 7.64-7.33 (m, 4H), 3.99-3.44 (m, 2H), 3.27-3.20 (m, 3H), 2.92-2.76 (m, 2H), 2.68-2.24 (m, 6H), 1.80-1.34 (m, 7H), 1.15-0.98 (m, 3H).

### Example 23

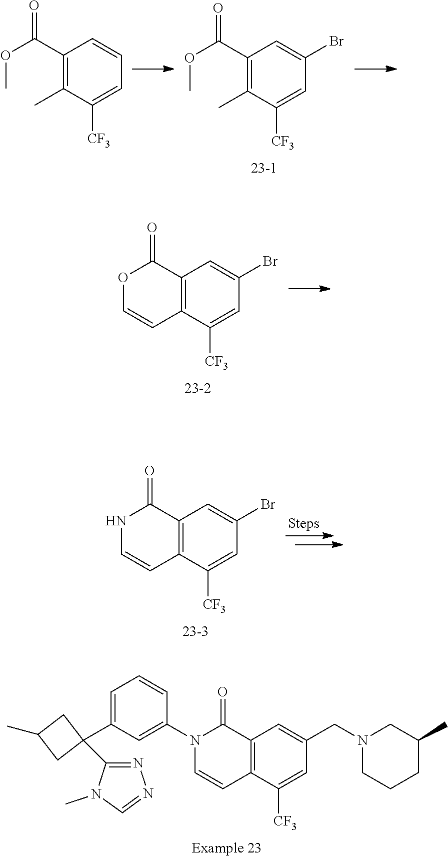

### Step 1: Preparation of Compound 23-1

To a solution of methyl 2-methyl-3-(trifluoromethyl)benzoate (5 g, 22.917 mmol) in AcOH (50 mL) were added Br₂ (1.4 mL, 25.209 mmol), HNO₃ (9.6 mL, 229.169 mmol) and AgNO₃ (5.06 g, 29.792 mmol). The reaction was stirred at room temperature for overnight. The reaction was partitioned between EtOAc (100 mL) and water (100 mL). The organic layer was separated, washed with saturated NaCl solution (3×50 mL), dried, filtered and concentrated to give a residue. The residue was purified by flash silica gel chromatography to afford Compound 23-1.

### Step 2: Preparation of Compound 23-2

To a solution of Compound 23-1 (4.2 g, 14.138 mmol) in DMF (40 mL) was added (dimethylamino)dimethoxymethane (5.05 g, 42.414 mmol). The reaction mixture was stirred at 110° C. for overnight. The reaction was partitioned between EtOAc (100 mL) and water (100 mL). The organic layer was washed with saturated NaCl solution (3×50 mL), dried, filtered and concentrated to give a residue. The residue was purified by flash silica gel chromatography to afford Compound 23-2.

### Step 3: Preparation of Compound 23-3

To a solution of 7-bromo-5-(trifluoromethyl)-1H-isochromen-1-one (200 mg, 0.683 mmol) in MeOH (2 mL) was added ammonia in MeOH (0.20 mL, 1.433 mmol). The reaction mixture was stirred at 60° C. for overnight. Then the mixture was partitioned between EtOAc (100 mL) and water (100 mL).

The organic layer was washed with saturated NaCl solution (3×50 mL), dried, filtered and concentrated to give a residue. The residue was purified by flash silica gel chromatography to afford Compound 23-3. LCMS: 291.0 [M−H]⁻.

### Step 4: Preparation of Example 23

According to the procedures in Example 1, Example 23 was synthesized with Compound 23-3 and potassium (S)-trifluoro((3-methylpiperidin-1-yl)methyl)borate as the substrates. LCMS: 550.3 [M+H]³⁰; ¹H NMR: (400 MHz, Chloroform-d) δ 8.81-8.56 (m, 2H), 8.21 (s, 1H), 7.63-7.48 (m, 2H), 7.44 (s, 1H), 7.40-7.29 (m, 2H), 6.89 (d, J=7.7 Hz, 1H), 4.37-4.27 (m, 2H), 3.63 (s, 1H), 3.55-3.15 (m, 4H), 3.02-2.75 (m, 2H), 2.73-2.46 (m, 3H), 2.41-2.19 (m, 1H), 2.14-1.71 (m, 4H), 1.37-1.23 (m, 1H), 1.26-1.10 (m, 3H), 1.08-0.98 (m, 1H), 0.94 (d, J=6.4 Hz, 3H).

### Example 24

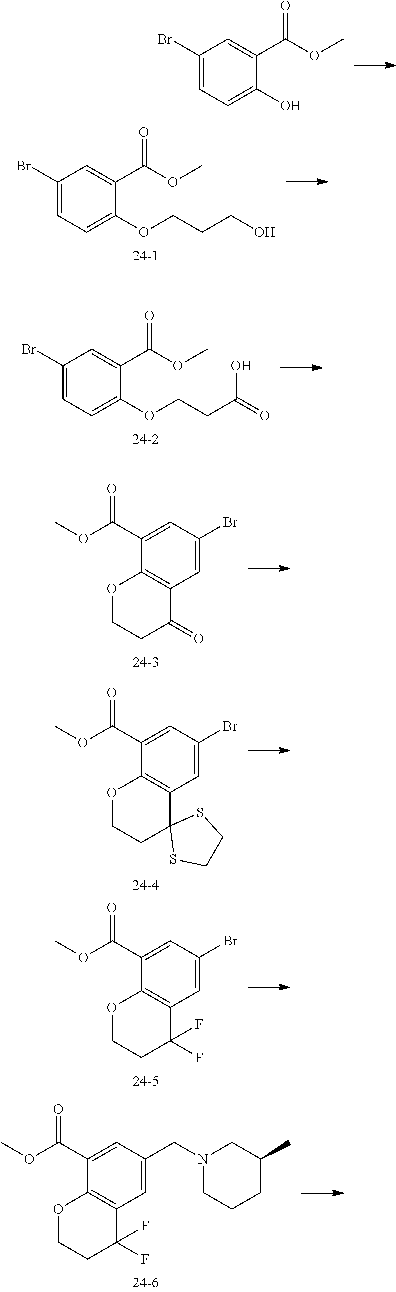

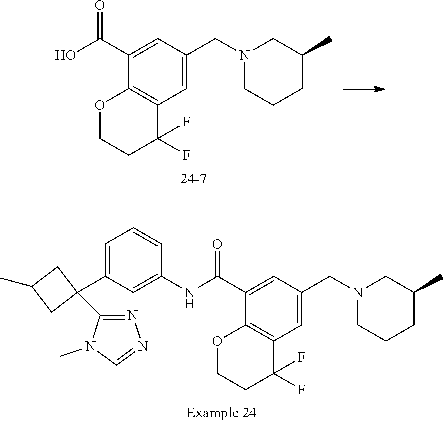

### Step 1: Preparation of Compound 24-1

To a solution of methyl 5-bromo-2-hydroxybenzoate (24 g, 103.878 mmol) in dry DMF (240 mL) was added K₂CO₃ (21.53 g, 155.817 mmol) and 3-bromopropan-1-ol (14 mL, 156 mmol) under N₂ atmosphere. The mixture was stirred at 60° C. for 4 h. The reaction was quenched by pouring into 100 mL sat. NH₄Cl and then the mixture was diluted with 100 mL EtOAc. The aqueous phase was extracted four times with EtOAc (150 mL×3). The combined organic phases were washed with 150 mL brine, dried, filtered to remove solids, and concentrated under reduced pressure. The residue was purified by silica gel chromatography to afford Compound 24-1. LCMS: 288.8 [M+H]⁺.

### Step 2: Preparation of Compound 24-2

To a solution of Compound 24-1 (15 g, 51.880 mmol) in MeCN (150 mL) was added water (12.5 mL), TEMPO (2.43 g, 15.564 mmol) and (Diacetoxyiodo)benzene (50.44 g, 155.639 mmol). The reaction mixture was stirred for 3 h at room temperature. Upon completion, 2-Methyl-2-butene (181.94 g, 2593.989 mmol), water (12.5 mL) and NaH₂PO₄ (62.23 g, 518.798 mmol) were added, respectively. The resulting mixture was cooled to 0 T followed by the addition of NaClO₂ (37.54 g, 415.038 mmol). The reaction was stirred vigorously for 1 h at 0 T. The reaction was quenched by pouring into 1100 mL sat. Na₂S₂O₃. The aqueous layer was extracted with EA (1500 mL×3). The combined organic layers were washed with brine (1000 mL), dried over anhydrous Na₂SO₄ and concentrated to afford a residue. The residue was purified by silica gel chromatography to afford Compound 24-2. LCMS: 300.8 [M−H]⁻.

### Step 3: Preparation of Compound 24-3

A mixture of Compound 24-2 (2 g, 6.598 mmol) in Chlorosulfonic acid (12 mL, 0.759 mmol) was stirred at 0° C. for 1 h. The resulting mixture was quenched by pouring into iced water. The mixture was extracted with DCM (25 mL×3). The combined organic layers were washed with brine (25 mL), dried over anhydrous Na₂SO₄ and concentrated to afford a residue. The residue was purified by silica gel chromatography to afford Compound 24-3. LCMS: 284.9 [M+H]⁺.

### Step 4: Preparation of Compound 24-4

To a solution of Compound 24-3 (680 mg, 2.385 mmol)) in DCM (10 mL) was added ethane-1,2-dithiol (562 mg, 5.962 mmol) and Boron trifluoride diethyl etherate (846 mg, 5.963 mmol). The mixture was stirred at r.t. for 16 h. The aqueous layer was extracted with EA (30 mL×3). The combined organic layers were washed with brine (30 mL), dried over anhydrous Na₂SO₄ and concentrated to afford a residue. The residue was purified by silica gel chromatography to afford Compound 24-4. LCMS: 360.7 [M+H]⁺.

### Step 5: Preparation of Compound 24-5

A mixture of NIS (423 mg, 1.882 mmol) in DCM (5 mL) was cooled to −78° C. followed by the addition of HF-Py (373 mg, 3.764 mmol) and a solution of Compound 24-4 (340 mg, 0.941 mmol) in DCM (5 mL). The reaction was stirred at −78° C. for 1 h, then warmed to 0° C. and stirred for an additional 30 min. The residue was purified by silica gel chromatography to afford Compound 24-5. LCMS: 306.8 [M+H]⁺.

### Step 6: Preparation of Compound 24-6

To a solution of Compound 24-5 (190 mg, 0.619 mmol) in dioxane (6 mL) and H₂O (1.5 mL) was added (S)-3-methyl-1-((trifluoro-14-boraneyl) methyl) piperidine, potassium salt (407 mg, 1.856 mmol), Cs₂CO₃ (605 mg, 1.856 mmol) and RuPhos Pd G₄ (53 mg, 0.062 mmol). The resulting mixture was stirred for 16 h at 90° C. under nitrogen atmosphere. The residue was purified by Reversed Phase Chromatography to afford Compound 24-6. LCMS: 340.5 [M+H]⁺.

### Step 7: Preparation of Compound 24-7

To a solution of Compound 24-6 (130 mg, 0.383 mmol) in MeOH (2 mL) and H₂O (0.5 mL) was added NaOH (45.97 mg, 1.149 mmol). The mixture was stirred at r.t. for 16 h. The mixture was acidified to pH=6 by 2N HCl, then concentrated to give a residue. The residue was purified by Reversed Phase Chromatography to afford Compound 24-7. LCMS: 326.3 [M+H]⁺.

### Step 8: Preparation of Example 24

To a solution of Compound 24-7 (80 mg, 0.246 mmol), 3-(3-methyl-1-(4-methyl-4H-1,2,4-triazol-3-yl)cyclobutyl)aniline (60 mg, 0.246 mmol) in Pyridine (2 mL) was added EDCI (71 mg, 0.369 mmol). The mixture was stirred at r.t. for 2 h. The residue was purified by Prep-HPLC to afford Example 24. LCMS: 550.5 [M+H]⁺; ¹H NMR: (400 MHz, DMSO-d₆) δ 10.19 (d, J=7.0 Hz, 1H), 9.39-7.36 (m, 5H), 7.32 (q, J=7.7, 7.0 Hz, 1H), 7.12-6.76 (m, 1H), 4.51-4.37 (m, 2H), 3.44 (s, 2H), 3.31 (s, 3H), 3.25-3.05 (m, 3H), 2.79-2.62 (m, 3H), 2.36-2.13 (m, 2H), 2.01-1.32 (m, 7H), 1.14-1.04 (m, 3H), 0.91-0.77 (m, 4H).

### Example 25

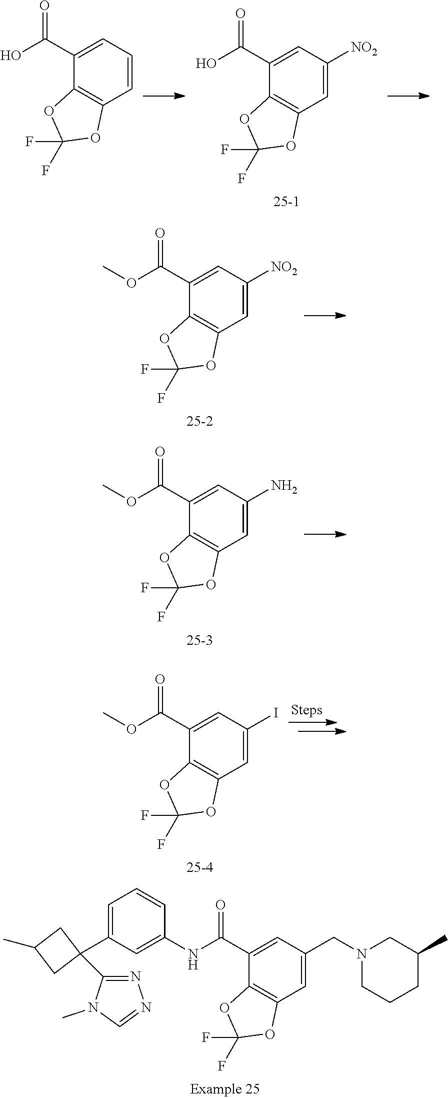

### Step 1: Preparation of Compound 25-1

A mixture of 2,2-difluorobenzo[d][1,3]dioxole-4-carboxylic acid (500 mg, 2.474 mmol) in H₂SO₄ (2.5 mL) was added HNO₃ (0.25 mL) and stirred at 0° C. for 3 h. Upon completion, the mixture was poured into ice water. The organic phase was separated, washed with water (10 mL), dried and filtered to afford Compound 25-1 which was used for next step without further purification. LCMS: 248.0 [M+H]⁺;

### Step 2: Preparation of Compound 25-2

To a solution of Compound 25-1 (200 mg, 0.809 mmol) in DMF (5 mL) was added iodomethane (0.2 mL), K₂CO₃ (335.56 mg, 2.428 mmol) under N₂. the mixture was stirred at room temperature for 2 h. The mixture was extracted with EtOAc (10 mL×3). The combined organic layers were washed with brine (10 mL), dried over anhydrous Na₂SO₄, filtered and concentrated to afford a residue. The residue was purified by flash silica gel chromatography to afford Compound 25-2. ¹H NMR (400 MHz, CDCl₃) δ 8.69 (d, J=2.3 Hz, 1H), 8.12 (d, J=2.3 Hz, 1H), 4.03 (s, 3H).

### Step 3: Preparation of Compound 25-3

A mixture of Compound 25-2 (2 g, 7.659 mmol), Fe (4.28 g, 76.587 mmol) and NH₄Cl (4.1 g, 76.587 mmol) in EtOH (20 mL) and H₂O (5 mL) was stirred at 80° C. for 2 h. The reaction mixture was filtered and the filtrate was concentrated under reduced pressure to remove solvent to afford Compound 25-3, which could be used for next step without further purification. LCMS: 232.1 [M+H]⁺;

### Step 4: Preparation of Compound 25-4

To a solution of Compound 25-3 (980 mg, 4.240 mmol) in H₂SO₄ (0.2 mL) and H₂O (5 mL) was added a mixture of sodium nitrite (438.74 mg, 6.360 mmol) in H₂O (5 mL). The resulting mixture was stirred at 0° C. for 1 hr. After the addition of a mixture of potassium iodide (2.1 g, 12.719 mmol) in H₂O (5 mL), the mixture was stirred at 80° C. for additional 15 min. The mixture was extracted with EtOAc (50 mL×3). The combined organic layers were washed with brine (80 mL), dried over anhydrous Na₂SO₄, filtered and concentrated to give a residue. The residue was purified by flash silica gel chromatography to afford Compound 25-4. ¹H NMR (400 MHz, CDCl₃) δ 8.02 (d, J=1.7 Hz, 1H), 7.54 (d, J=1.7 Hz, 1H), 3.97 (s, 3H).

### Step 5: Preparation of Example 25

According to the procedures in Example 24, Example 25 was synthesized with Compound 25-4 as the substrate. LCMS: 536.4 [M−H]⁻; ¹H NMR: (400 MHz, Chloroform-d) δ 8.26 (s, 1H), 7.96 (s, 1H), 7.83 (s, 11H), 7.76 (s, 1H), 7.51 (d, J=8.0 Hz, 1H), 7.40-7.31 (m, 2H), 7.11 (d, J=7.7 Hz, 1H), 3.51 (s, 2H), 3.23 (s, 3H), 2.95-2.83 (m, 2H), 2.79-2.60 (m, 4H), 2.04-1.54 (m, 7H), 1.15 (d, J=5.4 Hz, 3H), 0.94-0.80 (m, 4H).

### Example 26 and 27

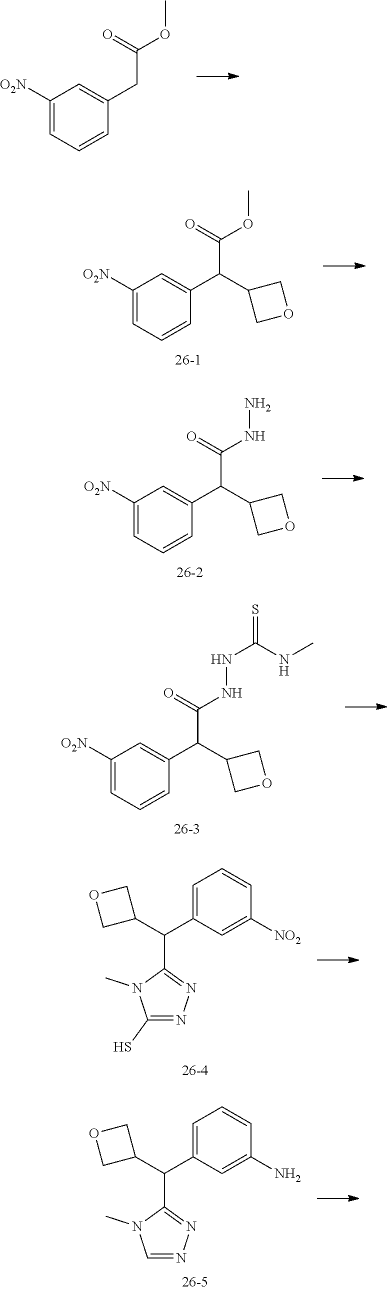 [SMILES: COC(=O)Cc1cccc([N+](=O)[O-])c1 ; COC(=O)C(c1cccc([N+](=O)[O-])c1)C1COC1 ; NNC(=O)C(c1cccc([N+](=O)[O-])c1)C1COC1 ; CNC(=S)NNC(=O)C(c1cccc([N+](=O)[O-])c1)C1COC1 ; Cn1c(S)nnc1C(c1cccc([N+](=O)[O-])c1)C1COC1 ; Cn1cnnc1C(c1cccc(N)c1)C1COC1]

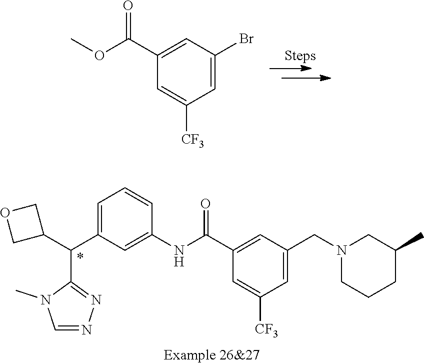 [SMILES: COC(=O)c1cc(Br)cc(C(F)(F)F)c1 ; C[C@H]1CCCN(Cc2cc(C(=O)Nc3cccc(C(c4nncn4C)C4COC4)c3)cc(C(F)(F)F)c2)C1]

### Step 1: Preparation of Compound 26-1

To a solution of methyl 2-(3-nitrophenyl)acetate (500 mg, 2.562 mmol) in DMF (10 mL) was added 3-iodooxetane (471.33 mg, 2.562 mmol) and potassium 2-methylpropan-2-olate (373.71 mg, 3.330 mmol) at 25° C. under N₂. The mixture was stirred at 25° C. for 1 hr. The mixture was added dropwise into aqueous solution of NH₄Cl (30 mL) while stirring at 0° C. over 10 min. The aqueous layer was extracted with ethyl acetate (40 mL×3). The combined organic layers were washed with brine (80 mL), dried over anhydrous Na₂SO₄, filtered, concentrated, and purified by silica gel chromatography to afford Compound 26-1. LCMS: 252.0 [M+H]⁺.

### Step 2: Preparation of Compound 26-2

To a solution of Compound 26-1 (570 mg, 2.269 mmol) in EtOH (2 mL) was added hydrazine hydrate (0.772 mL, 15.881 mmol). And then the mixture was stirred at 60° C. for 12 h. The mixture was concentrated under reduced pressure to remove the solvent. The residue was purified by flash silica gel chromatography to afford Compound 26-2. ¹H NMR: (400 MHz, CDCl3) δ 8.16 (d, J=7.9 Hz, 2H), 7.67 (d, J=7.6 Hz, 1H), 7.54 (t, J=7.7 Hz, 1H), 7.15 (s, 1H), 5.01-4.54 (m, 2H), 4.50-4.22 (m, 2H), 3.97-3.85 (m, 3H), 3.48 (s, 1H).

### Step 3: Preparation of Compound 26-3

To a solution of Compound 26-2 (356 mg, 1.417 mmol) in THF (10 mL) was added isothiocyanatomethane (414.38 mg, 5.668 mmol). And the mixture was stirred at 80 T for 2 h. The reaction mixture was filtered and concentrated under reduced pressure to give Compound 26-3 which was used for next step directly. LCMS: 325.2 [M+H]⁺.

### Step 4: Preparation of Compound 26-4

To a solution of KOH (397.00 mg, 7.075 mmol) in H₂O (2 mL) was added Compound 26-3 (459 mg, 1.415 mmol). The mixture was stirred at 25° C. for 12 h. The reaction mixture was partitioned between dichloromethane (15 mL) and H₂O (6 mL). The pH of the water layer was adjusted to 5-7 by 1M HCl (a.q.). The mixture was extracted with dichloromethane (20 ml×3). The combined organic layers were washed with brine (30 mL), dried over anhydrous Na₂SO₄, filtered and concentrated under reduced pressure to give a residue. The residue was purified by flash silica gel chromatography to afford Compound 26-4. LCMS: 307.0 [M+H]⁺.

### Step 5: Preparation of Compound 26-5

To a solution of Compound 26-4 (315 mg, 1.028 mmol) in ethanol (10 mL) was added Raney-Ni at room temperature. Then the mixture was stirred at 78° C. for 40 min. The mixture was filtered and the filtrate was concentrated under reduced pressure to give Compound 26-5. LCMS: 267.1 [M+Na]⁺.

### Step 6: Preparation of Compound 26-6

After the preparation of Compound 26-5, according to the procedures in Example 24, Example 26 and Example 27 were synthesized with methyl 3-bromo-5-(trifluoromethyl)benzoate (200 mg, 0.707 mmol) as the substrate. Chiral SFC analysis was performed on a DAICELCHIRALPAK®OD (250×4.6 mm, 4.0 μm) column, eluted with mobile phase (A: Supercritical CO₂; B: MeOH (0.10% DEA); Gradient: 5% of B for 0.5 min, then from 5% to 40% of B in 5 min, then 40% of B for 2.5 min), flowing at 1.5 mL/min, at 35° C., with pressure set at 1800 psi.

### Example 26

LCMS: 528.2 [M+H]⁺; ¹H NMR: (400 MHz, Chloroform-d) δ 8.64 (s, 1H), 8.22-7.95 (m, 3H), 7.89 (d, J=7.5 Hz, 1H), 7.73 (s, 1H), 7.58-7.32 (m, 2H), 7.00 (s, 1H), 5.01 (t, J=6.7 Hz, 1H), 4.69 (t, J=6.7 Hz, 1H), 4.61-4.49 (m, 1H), 4.42 (d, J=11.0 Hz, 1H), 4.35-4.26 (m, 1H), 4.08-3.95 (m, 1H), 3.67 (s, 2H), 3.40 (s, 3H), 3.14-2.70 (m, 2H), 1.93-0.97 (m, 6H), 0.98-0.74 (m, 4H). t_{R}=3.64 min; Chiral purity: 100%.

### Example 27

LCMS: 528.2 [M+H]⁺; ¹H NMR: (400 MHz, Chloroform-d) δ 8.86 (s, 1H), 8.12 (s, 1H), 8.04-7.87 (m, 3H), 7.74 (s, 1H), 7.49-7.33 (m, 2H), 7.03 (d, J=6.8 Hz, 1H), 5.00-4.95 (m, 1H), 4.69 (t, J=6.7 Hz, 1H), 4.59-4.48 (m, 1H), 4.41 (d, J=10.9 Hz, 1H), 4.32-4.21 (m, 1H), 4.12-3.90 (m, 1H), 3.61 (s, 2H), 3.40 (s, 3H), 2.97-2.65 (m, 2H), 2.09-1.19 (m, 6H), 0.97-0.78 (m, 4H). t_{R}=4.15 min; Chiral purity: 99.0%.

### Example 28 and 29

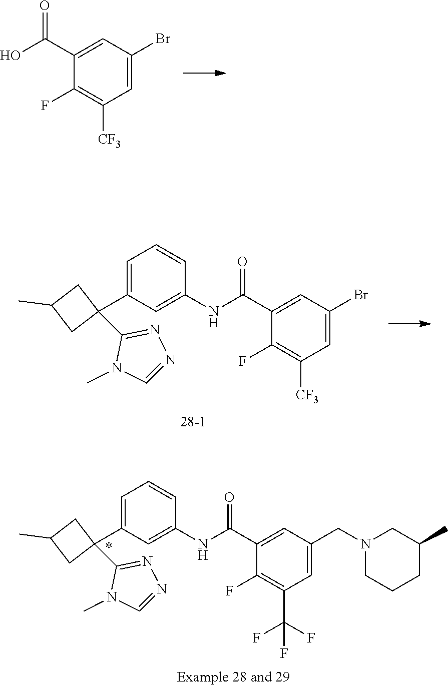

### Step 1: Preparation of Compound 28-1

A mixture of 5-bromo-2-fluoro-3-(trifluoromethyl)benzoic acid (200.0 mg, 696.8 mol), 3-(3-methyl-1-(4-methyl-4H-1,2,4-triazol-3-yl)cyclobutyl)aniline (185.7 mg, 766.5 mol), HATU (291.5 mg, 766.5 μmol) and DIEA (270.2 mg, 2.09 mmol, 364.1 μL) in DCM (5 mL) was degassed and purged with N₂ for 3 times, and then the mixture was stirred at 25° C. for 16 h under N₂ atmosphere. The reaction mixture was concentrated under reduced pressure to remove DCM. The residue was purified by flash silica gel chromatography to afford Compound 28-1. LCMS: 513.1 [M+H]⁺; ¹H NMR: (400 MHz, DMSO-d₆) δ 10.64-10.74 (m, 1H) 8.30-8.40 (m, 1H) 8.15-8.24 (m, 2H) 7.64-7.75 (m, 1H) 7.31-7.56 (m, 2H) 6.98-7.20 (m, 1H) 3.57-3.69 (m, 2H) 3.40-3.50 (m, 1H) 3.10-3.22 (m, 5H) 1.08 (dd, J=10.51, 6.75 Hz, 3H)

### Step 2: Preparation of Example 28 and 29

A mixture of Compound 28-1 (150.0 mg, 293.4 μmol), potassium (S)-trifluoro((3-methylpiperidin-1-yl)methyl)borate (105.6 mg, 586.7 μmol), PdCl₂(dtbpf) (9.56 mg, 14.7 mol) and K₃PO₄ (124.5 mg, 586.7 μmol) in dioxane (5 mL) and H₂O (1 mL) was degassed and purged with N₂ for 3 times, and then the mixture was stirred at 90° C. for 16 h under N₂ atmosphere. The reaction mixture was filtered and concentrated under reduced pressure. The residue was purified by Prep-HPLC to afford mixture, which was further separated by SFC to afford Example 28 and 29. Chiral SFC analysis was performed on a DAICELCHIRALPAK®OJ (100×3.0 mm, 3.0 m) column, eluted with mobile phase (A: Supercritical CO₂; B: MeOH (0.1% DEA); Gradient: 5% of B for 0.5 min, then from 5% to 40% of B in 5 min, then 40% of B for 2.5 min), flowing at 1.5 mL/min, at 35° C., with pressure set at 1800 psi.

### Example 28

LCMS: 544.3 [M+H]⁺; ¹H NMR: (400 MHz, CDCl₃) δ 8.32 (s, 1H), 7.95 (s, 1H), 7.80 (d, J=5.1 Hz, 1H), 7.79-7.49 (m, 2H), 7.40-7.27 (m, 2H), 7.11 (d, J=7.9 Hz, 1H), 3.58 (s, 2H), 3.22 (s, 3H), 2.88 (dd, J=10.1, 6.6 Hz, 2H), 2.70-2.58 (m, 3H), 2.39-1.87 (m, 2H), 1.84-1.60 (m, 6H), 1.14 (d, J=5.3 Hz, 3H), 0.87 (d, J=4.1 Hz, 4H). t_{R}=1.88 min; Chiral purity: 96.0%.

### Example 29

LCMS: 544.4 [M+H]⁺; ¹H NMR: (400 MHz, CDCl₃) δ 8.27 (s, 1H), 8.02 (s, 1H), 7.81 (d, J=5.3 Hz, 1H), 7.59 (d, J=7.7 Hz, 1H), 7.53 (d, J=8.2 Hz, 1H), 7.32 (dd, J=15.2, 7.2 Hz, 2H), 6.99 (d, J=8.2 Hz, 1H), 3.56 (s, 2H), 3.26 (s, 3H), 3.20-3.09 (m, 2H), 2.91-2.46 (m, 3H), 2.32 (t, J=10.6 Hz, 2H), 2.05-1.64 (m, 6H), 1.13 (d, J=6.6 Hz, 3H), 0.86 (d, J=5.4 Hz, 4H). t_{R}=2.20 min; Chiral purity: 100%.

### Example 30

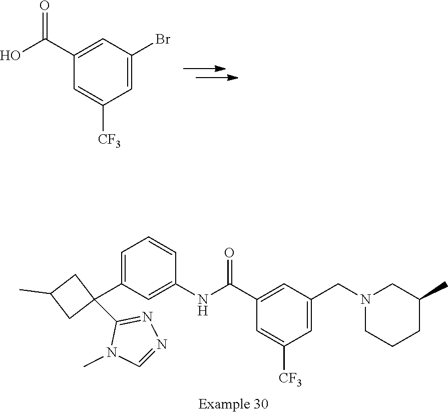

According to Example 28, Example 30 was synthesized with 3-bromo-5-(trifluoromethyl)benzoic acid as the substrate. LCMS: 526.4 [M+H]⁺; ¹H NMR: (400 MHz, DMSO-d₆) δ 10.45 (s, 1H), 8.38 (s, 1H), 8.15 (d, J=10.38 Hz, 2H), 7.84 (s, 1H), 7.74 (d, J=8.13 Hz, 1H), 7.51 (s, 1H), 7.36 (t, J=7.94 Hz, 1H), 6.98 (d, J=7.25 Hz, 1H), 3.60 (s, 2H), 3.22 (s, 3H), 3.06-3.15 (m, 2H), 2.68-2.76 (m, 2H), 2.32-2.47 (m, 2H), 2.15-2.30 (m, 2H), 1.92 (t, J=10.38 Hz, 1H), 1.54-1.70 (m, 4H), 1.44-1.53 (m, 1H), 1.10 (d, J=6.50 Hz, 3H), 0.82 (d, J=6.00 Hz, 3H).

### Example 31

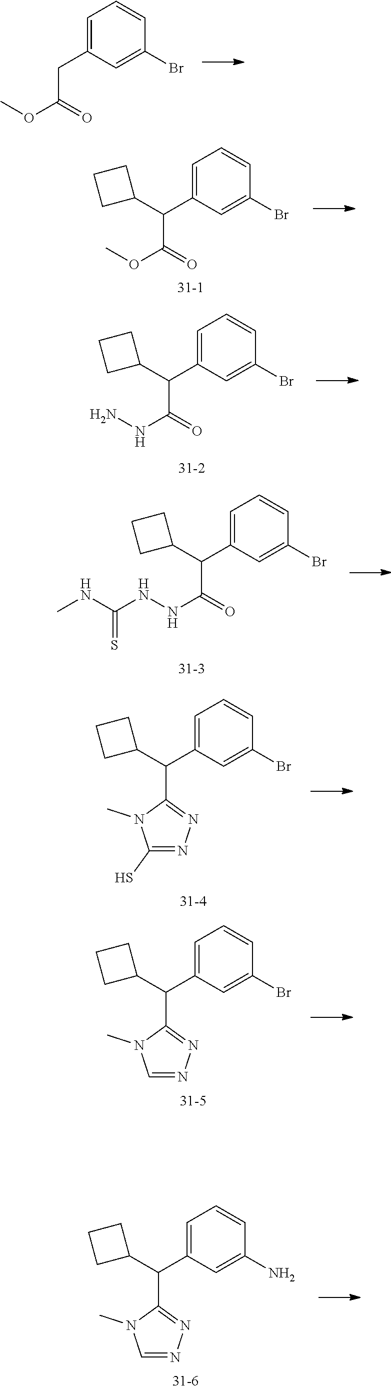

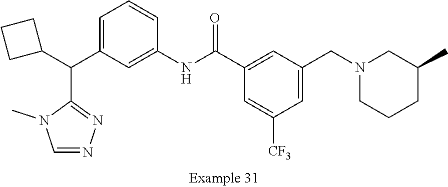 [SMILES: C[C@H]1CCCN(Cc2cc(C(=O)Nc3cccc(C(c4nncn4C)C4CCC4)c3)cc(C(F)(F)F)c2)C1]

### Step 1: Preparation of Compound 31-1

To a solution of methyl 2-(3-bromophenyl)acetate (2.00 g, 8.73 mmol) in DMF (20 mL) was added t-BuOK (1.27 g, 11.35 mmol) at 0° C. The mixture was stirred at 0° C. for 30 mins, and then bromocyclobutane (1.41 g, 10.5 mmol, 989.1 μL) was added at 0° C. The mixture was stirred at 25° C. for 16 h. The reaction mixture was diluted with water (50 mL) and extracted with Ethyl acetate (50 mL×2).

The combined organic layers were washed with brine (30 mL), dried over Na₂SO₄, filtered, and concentrated under reduced pressure to give a residue. The residue was purified by flash silica gel chromatography to afford Compound 31-1. LCMS: 283.2 [M+H]⁺; ¹H NMR: (400 MHz, CHLOROFORM-d) δ 7.34-7.49 (m, 2H), 7.15-7.26 (m, 2H), 3.68 (s, 3H), 3.52 (d, J=10.88 Hz, 1H), 2.88-3.06 (m, 1H), 2.12-2.28 (m, 1H), 1.79-1.95 (m, 4H), 1.59-1.68 (m, 1H).

### Step 2: Preparation of Compound 31-2

To a solution of Compound 31-1 (1.00 g, 3.53 mmol) in EtOH (10 mL) was added hydrazine hydrate (2.65 g, 53.0 mmol, 2.57 mL). The mixture was stirred at 80 T for 16 h. The reaction mixture was concentrated under reduced pressure to remove EtOH. The residue was diluted with water (100 mL) and extracted with Ethyl acetate (100 mL×3). The organic phase was dried over Na₂SO₄, filtered, and concentrated under reduced pressure to afford crude Compound 31-2, which was used into the next step without further purification. LCMS: 283.0 [M+H]⁺.

### Step 3: Preparation of Compound 31-3

To a solution of Compound 31-2 (600.0 mg, 2.12 mmol) in THE (10 mL) was added methylimino(thioxo)methane (619.7 mg, 8.48 mmol, 579.2 μL). The mixture was stirred at 25° C. for 16 h. The reaction mixture was concentrated under reduced pressure to remove THF to afford crude product Compound 31-3, which was used into the next step without further purification. LCMS: 357.8 [M+H]⁺.

### Step 4: Preparation of Compound 31-4

To a solution of Compound 31-3 (1.50 g, 4.21 mmol) in water (5 mL) was added NaOH (673.6 mg, 16.8 mmol). The mixture was stirred at 20° C. for 16 h. The reaction mixture was adjusted to pH=7 with 1 M HCl at 25° C., and then diluted with water (50 mL) and extracted with DCM (100 mL×3), dried over Na₂SO₄, filtered, and concentrated under reduced pressure to afford the crude Compound 31-4, which was used into the next step without further purification. LCMS: 339.9 [M+H]⁺.

### Step 5: Preparation of Compound 31-5

To a solution of Compound 31-4 (700.0 mg, 2.07 mmol) in DCM (10 mL) was added H₂O₂ (994.1 μL, 10.4 mmol, 30% purity) and AcOH (1.49 g, 24.8 mmol, 1.42 mL) at 0° C. The mixture was stirred at 20° C. for 12 h. The reaction was quenched by addition of aq. Na₂SO₃ (100 mL) at 25° C., and then the mixture was extracted with DCM (50 mL×3). The combined organic layers were washed with brine (100 mL), dried over Na₂SO₄, filtered, and concentrated under reduced pressure to afford the crude Compound 31-5, which was used into the next step without further purification. LCMS: 307.8 [M+H]⁺.

### Step 6: Preparation of Compound 31-6

To a solution of Compound 31-5 (300.0 mg, 979.7 mol) in ACN (5 mL) was added Cu₂O (28.0 mg, 195.9 μmol, 20.0 μL) and NH₃—H₂O (2.97 mL, 25.5 mmol, 33% purity). The mixture was stirred at 110° C. for 2 h under microwave irradiation. The reaction mixture was partitioned between water (50 mL) and Ethyl acetate (100 mL). The organic phase was separated, washed with brine (50 mL), dried over Na₂SO₄, filtered, and concentrated under reduced pressure to give a residue. The residue was purified by flash silica gel chromatography to afford Compound 31-6. LCMS: 242.9 [M+H]⁺; ¹H NMR: (400 MHz, DMSO-d₆) δ 8.30 (s, 1H), 6.92 (t, J=7.69 Hz, 1H), 6.25-6.45 (m, 3H), 5.03 (br s, 2H), 3.92 (d, J=10.51 Hz, 1H), 3.35 (s, 3H), 3.01-3.16 (m, 1H), 2.01-2.15 (m, 1H), 1.71-1.85 (m, 4H), 1.56-1.69 (m, 1H).

### Step 7: Preparation of Example 31

To a solution of Compound 31-6 (70.0 mg, 288.9 gmol) and Compound 26-7 (87.0 mg, 288.9 μmol) in DCM (1 mL) was added DIEA (112.0 mg, 866.6 gmol, 150.9 μL) and HATU (109.8 mg, 288.9 μmol). The mixture was stirred at 25° C. for 16 h. The reaction mixture was concentrated under reduced pressure to give a residue. The residue was purified by Prep-HPLC to afford Example 31. LCMS: 526.3 [M+H]⁺; ¹H NMR: (400 MHz, DMSO-d₆) δ 10.44 (s, 1H), 8.33 (s, 1H), 8.15 (d, J=9.90 Hz, 2H), 7.84 (s, 1H), 7.71 (d, J=8.68 Hz, 1H), 7.54 (s, 1H), 7.31 (t, J=7.82 Hz, 1H), 7.04 (d, J=7.70 Hz, 1H), 4.16 (d, J=10.64 Hz, 1H), 3.61 (br s, 2H), 3.40 (s, 4H), 3.09-3.24 (m, 1H), 2.61-2.77 (m, 2H), 2.06-2.17 (m, 1H), 1.93 (t, J=10.03 Hz, 1H), 1.74-1.83 (m, 4H), 1.54-1.71 (m, 5H), 1.48 (q, J=12.35 Hz, 1H), 0.72-0.85 (m, 3H).

### Intermediate 1

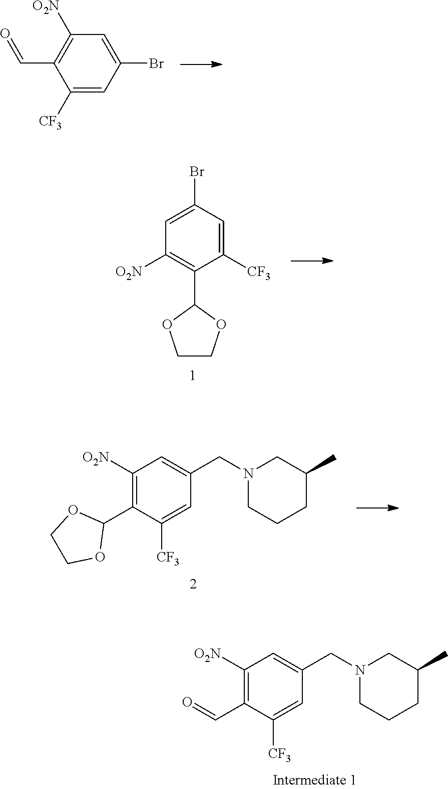 [SMILES: O=Cc1c([N+](=O)[O-])cc(Br)cc1C(F)(F)F ; O=[N+]([O-])c1cc(Br)cc(C(F)(F)F)c1C1OCCO1 ; C[C@H]1CCCN(Cc2cc([N+](=O)[O-])c(C3OCCO3)c(C(F)(F)F)c2)C1 ; C[C@H]1CCCN(Cc2cc([N+](=O)[O-])c(C=O)c(C(F)(F)F)c2)C1]

### Step 1: Preparation of Int 1-1

To a solution of Compound 4-bromo-2-nitro-6-(trifluoromethyl)benzene-1-carbaldehyde (1.0 g, 3.356 mmol) in methylbenzene (10 mL) was added PTSA (0.2 g, 1.007 mmol) and ethylene glycol (0.94 mL, 16.779 mmol) at room temperature. The solution was stirred at 110° C. for 12 hrs. Upon completion, water (30 mL) was added to quench the reaction. The mixture was extracted with Ethyl acetate (40 mL×3). The combined organic phase was washed with brine (150 mL), dried over anhydrous Na₂SO₄, filtered, concentrated in vacuo and purified by column chromatography to afford Int 1-1 (1.1 g, 95.8%). ¹H NMR (400 MHz, CDCl₃) δ 8.00 (d, J=1.7 Hz, 1H), 7.80 (d, J=1.6 Hz, 1H), 6.11 (s, 1H), 4.13-3.88 (m, 4H).

### Step 2: Preparation of Int 1-2

Int 1-2 was synthesized according to the Step 2 in Example 22. LCMS: 375.2 [M+H]⁺;

### Step 3: Preparation of Intermediate 1

To a solution Int 1-2 (430 mg, 1.149 mmol) in acetonitrile (6 mL) was added H₂SO₄ (0.24 mL, 4.594 mmol), then the mixture was stirred at 60 T for 2 hrs. After concentrated, the residue was resolved in DCM (10 mL). To the resulting solution was added 20 mL NH₃/MeOH (7 N). The mixture was stirred at room temperature for 30 min. The mixture was concentrated under reduced pressure to remove solvent to give a residue, which was purified by flash silica gel chromatography to afford the title intermediate (240 mg, 63.3%). LCMS: 331.1 [M+H]⁺.

### Example 32

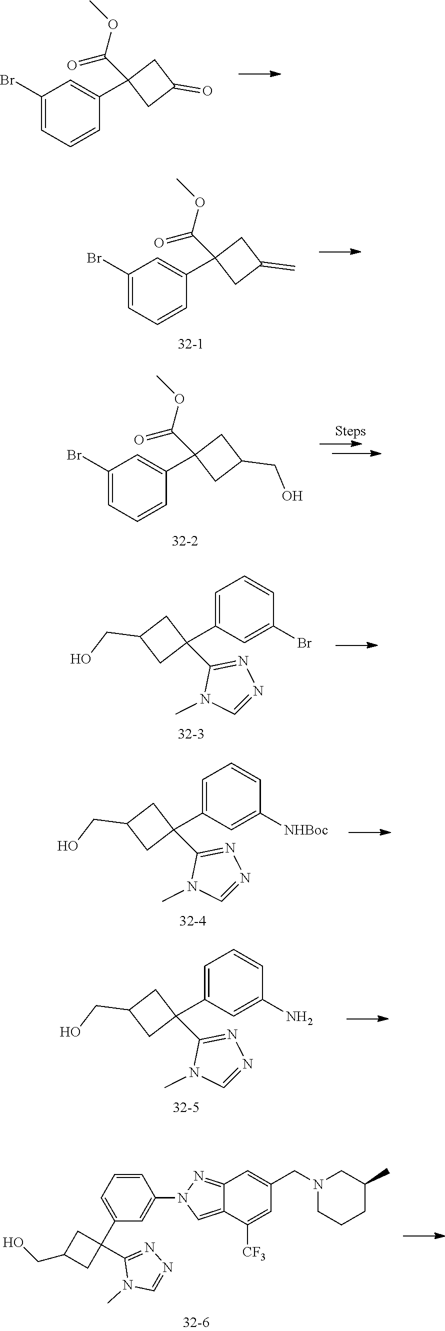

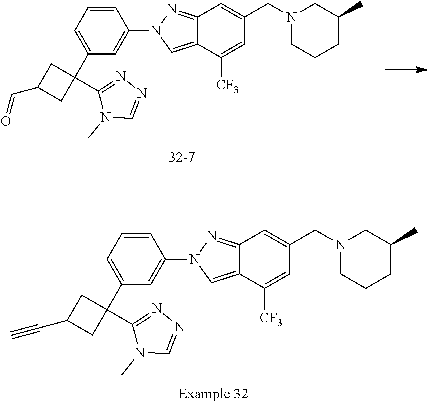

### Step 1: Preparation of Compound 32-1

To a solution of methyltriphenylphosphanium bromide (13.9 g, 38.85 mmol) in THF (50 mL) was added t-BuOK (31.79 mL, 1 M in THF, 31.79 mmol). The resulting mixture was heated to 40° C. for 45 min. After cooling to 0° C., methyl 1-(3-bromophenyl)-3-oxocyclobutane-1-carboxylate (10 g, 35.32 mmol) in THF (50 mL) was added. The reaction was stirred at room temperature for overnight. The reaction mixture was poured into the brine (30 mL), The mixture was diluted with EtOAc (60 mL) and water (100 mL). The organic phase was separated, dried over anhydrous sodium sulfate, filtered, and concentrated under reduced pressure to give a residue. The residue was purified by flash chromatography to give Compound 32-1. ¹H NMR: (400 MHz, CDCl₃) δ 7.46 (d, J=1.6 Hz, 1H), 7.38 (dd, J=7.6, 1.5 Hz, 1H), 7.22 (dt, J=15.4, 7.6 Hz, 2H), 4.92-4.80 (m, 2H), 3.67 (s, 3H), 3.57-3.46 (m, 2H), 3.22-3.09 (m, 2H).

### Step 2: Preparation of Compound 32-2

To a solution of Compound 32-1 (10.8 g, 38.41 mmol) in dry THF (150 mL) was added borane-THF (65.3 mL) at −10° C. The resulting mixture was stirred at room temperature for 4 hours. After cooled to −20° C., MeOH (43.2 mL) was added. After stirring for 15 min, sodium hydroxide (32.0 mL, 96.03 mmol) and hydrogen peroxide (0.89 mL, 1.78 mmol) were added. The mixture was stirred for another 2 hours, then saturated sodium sulfite solution (100 mL) was added. The mixture was diluted with EtOAc (70 mL) and water (100 mL). The organic phase was separated, dried over anhydrous sodium sulfate, filtered and concentrated under reduced pressure to give a residue, which was purified by silica chromatography to give Compound 32-2. LCMS: [M+H]⁺=299.0/301.0.

### Step 3: Preparation of Compound 32-3

With Compound 32-2 as the substrate, Compound 32-3 was synthesized according to the Step 2 to Step 5 in the preparation of Example 31.

### Step 4: Preparation of Compound 32-4

To a solution of Compound 32-3 (1300 mg, 4.03 mmol) in dioxane (25 mL) was added dicyclohexyl{3,6-dimethoxy-2-[2,4,6-tri(prop-2-yl)phenyl]phenyl}phosphane (433.2 mg, 0.807 mmol), tert-butyl carbamate (944.9 mg, 8.070 mmol), Cs₂CO₃ (3943.6 mg, 12.104 mmol) and Pd₂(dba)₃ (369.5 mg, 0.403 mmol). The mixture was stirred at 100° C. for overnight under N₂. Upon completion, the reaction mixture was partitioned between EA (100 mL) and H₂O (50 mL). The organic phase was separated, dried over anhydrous sodium sulfate, filtered and concentrated under reduced pressure to give a residue, which was purified by flash silica gel chromatography to afford Compound 32-4. LCMS: [M+H]⁺=359.2.

### Step 5: Preparation of Compound 32-5

A solution of Compound 32-4 (1147 mg, 3.200 mmol) in TFA (15 mL) and DCM (5 mL) was stirred at room temperature for 30 min. To the solution was add H₂O (100 mL) and EtOAc (300 ml.). The organic phase was separated and washed with brine, dried over anhydrous sodium sulfate, filtered and concentrated under reduced pressure. The resulting residue was purified by Reverse Phase Chromatography to afford Compound 32-5; LCMS: [M+H]⁺=259.2.

### Step 6: Preparation of Compound 32-6

To a solution of Compound 32-5 (200 mg, 0.774 mmol) in IPA (1.5 mL) was added Intermediate 1 (255.7 mg, 0.774 mmol). The mixture was stirred at 80° C. for 4 hrs. Then tributylphosphane (516.9 mg, 2.555 mmol) was added and the resulting mixture was stirred at 80° C. for another 16 hrs. Upon completion, the mixture was partitioned between H₂O (100 mL) and EtOAc (300 mL), the organic phase was separated and washed with brine, dried over anhydrous sodium sulfate, filtered and concentrated under reduced pressure. The resulting residue was purified by silica gel chromatography to give Compound 32-6. LCMS: [M+H]⁺=539.4.

### Step 7: Preparation of Compound 32-7

To a solution of Compound 32-6 (150 mg, 0.278 mmol) in DCM (3 mL) was added DMP (177.1 mg, 0.418 mmol), the mixture was stirred at 20° C. for 1 h. The reaction mixture was diluted with DCM (20 mL), then washed with saturated Na₂SO₃ (10 M), saturated aqueous NaHCO₃ (10 mL) and brine, filtered, and concentrated under reduce pressure to give a residue, which was purified by silica gel column chromatography to afford Compound 32-7. LCMS: [M+H]⁺=537.3.

### Step 8: Preparation of Example 32

To a solution of Compound 32-7 (100 mg, 0.186 mmol) in MeOH (3 mL) was added K₂CO₃ (51.5 mg, 0.373 mmol) and (1Z)-1-(diazyn-1-iumyl)-1-[dimethoxy(oxo)-λ5-phosphanyl]prop-1-en-2-olate (35.8 mg, 0.186 mmol). The mixture was stirred at 0° C. for 3 hrs under N₂. Then the mixture was filtered through a Celite pad, and the filtrate was concentrated to give a residue, which was purified by reverse phase prep-HPLC to afford Example 32. LCMS: [M+H]⁺=533.4; ¹H NMR (400 MHz, Chloroform-d) δ 8.49 (s, 1H), 8.13-7.97 (m, 2H), 7.91 (s, 1H), 7.84-7.76 (m, 1H), 7.63-7.48 (m, 2H), 7.40-7.28 (m, 1H), 3.76 (d, J=11.4 Hz, 2H), 3.42 (t, J=9.4 Hz, 2H), 3.35-3.16 (m, 6H), 3.16-3.07 (m, 1H), 2.96-2.87 (m, 2H), 2.26-2.20 (m, 1H), 1.90-1.47 (m, 4H), 0.88 (d, J=6.2 Hz, 4H).

### Example 33

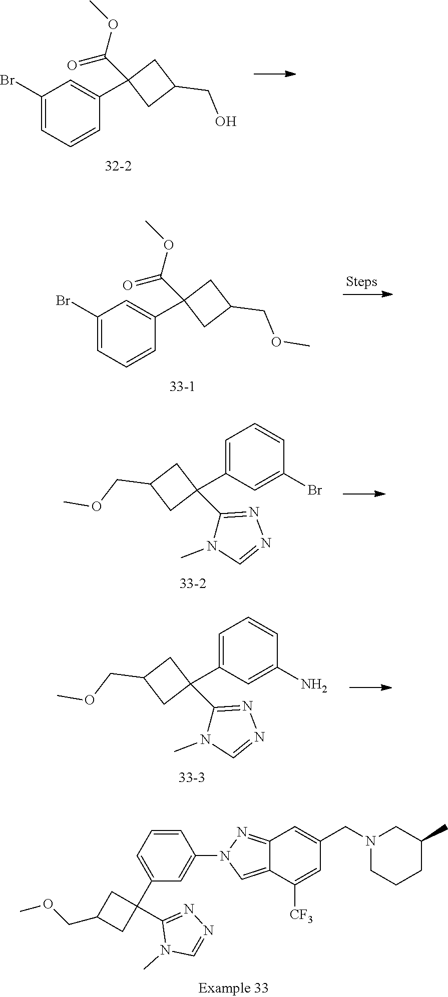

### Step 1: Preparation of Compound 33-1

To a solution of Compound 32-2 (1.5 g, 5.013 mmol) and CH₃1 (2845.6 mg, 20.053 mmol) in DMF (15 mL) was added NaH (802.1 mg, 20.053 mmol) at 0° C., the mixture was stirred at 0° C. for 3 hours. The reaction was diluted with EtOAc (30 mL) and water (50 mL). The organic phase was separated, dried over anhydrous sodium sulfate, filtered and concentrated under reduced pressure to give a residue. The residue was purified by flash silica gel chromatography to afford Compound 33-1.

### Step 2: Preparation of Compound 33-2

With Compound 33-1 as the substrate, Compound 33-2 was synthesized according to the Step 2 to Step 5 in the preparation of Example 31. LCMS: 336.1/338.1 [M+H]⁺.

### Step 3: Preparation of Compound 33-3

A solution of Compound 33-2 (482 mg, 1.434 mmol) and Cu₂O (202.1 mg, 1.434 mmol) in NH₄OH (1 mL, 26.000 mmol) and ACN (1 mL) was stirred at 110° C. for overnight. The reaction mixture was partitioned between water (30 mL) and EtOAc (30 mL). The organic phase was separated, dried over anhydrous sodium sulfate, filtered and concentrated under reduced pressure to give the residue. The residue was purified by reverse phase prep-HPLC to afford Compound 33-3. LCMS: 273.0 [M+H]⁺.

### Step 4: Preparation of Example 33

The title compound was synthesized according to Step 6 in Example 32 with Compound 33-3 as the substrate. LCMS: 553.3 [M+H]⁺; ¹H NMR (400 MHz, DMSO-d₆) δ 9.25 (s, 1H), 8.42-8.31 (m, 1H), 8.11-8.03 (m, 1H), 7.97 (s, 1H), 7.89 (s, 1H), 7.62-7.49 (m, 2H), 7.24 (d, J=8.0 Hz, 1H), 3.59 (s, 2H), 3.41-2.60 (m, 16H), 1.99-1.89 (m, 1H), 1.73-1.32 (m, 4H), 0.86-0.78 (m, 4H).

### Intermediate 2

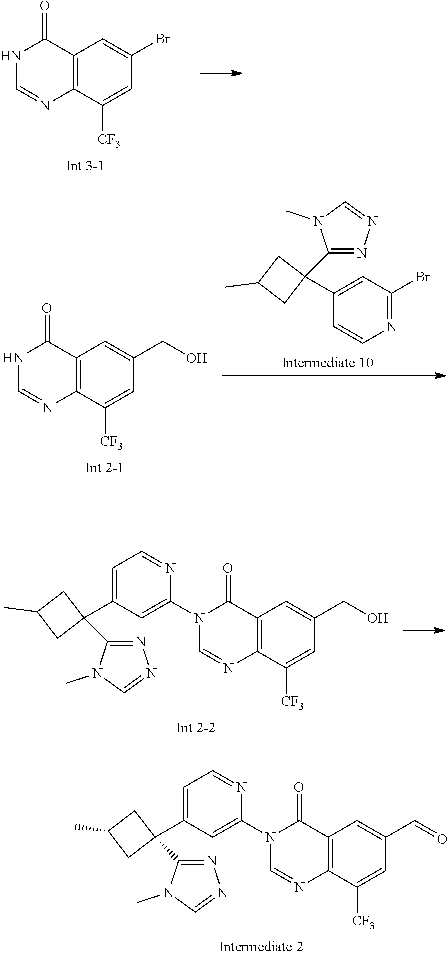 [SMILES: O=c1[nH]cnc2c(C(F)(F)F)cc(Br)cc12 ; O=c1[nH]cnc2c(C(F)(F)F)cc(CO)cc12 ; CC1CC(c2ccnc(Br)c2)(c2nncn2C)C1 ; CC1CC(c2ccnc(-n3cnc4c(C(F)(F)F)cc(CO)cc4c3=O)c2)(c2nncn2C)C1 ; Cn1cnnc1[C@]1(c2ccnc(-n3cnc4c(C(F)(F)F)cc(C=O)cc4c3=O)c2)C[C@H](C)C1]

### Step 1: Preparation of Int 2-1

Int 2-1 was synthesized according to Compound 10-1. LCMS: 245.1 [M+H]⁺;

### Step 2: Preparation of Int 2-2

Int 2-2 was synthesized according to Step 3 in Example 38. LCMS: 245.1 [M+H]⁺;

### Step 3: Preparation of Intermediate 2

To a solution of Int 2-2 (100 mg, 0.213 mmol) in DCM (3 mL) was added DMP (135.2 mg, 0.319 mmol) at 0° C. The mixture was stirred at room temperature for 1 h under N₂. The mixture was filtered and the filter cake was washed with DCM (5 mL×2), the filtration was concentrated to afford the crude product, which was further purified by reverse phase pre-HPLC to afford the title compound. LCMS: 469.1 [M+H]⁺.

The following compounds were synthesized according to step 4 in Example 10 with Intermediate 2 as the substrate.

| Example | LCMS | ¹H NMR |
|---|---|---|
| 34 | 588.2 | ¹H NMR (400 MHz, Chloroform-d) δ 8.75 (s, 1H), 8.57 (d, J = 5.3 Hz, 1H), |
| | [M + H]⁺ | 8.46 (s, 1H), 8.15 (s, 1H), 8.05 (s, 1H), 7.91 (s, 1H), 7.36-7.30 (m, 1H), 3.69 |
| | | (s, 2H), 3.33 (s, 3H), 2.97-2.86 (m, 2H), 2.83-2.62 (m, 5H), 2.47-2.31 |
| | | (m, 1H), 2.24-1.95 (m, 4H), 1.17 (d, J = 6.1 Hz, 3H), 1.01 (d, J = 6.2 Hz, |
| | | 3H). |
| 36 | 550.4 | ¹H NMR (400 MHz, Chloroform-d) δ 8.76 (s, 1H), 8.63-8.45 (m, 2H), 8.28 |
| | [M + H]⁺ | (s, 1H), 8.06 (s, 1H), 7.91 (s, 1H), 7.33 (d, J = 5.2 Hz, 1H), 3.98 (s, 2H), 3.33 |
| | | (s, 3H), 3.18-2.36 (m, 9H), 1.92 (t, J = 6.9 Hz, 2H), 1.22-1.06 (m, 3H), |
| | | 0.62 (s, 4H). |
| 37 | 564.4 | ¹H NMR (400 MHz, Chloroform-d) δ 8.77 (s, 1H), 8.62-8.47 (m, 2H), 8.31 |
| | [M + H]⁺ | (s, 1H), 8.06 (s, 1H), 7.91 (s, 1H), 7.40-7.30 (m, 1H), 3.92 (s, 2H), 3.33 (s, |
| | | 3H), 2.97-2.86 (m, 2H), 2.84-2.60 (m, 5H), 2.42 (s, 2H), 2.01-1.80 (m, |
| | | 2H), 1.50-1.33 (m, 2H), 1.17 (d, J = 6.1 Hz, 3H), 0.44 (d, J = 12.4 Hz, 4H). |
| 39 | 580.4 | ¹H NMR (400 MHz, Chloroform-d) δ 8.75 (s, 1H), 8.57 (d, J = 5.2 Hz, 1H), |
| | [M + H]⁺ | 8.47 (s, 1H), 8.20 (s, 1H), 8.04 (s, 1H), 7.91 (s, 1H), 7.32 (d, J = 4.2 Hz, 1H), |
| | | 3.66 (s, 4H), 3.33 (s, 3H), 2.96-2.87 (m, 2H), 2.80-2.73 (m, 2H), 2.49- |
| | | 2.36 (m, 4H), 2.08-1.99 (m, 4H), 1.84-1.77 (m, 1H), 1.63-1.49 (m, 2H), |
| | | 1.20-1.15 (m, 3H). |
| 61 | 562.3 | ¹H NMR (400 MHz, Chloroform-d) δ 8.76 (s, 1H), 8.57 (d, J = 5.2 Hz, 1H), |
| | [M + H]⁺ | 8.46 (s, 1H), 8.18 (s, 1H), 8.04 (s, 1H), 7.94 (s, 1H), 7.30 (d, J = 4.0 Hz, 1H), |
| | | 3.67 (s, 2H), 3.33 (s, 3H), 2.98-2.55 (m, 6H), 2.35-2.18 (m, 2H), 1.96- |
| | | 1.90 (m, 1H), 1.79-1.53 (m, 4H), 1.49-1.37 (m, 1H), 1.17 (d, J = 6.0 Hz, |
| | | 3H). |

Intermediate 3:

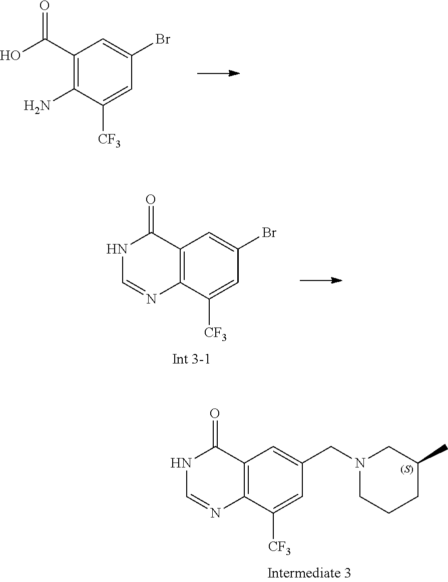

### Step 1: Preparation of Int 3-1

A mixture of 2-amino-5-bromo-3-(trifluoromethyl)benzoic acid (1 g, 3.521 mmol) in azanecarbaldehyde (10 mL) was stirred at 110° C. for overnight in a sealed tube. Upon completion, the mixture was partitioned between EtOAc (30 mL) and H₂O (30 mL). The aqueous phase was extracted with EtOAc (30 mL×3). The combined organic layer was dried over anhydrous sodium sulfate, filtered and concentrated under reduced pressure to give a residue. The residue was purified by flash silica gel chromatography to afford Int 3-1 (250 mg, 19.39%). LCMS: 292.9 [M+H]⁺.

### Step 2: Preparation of Intermediate 3

The title compound was synthesized according to Step 2 in Example 22. LCMS: 326.0 [M+H]⁺.

### Example 35

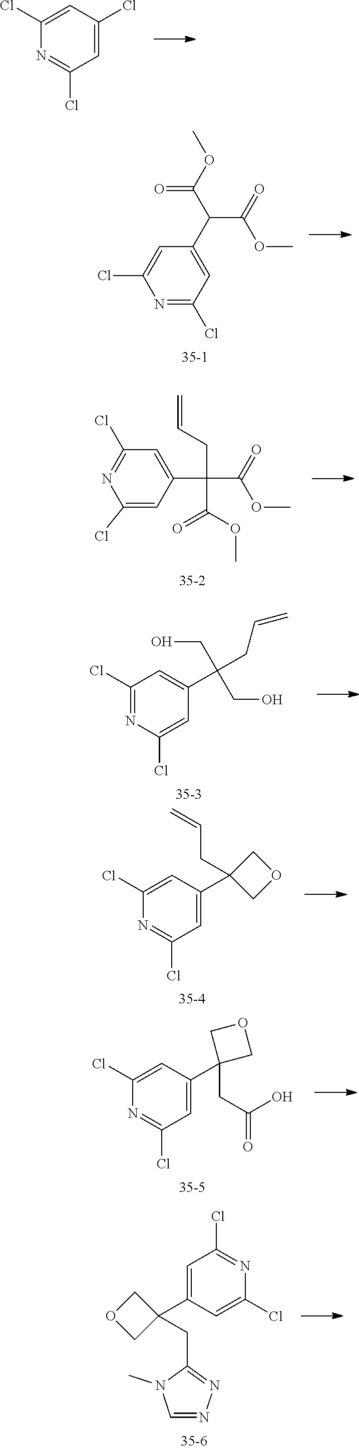

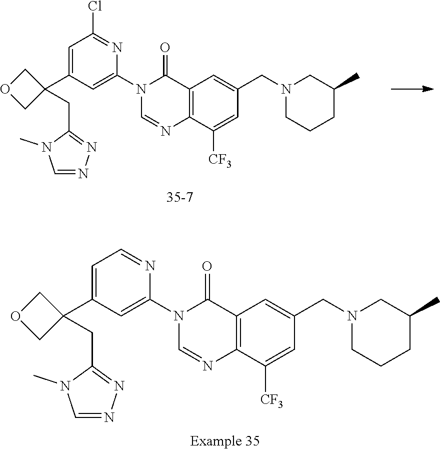 [SMILES: C[C@H]1CCCN(Cc2cc(C(F)(F)F)c3ncn(-c4cc(C5(Cc6nncn6C)COC5)cc(Cl)n4)c(=O)c3c2)C1 ; C[C@H]1CCCN(Cc2cc(C(F)(F)F)c3ncn(-c4cc(C5(Cc6nncn6C)COC5)ccn4)c(=O)c3c2)C1]

### Step 1: Preparation of Compound 35-1

To a solution of 2,4,6-trichloropyridine (3 g, 16.447 mmol) in DMF (50 mL) were added dimethyl malonate (2.4 g, 18.092 mmol) and Cs₂CO₃ (10.7 g, 32.895 mmol) at room temperature. The mixture was stirred at 70° C. for 18 h. The mixture was poured into water and extracted with EtOAc. The combined organic layers were washed with brine, dried over anhydrous Na₂SO₄, and concentrated under reduced pressure. The residue was purified by flash silica gel chromatography to afford Compound 3 (2.4 g, 52.5%). LCMS: 278.0 [M+H]⁺.

### Step 2: Preparation of Compound 35-2

To a mixture of Compound 35-1 (2.13 g, 7.659 mmol) and K₂CO₃ (3.2 g, 22.977 mmol) in DMF (20 mL) was added 3-bromoprop-1-ene (1.9 g, 15.702 mmol) dropwise at 0° C. The mixture was stirred at 0° C. for 2 h. The reaction was quenched by the addition of water and the resulting mixture was extracted with EtOAc. The combined organic layers were washed with brine, dried over anhydrous Na₂SO₄, and concentrated under reduced pressure. The residue was purified by flash silica gel chromatography to afford Compound 35-2 (2.20 g, 90.2%). LCMS: 318.0 [M+H]⁺.

### Step 3: Preparation of Compound 35-3

To a mixture of Compound 35-2 (2 g, 6.287 mmol) in THF (10 mL) was added DIBAL-H (25 mL, 25 mmol) dropwise at −20° C. The mixture was stirred at room temperature for 16 h. The reaction was quenched by the addition of MeOH dropwise at −20° C. and stirred for 10 min at −10° C. The mixture was poured to an aqueous solution of Rochelle's salt at 10° C. and stirred for another 15 min. The mixture was extracted with EtOAc. The combined organic layers were washed with brine, dried over anhydrous Na₂SO₄, and concentrated under reduced pressure. The residue was purified by flash silica gel chromatography to afford Compound 35-3 (1100 mg, 66.8%). LCMS: 262.0 [M+H]⁺.

### Step 4: Preparation of Compound 35-4

To a solution of Compound 35-3 (2.1 g, 8.012 mmol) in Toluene (20 mL) was added PPh₃ (4.2 g, 16.024 mmol) at room temperature. After stirring for 10 min. DEAD (1608.4 mg, 6.868 mmol) and zinc bis[(diethylamino)methanedithioate](4.3 g, 12.018 mmol) in toluene (60 mL) was added dropwise to the solution. The mixture was stirred at room temperature for 16 h. The solids were filtered off and the filtrate was concentrated under reduced pressure. The residue was purified by flash silica gel chromatography to afford Compound 35-4 (1.5 g, 76.7%). LCMS: 244.1 [M+H]⁺.

### Step 5: Preparation of Compound 35-5

To a solution of Compound 35-4 (852 mg, 3.49 mmol) in EtOAc (4 mL), CH₃CN (4 mL) and H₂O (6.4 mL) was added RuCl₃ (36 mg, 0.175 mmol). After stirring for 20 min at room temperature, NaIO₄ (2.9 g, 13.961 mmol) was added in batches under ice bath. The reaction mixture was stirred for another 1.5 h. Upon completion, 50 mL water was added to the mixture, then the aqueous layer was extracted with EtOAc (100 mL×3). The combined organic layer was washed with brine (100 mL), dried over anhydrous Na₂SO₄, filtered and concentrated under reduced pressure to afford a residue, which was purified by flash silica gel chromatography to afford Compound 35-5. LCMS: 262.0 [M+H]⁺.

### Step 6: Preparation of Compound 35-6

With Compound 35-5 as the substrate, Compound 35-6 was synthesized according to the Step 2 to Step 5 in the preparation of Example 31. LCMS: 299.0 [M+H]⁺.

### Step 7: Preparation of Compound 35-7

To a solution of Compound 35-6 (20 mg, 0.067 mmol) and Intermediate 3 (22 mg, 0.067 mmol) in dioxane (2 mL) was added methyl[(1R,2R)-2-(methylamino)cyclohexyl]amine (9 mg, 0.067 mmol), K₂CO₃ (18.5 mg, 0.135 mmol) and CuI (6.4 mg, 0.034 mmol) at 25° C. under N₂. The reaction was stirred at 120° C. for 16 h. The mixture was filtered through a Celite pad, and the filtrate was concentrated to give a residue. The residue was purified by reverse phase pre-HPLC to afford Compound 35-7. LCMS: 588.4 [M+H]⁺.

### Step 8: Preparation of Example 35

To a solution of Compound 35-7 (120 mg, 0.204 mmol) in DMF (2 mL) was added TEA (0.14 mL, 1.020 mmol), Pd(dppf)Cl₂ (74.7 mg, 0.102 mmol) and HCOOH (46.9 mg, 1.020 mmol) at 25° C. under N₂. The reaction mixture was stirred at 70° C. for 1 h. Upon completion, the mixture was filtered through a Celite pad, and the filtrate was concentrated to give a residue, which was purified by reverse phase pre-HPLC to afford the title compound. LCMS: 554.0 [M+H]⁺; H NMR (400 MHz, Chloroform-d) δ 8.64 (s, 1H), 8.50 (d, J=5.1 Hz, 1H), 8.42 (s, 1H), 8.15 (s, 1H), 7.97 (s, 1H), 7.46 (s, 1H), 7.05 (d, J=5.0 Hz, 1H), 5.09 (dd, J=31.3, 6.4 Hz, 4H), 3.61 (s, 3H), 3.18 (s, 2H), 2.75 (dd, J=19.6, 9.3 Hz, 2H), 2.01-1.87 (m, 2H), 1.81-1.49 (m, 6H), 0.92-0.76 (m, 4H).

### Intermediate 4

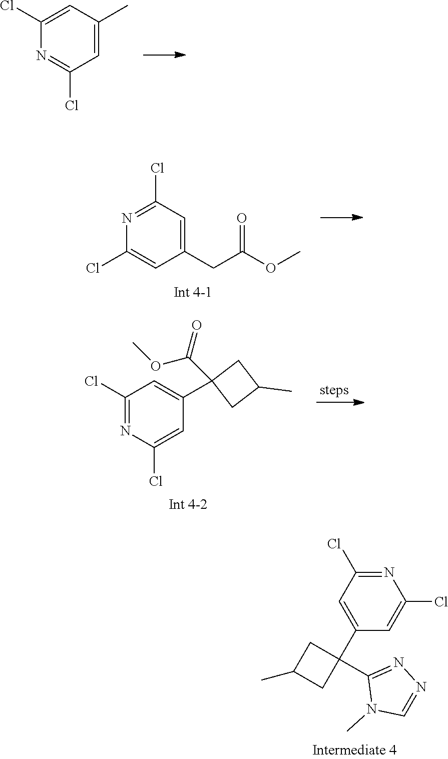

### Step 1: Preparation of Int 4-1

To a stirred solution of 2,6-dichloro-4-methylpyridine (63 g, 388.889 mmol) in anhydrous THF (500 mL) was slowly added LDA (388.89 mL, 777.778 mmol) at −78° C. under N₂. The mixture stirred at −78° C. for 0.5 h. A solution of methyl methoxymethanoate (81.94 mL, 972.222 mmol) in dry THF (130 mL) was added dropwise. The solution was stirred at 0° C. for 1 h. The reaction was quenched with aq. NH₄Cl (500 mL). The resulting mixture was extracted with EtOAc (200 mL×3). The combined organic phase was washed with aqueous sodium bicarbonate, brine, and dried over anhydrous sodium sulfate.

After filtration, the organic layer was concentrated under reduced pressure to give a residue, which was purified by silica gel column chromatography to afford Int 4-1. LCMS: 220.1 [M+H]⁺.

### Step 2: Preparation of Int 4-2

To a solution of Int 4-1 (50.0 g, 231.364 mmol) in DMF (510 mL) was added NaH (20.4 g, 509 mmol) in batches at 0° C. The mixture was stirred at 0° C. for 30 mins. Then the solution was stirred at 25° C. for 2 hrs. Upon completion, the mixture was poured into ammonium chloride aqueous solution (700 mL). Then the mixture was extracted with Ethyl acetate (300 mL×3). The combined organic phase was washed with brine (100 mL), dried over anhydrous Na₂SO₄, filtered, concentrated in vacuo, and purified by column chromatography to afford Int 4-2. LCMS: 274.1 [M+H]⁺.

### Step 3: Preparation of Intermediate 4

With Int 4-2 as the substrate, Intermediate 4 was synthesized according to the Step 2 to Step 5 in the preparation of Example 31. LCMS: 297.1 [M+H]⁺.

### Example 38

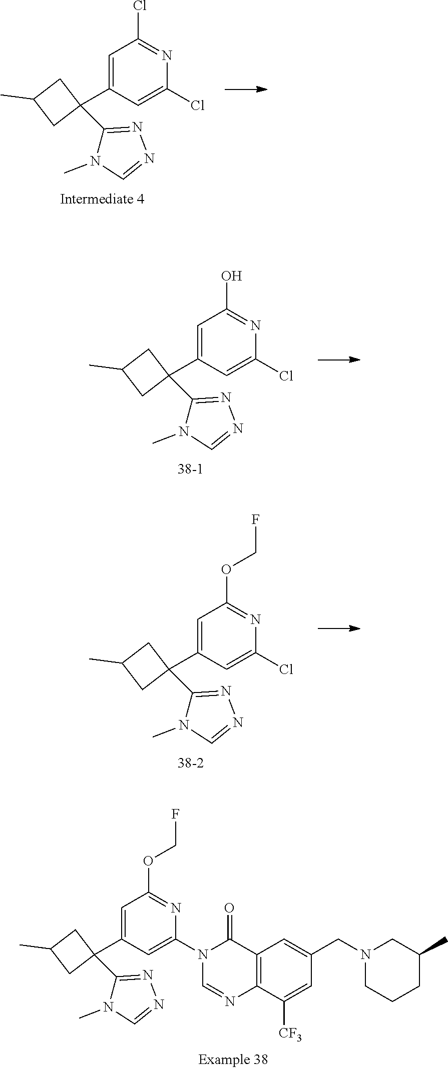

### Step 1: Preparation of Compound 38-1

To a solution of Intermediate 4 (400 mg, 1.346 mmol) in DMSO (6 mL) was added K₂CO₃ (558.0 mg, 4.038 mmol) and N-hydroxyacetamide (303.2 mg, 4.038 mmol). The mixture was stirred at 80° C. for 18 hrs. The mixture was purified by reverse phase prep-HPLC to afford Compound 38-1. LCMS: 279.2 [M+H]⁺.

### Step 2: Preparation of Compound 38-2

To a solution of Compound 38-1 (250 mg, 0.897 mmol) in DMF (3 mL) was added Cs₂CO₃ (292.2 mg, 0.897 mmol). The mixture was stirred at 0° C. for 10 min and then fluoromethyl 4-methylbenzenesulfonate (366.3 mg, 1.794 mmol) was added. The resulting mixture was heated to 80° C. for 2 hrs under N₂. The mixture was purified by reverse phase prep-HPLC to afford Compound 38-2. LCMS: 311.2 [M+H]⁺.

### Step 3: Preparation of Example 38

To a solution of Compound 38-2 (100 mg, 0.322 mmol) in dioxane (2 mL) was added Intermediate 3 (73.3 mg, 0.225 mmol), CuI (30.6 mg, 0.161 mmol), K₂CO₃ (88.9 mg, 0.644 mmol) and methyl[(1R,2R)-2-(methylamino)cyclohexyl]amine (45.8 mg, 0.322 mmol). The mixture was stirred at 120° C. for 18 hrs under N₂. After concentrated under reduced pressure, the residue was purified by reverse phase prep-HPLC to afford the title compound. LCMS: 600.4 [M+H]⁺; ¹H NMR (400 MHz, CDCl₃) δ 8.74 (s, 1H), 8.53-8.38 (m, 1H), 8.22-8.00 (m, 2H), 7.74-7.56 (m, 1H), 6.98-6.69 (m, 1H), 6.14-5.88 (m, 2H), 3.66-3.31 (m, 5H), 3.29-2.85 (m, 2H), 2.82-2.66 (m, 4H), 2.38-1.89 (m, 2H), 1.83-1.13 (m, 8H), 0.94-0.81 (m, 4H).

### Intermediate 5

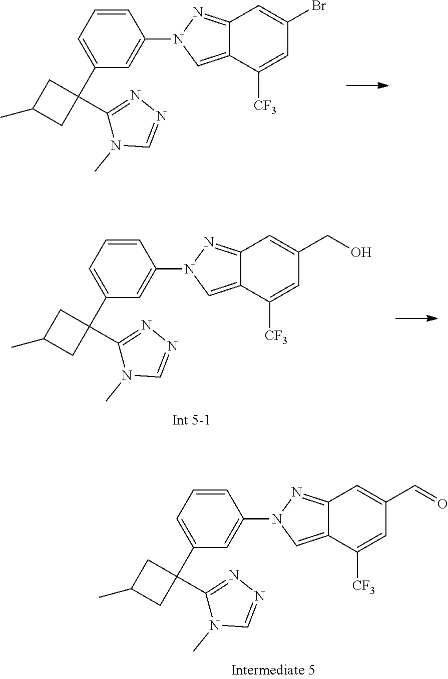 [SMILES: CC1CC(c2cccc(-n3cc4c(C(F)(F)F)cc(Br)cc4n3)c2)(c2nncn2C)C1 ; CC1CC(c2cccc(-n3cc4c(C(F)(F)F)cc(CO)cc4n3)c2)(c2nncn2C)C1 ; CC1CC(c2cccc(-n3cc4c(C(F)(F)F)cc(C=O)cc4n3)c2)(c2nncn2C)C1]

### Step 1: Preparation of Int 5-1

To a solution of 6-bromo-2-(3-(3-methyl-1-(4-methyl-4H-1,2,4-triazol-3-yl)cyclobutyl)phenyl)-4-(trifluoromethyl)-2H-indazole (288 mg, 0.587 mmol) in dioxane (5 mL) was added (tributylstannyl)methanol (565.79 mg, 1.762 mmol) and XPhos Pd G₂ (3.21 mg, 0.004 mmol). The mixture was stirred at 80° C. for 5 hrs. Then the reaction mixture was concentrated under reduced pressure to give a residue, which was purified by silica gel chromatography to afford Compound 3-5 (145 mg). LCMS: 442.2 [M+H]⁺.

### Step 6: Preparation of Intermediate 5

To a solution of Int 5-1 (50 mg, 0.113 mmol) in DCM (2 mL) was added 1,1-diacetoxy-3-oxo-1,3-dihydrobenzo[d][1,2]iodoxole-1-yl acetate (72.06 mg, 0.170 mmol). The mixture was stirred at 20° C. for 1 hr. The reaction was diluted with DCM (20 mL), washed with saturated Na₂SO₃ (10 M), saturated aqueous NaHCO₃ (10 mL) and brine (10 mL), and then concentrated to give Intermediate 5, which could be used directly without further purification. LCMS: 440.2 [M+H]⁺.

The following compounds were synthesized according to step 4 in Example 10 with Intermediate 5 or Intermediate 8 as the substrate.

| Example | LCMS | ¹H NMR |
|---|---|---|
| 40 | 551.3 | ¹H NMR (400 MHz, DMSO-d₆) δ 9.29 (s, 1H), 8.31 (s, 1H), 8.09 (s, 2H), 7.93 |
| | [M + H]⁺ | (s, 1H), 7.68-7.55 (m, 2H), 7.49-7.32 (m, 1H), 3.65 (s, 2H), 3.53 (s, 2H), |
| | | 3.21 (s, 3H), 2.99-2.91 (m, 2H), 2.72-2.26 (m, 6H), 2.03-1.85 (m, 4H), |
| | | 1.80-1.64 (m, 1H), 1.54-1.21 (m, 2H), 1.11 (d, J = 5.2 Hz, 3H). |
| 56 | 533.5 | ¹H NMR (400 MHz, Chloroform-d) δ 8.59 (s, 1H), 8.22 (s, 1H), 8.05 (s, 2H), |
| | [M + H]⁺ | 7.80 (d, J = 8.1 Hz, 1H), 7.61-7.51 (m, 2H), 7.46 (d, J = 8.1 Hz, 1H), 4.42- |
| | | 4.27 (m, 1H), 3.64 (s, 2H), 3.31 (s, 3H), 3.10 (s, 1H), 2.98 (s, 2H), 2.78-2.57 |
| | | (m, 5H), 2.26-1.80 (m, 5H), 1.54-1.43 (m, 1H), 1.18 (d, J = 4.5 Hz, 3H). |
| 64 | 551.4 | ¹H NMR (400 MHz, Chloroform-d) δ 8.44 (s, 1H), 7.91-7.80 (m, 2H), 7.76 |
| | [M + H]⁺ | (d, J = 7.4 Hz, 1H), 7.60-7.51 (m, 2H), 7.43 (t, J = 8.0 Hz, 1H), 6.85 (s, 1H), |
| | | 5.21-5.10 (m, 4H), 3.77-3.50 (m, 3H), 2.97-2.87 (m, 4H), 2.81-2.47 (m, |
| | | 2H), 2.48-1.67 (m, 6H), 1.61-1.18 (m, 6H). |
| 69 | 535.4 | ¹H NMR (400 MHz, Chloroform-d) δ 8.44 (s, 1H), 8.04-7.79 (m, 2H), 7.76 |
| | [M + H]⁺ | (dd, J = 7.8, 1.7 Hz, 1H), 7.61-7.51 (m, 2H), 7.42 (t, J = 8.0 Hz, 1H), 6.85 |
| | | (d, J = 7.8 Hz, 1H), 5.18 (d, J = 6.3 Hz, 2H), 5.13 (d, J = 6.2 Hz, 2H), 3.61 (s, |
| | | 3H), 3.10-2.83 (m, 4H), 2.16-0.73 (m, 9H). |
| 74 | 575.4 | — |
| | [M + H]⁺ | |
| 79 | 521.2 | ¹H NMR (400 MHz, Chloroform-d) δ 8.47 (s, 1H), 8.16-7.99 (m, 1H), 7.87 |
| | [M + H]⁺ | (s, 2H), 7.79 (d, J = 8.3 Hz, 1H), 7.57 (s, 1H), 7.53-7.44 (m, 1H), 7.44- |
| | | 7.30 (m, 1H), 5.54-5.35 (m, 1H), 3.74 (s, 2H), 3.48 (s, 3H), 3.33-3.10 (m, |
| | | 2H), 2.99-2.72 (m, 4H), 2.74-2.57 (m, 2H), 2.16 (s, 2H), 1.83-1.60 (m, |
| | | 7H). |
| 86 | 537.0 | ¹H NMR (400 MHz, Chloroform-d) δ 8.45 (d, J = 13.3 Hz, 1H), 8.21-7.90 |
| | [M + H]⁺ | (m, 3H), 7.76 (s, 1H), 7.58-7.37 (m, 3H), 4.86-4.58 (m, 2H), 3.58-3.46 |
| | | (m, 3H), 3.32-3.17 (m, 2H), 2.97-2.77 (m, 2H), 2.73-1.86 (m, 4H), 1.77- |
| | | 1.56 (m, 3H), 1.50-1.30 (m, 3H), 1.27-1.13 (m, 2H), 1.11-0.96 (m, 6H). |
| 89 | 539.3 | ¹H NMR (400 MHz, Chloroform-d) δ 8.43 (s, 1H), 8.16-7.94 (m, 3H), 7.79- |
| | [M + H]⁺ | 7.69 (m, 1H), 7.58-7.31 (m, 3H), 4.19-3.82 (m, 2H), 3.27 (s, 3H), 3.15- |
| | | 3.03 (m, 1H), 3.03-2.85 (m, 2H), 2.78-2.62 (m, 2H), 2.55-2.23 (m, 3H), |
| | | 2.11-1.66 (m, 4H), 1.48-1.32 (m, 1H), 1.22 (s, 3H), 1.19-1.13 (m, 3H). |

### Example 41

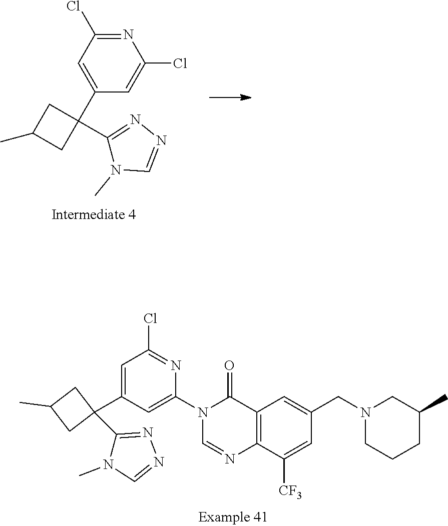

The title compound was synthesized according to Step 3 in Example 38. LCMS: 586.0 [M+H]⁺; ¹H NMR (400 MHz, DMSO-d₆) δ 8.72-8.57 (m, 1H), 8.50-8.31 (m, 2H), 8.16 (s, 1H), 7.94-7.42 (m, 2H), 4.76-4.58 (m, 2H), 3.58 (s, 3H), 3.30-3.11 (m, 4H), 2.97-2.58 (m, 4H), 2.04-1.84 (m, 1H), 1.70-1.41 (m, 5H), 1.30-1.03 (m, 2H), 0.91-0.73 (m, 4H).

### Example 42

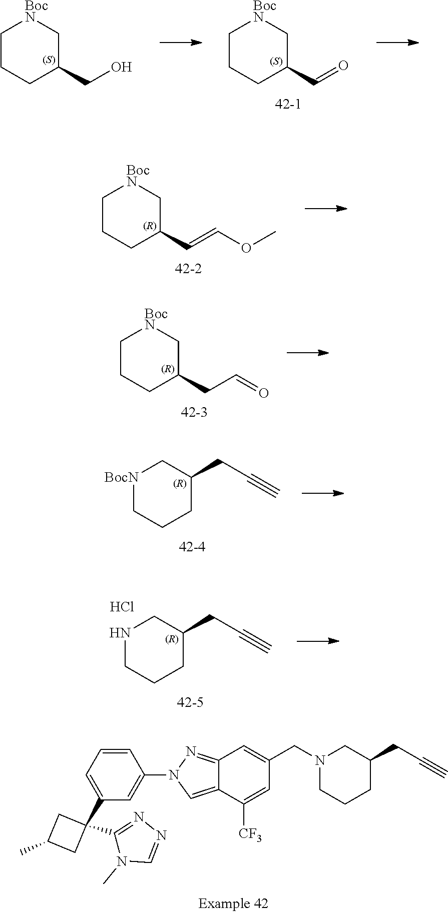

### Step 1: Preparation of Compound 42-1

To a solution of tert-butyl (S)-3-(hydroxymethyl)piperidine-1-carboxylate (5 g, 23.223 mmol) in DCM (20 mL) was added DMP (11.8 mg, 27.868 mmol) at 0° C. The mixture was stirred at room temperature for 2 hrs under N₂. The mixture was filtered and the cake was washed with DCM, and the filtrate was concentrated to give a residue, which was further purified by flash silica gel chromatography to give Compound 42-1 (4.375 g, 88.3%). ¹H NMR: (400 MHz, CDCl₃) δ 9.69 (s, 1H), 3.90 (s, 1H), 3.64 (d, J=12.3 Hz, 1H), 3.32 (dd, J=13.5, 8.3 Hz, 1H), 3.08 (ddd, J=13.0, 9.3, 3.3 Hz, 11H), 2.42 (s, 1H), 1.95 (d, J=6.4 Hz, 1H), 1.77-1.52 (m, 3H), 1.45 (s, 9H).

### Step 2: Preparation of Compound 42-2

To a solution of Compound MTPPC (10.5 g, 30.767 mmol) in THE (20 mL) was added KHMDS (3.5 g, 30.767 mmol) at 0° C. The mixture was stirred at 0° C. for 30 mins. Then Compound 42-1 (4.375 g, 20.511 mmol) in THE (13 mL) was added to the mixture at −20° C. The mixture was stirred at 25° C. for 12 hrs. The reaction mixture was partitioned between water (20 mL) and EtOAc (15 mL). The organic phase was separated, washed with brine 15 mL (15 mL×3), dried over anhydrous sodium sulfate, filtered, and concentrated under reduced pressure to give a crude. The combined crude product was purified by flash silica gel chromatography to give Compound 42-2 (3.761 g, 70.0%).

### Step 3: Preparation of Compound 42-3

To a solution of Compound 42-2 (3 g, 12.433 mmol) in MeOH (30 mL) was added HCl (1 ml, 1.000 mmol), the mixture was stirred at room temperature for 6 hrs under N₂. The reaction mixture was partitioned between water (20 mL) and EA (15 mL). The organic phase was separated, washed with brine 15 mL (3 times), dried over anhydrous sodium sulfate, filtered, and concentrated under reduced pressure to give a crude. The crude product was used into the next step without further purification.

### Step 4: Preparation of Compound 42-4

To a solution of Compound 42-3 (2700 mg, 11.879 mmol) in MeOH (30 mL) was added K₂CO₃ (3283.2 mg, 23.757 mmol) and Bestmann reagent (2738.2 mg, 14.254 mmol) at 0° C. The mixture was stirred at room temperature for 12 hrs under N₂. The reaction mixture was partitioned between water (30 mL) and EtOAc (30 mL). The organic phase was separated, dried over anhydrous sodium sulfate, filtered, and concentrated under reduced pressure to give the residue. The residue was purified by flash silica gel chromatography to give Compound 42-4 (471 mg, 17.8%).

### Step 5: Preparation of Compound 42-5

A mixture of Compound 42-4 (200 mg, 0.896 mmol) in HCl/dioxane (4M, 6 mL) was stirred at room temperature for 1 hr. The mixture reaction was concentrated under reduced pressure to give the crude Compound 42-5. LCMS: 124.1 [M+H]⁺; The crude product was used to the next step without further purification.

### Step 6: Preparation of Example 42

The title compound was synthesized according to the procedure of step 4 in Example 10 with Compound 42-5 and Intermediate 5 as the substrates. LCMS: 547.4 [M+H]⁺; ¹H NMR (400 MHz, Methanol-d₄) δ 9.10 (s, 1H), 8.52 (s, 1H), 8.19 (s, 1H), 8.11 (s, 1H), 8.06-7.98 (m, 1H), 7.71-7.64 (m, 2H), 7.63-7.57 (m, 1H), 4.55 (s, 2H), 3.66-3.52 (m, 2H), 3.38 (s, 3H), 3.08-2.82 (m, 4H), 2.75-2.63 (m, 3H), 2.37-2.21 (m, 2H), 2.13-1.92 (m, 4H), 1.85-1.69 (m, 1H), 1.18 (d, J=5.9 Hz, 3H).

### Intermediate 6

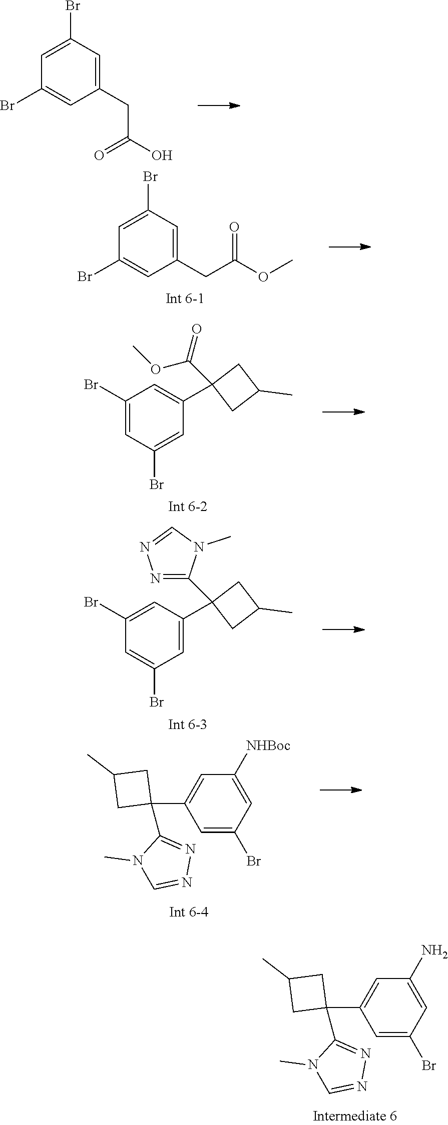

### Step 1: Preparation of Intermediate 6-1

(3,5-dibromophenyl)acetic acid (40 g, 136.082 mmol) was dissolved in MeOH (400 mL) and stirred at 0° C. While stirring, H₂SO₄ (7.253 mL, 136.082 mmol) was added dropwise. The mixture was then heated to reflux for 3 hrs. The reaction mixture was concentrated under reduced pressure to remove solvent. The residue was diluted with water 200 mL and extracted with solvent EtOAc (300 mL×3). The combined organic layers were dried over anhydrous sodium sulfate, filtered, and concentrated under reduced pressure to give Int 6-1 (41.9 g, 100.0%). ¹H NMR (400 MHz, CDCl₃) δ 7.58 (t, J=1.7 Hz, 1H), 7.38 (d, J=1.7 Hz, 2H), 3.72 (s, 3H), 3.57 (s, 2H).

### Step 2: Preparation of Intermediate 6-2

Int 6-2 was synthesized according to Step 2 in Intermediate 7.

### Step 3: Preparation of Intermediate 6-3

Int 6-3 was synthesized according to Step 2 to Step 5 in the preparation of Example 31. LCMS: 386.0 [M+H]⁺.

### Step 4: Preparation of Intermediate 6-4

A mixture of Int 6-3 (10.6 g, 27.525 mmol), NH₂Boc (3.2 g, 27.525 mmol), Xantphos (3.2 g, 5.505 mmol), Pd₂(dba)₃ (2.5 g, 2.753 mmol) and Cs₂CO₃ (17.9 g, 55.051 mmol) in dioxane (100 mL) was degassed and purged with N₂ for 3 times. And then the mixture was stirred at 100° C. for overnight under N₂ atmosphere. The mixture was filtered through a Celite pad, the reaction mixture was concentrated under reduced pressure to remove solvent. The residue was purified by silica gel column chromatography to afford Int 6-4 (7 g, 60.4%). LCMS: 423.2 [M+H]⁺.

### Step 5: Preparation of Intermediate 6

To a solution of Int 6-4 (2 g, 4.747 mmol) in DCM (15 mL) was added TFA (5 mL) at 25° C., and the mixture was stirred at 25° C. for 1 hr. The mixture was concentrated to afford Intermediate 6 (1.4 g, 91.8%). LCMS: 321.1 [M+H]⁺.

### Example 43

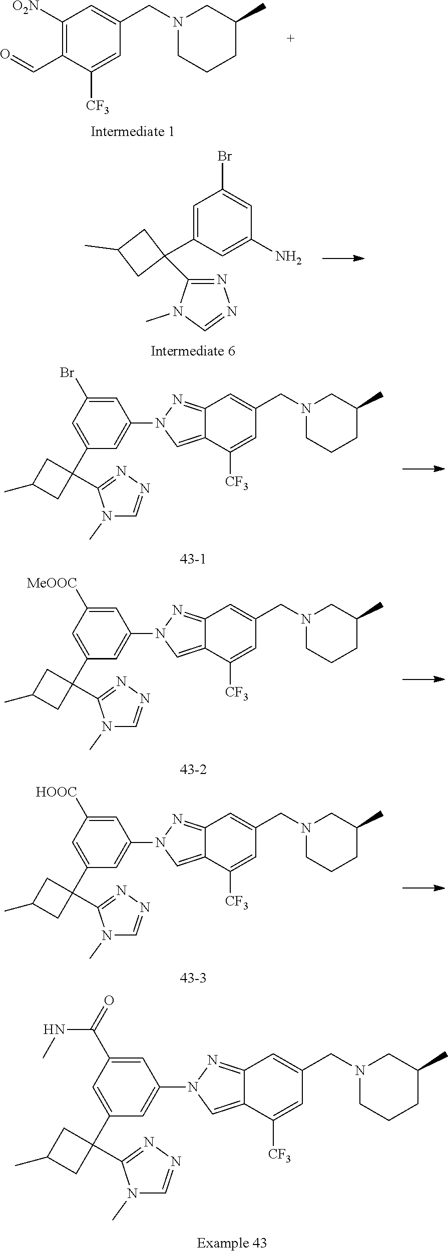

### Step 1: Preparation of Compound 43-1

Compound 43-1 was synthesized according to the Step 6 in Example 32. LCMS: 603.1 [M+H]⁺.

### Step 2: Preparation of Compound 43-2

A solution of Compound 43-1 (240 mg, 0.399 mmol), Pd(dppf)Cl₂ (146.0 mg, 0.200 mmol) and TEA (0.41 mL, 2.993 mmol) in MeOH (3 mL) was stirred at 60° C. for 12 hrs under CO (50 Psi). The residue was purified by flash silica gel chromatography to afford Compound 42-2. LCMS: 581.4 [M+H]⁺.

### Step 3: Preparation of Compound 43-3

A mixture of Compound 43-2 (204 mg, 0.351 mmol) and LiOH (14.7 mg, 0.351 mmol) in EtOH (2 mL) and H₂O (1 mL) was stirred at 25° C. for 2 hrs. The mixture was adjusted to pH=2-3 with 1 M HCl and concentrated under reduced pressure to give a residue. The residue was purified by reversed-phase chromatography to afford Compound 43-3. LCMS: 567.6 [M+H]⁺.

### Step 4: Preparation of Example 43

To a solution of Compound 43-3 (50 mg, 0.088 mmol) and HATU (50.3 mg, 0.132 mmol) in DMF (1.5 mL) was added methylammonium chloride (7.1 mg, 0.106 mmol) and DIPEA (0.06 mL, 0.353 mmol). The reaction was stirred at 25° C. for 2 hrs. The mixture was purified by reverse phase prep-HPLC to afford the title compound. LCMS: 580.6 [M+H]⁺; ¹H NMR (400 MHz, Chloroform-d) δ 8.59 (s, 1H), 8.34-8.10 (m, 2H), 8.07-7.93 (m, 1H), 7.91-7.72 (m, 2H), 7.65-7.45 (m, OH), 6.73-6.43 (m, 1H), 3.61 (s, 2H), 3.39-3.18 (m, 5H), 3.12-2.91 (m, 6H), 2.88-2.52 (m, 4H), 2.08-1.89 (m, 1H), 1.85-1.53 (m, 4H), 1.20-1.11 (m, 3H), 0.95-0.76 (m, 4H).

### Example 44

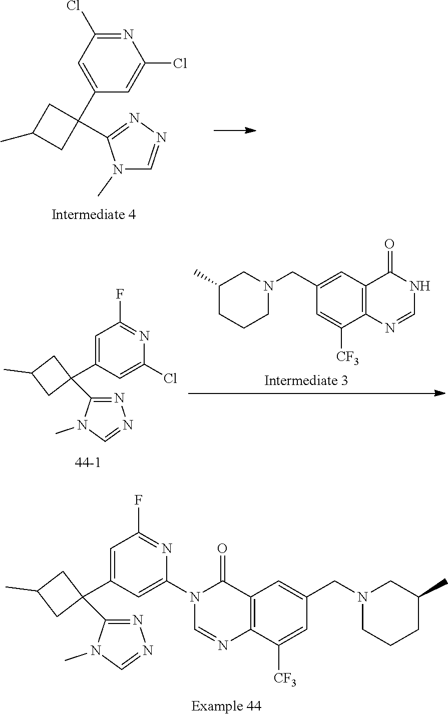

### Step 1: Preparation of Compound 44-1

To a solution of Intermediate 4 (300 mg, 1.009 mmol) in DMSO (5 mL) was added potassium fluoride (58.6 mg, 1.009 mmol). The mixture was stirred at 140° C. for 6 hrs. The mixture was purified by reverse phase chromatography to afford Compound 44-1. LCMS: 281.0 [M+H]⁺.

### Step 2: Preparation of Example 44

The title compound was synthesized according to the Step 3 in Example 38. LCMS: 570.6 [M+H]⁺; H NMR (400 MHz, Chloroform-d) δ 8.69 (s, 1H), 8.44 (s, 1H), 8.31-8.04 (m, 2H), 7.91-7.69 (m, 1H), 7.16-6.86 (m, 1H), 3.78-3.32 (m, 5H), 3.30-3.11 (m, 1H), 2.97-2.52 (m, 5H), 2.42-1.92 (m, 2H), 1.87-1.54 (m, 7H), 1.23-1.11 (m, 1H), 1.01-0.78 (m, 4H).

### Example 45

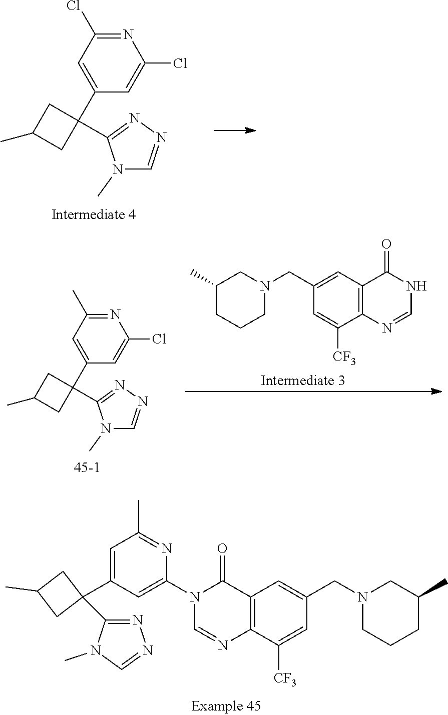

### Step 1: Preparation of Compound 45-1

To a solution of Intermediate 4 (500 mg, 1.682 mmol) and methylboranediol (90.7 mg, 1.514 mmol) in dioxane (4 mL) and H₂O (1 mL) was added Pd(PPh₃)₄ (209.3 mg, 0.168 mmol), Potassium carbonate (581.3 mg, 4.206 mmol) at room temperature. The mixture was stirred at 80° C. for 14 hrs. The mixture was filtered through a Celite pad, and the filtrate was concentrated to give the crude product. The crude product was purified by reversed-phase chromatography to afford Compound 45-1. LCMS: 277.2 [M+H]⁺.

### Step 2: Preparation of Example 45

The title compound was synthesized according to the Step 3 in Example 38. LCMS: 566.4 [M+H]⁺; ¹H NMR (400 MHz, Chloroform-d) δ 8.76-8.68 (m, 1H), 8.44 (s, 1H), 8.20-8.02 (m, 2H), 7.81-7.55 (m, 1H), 7.21-6.97 (m, 1H), 3.67-3.29 (m, 5H), 3.19 (dd, J=14.5, 7.3 Hz, 1H), 2.95-2.86 (m, 2H), 2.83-2.69 (m, 3H), 2.54 (s, 3H), 2.01-1.92 (m, 1H), 1.76-1.58 (m, 8H), 1.16-1.14 (m, 1H), 0.90-0.81 (m, 4H).

### Example 46

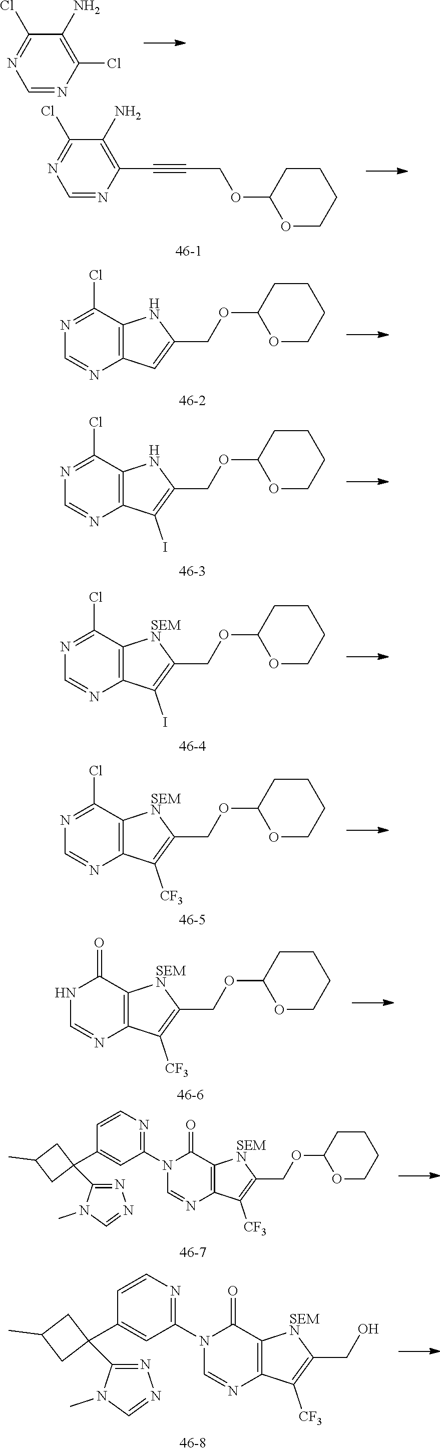

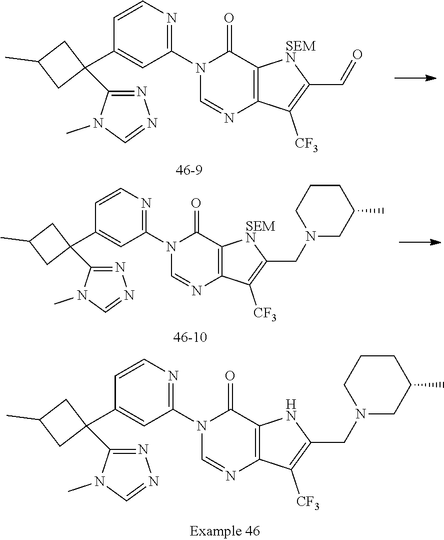

### Step 1: Preparation of Compound 46-1

To a solution of 4,6-dichloropyrimidin-5-amine (11.70 g, 71.337 mmol) in EtOAc (100 mL) was added 2-(prop-2-yn-1-yloxy)tetrahydro-2H-pyran (5 g, 35.668 mmol), Tetrakis(triphenylphosphine)palladium (2.06 g, 1.783 mmol) and CuI (0.34 g, 1.783 mmol). The reaction was stirred at 80° C. for 2 hrs under N₂. After cooling to room temperature, the reaction was diluted with EtOAc. The organic phase was separated, washed with brine (50 mL×3), dried over anhydrous sodium sulfate, filtered, and concentrated under reduced pressure to give a residue. The residue was purified by flash silica gel chromatography to afford Compound 46-1 (3.1 g, 32.46%). LCMS: 268.0 [M+H]⁺.

### Step 2: Preparation of Compound 46-2

To a solution of Compound 46-1 (3.1 g, 11.580 mmol) in NMP (60 mL) was added ^{t}BuOK (2.60 g, 23.159 mmol). And the reaction was stirred at 0° C. for 3 hrs under N₂. The reaction was diluted with EtOAc. The organic phase was separated, washed with brine (40 mL×3), dried over anhydrous sodium sulfate, filtered, and concentrated under reduced pressure to give a residue. The residue was purified by flash silica gel chromatography to afford Compound 46-2 (2.7 g, 87.10%). LCMS: 268.0 [M+H]⁺.

### Step 3: Preparation of Compound 46-3

To a solution of Compound 46-2 (6 g, 22.412 mmol) in MeCN (120 mL) was added NIS (6.05 g, 26.895 mmol), and the reaction was stirred at 0° C. for 2 hrs under N₂. The reaction mixture was diluted with EtOAc. The organic phase was separated, washed with brine (50 mL×3), dried over anhydrous sodium sulfate, filtered, and concentrated under reduced pressure to give a residue. The residue was purified by flash silica gel chromatography to afford Compound 46-3 (6.6 g, 74.8%). LCMS: 394.0 [M+H]⁺.

### Step 4: Preparation of Compound 46-4

To a solution of Compound 46-3 (2 g, 5.081 mmol) in DMF (40 mL) was added Cs₂CO₃ (5.0 g, 15.244 mmol) and SEMCl (1.3 g, 7.622 mmol). The reaction was stirred at 0° C. for 2 hrs under N₂. The reaction was diluted with EtOAc. The organic phase was separated, washed with brine (50 mL×3), dried over anhydrous sodium sulfate, filtered, and concentrated under reduced pressure to give a residue. The residue was purified by flash silica gel chromatography to afford Compound 46-4 (2 g, 75.10%). LCMS: 524.0 [M+H]⁺;

### Step 5: Preparation of Compound 46-5

To a solution of Compound 46-4 (1 g, 1.909 mmol) in DMF (8 mL) was added methyl 2,2-difluoro-2-(fluorosulfonyl)acetate (340.0 mg, 1.909 mmol) and Cut (0.4 g, 2.291 mmol). The reaction was stirred at 80° C. for 16 hrs under N₂. the reaction was diluted with EtOAc. The organic phase was separated, washed with brine (30 mL×3), dried over anhydrous sodium sulfate, filtered, and concentrated under reduced pressure to give a residue. The residue was purified by flash silica gel chromatography to afford Compound 46-5 (300 mg, 33.7%).

### Step 6: Preparation of Compound 46-6

To a solution of Compound 46-5 (850 mg, 1.824 mmol) in DMSO (6 mL) was added K₂CO₃ (756.2 mg, 5.472 mmol) and N-hydroxyacetamide (274.0 mg, 3.648 mmol). The reaction was stirred at 80° C. for 2 hrs under N₂. After cooling to room temperature, the reaction was diluted with EtOAc. The organic phase was separated, washed with brine (20 mL×3), dried over anhydrous sodium sulfate, filtered, and concentrated under reduced pressure to give a residue. The residue was purified by flash silica gel chromatography to afford Compound 46-6 (550 mg, 67.4%). LCMS: 448.1 [M+H]⁺;

### Step 7: Preparation of Compound 46-7

To a solution of Compound 46-6 (1300.0 mg, 2.905 mmol) and Intermediate 10 (937.0 mg, 3.050 mmol) in dioxane (10 mL) was added CuI (166.0 mg, 0.872 mmol), DMEDA (204.9 mg, 2.324 mmol) and K₂CO₃ (1084.0 mg, 7.844 mmol). The mixture was stirred at 110° C. for 12 hrs under N₂. The reaction mixture was partitioned between water (50 mL) and EtOAc (30 mL). The organic phase was separated, dried over anhydrous sodium sulfate, filtered, and concentrated under reduced pressure to give the residue. The residue was purified by flash silica gel chromatography to give Compound 46-7 (1.7 g, 86.8%). LCMS: 674.3 [M+H]⁺.

### Step 8: Preparation of Compound 46-8

To a solution of Compound 46-7 (1.6 g, 2.375 mmol) in THF (5 mL) was added HCl (1 N, 10 mL) at 0° C. The mixture was stirred at room temperature for 2 hrs under N₂. The mixture was filtered and the filtrate was concentrated to afford the crude Compound 46-8 (1.35 g, 96.4%). The crude product was used directly for the next step without purification. LCMS: 590.2 [M+H]⁺.

### Step 9: Preparation of Compound 46-9

To a solution of Compound 46-8 (1.3 g, 2.205 mmol) in DCM (15 mL) was added Dess-Martin periodinane (1.2 g, 2.866 mmol) at 0° C. The mixture was stirred at room temperature for 3 hrs under N₂. The reaction mixture was quenched with Na₂S₂O₃ (sat. aq. 10 mL) and NaHCO₃ (sat. aq. 10 mL). The mixture was extracted with DCM (10 mL×3). The organic phase was separated, washed with water (10 mL×2), dried over anhydrous sodium sulfate, filtered, and concentrated under reduced pressure to give a residue. The residue was purified by flash silica gel chromatography to afford Compound 46-9 (1.25 g, 96.5%). LCMS: 588.2 [M+H]⁺.

### Step 10: Preparation of Compound 46-10

The Compound 46-10 was synthesized according to step 4 in Example 10. LCMS: 671.5 [M+H]⁺;

### Step 11: Preparation of Example 46

To a solution of Compound 46-10 (66 mg, 0.098 mmol) in DCM (0.5 mL) was added TFA (2 mL, 0.015 mmol) at room temperature. The mixture was stirred at room temperature for 6 hrs. The mixture was concentrated under reduced pressure to give a residue. The residue was purified by prep-HPLC to afford the title compound. LCMS: 541.1 [M+H]⁺.

### Example 47

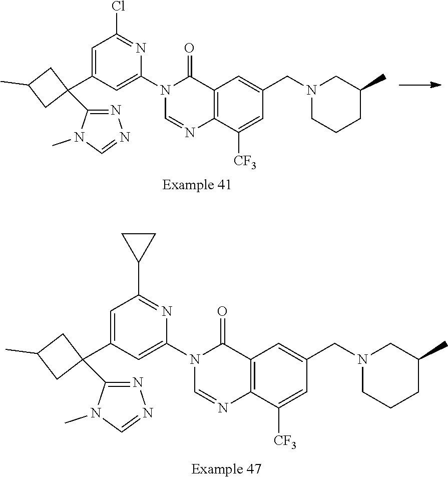

To a solution of Example 41 (30 mg, 0.051 mmol), cyclopropylboronic acid (14 mg, 0.154 mmol), tricyclohexylphosphane (14.4 mg, 0.051 mmol) in dioxane/water (3/1, 2 mL) was added K₃PO₄ (11 mg, 0.051 mmol) and Pd(OAc)₂ (6 mg, 0.026 mmol). The resulting mixture was stirred at 90° C. for 16 hrs. The mixture was filtered through a Celite pad, and the filtrate was concentrated to give the crude product. The crude was purified by reverse phase pre-HPLC to afford the title compound. LCMS: 592.0 [M+H]⁺; ¹H NMR (400 MHz, Chloroform-d) δ 8.77-8.71 (m, 1H), 8.47 (s, 1H), 8.18-8.03 (m, 2H), 7.69-7.51 (m, 1H), 7.23 (s, 1H), 3.65-3.32 (m, 5H), 3.25-3.13 (m, 2H), 3.01-2.64 (m, 4H), 2.03-1.44 (m, 10H), 1.15 (d, J=6.3 Hz, 1H), 1.01 (d, J=8.2 Hz, 4H), 0.90-0.82 (m, 4H).

### Intermediate 7

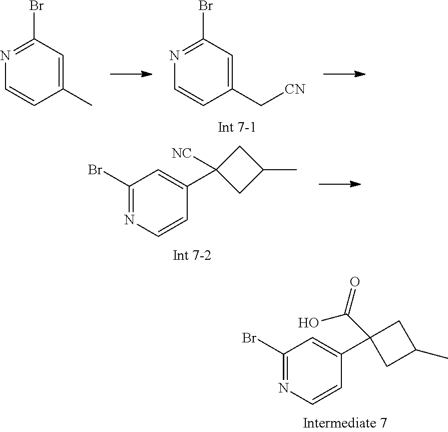

### Step 1: Preparation of Intermediate 7-1

A solution of 2-bromo-4-methylpyridine (20 g, 116 mmol) and t-butoxybisdimethylaminomethane (40.5 g, 232 mmol) was stirred at 110° C. for 15 hours. The reaction solution was cooled and concentrated under reduced pressure to afford a residue. After the addition of water (20 mL) and aminooxidanesulfonic acid (2.5 g, 22.106 mmol), the mixture was stirred at room temperature for 1 hour. After filtration, the mixture was extracted with DCM (100 mL×2). The organic phase was washed with brine, dried over anhydrous Na₂SO₄, filtered and concentrated to give Intermediate 7-1. LCMS: 198.0 [M+H]⁺.

### Step 2: Preparation of Intermediate 7-2

To a solution of Intermediate 7-1 (9.00 g, 45.7 mmol) and 1,3-dibromo-2-methylpropane (9.90 g, 45.7 mmol) in N,N-dimethylmethanamide (6 mL) at 0° C. was added sodium hydride (2.20 g, 91.4 mmol). The reaction mixture was stirred at room temperature for 2 hours. The reaction was quenched by addition of saturated aqueous NH₄Cl. The reaction mixture was extracted with EtOAc (300 mL×2). The combined organic phase was washed with brine, dried over anhydrous Na₂SO₄, filtered and concentrated under reduced pressure to afford a residue, which was purified by silica gel column chromatography to give Intermediate 7-2. LCMS: 252.0 [M+H]⁺.

### Step 3: Preparation of Intermediate 7

A solution of Intermediate 7-2 (7.40 g, 29.5 mmol) and sodium hydroxide (3.5 g, 88.4 mmol) in ethanol (68 mL) and H₂O (13 mL) was stirred at 80° C. for 16 hours. Upon completion, the pH was adjusted to 5 by addition of 1 M HCl at 0° C. The aqueous layer was extracted with EtOAc (400 mL×3). The combined organic layer was washed with brine (100 mL), dried over anhydrous Na₂SO₄, filtered, and concentrated to afford the title compound. LCMS: 271.9 [M+H]⁺.

### Example 48

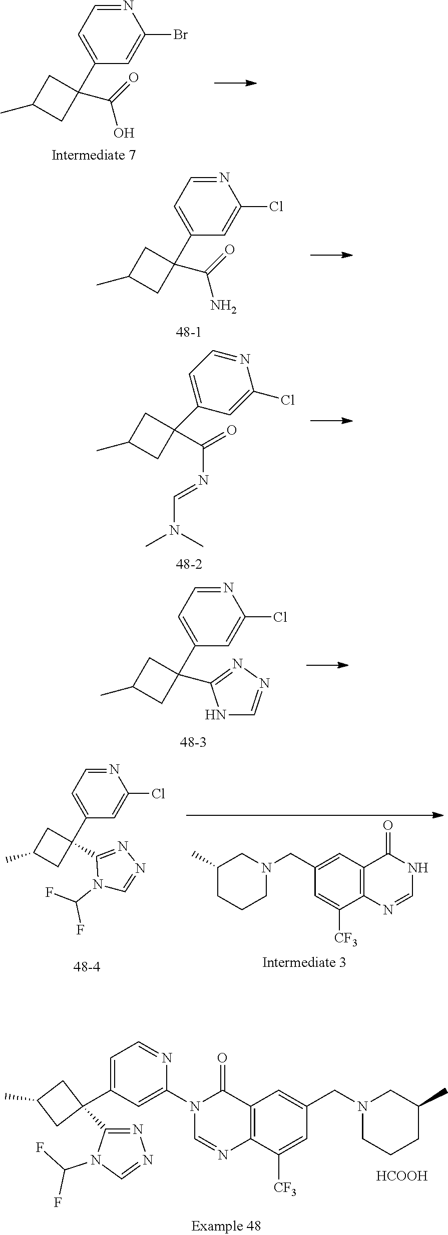

### Step 1: Preparation of Compound 48-1

A solution Intermediate 7 (8.50 g, 31.5 mmol) in SOCl₂ (35 mL) was stirred at 80° C. for 1 hr. Then the mixture was concentrated to give a residue. A solution of the above residue in DCM (35 mL) was added to NH₃/MeOH (7 N, 35 mL) dropwise. The mixture was stirred at room temperature for 30 min. The reaction mixture was concentrated under reduced pressure to remove solvent. The crude product was purified by flash silica gel column chromatography to afford Compound 48-1 (5.0 g, 70.7%). LCMS: 225.0 [M+H]⁺.

### Step 2: Preparation of Compound 48-2

A solution of Compound 48-1 (5.0 g, 22.3 mmol) in DMF-DMA (20 mL) was stirred at 100° C. for 12 hrs. After being cooled to room temperature, the mixture was concentrated under reduced pressure to give the crude Compound 48-2 (5.0 g, crude), which was used to the next step without further purification. LCMS: 280.2 [M+H]⁺.

### Step 3: Preparation of Compound 48-3

To a solution of Compound 48-2 (5.0 g, crude) in HOAc (14 mL) was added hydrazine hydrate (14 mL). The mixture was stirred at 80° C. for 4 hrs. After being cooled to room temperature, the reaction mixture was concentrated to give a residue, which was diluted with water (200 mL) and extracted with EtOAc (300 mL×3). The combined organic layers were washed with brine (100 mL), dried over anhydrous sodium sulfate, filtered, and concentrated under reduced pressure to give a residue, which was purified by reversed-phase HPLC to afford Compound 48-3 (4 g, 72.3%). LCMS: 249.0 [M+H]⁺;

### Step 4: Preparation of Compound 48-4

To a solution of Compound 48-3 (2.00 g, 8.07 mmol) in DMF (20 mL) was added sodium 2-chloro-2,2-difluoroacetate (2.50 g, 16.1 mmol) and K₂CO₃ (2.20 g, 16.1 mmol). The mixture was stirred at 140° C. for 5 hrs. After being cooled to room temperature, the reaction mixture was diluted with water (200 mL) and extracted with EtOAc (200 mL×3). The combined organic layers were washed with brine (200 mL), dried over anhydrous sodium sulfate, filtered, and concentrated under reduced pressure to give the residue, which was purified by reversed-phase HPLC to afford Compound 48-4 (0.15 g, 5.0%). LCMS: 299.0 [M+H]⁺.

### Step 5: Preparation of Example 48

The title compound was synthesized according to the Step 3 in Example 38. LCMS: 588.4 [M+H]⁺; ¹H NMR (400 MHz, DMSO-d₆) δ 8.98 (s, 1H), 8.66 (s, 1H), 8.64 (d, J=5.3 Hz, 1H), 8.39 (s, 1H), 8.18-8.11 (m, 1H), 7.86 (s, 1H), 7.47 (dd, J=5.3, 1.7 Hz, 1H), 7.45 (t, J=58.7 Hz, 1H), 3.66 (s, 2H), 2.87 (p, J=6.2, 5.5 Hz, 2H), 2.77-2.69 (m, 2H), 2.68-2.55 (m, 3H), 1.95 (t, J=11.0 Hz, 1H), 1.71-1.56 (m, 4H), 1.53-1.43 (m, 1H), 1.09 (d, J=5.6 Hz, 3H), 0.93-0.84 (m, 1H), 0.82 (d, J=6.1 Hz, 3H).

### Example 49

The title compound was synthesized according to Example 45 with potassium vinyltrifluoroborate and Intermediate 4 as the substrates. LCMS: 578.4 [M+H]⁺; H NMR (400 MHz, Chloroform-d) δ 8.84 (s, 1H), 8.47 (s, 1H), 8.22-8.06 (m, 2H), 7.71 (d, J=6.3 Hz, 1H), 7.27 (s, 1H), 6.73 (dd, J=17.2, 10.6 Hz, 1H), 6.33 (d, J=17.2 Hz, 1H), 5.54 (d, J=10.7 Hz, 1H), 3.67 (s, 2H), 3.57 (s, 3H), 3.21 (dd, J=15.0, 7.2 Hz, 1H), 2.92 (dd, J=14.1, 8.0 Hz, 1H), 2.87-2.69 (m, 4H), 2.00 (s, 2H), 1.82-1.58 (m, 7H), 1.19-1.12 (m, 1H), 0.97-0.87 (m, 1H), 0.86 (d, J=5.9 Hz, 3H).

### Intermediate 8

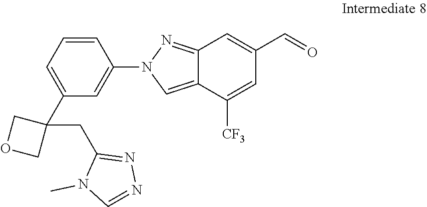 [SMILES: Cn1cnnc1CC1(c2cccc(-n3cc4c(C(F)(F)F)cc(C=O)cc4n3)c2)COC1]

The title compound was synthesized according to the procedure in Intermediate 5. LCMS: 442.0 [M+H]⁺.

### Example 51

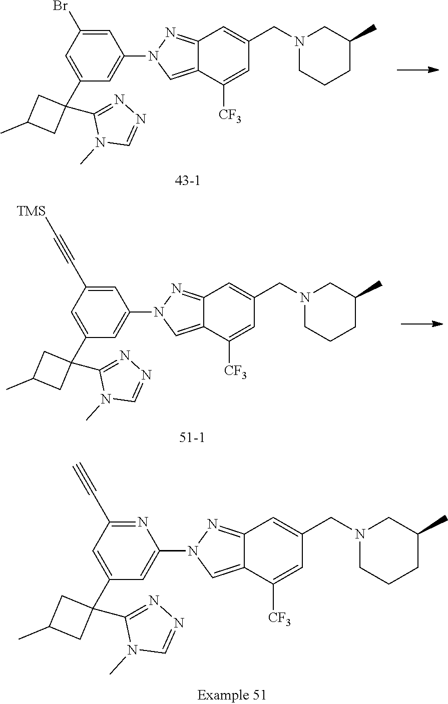

### Step 1: Preparation of Compound 51-1

To a solution of Compound 43-1 (60 mg, 0.100 mmol) in THF (2 mL) was added CuI (1.9 mg, 0.010 mmol), Trimethylsilylacetylene (195.9 mg, 1.995 mmol), Pd(PPh₃)₂Cl₂ (7.8 mg, 0.010 mmol) and triethylamine (0.07 mL, 0.499 mmol). The mixture was stirred at 70° C. for overnight under N₂. The reaction solution proceeds directly to the next step. LCMS: 619.8 [M+H]⁺.

### Step 2: Preparation of Example 51

To a solution of Compound 51-1 (60 mg, 0.097 mmol) in MeOH (2 mL) was added K₂CO₃ (40.2 mg, 0.291 mmol). The mixture was stirred at 25° C. for 1 hour. The reaction mixture was filtered and concentrated under reduced pressure to give a residue. The residue was purified by reverse phase prep-HPLC to afford Example 51. LCMS: 547.6 [M+H]⁺; ¹H NMR (400 MHz, Chloroform-d) δ 8.59 (s, 2H), 7.97 (d, J=33.1 Hz, 3H), 7.58 (d, J=20.0 Hz, 2H), 4.33 (q, J=13.3 Hz, 2H), 3.57 (dd, J=67.3, 10.4 Hz, 2H), 3.35 (s, 3H), 3.27-2.65 (m, 4H), 2.64-2.34 (m, 2H), 2.33-1.80 (m, 5H), 1.18 (s, 3H), 1.10-1.00 (m, 1H), 0.94 (d, J=6.1 Hz, 3H).

### Example 52

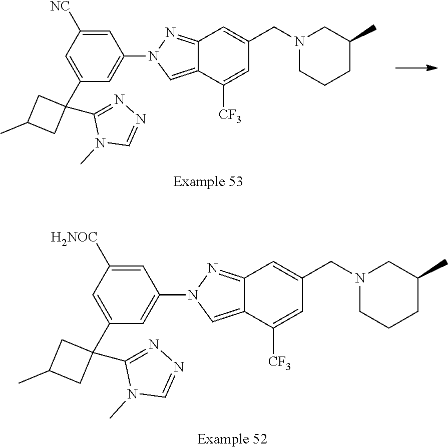

To a solution of Example 53 (30 mg, 0.055 mmol) in DMSO (2 mL) was added K₂CO₃ (15.mg, 0.110 mmol). Then the mixture was stirred at room temperature under nitrogen atmosphere. Then H₂O₂ (0.1 mL, 0.882 mmol) was added. The resulting mixture was stirred for 16 hrs at 25° C. The mixture was filtered through a Celite pad, and the filtrate was concentrated to give the crude product. The crude was purified by reverse phase pre-HPLC to afford the title compound. LCMS: 566.2 [M+H]⁺; ¹H NMR (400 MHz, DMSO-d₆) δ 9.32 (s, 1H), 8.55-8.45 (m, 1H), 8.40-8.30 (m, 1H), 8.25 (s, 1H), 8.21-8.01 (m, 1H), 7.95-7.74 (m, 2H), 7.70-7.48 (m, 2H), 3.59 (s, 2H), 3.25-3.19 (m, 3H), 2.99-2.94 (m, 1H), 2.78-2.67 (m, 2H), 2.64-2.56 (m, 2H), 2.41-2.26 (m, 1H), 1.97-1.88 (m, 1H), 1.70-1.44 (m, 5H), 1.23 (s, 1H), 1.19-0.98 (m, 3H), 0.99-0.67 (m, 4H).

### Example 53

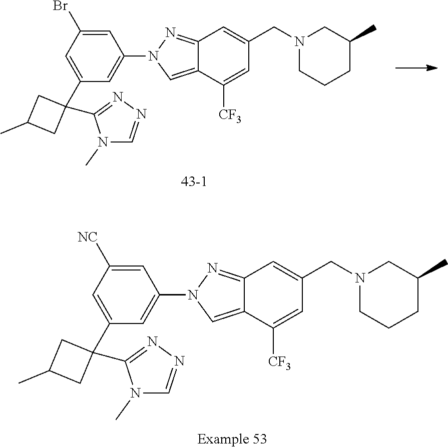

To a solution of Compound 43-1 (40 mg, 0.067 mmol) in DMF (2 mL) was added Zn(CN)₂ (15.6 mg, 0.133 mmol) and Pd(PPh₃)₄ (7.7 mg, 0.007 mmol) at 0° C. under N₂. The reaction was stirred at 120° C. for 18 hrs. The mixture was purified by reverse phase pre-HPLC to afford the title compound. LCMS: 548.3 [M+H]⁺; ¹H NMR (400 MHz, Chloroform-d) δ 8.51 (s, 1H), 8.25-7.93 (m, 4H), 7.75-7.55 (m, 2H), 3.85 (s, 2H), 3.33-3.28 (m, 3H), 3.15-2.88 (m, 4H), 2.83-2.61 (m, 2H), 2.39-2.22 (m, 2H), 1.99-1.70 (m, 4H), 1.27-1.15 (m, 4H), 0.98-0.85 (m, 4H).

### Intermediate 9

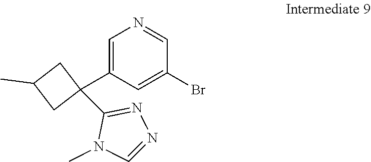 [SMILES: CC1CC(c2cncc(Br)c2)(c2nncn2C)C1]

The title compound was synthesized according to the procedure of preparation of Intermediate 10.

### Example 54

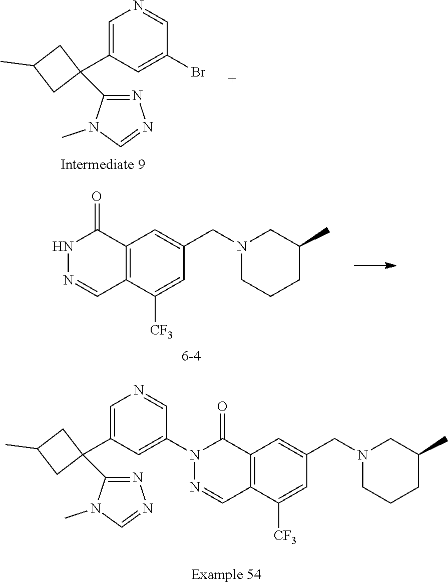

The title compound was synthesized according to the Step 5 in Example 6 with Intermediate 9 and Compound 6-4 as the substrates. LCMS: 552.4 [M+H]⁺; ¹H NMR (400 MHz, Chloroform-d) δ 8.94 (s, 1H), 8.73-8.46 (m, 3H), 8.25 (s, 1H), 8.14-7.85 (m, 2H), 3.70 (s, 2H), 3.35-3.22 (m, 3H), 3.01-2.58 (m, 6H), 2.24-1.90 (m, 2H), 1.81-1.61 (m, 5H), 1.29-1.10 (m, 3H), 1.06-0.77 (m, 4H).

### Intermediate 10

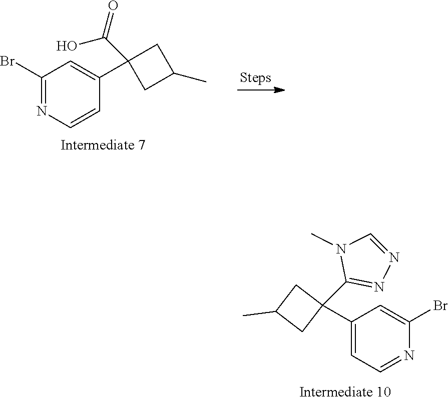 [SMILES: CC1CC(C(=O)O)(c2ccnc(Br)c2)C1 ; CC1CC(c2ccnc(Br)c2)(c2nncn2C)C1]

The title compound was synthesized according to Step 2 to Step 5 in the preparation of Example 31 with Intermediate 7 as the substrate. LCMS: 308.9 [M+H]⁺.

### Example 55

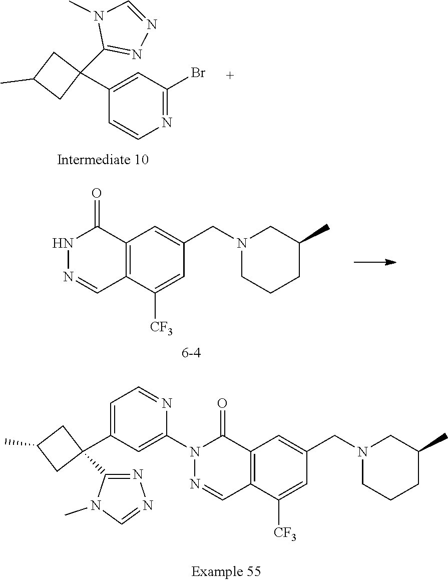

The title compound was synthesized according to the Step 5 in Example 6 with Intermediate 10 and Compound 6-4 as the substrates. LCMS: 552.4 [M+H]³⁰; ¹H NMR (400 MHz, MeOD) δ 8.64-8.60 (m, 3H), 8.36-8.34 (m, 2H), 7.78 (s, 1H), 7.52 (d, J=3.9 Hz, 1H), 3.81 (s, 2H), 3.37 (s, 3H), 3.07-2.94 (m, 2H), 2.90-2.79 (m, 211), 2.75-2.69 (m, 3H), 2.09-2.06 (m, 11H), 1.80-1.59 (m, 5H), 1.17 (d, J=5.9 Hz, 3H), 0.96-0.94 (m, 1H), 0.88 (d, J=6.2 Hz, 3H).

### Example 57

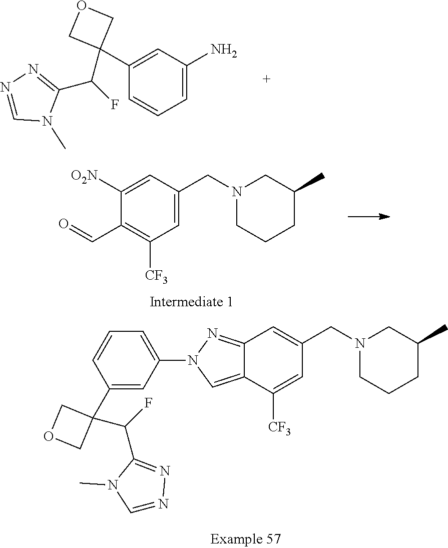

The title compound was synthesized according to the Step 6 in Example 32 with Intermediate 1 and 3-(3-(fluoro(4-methyl-4H-1,2,4-triazol-3-yl)methyl)oxetan-3-yl)aniline (synthesized according the literature) as the substrates. LCMS: 543.3 [M+H]⁺; ¹H NMR (400 MHz, Chloroform-d) δ 8.42 (s, 1H), 7.91 (s, 1H), 7.85-7.80 (m, 2H), 7.60 (s, 1H), 7.53 (s, 1H), 7.46 (t, J=8.0 Hz, 1H), 7.01 (d, J=7.3 Hz, 1H), 6.48 (d, J=46.2 Hz, 1H), 5.34 (d, J=6.5 Hz, 1H), 5.32-5.25 (m, 3H), 5.08-5.03 (m, 1H), 3.59 (s, 2H), 3.10 (s, 3H), 2.87-2.70 (m, 2H), 2.04-1.88 (m, 1H), 1.77-1.60 (m, 5H), 0.89-0.79 (m, 4H).

### Intermediate 11

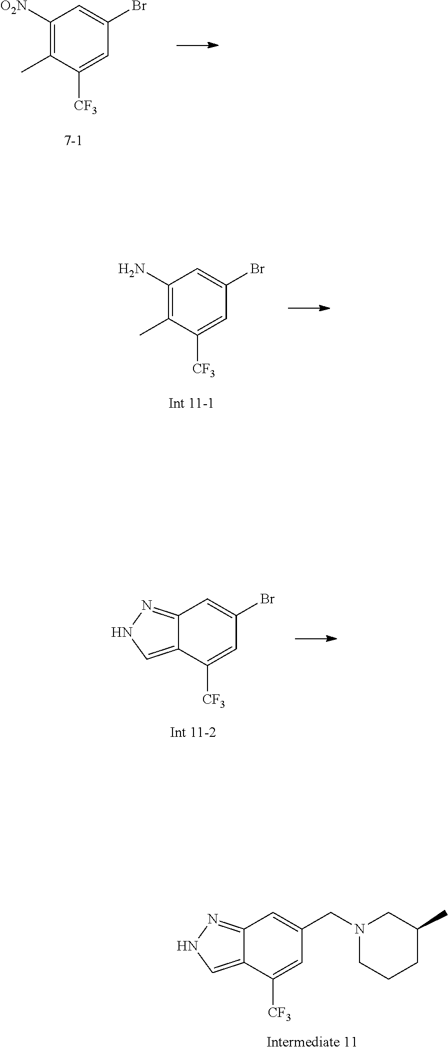 [SMILES: Cc1c([N+](=O)[O-])cc(Br)cc1C(F)(F)F ; Cc1c(N)cc(Br)cc1C(F)(F)F ; FC(F)(F)c1cc(Br)cc2n[nH]cc12 ; C[C@H]1CCCN(Cc2cc(C(F)(F)F)c3c[nH]nc3c2)C1]

### Step 1: Preparation of Int 11-1

To a solution of Compound 7-1 (1.00 g, 3.521 mmol) in H₂O (3 mL) and EtOH (5 mL) was added NH₄Cl (0.56 g, 10.562 mmol) and Fe (0.98 g, 17.604 mmol) at 25° C. under N₂. The reaction was stirred at 80° C. for 2 hrs. The mixture was filtered through a Celite pad, and the filtrate was concentrated to afford Int 11-1 (500 mg, 55.90%). LCMS: 254.0 [M+H]⁺.

### Step 2: Preparation of Int 11-2

To a solution of Int 11-1 (900 mg, 3.543 mmol) in acetic acid (10 mL) was added NaNO₂ (611.01 mg, 8.857 mmol), the mixture was stirred at 40° C. for 1 hr. The reaction mixture was then poured into water (30 mL) and extracted with ethyl acetate (50 mL×2), the combined organic layer was washed with brine (30 mL), dried over anhydrous Na₂SO₄ and concentrated to afford Int 11-2 (800 mg, 85.21%). LCMS: 265.2 [M+H]⁺.

### Step 3: Preparation of Intermediate 11

The title compound was synthesized according to Step 2 in Example 22. LCMS: 298.2 [M+H]⁺;

### Example 58

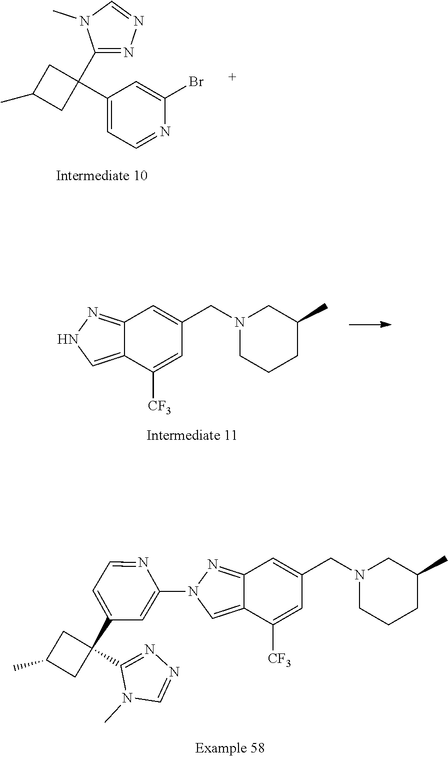

A solution of Intermediate 10 (286 mg, 0.931 mmol), Intermediate 11 (277 mg, 0.932 mmol) and Cs₂CO₃ (909 mg, 2.79 mmol) in DMSO (2 mL) was stirred at 120° C. for 16 hours. The mixture was purified by reverse phase prep-HPLC (2 times) to give the title compound. LCMS: 524.4 [M+H]⁺;

### Example 59

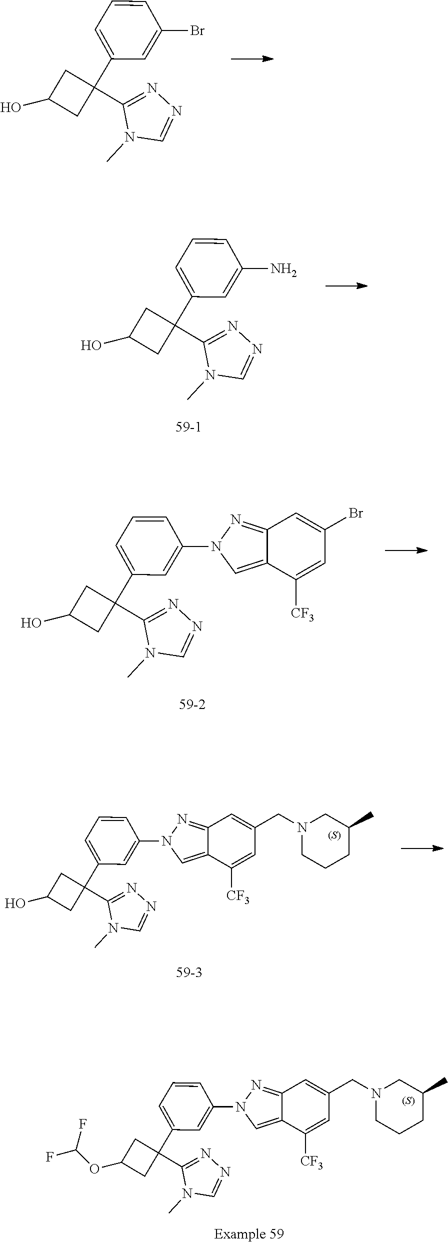

### Step 1: Preparation of Compound 59-1

To a solution of 3-(3-bromophenyl)-3-(4-methyl-4H-1,2,4-triazol-3-yl)cyclobutan-1-ol (500 mg, 1.396 mmol) in NH₄OH (9 mL) and ACN (3 mL) was added Cu₂O (235.4 mg, 1.622 mmol). The mixture was stirred at 110° C. for 3 hrs. After filtration, the filter cake was washed with NH₄Cl (sat. aq. 10 mL) and DCM:MeOH=10:1 (20 mL×3). The combined organic layers was concentrated under reduced pressure to afford residue. The residue was purified by flash silica gel chromatography to afford Compound 59-1 (380 mg, 95.9%). LCMS: 245.2 [M+H]⁺.

### Step 2: Preparation of Compound 59-2

Compound 59-2 was synthesized according to the Step 6 in Example 32. LCMS: 492.1/494.1 [M+H]⁺.

### Step 3: Preparation of Compound 59-3

Compound 59-3 was synthesized according to the Step 2 in Example 22. LCMS: 525.5 [M+H]⁺.

### Step 4: Preparation of Example 59

To a solution of (bromodifluoromethyl)trimethylsilane (92.9 mg, 0.457 mmol) and Compound 59-3 (60 mg, 0.114 mmol) in DCM (1 mL) and water (1 mL) was added potassium fluoride fluorane (71.5 mg, 0.915 mmol). The reaction was stirred at 25° C. for 16 hrs. The reaction mixture was concentrated in vacuum to remove DCM. The residue was purified by reverse phase prep-HPLC to afford the title compound. LCMS: 575.4 [M+H]⁺; ¹H NMR (400 MHz, Methanol-d₄) δ 9.20-9.06 (m, 1H), 8.24-8.05 (m, 4H), 8.04-7.85 (m, 1H), 7.76-7.66 (m, 2H), 7.64-7.43 (m, 1H), 4.51 (s, 2H), 4.61-4.34 (m, 1H), 3.61 (s, 3H), 3.54-3.30 (m, 4H), 3.06-2.92 (m, 2H), 2.90-2.65 (m, 2H), 2.02-1.71 (m, 4H), 1.34-1.18 (m, 1H), 1.18-0.94 (m, 3H).

### Example 60

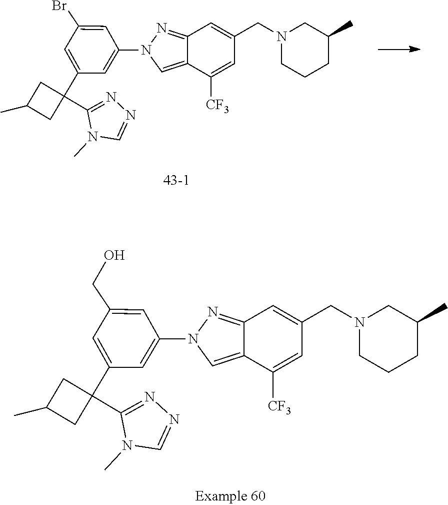

To a solution of Compound 43-1 (20 mg, 0.033 mmol) in dioxane (2 mL) was added (Tributylstannyl)methanol (32.0 mg, 0.100 mmol) and XPhos-Pd-G₂ (2.6 mg, 0.003 mmol) at 25° C. under N₂. The resulting mixture was stirred at 100° C. for 3 hrs. The reaction mixture was filtered and concentrated under reduced pressure to give a residue. The residue was purified by reverse phase prep-HPLC to afford the title compound. LCMS: 553.4 [M+H]⁺; ¹H NMR (400 MHz, Chloroform-d) δ 8.50 (s, 1H), 8.16-7.72 (m, 4H), 7.59-7.22 (m, 2H), 4.79 (s, 2H), 3.69 (s, 2H), 3.34-3.17 (m, 4H), 3.01-2.57 (m, 6H), 2.18-1.50 (m, 6H), 1.38-1.24 (m, 1H), 1.19-1.13 (m, 3H), 0.97-0.84 (m, 4H).

### Example 62

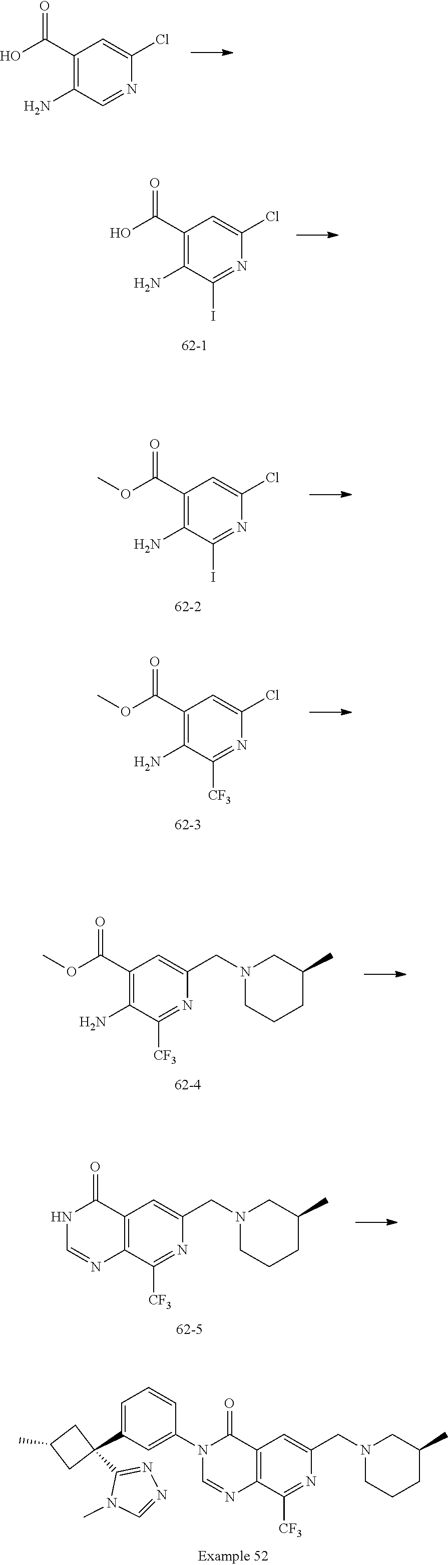

### Step 1: Preparation of Compound 62-1

To a solution of 5-amino-2-chloroisonicotinic acid (5 g, 28.974 mmol) in DMF (100 mL) was added NIS (9.8 g, 43.461 mmol), and the reaction was stirred for 10 min at 0° C. The resulting solution was stirred at 80° C. for 14 hrs. After cooling to room temperature, the reaction mixture was poured to saturated aqueous Na₂S₂O₃ solution. The mixture was diluted with EtOAc (50 mL) and water (300 mL).

The organic phase was separated and the aqueous phase was washed with EtOAc (50 mL×3). The combined organic phase was dried over anhydrous sodium sulfate, filtered, and concentrated under reduced pressure to give a residue. The residue was purified by flash silica gel chromatography to afford Compound 62-1 (7.5 g, 86.7%). LCMS: 298.8 [M+H]⁺.

### Step 2: Preparation of Compound 62-2

To a solution of Compound 62-1 (1 g, 3.350 mmol) in THF (1.5 mL) and MeOH (0.5 mL) was added TMSCHN₂ (3.35 mL, 6.700 mmol) at 0° C., and the reaction was stirred at 0° C. for 3 hrs under N₂.

The reaction was diluted with EtOAc (20 mL) and water (40 mL). The aqueous phase was extracted with EtOAc (10 mL×3). The combined organic phase was dried over anhydrous sodium sulfate, filtered, and concentrated under reduced pressure to give a residue. The residue was purified by flash silica gel chromatography to afford Compound 62-2 (800 mg, 76.4%). LCMS: 312.9 [M+H]⁺.

### Step 3: Preparation of Compound 62-3

To a solution of Compound 62-2 (710 mg, 2.272 mmol) in DMF (6 mL) was added CuI (519.4 mg, 2.726 mmol) and methyl 2,2-difluoro-2-(fluorosulfonyl)acetate (2182.3 mg, 11.360 mmol). Then the reaction was stirred at 80° C. for 5 hrs under N₂. The reaction was diluted with EtOAc (15 mL) and water (30 mL). The aqueous phase was extracted with EtOAc (10 mL×3). The combined organic phase was dried over anhydrous sodium sulfate, filtered, and concentrated under reduced pressure to give a residue.

The residue was purified by flash silica gel chromatography to afford Compound 62-3 (162 mg, 28.0%). LCMS: 437.1 [M+H]⁺;

### Step 4: Preparation of Compound 62-4

The title compound was synthesized according to Step 2 in Example 22. LCMS: 332.1 [M+H]⁺;

### Step 5: Preparation of Compound 62-5

To a solution of Compound 62-4 (200 mg, 0.604 mmol) in formamide (3 mL) was added FA (0.3 mL) at 0° C. The mixture was stirred at 180° C. for 14 hrs. The reaction mixture was diluted with water (10 mL). The aqueous phase was extracted with EtOAc (10 mL×3). The combined organic phase was dried over anhydrous sodium sulfate, filtered, and concentrated under reduced pressure to give a residue. The residue was purified by flash silica gel chromatography to afford Compound 62-5. LCMS: 327.1 [M+H]⁺.

### Step 6: Preparation of Example 62

The title compound was synthesized according to Step 3 in Example 38. LCMS: 552.1 [M+H]⁺; ¹H NMR (400 MHz, Chloroform-d) δ 8.63 (s, 1H), 8.25 (s, 1H), 8.01 (s, 1H), 7.61-7.53 (m, 2H), 7.40 (s, 1H), 7.30 (s, 1H), 4.09 (s, 2H), 3.26 (s, 3H), 3.14-2.83 (m, 3H), 2.75-2.63 (m, 3H), 2.08-1.98 (m, 2H), 1.83-1.54 (m, 4H), 1.26 (s, 1H), 1.15 (d, J=5.8 Hz, 3H), 1.06-0.96 (m, 1H), 0.90 (d, J=6.2 Hz, 3H).

### Example 63

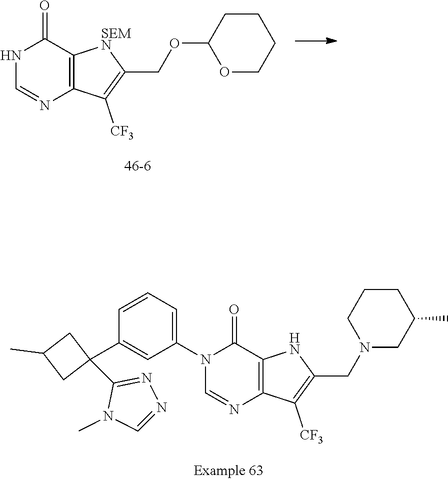

The title compound was synthesized according to the procedure in Example 46. LCMS: 540.2 [M+H]⁺; ¹H NMR (400 MHz, DMSO-d₆) δ 13.22 (s, 1H), 8.47-8.25 (m, 2H), 7.61-7.22 (m, 5H), 4.48 (s, 2H), 3.29-3.24 (m, 3H), 3.17-3.10 (m, 1H), 3.03-2.76 (m, 3H), 2.63-2.47 (m, 2H), 2.41-2.14 (m, 2H), 1.89-1.61 (m, 5H), 1.11-0.96 (m, 4H), 0.89 (d, J=6.4 Hz, 3H).

### Example 65

Example 65 was a cis-trans-isomer of Example 55, which could be separated by pre-HPLC. LCMS: 552.4 [M+H]⁺; ¹H NMR (400 MHz, Methanol-d₄) δ 8.84 (s, 1H), 8.82-8.52 (m, 3H), 8.50 (s, 1H), 7.81-7.41 (m, 2H), 4.62 (s, 2H), 3.45 (s, 3H), 3.28-3.10 (m, 2H), 3.08-2.89 (m, 2H), 2.81-2.64 (m, 2H), 2.61-2.41 (m, 2H), 2.03-1.72 (m, 5H), 1.24-1.14 (m, 4H), 1.00 (d, J=6.4 Hz, 3H).

### Example 66

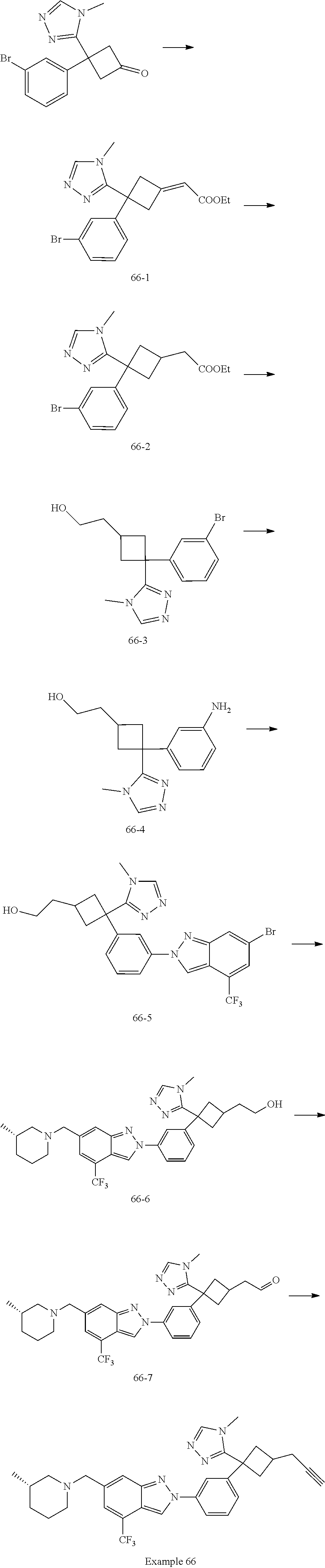

### Step 1: Preparation of Compound 66-1

To a mixture of LiBr (624 mg, 7.186 mmol) and TEA (1.8 mL, 13.065 mmol) in THF (5 mL) was added diethyl (2-methoxy-2-oxoethyl)phosphonate (1441 mg, 6.859 mmol) at 0° C. Stirring was continued at 25° C. for 2 hrs. To this mixture was added a solution of the 3-(3-bromophenyl)-3-(4-methyl-4H-1,2,4-triazol-3-yl)cyclobutan-1-one (1000 mg, 3.266 mmol) in THF (5 mL) at 0° C. The mixture was then stirred at 25° C. for 14 hrs. After concentrated. The residue was purified by chromatography to afford Compound 66-1 (1100 mg, 89.52%). LCMS: 378.3/376.3 [M+H]⁺.

### Step 2: Preparation of Compound 66-2

To a solution of Compound 66-1 (1.4 g, 3.721 mmol) and CuCl (0.22 g, 2.233 mmol) in MeOH (5 mL) was added NaBH₄ (0.56 g, 14.884 mmol) at 25° C. under N₂. The reaction was stirred at 25° C. for 1 h. Upon completion, the mixture was filtered through a Celite pad, and the filtrate was concentrated to give the crude product. The residue was purified by flash silica gel chromatography to afford Compound 66-2 (600 mg, 42.6%). LCMS: 379.9/378.2 [M+H]⁺.

### Step 3: Preparation of Compound 66-3

To a solution of Compound 66-2 (600 mg, 1.586 mmol) in anhydrous DCM (10 mL) was added bis(2-methylpropyl)aluminum hydride (4.76 mL, 4.759 mmol) in portions at 0° C. The mixture was stirred at 0° C. for 4 hrs. The reaction was quenched by the addition of water at 0° C., The mixture was filtered through a Celite pad, and the filtrate was concentrated to give a residue. The residue was purified by Chromatography to afford Compound 66-3 (300 mg, 56.3%). LCMS: 336.3/338.2 [M+H]⁺;

### Step 4: Preparation of Compound 66-4

Compound 66-3 (1.62 g, 4.818 mmol) and Cu₂O (0.7 g, 4.818 mmol) were dissolved in CH₃CN (5 mL) and NH₃·H₂O (10 mL). The mixture was stirred at 110° C. for 1.5 hrs under microwave. The mixture was filtered through a Celite pad, and the filtrate was concentrated to give the crude product. The crude product was purified by reversed-phase chromatography to afford Compound 66-4 (1.1 g, 83.8%). LCMS: 273.4 [M+H]⁺.

### Step 5: Preparation of Compound 66-5

To a solution of Compound 66-4 (990 mg, 3.322 mmol) in Isopropanol (12 mL) was added 4-bromo-2-nitro-6-(trifluoromethyl)benzaldehyde (904 mg, 3.322 mmol), and the mixture was stirred at 80° C. for 4 hrs. Then tributylphosphane (2013 mg, 9.966 mmol) was added and the resulting mixture was stirred at 80° C. for 16 hrs. The mixture was cooled to room temperature and diluted with EtOAc (50 mL). The organics phase was washed with brine (50 mL), dried over Na₂SO₄, filtered, and concentrated in vacuo. The residue was purified by flash silica gel chromatography to afford Compound 66-5 (400 mg, 23.1%). LCMS: 520.1/522.1 [M+H]⁺.

### Step 6: Preparation of Compound 66-6

Compound 66-6 was synthesized according to step 2 in Example 22. LCMS: 553.4 [M+H]⁺;

### Step 7: Preparation of Compound 66-7

To a solution of DMP (107 mg, 0.253 mmol) in DCM (3 mL) was added Compound 66-6 (70 mg, 0.127 mmol) at 0° C. under nitrogen, the mixture was stirred at room temperature for 16 hrs. The reaction mixture was partitioned between DCM (20 mL) and sat. NaHCO₃ (aq., 20 mL). The organic phase was separated, dried over anhydrous sodium sulfate, filtered, and concentrated under reduced pressure to give a residue. The residue was purified by silica gel column to afford Compound 66-7. LCMS: 551.4 [M+H]⁺.

### Step 8: Preparation of Example 66

To a solution of Compound 66-7 (50 mg, 0.091 mmol) and K₂CO₃ (25 mg, 0.182 mmol) in MeOH (3 mL) was added (1Z)-1-(diazyn-1-iumyl)-1-[dimethoxy(oxo)-λ5-phosphanyl]prop-1-en-2-olate (54 mg, 0.18 mmol) at 0° C. under N₂. The reaction was stirred at 25° C. for 3 hrs. The mixture was filtered through a Celite pad, and the filtrate was concentrated to give a residue. The residue was purified by prep-HPLC to afford the title compound. LCMS: 547.4 [M+H]⁺.

### Example 67

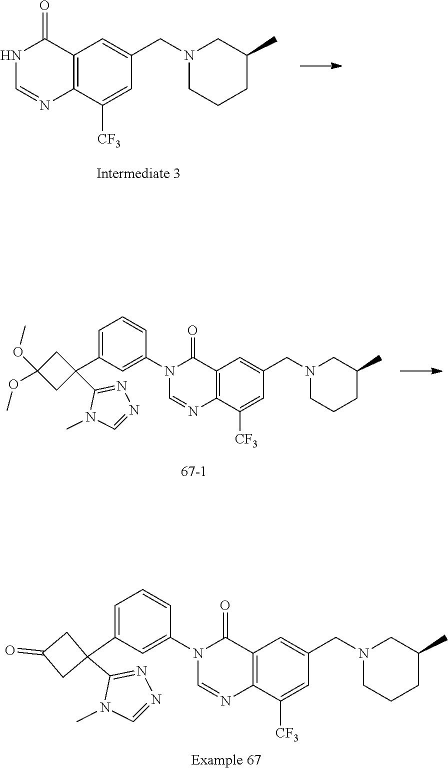

### Step 1: Preparation of Compound 67-1

Compound 67-1 was synthesized according to step 3 in Example 38 with the corresponding starting materials. LCMS: 597.5 [M+H]J.

### Step 2: Preparation of Example 67

To a solution of Compound 67-1 (50 mg, 0.084 mmol) in MeCN (3 mL) were added H₂SO₄ (0.01 mL, 0.251 mmol), the mixture was stirred at 25° C. for 3 hrs. The mixture was poured into water (10 mL) and the aqueous phase was basified with 1 NaHCO₃ (aq.) to pH=8-9, extracted with DCM (10 mL×3), the combined organic layers was concentrated under reduced pressure to give the crude product.

The crude product was purified by pre-HPLC to afford the title compound. LCMS: 551.4 [M+H]⁺; ¹H NMR (400 MHz, CDCl₃) δ 8.59 (s, 1H), 8.34-8.22 (m, 3H), 7.67-7.63 (m, 1H), 7.52-7.50 (m, 1H), 7.35-7.29 (m, 2H), 4.33-4.24 (m, 4H), 3.82-3.77 (m, 2H), 3.65-3.60 (m, 1H), 3.50-3.40 (m, 4H), 2.56 (t, J=11.1 Hz, 1H), 2.27-2.14 (m, 2H), 2.10-2.02 (m, 1H), 1.97-1.94 (m, 2H), 1.10-1.02 (m, 1H), 0.96 (d, J=5.5 Hz, 3H).

### Example 68

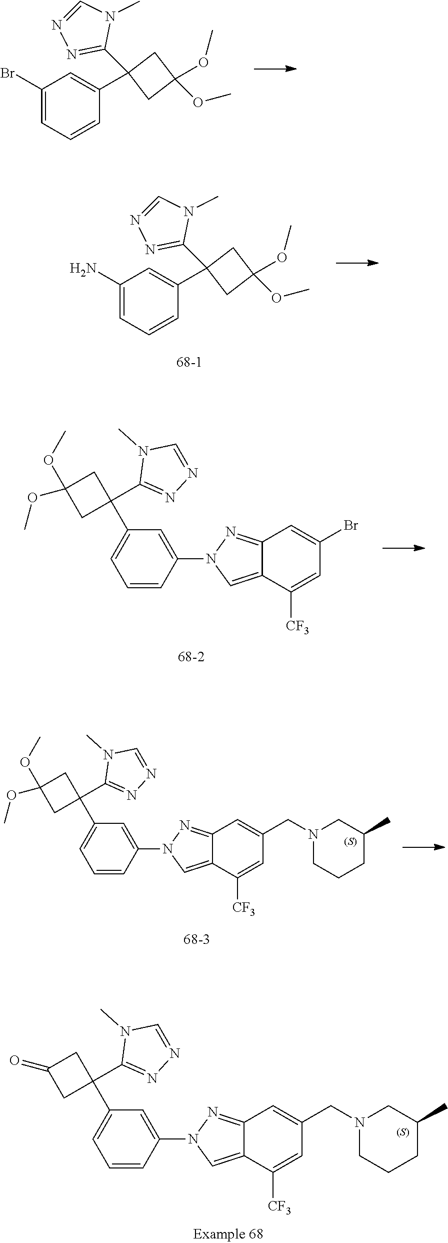

### Step 1-3: Preparation of Compound 68-3

Compound 68-3 was synthesized according to step 4 to step 6 in Example 66. LCMS: 569.4 [M+H]⁺.

### Step 4: Preparation of Example 68

The title compound was synthesized according to step 2 in Example 67. LCMS: 523.2 [M+H]⁺.

### Example 70

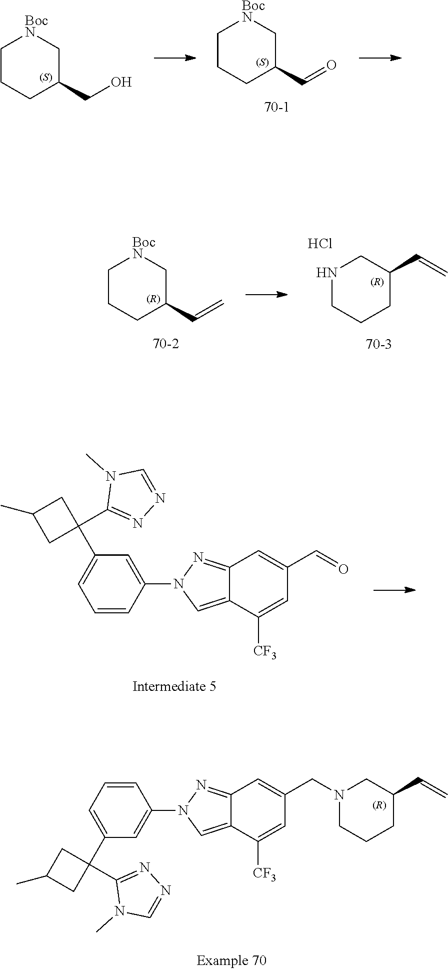

### Step 1: Preparation of Compound 70-1

To a solution tert-butyl (S)-3-(hydroxymethyl)piperidine-1-carboxylate (5 g, 23.223 mmol) in DCM (50 mL) was added DMP (12.8 g, 30.190 mmol). The resulting mixture was stirred at 0° C. for 0.5 hr. the reaction mixture was further stirred at 25° C. for 1.5 hrs. The reaction mixture was diluted with a mixture of 1M Na₂S₂O₃ (50 mL) and sat. aqueous NaHCO₃ (50 mL) and stirred for 30 min. DCM (30 mL) was added and the organic phase was separated, dried over anhydrous Na₂SO₄ and concentrated to afford Compound 70-1 (2.4 g, 48.5%).

### Step 2: Preparation of Compound 70-2

To a solution of methyltriphenylphosphanium bromide (5.1 g, 14.177 mmol) in THE (12 mL) was added butyllithium (5.67 mL, 14.177 mmol) and the resulting mixture was stirred at −78° C. After stirring at 0° C. for 30 min, the mixture was cooled to −78° C., and then a solution of Compound 70-1 (2.4 g, 11.252 mmol) in THE (12 mL) was added and stirred at room temperature for overnight. Upon completion, the reaction was quenched by the addition of brine (30 mL). The mixture was extracted with EtOAc (120 mL) and the organic phase was dried over Na₂SO₄ and concentrated in vacuo to give a residue, which was further purified by flash silica gel chromatography to give Compound 70-2 (420 mg, 17.7%). LCMS: 212.3 [M+H]⁺.

### Step 3: Preparation of Compound 70-3

A solution of Compound 70-2 (100 mg, 0.473 mmol) in DCM (1.5 mL) and TFA (0.5 mL) was stirred at 25° C. for 1 hr. Upon completion, the mixture was concentrated to afford Compound 70-3 (50 mg, 95.0%). LCMS: 112.3 [M+H]⁺.

### Step 4: Preparation of Example 70

The title compound was synthesized according to step 4 in Example 10 with Intermediate 5 as the substrate. LCMS: 535.1 [M+H]⁺; ¹H NMR (400 MHz, Chloroform-d) δ 8.50 (s, 1H), 8.13-7.67 (m, 4H), 7.60-7.31 (m, 3H), 5.80-5.57 (m, 1H), 5.14-4.94 (m, 2H), 3.53-3.26 (m, 2H), 3.24 (s, 3H), 3.05-2.82 (m, 2H), 2.79-2.51 (m, 3H), 2.45-2.31 (m, 1H), 2.01-1.64 (m, 7H), 1.28-1.08 (m, 4H).

### Example 71

The title compound was the cis-trans-isomer of Example 58, which could be separated by pre-HPLC. LCMS: 524.4 [M+H]⁺; ¹H NMR (400 MHz, DMSO-d₆) δ 9.26 (s, 1H), 8.64 (d, J=5.2 Hz, 1H), 8.46 (s, 1H), 8.28 (s, 1H), 7.84 (s, 1H), 7.78 (s, 1H), 7.64-7.52 (m, 1H), 4.46 (s, 2H), 3.26 (s, 3H), 2.90-2.85 (m, 2H), 2.68-2.61 (m, 3H), 2.38-2.32 (m, 3H), 1.87-1.65 (m, 5H), 1.13 (d, J=6.0 Hz, 3H), 1.10-1.02 (m, 1H), 0.88 (d, J=6.4 Hz, 3H).

### Example 72

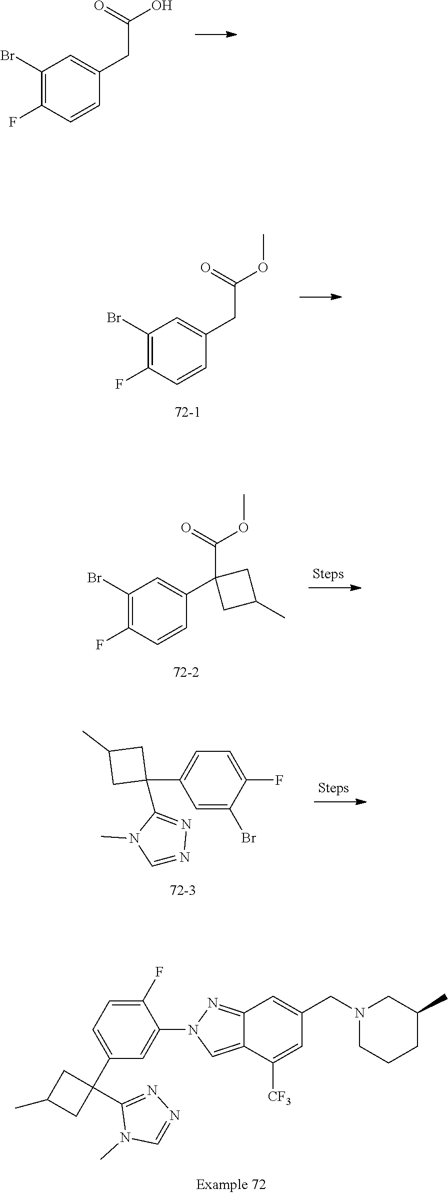

### Step 1: Preparation of Compound 72-1

A mixture of 2-(3-bromo-4-fluorophenyl)acetic acid (10 g, 42.911 mmol) in MeOH (100 mL) was stirred at 0° C. While stirring, conc·H₂SO₄ (2.287 mL, 42.911 mmol) was added dropwise, and the mixture was then heated to reflux for 3 hrs. The reaction mixture was concentrated under reduced pressure to remove solvent. The residue was diluted with H₂O (50 mL) and extracted with EtOAc (100 mL×3). The combined organic layers were dried over anhydrous sodium sulfate, filtered, and concentrated under reduced pressure to give Compound 72-1 (10.3 g, 97.16%). LCMS: 524.4 [M+H]⁺.

### Step 2: Preparation of Compound 72-2

To a solution of Compound 72-1 (8.7 g, 35.214 mmol) and 1,3-Dibromo-2-methylpropane (7.60 g, 35.214 mmol) in DMF (5 mL) was added NaH (3.10 g, 77.471 mmol) at 0° C. under N₂. The mixture was stirred at 25° C. for 2 hrs. The reaction mixture was poured slowly into saturated solution of ammonium chloride (300 mL). The mixture was extracted with EtOAc (100 mL×3). The combined organic phase was washed with brine (150 mL), dried over anhydrous Na₂SO₄, filtered, concentrated to afford Compound 72-2 (7.4 g, 69.8%). LCMS: 301/303.0 [M+H]⁺.

### Step 3: Preparation of Compound 72-3

Compound 72-3 was synthesized according to Step 2 to Step 5 in Example 31. LCMS: 324.0/326.0 [M+H]⁺;

### Step 4: Preparation of Example 72

The title compound was synthesized according to step 4 to step 6 in Example 66. LCMS: 541.1 [M+H]⁺.

### Example 73

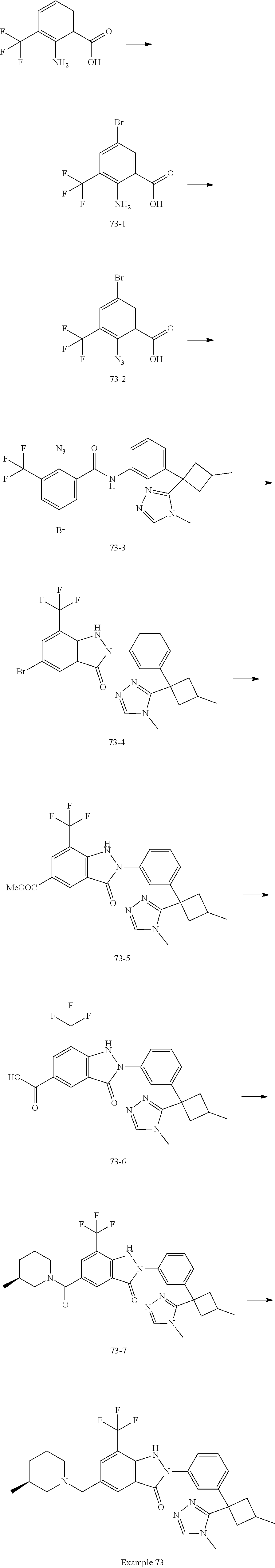

### Step 1: Preparation of Compound 73-1

To a solution of 2-amino-3-(trifluoromethyl)benzoic acid (10 g, 48.747 mmol) in DCM (100 mL) were added NBS (9.54 g, 53.622 mmol) and the reaction was stirred at 20° C. for 1 hr under N₂. After cooling to room temperature, the reaction mixture was poured into sat. NaHCO₃ (aq. 150 mL) and extracted with DCM (100 mL×3). The combined organic phase was washed with brine (100 mL), then dried over anhydrous Na₂SO₄, filtered and concentrated under reduced pressure to give a residue. The residue was triturated with ethyl acetate/petroleum ether (1/3, 20 mL) at 25° C. for 20 min, filtered and the filter cake was dried to afford Compound 73-1 (13.6 g, 98.23%). LCMS: 284.0/286.0 [M+H]⁺.

### Step 2: Preparation of Compound 73-2

To a solution of Compound 73-1 (100 mg, 0.373 mmol) in TFA (30 mL) at 0° C. was added NaNO₂ (1.09 g, 15.843 mmol), the mixture was stirred at 0 T for 1 hr, then NaN₃ (1.03 g, 15.843 mmol) was added to the mixture, and the mixture was further stirred at 0° C. for 30 min. The resulting mixture was poured into water and the precipitate was collected by filtration to afford the Compound 73-2 (3.5 g, 96.19%). LCMS: 310.0/312.0 [M+H]⁺.

### Step 3: Preparation of Compound 73-3

To a solution of Compound 73-2 (640 mg, 2.064 mmol) in DMF (10 mL) was added HATU (1020.42 mg, 2.684 mmol), DIEA (1.024 mL, 6.193 mmol) and 3-(3-methyl-1-(4-methyl-4H-1,2,4-triazol-3-yl)cyclobutyl)aniline (500.25 mg, 2.064 mmol), the reaction mixture was stirred at r.t. for 16 hrs. The reaction mixture was poured into sat. NaHCO₃ (aq. 40 mL) and extracted with EtOAc (30 mL×3). The combined organic layers was washed with brine (40 mL), dried over anhydrous sodium sulfate, filtered, and concentrated to afford a residue. The residue was purified by flash silica gel chromatography to afford Compound 73-3 (776 mg, 70.35%). LCMS: 533.9/535.9 [M+H]⁺.

### Step 4: Preparation of Compound 73-4

To a solution of Compound 73-3 (776 mg, 1.452 mmol) in DMF (10 mL) were added NaH (69.71 mg, 1.743 mmol) at r.t., and the reaction was stirred at 80° C. for 16 hrs under N₂. After cooling to r.t., the reaction mixture was poured into sat. NaHCO₃ (aq. 50 mL) and extracted with EA (40 mL×3). The combined organic layers was washed with brine (40 mL), dried over anhydrous sodium sulfate, filtered, and concentrated to afford a residue. The residue was purified by flash silica gel chromatography to afford Compound 73-4 (764 mg, 103.90%). LCMS: 506.1/508.1 [M+H]⁺.

### Step 5: Preparation of Compound 73-5

A mixture of Compound 73-4 (760 mg, 1.501 mmol), Pd(dppf)Cl₂ (177.33 mg, 0.242 mmol) and TEA (0.626 mL, 4.506 mmol) in MeOH (15 mL) was stirred at 60° C. for 12 hrs under CO (50 Psi). The reaction mixture was filtered and concentrated to give the residue. The residue was purified by flash silica gel chromatography to afford Compound 73-4 (610 mg, 78.7%). LCMS: 486.2 [M+H]⁺;

### Step 6: Preparation of Compound 73-6

A solution of Compound 73-5 (100 mg, 0.206 mmol) and LiOH (43.3 mg, 1.030 mmol) in MeOH (1 mL), THF (1 mL) and Water (1 mL) was stirred at r.t. for 12 hrs. The mixture was poured into water (15 mL), the aqueous phase was washed with EtOAc (10 mL×3), then acidified with 2 N aq. HCl to pH=3-5, the aqueous phase was further extracted with EtOAc (10 mL×3), and the combined organic layers was concentrated to give the crude Compound 73-6, which could be directly used in next step. LCMS: 472.3 [M+H]⁺.

### Step 7: Preparation of Compound 73-7

To a solution of Compound 73-6 (58 mg, 0.123 mmol) and (S)-3-methylpiperidine (20.0 mg, 0.148 mmol) in DMF (2 mL) was added TEA (0.05 mL, 0.369 mmol), HATU (70.2 mg, 0.185 mmol). The reaction mixture was stirred at r.t. for 12 hrs under N₂. The residue was purified by prep-HPLC to afford Compound 73-7. LCMS: 553.2 [M+H]⁺.

### Step 8: Preparation of Example 73

To a solution of Compound 73-7 (133 mg, 0.241 mmol) in THE (3 mL) was added LiAlH₄ (0.2 mL, 0.481 mmol) at 0° C. The mixture was stirred at r.t. for 2 hrs under N₂. After the reaction mixture was cooled to 0° C., the mixture was quenched by addition of water (0.2 mL). The mixture was filtered and concentrated under reduced pressure to give the residue. The residue was purified by prep-HPLC to afford the title compound. LCMS: 539.4 [M+H]⁺; ¹H NMR (400 MHz, Chloroform-d) δ 8.19-7.71 (m, 4H), 7.70-7.29 (m, 2H), 7.21-7.02 (m, 1H), 3.77-3.49 (m, 2H), 3.36-3.15 (m, 3H), 2.92-2.65 (m, 4H), 2.36-2.18 (m, 1H), 2.03-1.99 (m, 1H), 1.89-1.50 (m, 6H), 1.31-1.12 (m, 4H), 0.96-0.77 (m, 4H).

### Example 75

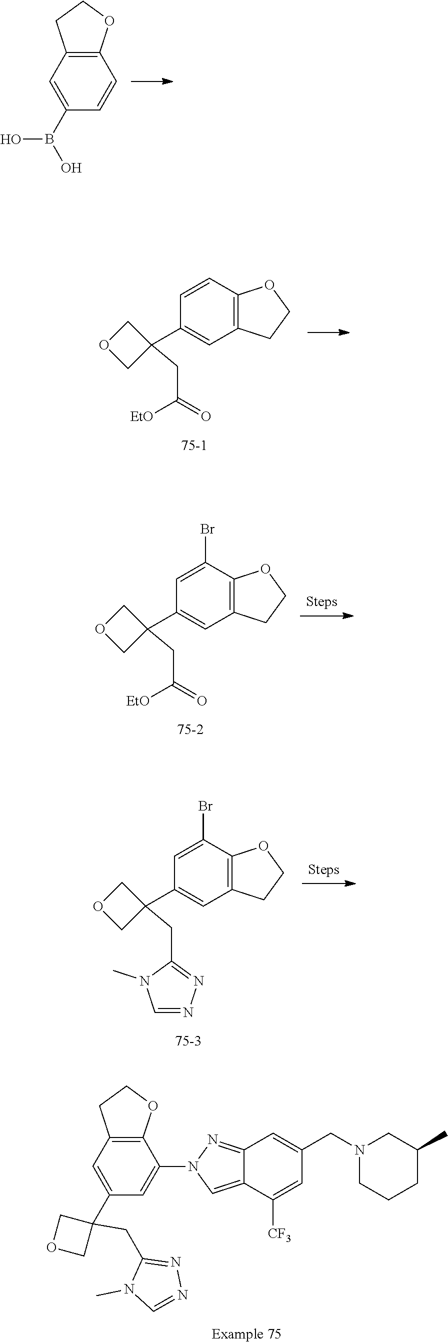 [SMILES: OB(O)c1ccc2c(c1)CCO2 ; CCOC(=O)CC1(c2ccc3c(c2)CCO3)COC1 ; CCOC(=O)CC1(c2cc(Br)c3c(c2)CCO3)COC1 ; Cn1cnnc1CC1(c2cc(Br)c3c(c2)CCO3)COC1 ; C[C@H]1CCCN(Cc2cc(C(F)(F)F)c3cn(-c4cc(C5(Cc6nncn6C)COC5)cc5c4OCC5)nc3c2)C1]

### Step 1: Preparation of Compound 75-1

To a solution of bis [chlororhodium(I)]bis(cycloocta-1,5-diene) (0.31 g, 0.633 mmol) in dioxane (40 mL) was added a solution of KOH (1.78 g, 31.657 mmol) in H₂O (8 mL). ethyl oxetan-3-ylideneacetate (3 g, 21.104 mmol) and (2,3-dihydrobenzofuran-5-yl)boronic acid (4.50 g, 27.436 mmol) was added into the mixture. The reaction was stirred at 25° C. for 12 hrs. 50 mL of water was added into the reaction mixture and the mixture was extracted with ethyl acetate (50 mL×3). The combined organic phase was washed with brine (30 mL), dried over anhydrous Na₂SO₄, filtered and concentrated in vacuum. The residue was purified by flash silica gel chromatography to afford Compound 75-1 (4.2 g, 75.87%). LCMS: 263.2 [M+H]⁺.

### Step 2: Preparation of Compound 75-2

To a solution of Compound 75-1 (4.2 g, 16.012 mmol) in AcOH (50 mL) was added sodium acetate (1.31 g, 16.012 mmol) and bromine (1.66 g, 20.815 mmol) at 0° C. The reaction was stirred at ambient temperature for 2 hrs. Water (50 mL) was added, and the mixture was extracted with EtOAc (50 mL×2). The combined organic phase was washed with brine, then dried over anhydrous Na₂SO₄, filtered and concentrated in vacuo. The residue was purified by flash silica gel chromatography to afford Compound 75-2 (3.1 g, 56.74%). LCMS: 341.0 [M+H]⁺.

### Step 3: Preparation of Compound 75-3

Compound 75-3 was synthesized according to Step 2 to Step 5 in Example 31. LCMS: 350.0/352.0 [M+H]⁺;

### Step 4: Preparation of Example 75

The title compound was synthesized according to Step 4 to Step 6 in Example 66. LCMS: 567.3 [M+H]⁺; ¹H NMR (400 MHz, Chloroform-d) δ 8.90 (s, 1H), 7.89 (s, 1H), 7.82 (s, 1H), 7.67 (s, 1H), 7.50 (s, 1H), 6.65 (s, 1H), 5.18-4.99 (m, 4H), 4.79 (t, J=8.7 Hz, 2H), 3.71-3.49 (m, 4H), 3.24 (t, J=8.7 Hz, 2H), 3.03 (s, 3H), 2.92-2.66 (m, 2H), 2.06-1.24 (m, 6H), 0.94-0.77 (m, 4H).

### Example 76

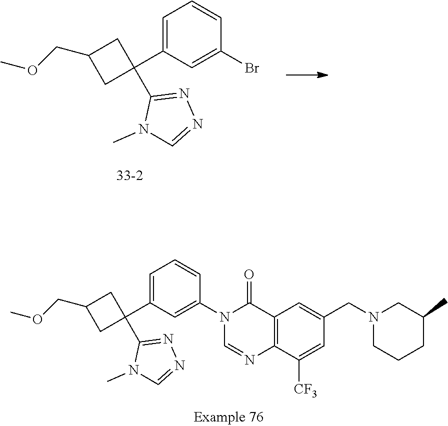

The title compound was synthesized according to Step 3 in Example 38. LCMS: 581.0 [M+H]⁺;

### Example 77

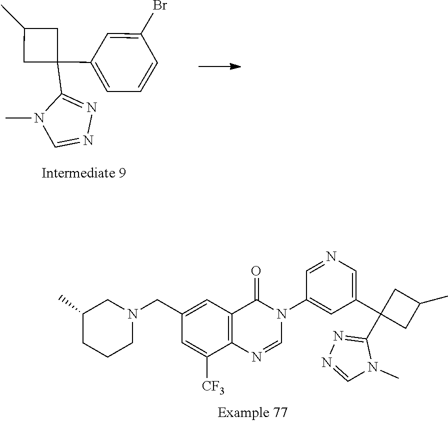

The title compound was synthesized according to Step 3 in Example 38. LCMS: 552.3 [M+H]⁺; ¹H NMR (400 MHz, Chloroform-d) δ 8.82 (s, 1H), 8.60 (s, 1H), 8.42 (s, 2H), 8.21-8.15 (m, 1H), 8.03 (s, 1H), 7.79 (t, J=2.3 Hz, 1H), 3.63 (s, 2H), 3.31 (s, 3H), 2.98-2.89 (m, 2H), 2.83-2.70 (m, 4H), 2.42-2.33 (m, 1H), 2.05-1.90 (m, 1H), 1.80-1.51 (m, 5H), 1.21-1.14 (m, 3H), 0.94-0.76 (m, 4H).

### Example 78

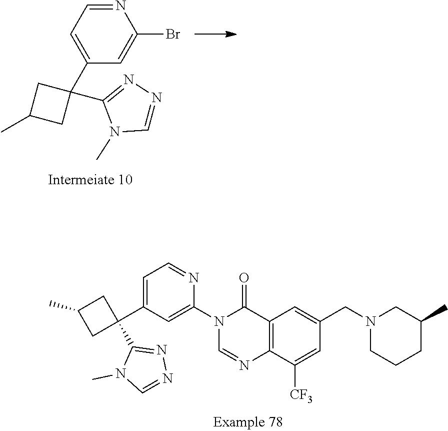

The title compound was synthesized according to Step 3 in Example 38. LCMS: 552.4 [M+H]⁺; ¹H NMR (400 MHz, DMSO-d₆) δ 8.69 (s, 1H), 8.64 (d, J=5.2 Hz, 1H), 8.40-8.38 (m, 1H), 8.38 (s, 1H), 8.16 (d, J=1.9 Hz, 1H), 7.84 (d, J=1.7 Hz, 1H), 7.47 (dd, J=5.3, 1.8 Hz, 1H), 3.66 (s, 2H), 3.25 (s, 3H), 2.93-2.85 (m, 2H), 2.73 (t, J=10.2 Hz, 2H), 2.66-2.58 (m, 3H), 1.94 (t, J=10.8 Hz, 1H), 1.71-1.57 (m, 4H), 1.52-1.44 (m, 1H), 1.10 (d, J=5.2 Hz, 3H), 0.88 (s, 1H), 0.82 (d, J=6.0 Hz, 3H).

### Example 80: Preparation of (S)-3-(3-(3-((4-methyl-4H-1,2,4-triazol-3-yl)methyl)oxetan-3-yl)phenyl)-6-((3-methylpiperidin-1-yl)methyl)-8-(trifluoromethyl)quinazolin-4(3H)-one

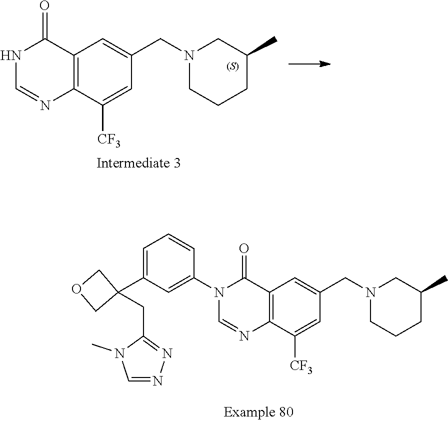

The title compound was synthesized according to Step 3 in Example 38. LCMS: 553.3 [M+H]⁺; ¹H NMR (400 MHz, DMSO-d₆) δ 8.40 (s, 1H), 8.36 (s, 1H), 8.22 (s, 1H), 8.19-8.13 (m, 1H), 7.52-7.39 (m, 2H), 7.24 (s, 1H), 7.12-6.98 (m, 1H), 4.94 (d, J=6.1 Hz, 2H), 4.89 (d, J=6.2 Hz, 2H), 3.67 (s, 2H), 3.50 (s, 2H), 2.95 (s, 3H), 2.81-2.68 (m, 2H), 2.16-1.76 (m, 1H), 1.76-1.25 (m, 5H), 0.93-0.85 (m, 1H), 0.82 (d, J 6.2 Hz, 3H).

### Example 81

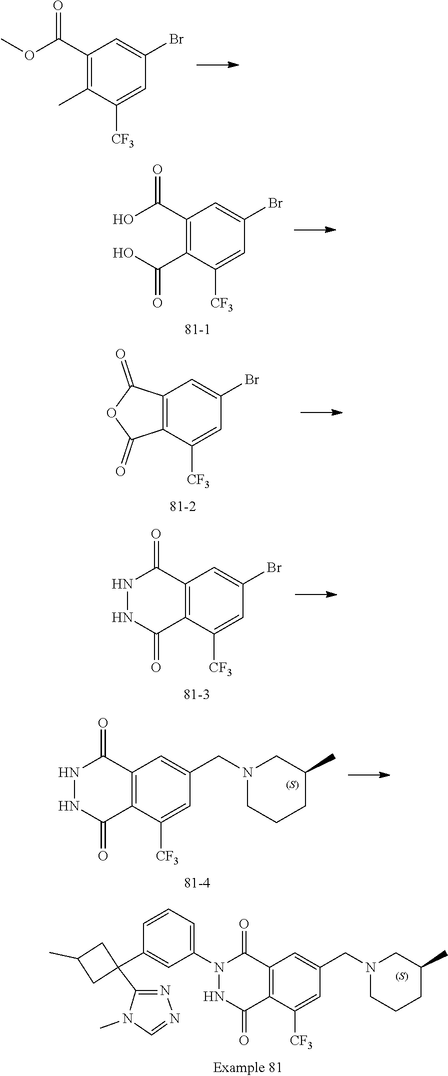

### Step 1: Preparation of Compound 81-1

To a mixture of methyl 5-bromo-2-methyl-3-(trifluoromethyl)benzoate (3.5 g, 11.782 mmol) in H₂O (30 mL) was added NaOH (0.94 g, 23.563 mmol). The mixture was heated to 80° C., then KMnO₄ (3.72 g, 23.563 mmol) was added in portions during 3 hours. Then the mixture was stirred for 30 minutes. The reaction mixture was filtered and the cake was washed with hot water (200 mL). The filtrate was acidified with 2 N HCl to pH=1, then the mixture was extracted with EtOAc (300 mL×3), the combined organic layers was dried over Na₂SO₄ and concentrated in vacuum to give Compound 81-1 (0.74 g, 20.06%). LCMS: 313.1/315.1 [M+H]⁺.

### Step 2: Preparation of Compound 81-2

To a solution of Compound 81-1 (200 mg, 0.639 mmol) in Toluene (5 mL) and DMF (0.2 mL) was added AC₂O (0.300 mL, 3.195 mmol), and the mixture was stirred at 100° C. under N₂ for 3 hr. The reaction mixture was concentrated under reduced pressure to give Compound 81-2 (200 mg, 95.50%).

### Step 3: Preparation of Compound 81-3

To a solution of Compound 81-2 (462 mg, 1.566 mmol) in AcOH (8 mL) was added hydrazine hydrate (244.98 mg, 3.915 mmol). The mixture was stirred at 125° C. for 4 h. The mixture was filtered through celite, and the filter cake was washed with DCM. The filtrate was concentrated in vacuo to afford Compound 81-3 (200 mg, 89.36%). LCMS: 309.0/311.0 [M+H]⁺.

### Step 4: Preparation of Compound 81-4

Compound 81-4 was synthesized according to Step 2 in Example 22. LCMS: 342.4 [M+H]⁺;

### Step 5: Preparation of Example 81

The title compound was synthesized according to Step 3 in Example 38. LCMS: 567.3 [M+H]⁺;

### Example 82

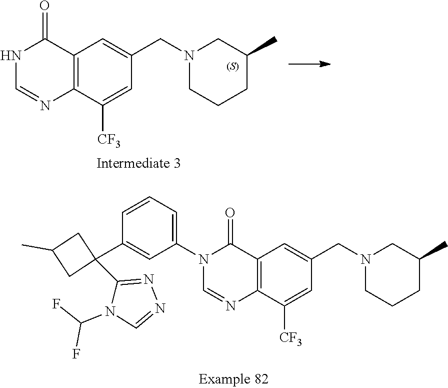

The title compound was synthesized according to Step 3 in Example 38 with corresponding substrates. LCMS: 628.4 [M+ACN+H]⁺.

### Example 83

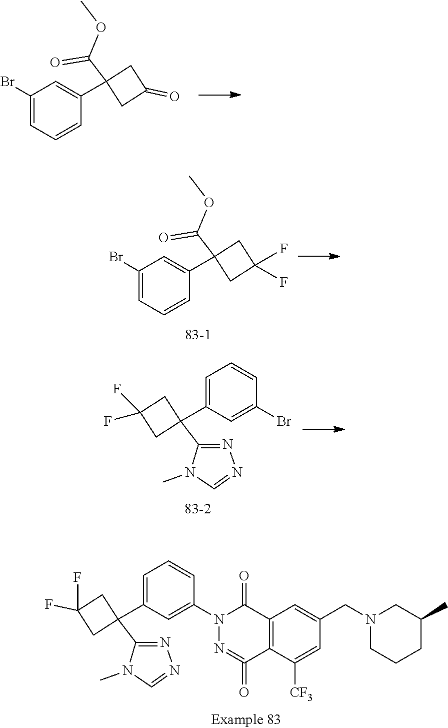

### Step 1: Preparation of Compound 83-1

To a solution of methyl 1-(3-bromophenyl)-3-oxocyclobutane-1-carboxylate (400 mg, 1.413 mmol) in DCM (10 mL) was added DAST (501.01 mg, 3.108 mmol) at 0° C. under N₂. The reaction was stirred at 25° C. for 12 hrs. The aqueous layer was extracted with EtOAc (100 mL×3). The combined organic layer was washed with brine (100 mL), dried over anhydrous Na₂SO₄ and concentrated under reduce pressure to give a residue. The residue was purified by flash silica gel chromatography to afford Compound 83-1 (400 mg, 92.79%). ¹H NMR (400 MHz, CDCl₃): 7.52-7.36 (m, 2H), 7.32-7.11 (m, 2H), 3.70 (s, 3H), 3.54-3.37 (m, 2H), 3.11-2.94 (m, 2H).

### Step 2: Preparation of Compound 83-2

The Compound 83-2 was synthesized according to Step 2 to Step 5 in Example 31. LCMS: 328.0 [M+H]⁺;

### Step 3: Preparation of Example 83

The title compound was synthesized according to Step 3 in Example 38. LCMS: 573.2 [M+H]⁺; ¹H NMR (400 MHz, DMSO-d₆) δ 8.56 (s, 2H), 8.42 (s, 1H), 8.28 (s, 1H), 7.66-7.53 (m, 3H), 7.51-7.35 (m, 1H), 3.75 (q, J=13.2 Hz, 4H), 3.32 (s, 7H), 2.91-2.61 (m, 2H), 1.59 (d, J=56.4 Hz, 4H), 1.02-0.86 (m, 1H), 0.83 (d, J=6.0 Hz, 3H).

### Example 84

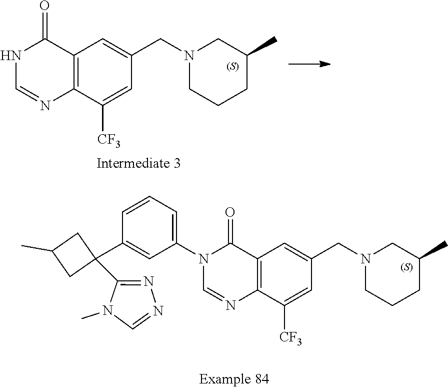

The title compound was synthesized according to Step 3 in Example 38. LCMS: 551.0 [M+H]⁺; ¹H NMR (400 MHz, Chloroform-d) δ 8.47 (s, 1H), 8.27 (s, 11H), 8.24 (s, 1H), 8.12-8.01 (m, 1H), 7.59-7.29 (m, 4H), 3.92 (s, 2H), 3.27 (s, 3H), 3.19-3.01 (m, 2H), 2.95-2.80 (m, 2H), 2.76-2.61 (m, 3H), 2.29-2.15 (m, 1H), 1.92-1.66 (m, 5H), 1.22-1.09 (m, 3H), 0.96-0.85 (m, 4H).

### Example 85

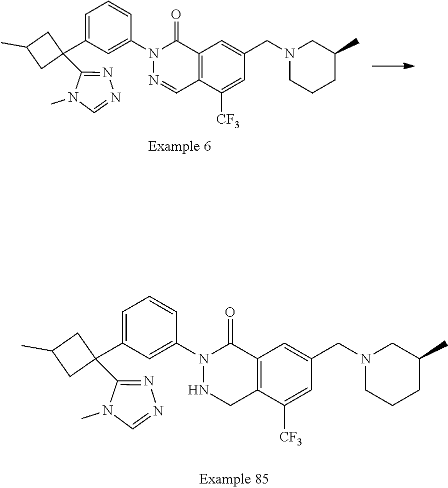

To a solution of Example 6 (50 mg, 0.107 mmol) in Acetic acid (5 mL) and H₂O (0.5 mL) were added Zn (237 mg, 3.632 mmol), and the reaction was stirred at 120° C. for 1 h. After cooling to room temperature, the mixture was filtered and then the filtrate was concentrated to give a residue, the residue was purified by prep-HPLC to afford Example 85. LCMS: 553.3 [M+H]⁺; ¹H NMR (400 MHz, DMSO-d₆) δ 8.62-8.13 (m, 3H), 7.89-7.54 (m, 3H), 7.45-7.02 (m, 2H), 6.74-6.57 (m, 1H), 4.35 (d, J=7.7 Hz, 2H), 3.58 (s, 2H), 3.22 (s, 3H), 2.89-2.67 (m, 4H), 2.59-2.47 (m, 3H), 2.24-1.87 (m, 1H), 1.73-1.39 (m, 5H), 1.13-1.04 (m, 3H), 0.88-0.78 (m, 4H).

### Example 87

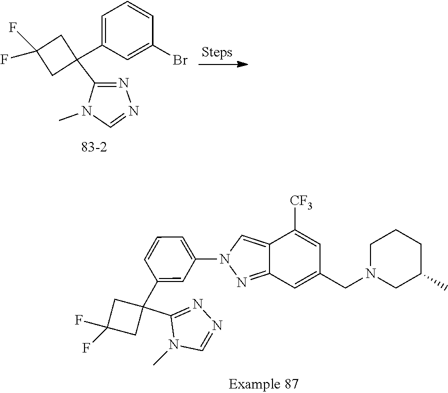 [SMILES: Cn1cnnc1C1(c2cccc(Br)c2)CC(F)(F)C1 ; C[C@H]1CCCN(Cc2cc(C(F)(F)F)c3cn(-c4cccc(C5(c6nncn6C)CC(F)(F)C5)c4)nc3c2)C1]

The title compound was synthesized according to Step 4 to Step 6 in Example 66. LCMS: 545.3 [M+H]³⁰; ¹H NMR (400 MHz, Chloroform-d) δ 8.54 (s, 1H), 8.32 (s, 1H), 8.11 (s, 1H), 7.97 (s, 1H), 7.81 (d, J=8.4 Hz, 1H), 7.67 (s, 1H), 7.59 (t, J=7.8 Hz, 1H), 7.41 (d, J=7.9 Hz, 1H), 4.33 (s, 2H), 3.88-3.77 (m, 2H), 3.62-3.40 (m, 3H), 3.35 (s, 3H), 2.78-2.24 (m, 3H), 1.97-1.37 (m, 4H), 0.99-0.93 (m, 3H), 0.89-0.78 (m, 1H).

### Example 88

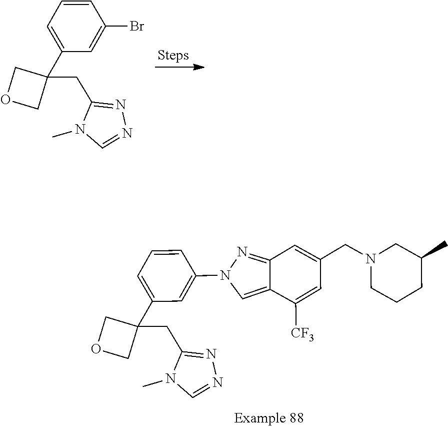 [SMILES: Cn1cnnc1CC1(c2cccc(Br)c2)COC1 ; C[C@H]1CCCN(Cc2cc(C(F)(F)F)c3cn(-c4cccc(C5(Cc6nncn6C)COC5)c4)nc3c2)C1]

The title compound was synthesized according to Step 4 to Step 6 in Example 66. LCMS: 525.0 [M+H]⁺; ¹H NMR (400 MHz, Chloroform-d) δ 8.49 (s, 1H), 8.27 (s, 1H), 7.92 (s, 1H), 7.75 (d, J=8.0 Hz, 1H), 7.64 (s, 11H), 7.61 (s, 1H), 7.46 (t, J=7.9 Hz, 1H), 7.04-6.88 (m, 1H), 5.22-5.10 (m, 4H), 4.19 (s, 2H), 3.63 (s, 2H), 3.44-3.23 (m, 1H), 3.01 (s, 3H), 2.60-2.47 (m, 1H), 2.42-2.10 (m, 2H), 1.96-1.39 (m, 4H), 0.94 (d, J=6.5 Hz, 3H), 1.12-0.79 (m, 1H).

### Example 90

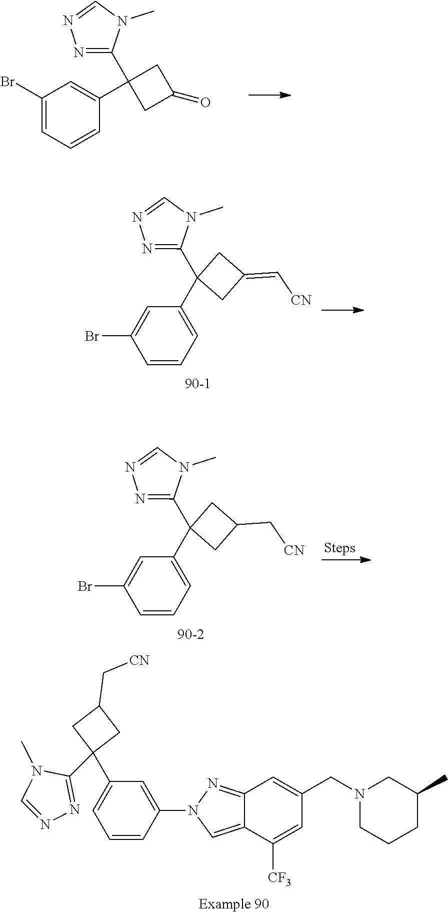

### Step 1: Preparation of Compound 90-1

To a mixture of LiBr (312.01 mg, 3.593 mmol) and TEA (0.908 mL, 6.533 mmol) in THF (20 mL) was added diethyl (cyanomethyl)phosphonate (607.52 mg, 3.430 mmol) at 0 T. After stirring at 25° C. for 2 hrs, to this mixture was added a solution of the 3-(3-bromophenyl)-3-(4-methyl-4H-1,2,4-triazol-3-yl)cyclobutan-1-one (500 mg, 1.633 mmol) in THF (2 mL) at 0 T. The mixture was then stirred at 25° C. for 16 hrs. After concentrated, the residue was purified by flash silica gel chromatography to afford Compound 90-1. LCMS: 329.0 [M+H]⁺.

### Step 2: Preparation of Compound 90-2

To a solution of Compound 90-1 (910 mg, 2.764 mmol) in THE (9 mL) and i-PrOH (3 mL) was added NaBH₄ (115.03 mg, 3.041 mmol) at 25° C. under N₂. The reaction was stirred at 25° C. for 1 h. The mixture was poured into NH₄Cl (aq), The aqueous layer was extracted with EtOAc (100 mL×3). The combined organic layer was washed with brine (100 mL), dried over anhydrous Na₂SO₄ and concentrated to afford a residue, which was purified by flash silica gel chromatography to afford Compound 90-2 (850 mg, 92.84%). LCMS: 331.0 [M+H]⁺.

### Step 3: Preparation of Example 90

The title compound was synthesized according to Step 4 to Step 6 in Example 66. LCMS: 548.2 [M+H]⁺; ¹H NMR (400 MHz, Chloroform-d) δ 8.58 (s, 1H), 8.13-7.87 (m, 2H), 7.85-7.76 (m, 1H), 7.63-7.54 (m, 2H), 7.49-7.26 (m, 2H), 4.43-4.24 (m, 2H), 3.72-3.44 (m, 2H), 3.39-3.09 (m, 5H), 3.06-2.88 (m, 2H), 2.77-2.70 (m, 1H), 2.64-2.53 (m, 3H), 2.31-1.92 (m, 5H), 1.09-0.83 (m, 1H), 0.95 (d, J=5.9 Hz, 3H).

### Example 91

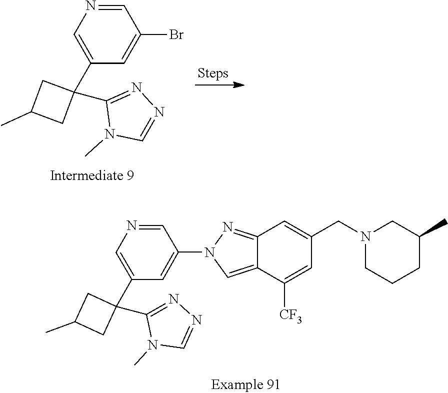

The title compound was synthesized according to Step 4 to Step 6 in Example 66. LCMS: 524.3 [M+H]⁺.

### Example 92

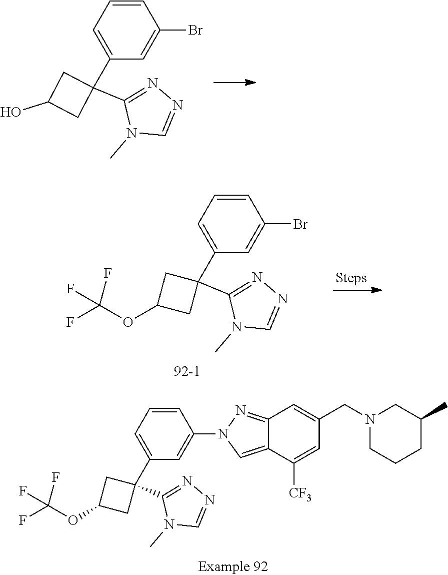 [SMILES: Cn1cnnc1C1(c2cccc(Br)c2)CC(O)C1 ; Cn1cnnc1C1(c2cccc(Br)c2)CC(OC(F)(F)F)C1 ; C[C@H]1CCCN(Cc2cc(C(F)(F)F)c3cn(-c4cccc([C@]5(c6nncn6C)C[C@@H](OC(F)(F)F)C5)c4)nc3c2)C1]

### Step 1: Preparation of Compound 92-1

To a solution of 3-(3-bromophenyl)-3-(4-methyl-4H-1,2,4-triazol-3-yl)cyclobutan-1-ol (200 mg, 0.649 mmol) in EA (3 mL) was added silver trifluoromethanesulfonate (666.96 mg, 2.596 mmol), Selectfluor (344.60 mg, 0.973 mmol) and KF (150.56 mg, 2.596 mmol). After stirring for 5 min, 2-fluoropyridine (252.03 mg, 2.596 mmol) and TMSCF₃ (230.71 mg, 1.622 mmol) was added. After 36 hours, the reaction mixture was filtered through Celite and the filtrate was concentrated in vacuo to give the crude product, which was purified by flash silica gel chromatography to give the Compound 92-1 (160 mg, 65.5%). LCMS: 376.0/378.0 [M+H]⁺;

### Step 2: Preparation of Example 92

The title compound was synthesized according to Step 4 to Step 6 in Example 66. LCMS: 634.4 [M+H+MeCN]⁺.

### Example 93

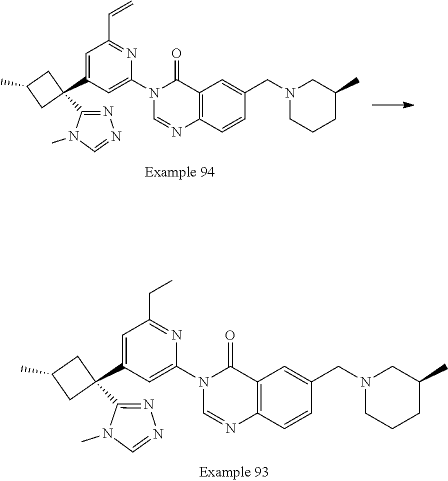

To a solution of Example 94 (100 mg, 0.196 mmol) in EtOAc (3 mL) was added Pd/C 10% (21 mg, 0.020 mmol) under N₂. The suspension was degassed under vacuum and purged with H₂ three times. The mixture was stirred under H₂ balloon at 25° C. for 2 hr. The mixture was filtered through a Celite pad, and the filtrate was concentrated to give the crude product. The residue was purified by prep-HPLC to afford the title compound. LCMS: 512.4 [M+H]⁺; ¹H NMR (400 MHz, Chloroform-d) δ 8.71-8.62 (m, 1H), 8.26 (s, 1H), 8.15-7.91 (m, 2H), 7.88-7.59 (m, 2H), 7.14-6.92 (m, 1H), 3.88 (s, 2H), 3.43-3.29 (m, 3H), 3.26-3.02 (m, 2H), 3.04-2.83 (m, 3H), 2.83-2.64 (m, 4H), 2.15 (t, J=11.6 Hz, 1H), 1.97-1.62 (m, 5H), 1.29 (t, J=7.5 Hz, 3H), 1.19-1.07 (m, 3H), 0.97-0.89 (m, 1H), 0.87 (d, J=6.0 Hz, 3H).

The following compounds was synthesized according to the procedures of Example 45.

| Ex. | LCMS | ¹H NMR |
|---|---|---|
| 94 | 510.2 | ¹H NMR (400 MHz, Chloroform-d) δ 8.71 (s, 1H), 8.26 (s, 1H), 8.03 (s, 1H), 7.88 (s, |
| | [M + H]⁺ | 1H), 7.92-7.74 (m, 2H), 7.19 (s, 1H), 6.74 (dd, J = 16.8, 10.4 Hz, 1H), 6.32 (d, J = |
| | | 17.3 Hz, 1H), 5.54 (d, J = 10.3 Hz, 1H), 3.85-3.50 (m, 2H), 3.35 (s, 3H), 2.96- |
| | | 2.89 (m, 2H), 2.82-2.63 (m, 2H), 2.06-1.42 (m, 9H), 1.16 (d, J = 6.1 Hz, 3H), 0.93- |
| | | 0.81 (m, 4H). |
| 96 | 524.5 | ¹H NMR (400 MHz, DMSO-d₆) δ 8.50 (s, 1H), 8.36 (s, 1H), 8.10 (s, 1H), 7.81 (dd, J = |
| | [M + H]⁺ | 8.3, 1.5 Hz, 1H), 7.70 (d, J = 8.3 Hz, 1H), 7.51 (s, 1H), 7.41 (s, 1H), 3.58 (s, 2H), |
| | | 3.28 (s, 3H), 2.93-2.85 (m, 2H), 2.76-2.69 (m, 2H), 2.63-2.56 (m, 3H), 2.22 (p, J = |
| | | 6.5 Hz, 1H), 1.90 (t, J = 10.2 Hz, 1H), 1.68-1.56 (m, 4H), 1.51-1.41 (m, 1H), |
| | | 1.09 (d, J = 4.9 Hz, 3H), 0.99 (d, J = 6.3 Hz, 4H), 0.91-0.84 (m, 1H), 0.81 (d, J = |
| | | 5.6 Hz, 3H). |

### Example 95

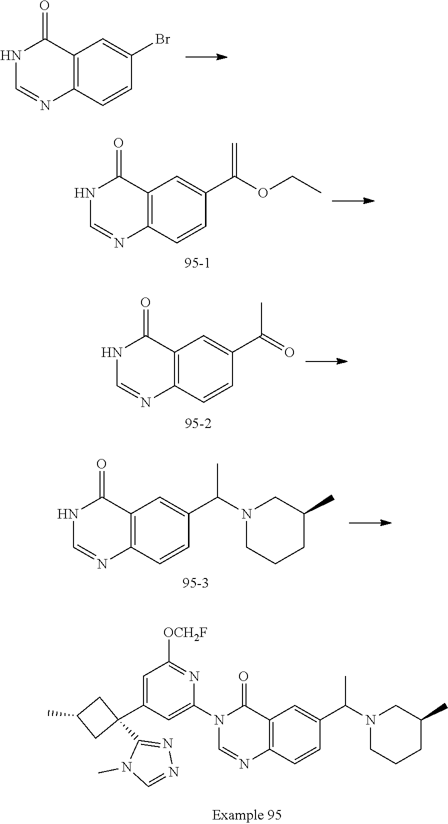

### Step 1: Preparation of Compound 95-1

To a solution of 6-bromoquinazolin-4(3H)-one (500 mg, 2.22 mmol) in dioxane (10 mL) was added 1-Ethoxyvinyltri-n-butyltin (1605.3 mg, 4.44 mmol) and Pd(PPh₃)₄ (128.4 mg, 0.11 mmol). Then the mixture was stirred at 110° C. for 3 hrs under N₂. The reaction mixture was concentrated under reduced pressure to remove dioxane. The residue was diluted with H₂O (200 mL) and extracted with EtOAc (100 mL×2). The combined organic layers were washed with brine (50 mL×2), dried over anhydrous sodium sulfate, filtered, and concentrated under reduced pressure to give Compound 95-1 (400 mg, 83.3%), which was used directly into next step without purification. LCMS: 217.2 [M+H]⁺.

### Step 2: Preparation of Compound 95-2

To a solution of Compound 95-1 (500 mg, 1.759 mmol) in THE (10 mL) was added HCl (1M, 11 mL, 23.127 mmol) at 25° C. under N₂. Then the reaction mixture was stirred at 25° C. for 1 h. The reaction mixture was purified by reverse phase chromatography to afford (400 mg, 91.9%). LCMS: 189.0 [M+H]⁺. H NMR (400 MHz, DMSO) δ 8.66 (d, J=1.9 Hz, 1H), 8.30 (d, J=8.5, 2.0 Hz, 1H), 8.25 (s, 1H), 7.76 (d, J=8.5 Hz, 1H), 2.51 (s, 3H).

### Step 3: Preparation of Compound 95-3

A mixture of Compound 95-2 (350 mg, 1.860 mmol), (3S)-3-methylhexahydropyridine hydrochloride (378.3 mg, 2.790 mmol and Ti(OⁱPr)₄ (2.1 g, 7.439 mmol) in THE (15 mL) was stirred at 80° C. for 16 hr under N₂ atmosphere. After being cooled to room temperature, STAB (1.17 g, 5.579 mmol) was added. The resulting mixture was stirred at 80 T for 6 hr. The reaction mixture was quenched by addition of H₂O (100 ml). The resulting mixture was filtered and the filtrate was extracted with DCM (2×200 mL). The combined organic layers were dried over anhydrous sodium sulfate, filtered and the filtrate was concentrated under reduced pressure to give a residue. The residue was purified by flash silica gel chromatography to give Compound 95-3 (100 mg, 19.8%). LCMS: 272.2 [M+H]⁺.

### Step 4: Preparation of the Tile Compound

The title compound was synthesized according to the step 3 in Example 38. LCMS: 546.4 [M+H]⁺. ¹H NMR (400 MHz, Chloroform-d) δ 8.60 (s, 1H), 8.22 (s, 1H), 8.06 (s, 1H), 7.85 (s, 1H), 7.80-7.51 (m, 2H), 6.84 (s, 1H), 6.04 (d, J=51.9 Hz, 2H), 3.68-3.50 (m, 1H), 3.35 (s, 3H), 2.94-2.88 (m, 2H), 2.78-2.65 (m, 3H), 2.01-1.75 (m, 2H), 1.73-1.49 (m, 7H), 1.45-1.35 (m, 2H), 1.16 (d, J=5.8 Hz, 3H), 0.93-0.71 (m, 4H).

### Example 97

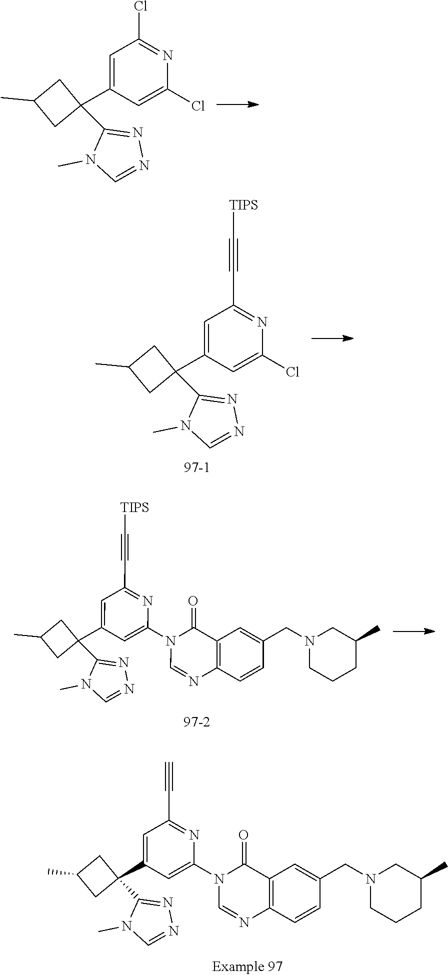

### Step 1: Preparation of Compound 97-1

To a solution of Trimethylsilylacetylene (128.9 mg, 0.707 mmol) and 2,6-dichloro-4-(3-methyl-1-(4-methyl-4H-1,2,4-triazol-3-yl)cyclobutyl)pyridine (200 mg, 0.673 mmol) in THF (2 mL) was added CuI (12.8 mg, 0.067 mmol), Pd(PPh₃)₂Cl₂ (52.4 mg, 0.067 mmol) and TEA (0.47 mL, 3.365 mmol) at room temperature. The mixture was stirred at 50° C. for 4 hrs. The mixture was concentrated under reduced pressure to give a residue. The residue was purified by reverse phase chromatography to afford Compound 97-1 (162 mg, 54.3%). LCMS: 443.4 [M+H]⁺;

### Step 2: Preparation of Compound 97-2

The title compound was synthesized according to the step 3 in Example 38. LCMS: 664.5 [M+H]⁺.

### Step 3: Preparation of the Title Compound

To a solution of Compound 97-2 (74 mg, 0.111 mmol) in THF (10 mL) was added TBAF solution (0.28 mL, 0.279 mmol) at room temperature. The mixture was stirred at room temperature for 3 hrs. The reaction mixture was concentrated under reduced pressure to give a residue. The residue was purified by prep-HPLC to afford Example 97. LCMS: 508.3 [M+H]⁺; ¹H NMR (400 MHz, DMSO-d₆) δ 8.50 (s, 1H), 8.38 (s, 1H), 8.11 (s, 1H), 7.93-7.75 (m, 2H), 7.74-7.68 (m, 1H), 7.54 (s, 1H), 4.55 (s, 1H), 3.59 (s, 2H), 3.27 (s, 3H), 2.97-2.82 (m, 2H), 2.80-2.56 (m, 3H), 1.93-1.44 (m, 8H), 1.08 (d, J=4.8 Hz, 3H), 0.89-0.83 (m, 1H), 0.81 (d, J=5.6 Hz, 3H).

The following compounds was synthesized according to the previous procedures and step 4 in Example 10.

| Ex. | LCMS | ¹H NMR |
|---|---|---|
| 98 | 536.4 | — |
| | [M + H]⁺ | |
| 102 | 604.4 | ¹H NMR (400 MHz, Chloroform-d) δ 8.81 (s, 1H), 8.64 (s, 1H), 8.56 (s, 1H), |
| | [M + H]⁺ | 8.32 (s, 1H), 7.50 (s, 1H), 7.06 (s, 1H), 6.05 (d, J = 51.7 Hz, 2H), 5.02 (d, J = |
| | | 46.8 Hz, 1H), 4.54 (s, 2H), 3.47 (s, 3H), 3.36-3.12 (m, 4H), 3.02-2.87 (m, |
| | | 2H), 2.79-2.66 (m, 3H), 2.30-1.82 (m, 4H), 1.18 (d, J = 3.9 Hz, 3H). |
| 103 | 614.4 | ¹H NMR (400 MHz, DMSO-d₆) δ 8.86 (s, 1H), 8.67 (s, 1H), 8.45 (s, 1H), 8.37 |
| | [M + H]⁺ | (d, J = 2.0 Hz, 1H), 7.47 (s, 1H), 7.22 (s, 1H), 6.16 (d, J = 51.7 Hz, 2H), 3.80- |
| | | 3.76 (m, 1H), 3.29 (s, 3H), 3.19 (s, 2H), 2.93-2.88 (m, 1H), 2.70-2.30 (m, |
| | | 4H), 1.95-1.57 (m, 9H), 1.15-1.07 (m, 3H), 1.07-0.99 (m, 1H), 0.97-0.81 |
| | | (m, 3H). |
| 105 | 570.1 | ¹H NMR (400 MHz, DMSO-d₆) δ 8.71-8.59 (m, 1H), 8.44-8.32 (m, 2H), 8.17- |
| | [M + H]⁺ | 8.03 (m, 1H), 7.97-7.81 (m, 1H), 7.65-7.43 (m, 1H), 7.18-7.03 (m, 1H), |
| | | 4.42-4.18 (m, 2H), 3.73-3.43 (m, 2H), 3.26-3.20 (m, 3H), 2.92-2.76 (m, |
| | | 3H), 2.63-2.28 (m, 4H), 2.10-1.80 (m, 3H), 1.70-1.43 (m, 3H), 1.22-0.94 |
| | | (m, 4H). |
| 106 | 618.3 | ¹H NMR (400 MHz, Chloroform-d) δ 8.77 (s, 1H), 8.71-8.11 (m, 2H), 8.06 (s, |
| | [M + H]⁺ | 1H), 7.63 (s, 1H), 6.93 (s, 1H), 6.04 (d, J = 51.7 Hz, 2H), 4.28-4.02 (m, 1H), |
| | | 3.63 (s, 2H), 3.35 (s, 3H), 2.97-2.66 (m, 7H), 2.37-1.75 (m, 4H), 1.53-1.29 |
| | | (m, 1H), 1.17 (d, J = 5.8 Hz, 3H), 1.12-0.86 (m, 3H). |
| 108 | 618.3 | ¹H NMR (400 MHz, Chloroform-d) δ 8.76 (s, 1H), 8.48 (s, 1H), 8.23 (s, 1H), |
| | [M + H]⁺ | 8.06 (s, 1H), 7.66 (s, 1H), 6.91 (s, 1H), 6.03 (d, J = 51.8 Hz, 2H), 3.78 (s, 2H), |
| | | 3.35 (s, 3H), 2.97-2.85 (m, 2H), 2.81-2.54 (m, 5H), 2.47-1.50 (m, 6H), 1.38 |
| | | (d, J = 21.6 Hz, 3H), 1.17 (d, J = 5.9 Hz, 3H). |
| 109 | 636.3 | ¹H NMR (400 MHz, Chloroform-d) δ 8.76 (s, 1H), 8.48 (s, 1H), 8.33-8.13 (m, |
| | [M + H]⁺ | 1H), 8.06 (s, 1H), 7.63 (s, 1H), 6.93 (s, 1H), 6.04 (d, J = 51.7 Hz, 2H), 3.76 (s, |
| | | 2H), 3.35 (s, 3H), 3.00-2.63 (m, 7H), 2.54-2.02 (m, 5H), 1.17 (d, J = 5.8 Hz, |
| | | 3H), 1.02 (d, J = 5.8 Hz, 3H). |
| 120 | 566.2 | ¹H NMR (400 MHz, Chloroform-d) δ 8.75 (s, 1H), 8.57 (d, J = 5.3 Hz, 1H), 8.48 |
| | [M + H]⁺ | (s, 1H), 8.18 (s, 1H), 8.06 (s, 1H), 7.91 (s, 1H), 7.32 (dd, J = 5.3, 1.5 Hz, 1H), |
| | | 4.45 (dt, J = 13.3, 6.7 Hz, 2H), 3.90-3.77 (m, 2H), 3.34 (s, 3H), 3.04-2.99 (m, |
| | | 1H), 2.95-2.86 (m, 3H), 2.80-2.66 (m, 7H), 2.40-2.30 (m, 1H), 2.20-2.10 |
| | | (m, 1H), 1.17 (d, J = 6.1 Hz, 3H). |
| 122 | 568.4 | ¹H NMR (400 MHz, Chloroform-d) δ 8.61 (s, 1H), 8.26 (s, 1H), 8.05 (s, 1H), |
| | [M + H]⁺ | 7.98-7.52 (m, 3H), 6.88 (s, 1H), 6.04 (d, J = 51.9 Hz, 2H), 3.66 (s, 2H), 3.35 (s, |
| | | 3H), 2.94-2.86 (m, 2H), 2.85-2.57 (m, 5H), 2.48-2.30 (m, 1H), 2.30-1.76 |
| | | (m, 3H), 1.17 (d, J = 5.9 Hz, 3H), 1.06-0.96 (m, 4H). |
| 123 | 550.3 | ¹H NMR (400 MHz, Chloroform-d) δ 8.61 (s, 1H), 8.24 (s, 1H), 8.05 (s, 1H), |
| | [M + H]⁺ | 8.02-7.45 (m, 3H), 6.89 (s, 1H), 6.04 (d, J = 51.9 Hz, 2H), 4.12 (d, J = 50.8 Hz, |
| | | 1H), 3.61 (s, 2H), 3.35 (s, 3H), 3.01-2.64 (m, 7H), 2.48-1.73 (m, 4H), 1.17 (d, |
| | | J = 5.8 Hz, 3H), 1.10-0.90 (m, 4H). |

### Example 99

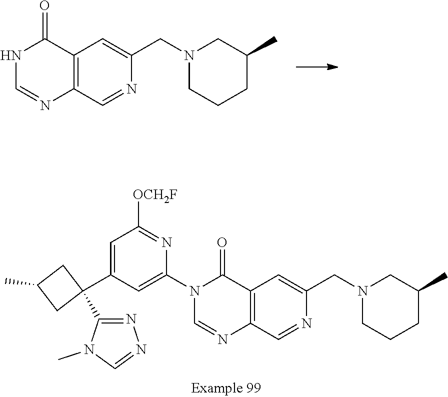

The title compound was synthesized according to Step 3 in Example 38 with (S)-6-((3-methylpiperidin-1-yl)methyl)pyrido[3,4-d]pyrimidin-4(3H)-one (which was synthesized according to Intermediate 3) as the substrate. LCMS: 533.3 [M+H]⁺; ¹H NMR (400 MHz, DMSO-d₆) δ 9.05 (s, 1H), 8.68 (s, 1H), 8.38 (s, 1H), 8.05 (s, 1H), 7.53 (s, 1H), 7.10 (s, 1H), 6.13 (d, J=52.4 Hz, 2H), 3.73 (s, 2H), 3.27 (s, 3H), 2.91-2.87 (m, 2H), 2.78-2.74 (m, 2H), 2.67 (s, 1H), 2.63-2.58 (m, 4H), 1.70-1.59 (m, 4H), 1.09 (d, J=4.2 Hz, 3H), 0.85-0.79 (m, 4H).

### Example 100

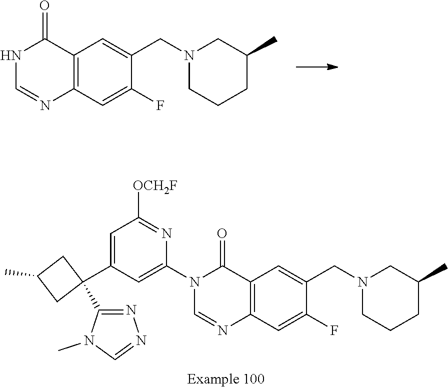

The title compound was synthesized according to Step 3 in Example 38. LCMS: 550.4 [M+H]⁺; ¹H NMR (400 MHz, DMSO-d₆) δ 8.61 (s, 1H), 8.37 (s, 1H), 8.23 (d, J=7.9 Hz, 1H), 7.57-7.49 (m, 2H), 7.05 (s, 1H), 6.12 (d, J=52.2 Hz, 2H), 3.62 (s, 2H), 3.27 (s, 3H), 2.95-2.85 (m, 2H), 2.75 (t, J=9.2 Hz, 2H), 2.66-2.53 (m, 4H), 1.93 (t, J=10.3 Hz, 1H), 1.67-1.55 (m, 4H), 1.08 (d, J=4.9 Hz, 3H), 0.87-0.79 (m, 4H).

### Example 101

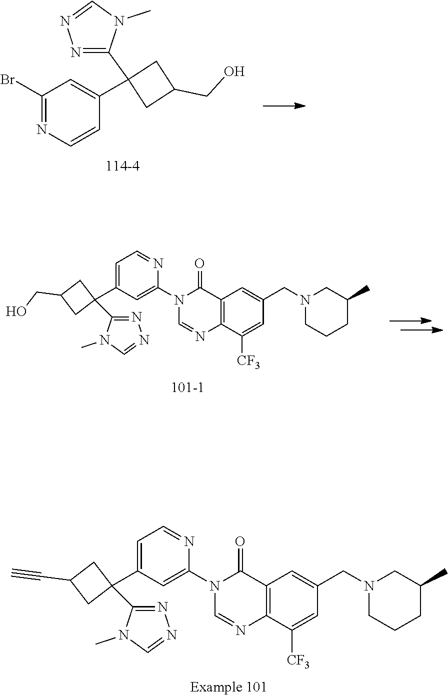

### Step 1: Preparation of Compound 101-1

The Compound 101-1 was synthesized according to Step 3 in Example 38 with Compound 114-4 as the substrate. LCMS: 568.3 [M+H]⁺;

### Step 2: Preparation of Example 101

The title compound was synthesized according to step 7 to step 8 in Example 32. LCMS: 562.3 [M+H]⁺; ¹H NMR (400 MHz, Chloroform-d) δ 8.77 (s, 1H), 8.67-8.40 (m, 2H), 8.14 (s, 1H), 7.71 (s, 1H), 7.26 (s, 2H), 4.34-3.57 (m, 2H), 3.57-3.41 (m, 2H), 3.39 (s, 3H), 3.32-3.23 (m, 1H), 2.94-2.81 (m, 2H), 2.25 (s, 1H), 1.85-1.41 (m, 8H), 1.00-0.78 (m, 4H).

### Example 104

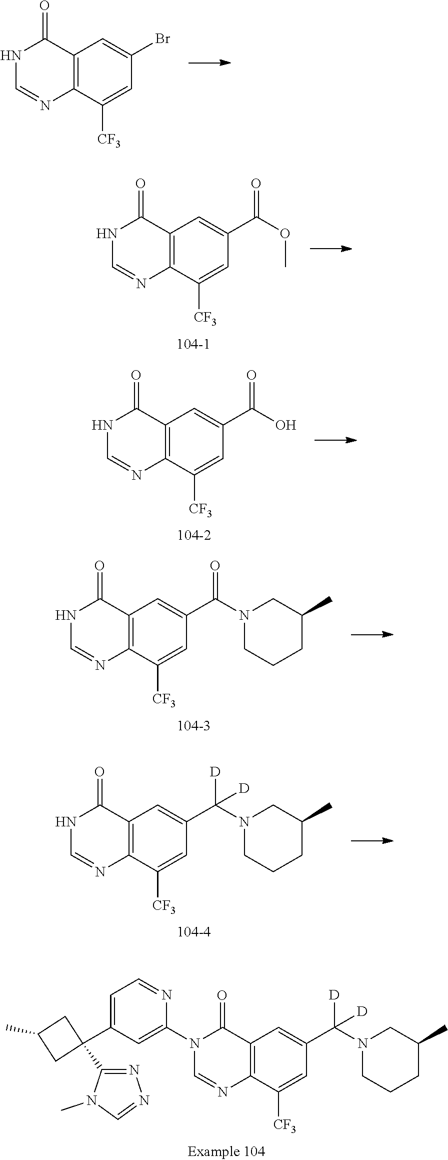

### Step 1: Preparation of Compound 104-1

To a solution of 6-bromo-8-(trifluoromethyl)quinazolin-4(3H)-one (500 mg, 6.826 mmol) in MeOH (5 mL)/DMF (5 mL)/TEA (5 mL) was added Pd(OAc)₂ (92 mg, 0.410 mmol) and Xantphos (474 mg, 0.819 mmol) at 25° C. The reaction was stirred at 60° C. for 48 hrs under CO (50 Psi). The mixture was filtered through a Celite pad, and the filtrate was concentrated to give the crude product. The crude was purified by flash silica gel chromatography to afford Compound 104-1 (400 mg, 86.1%). LCMS: 273.0 [M+H]⁺;

### Step 2: Preparation of Compound 104-2

To a solution of Compound 104-1 (400 mg, 1.470 mmol) in EtOH (2 mL) and H₂O (1 mL) was added NaOH (294 mg, 7.348 mmol) at 25° C. The reaction was stirred at 25° C. for 18 hrs. The pH was adjusted to around 3 by progressively adding HCl (1 M). The mixture was filtered through a filter, collect the filter cake to give Compound 104-2 (280 mg, 73.8%). LCMS: 300.0 [M+ACN+H]⁺;

### Step 3: Preparation of Compound 104-3

To a solution of Compound 104-2 (250 mg, 0.968 mmol) and (3S)-3-methylhexahydropyridine (96.0 mg, 0.968 mmol) in DMF (4 mL) was added EDCI (278.4 mg, 1.452 mmol) at 25° C. The reaction was stirred at 25° C. for 2 hrs. The reaction mixture was purified by reverse phase chromatography to afford Compound 104-3. LCMS: 340.1 [M+H]⁺;

### Step 4: Preparation of Compound 104-4

To a solution of Compound 104-3 (60 mg, 0.177 mmol) in THE (2 mL) was added Lithium Aluminum Deuteride (18.6 mg, 0.442 mmol). The mixture was stirred at room temperature for 2 hrs under N₂. The reaction mixture was quenched by addition of NH₄Cl (sat. aq. 10 mL) and extracted with DCM (10 mL×3). The combined organic phase was washed with brine (10 mL), dried over anhydrous Na₂SO₄, filtered and concentrated under reduced pressure to give a residue. The residue was purified by reverse phase chromatography to afford Compound 104-4. LCMS: 328.2 [M+H]⁺;

### Step 5: Preparation of the title compound

The title compound was synthesized according to Step 3 in Example 38. LCMS: 554.3 [M+H]⁺; ¹H NMR (400 MHz, Chloroform-d) δ 8.77 (s, 1H), 8.67-8.41 (m, 2H), 8.22-8.03 (m, 1H), 7.91 (s, 1H), 7.46-7.26 (m, 2H), 3.33 (s, 3H), 2.97-2.66 (m, 5H), 1.87-1.52 (m, 8H), 1.17 (d, J=6.1 Hz, 3H), 1.04-0.79 (m, 4H).

### Example 107

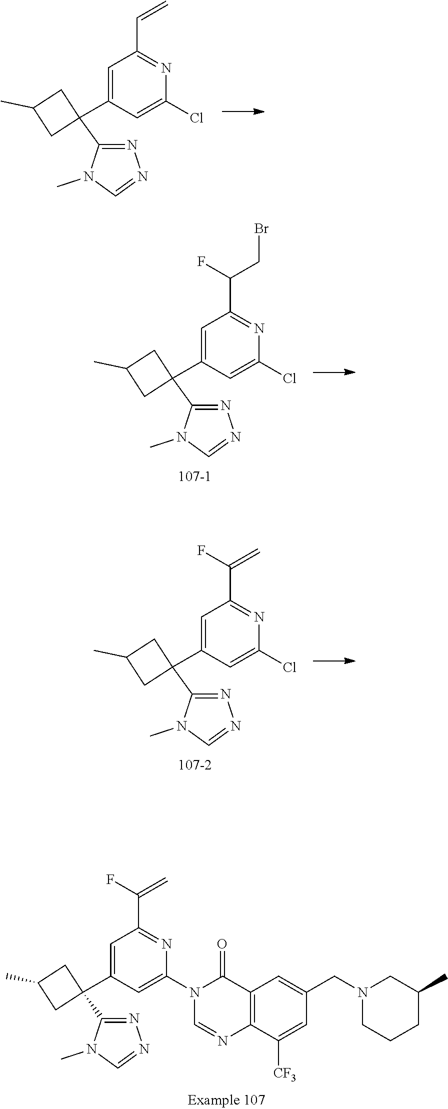

### Step 1: Preparation of Compound 107-1

To a solution of 2-chloro-4-(3-methyl-1-(4-methyl-4H-1,2,4-triazol-3-yl)cyclobutyl)-6-vinylpyridine (350 mg, 1.212 mmol) in DCM (15 mL) was added NBS (1.07 g, 6.060 mmol) at 0° C. Triethylamine trihydrofluoride (1.95 g, 12.119 mmol) was added dropwise, and the reaction was warmed to room temperature, then stirred at room temperature for overnight. The reaction mixture was quenched with water and solid sodium bicarbonate (1.06 g, 12.68 mmol) in small portions. The biphasic mixture was separated and the aqueous layer was extracted with DCM (15 mL×2). The combined organic layers were dried over sodium sulfate, filtered, and concentrated in vacuum to give residue. The residue was purified by flash silica gel chromatography to afford Compound 107-1 (250 mg, 53.2%). LCMS: 387.0/389.0 [M+H]⁺;

### Step 2: Preparation of Compound 107-2

To a solution of Compound 107-1 (250 mg, 0.645 mmol) in DMF (2 mL) was added K₂CO₃ (178.2 mg, 1.290 mmol) at 25° C. The reaction mixture was stirred at 100° C. for 16 hrs. The reaction mixture was filtered and the filter cake was washed with methanol. The filtrate was concentrated in vacuo to give the crude product. The crude was purified by flash silica gel chromatography to afford Compound 107-2. LCMS: 307.2 [M+H]⁺;

### Step 3: Preparation of the title compound

The title compound was synthesized according to the step 3 in Example 38. LCMS: 596.4 [M+H]⁺;

### Example 110

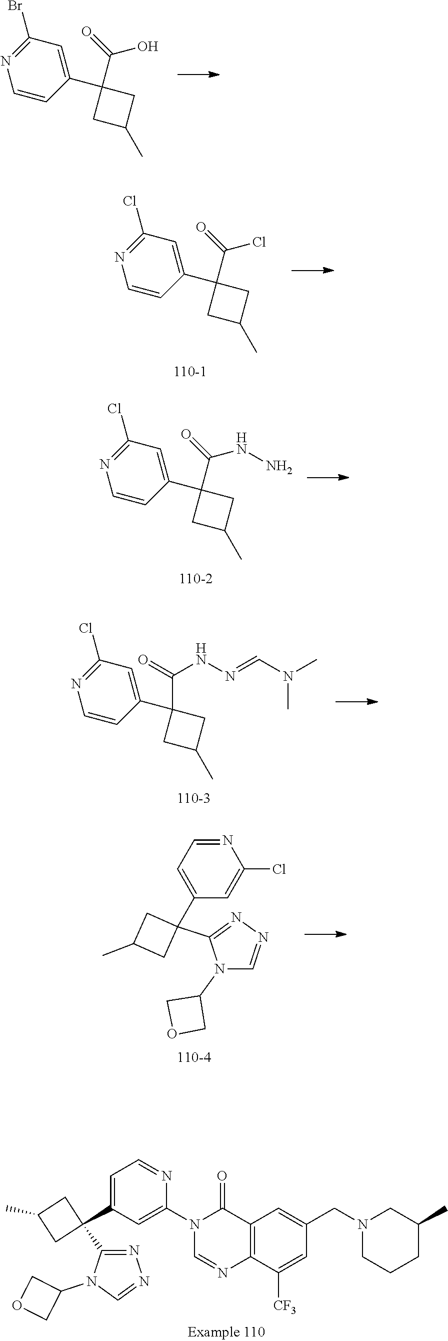

### Step 1: Preparation of Compound 110-1

A mixture of 1-(2-bromopyridin-4-yl)-3-methylcyclobutane-1-carboxylic acid (2000 mg, 7.405 mmol) in SOC₂ (30 mL) was stirred at 80° C. for 1 hr. The reaction mixture was concentrated to afford Compound 110-1 (2100 mg, 7.277 mmol), which was used to next step without further purification.

### Step 2: Preparation of Compound 110-2

Compound 110-1 (2100 mg, 7.277 mmol) in DCM (30 mL) was added dropwise to a solution of hydrazine hydrate (25 mL, 792.308 mmol) in DCM (50 mL) at 0° C., the mixture was stirred at 0° C. for 30 min. The reaction mixture was diluted with water (50 mL) and extracted with DCM. The combined organic phase was dried over anhydrous Na₂SO₄, filtered, and concentrated under reduced pressure to give a residue. The residue was purified by flash silica gel chromatography to afford Compound 110-2 LCMS: 240.2 [M+H]⁺;

### Step 3: Preparation of Compound 110-3

To a solution of Compound 110-2 (1600 mg, 6.675 mmol) in DCM (15 mL) was added DMF-DMA (1591.3 mg, 13.350 mmol), the mixture was stirred at 50° C. for 4 hrs. The reaction mixture was concentrated under reduced pressure to afford Compound 110-3. LCMS: 295.2 [M+H]⁺;

### Step 4: Preparation of Compound 110-4

To a solution of Compound 110-3 (1300 mg, 4.410 mmol) in THF (8 mL) was added oxetan-3-amine hydrochloride (1611.8 mg, 22.049 mmol), TEA (3.12 mL, 22.490 mmol) and AcOH (0.25 mL, 4.410 mmol). The mixture was stirred at 90° C. for overnight. The mixture was partitioned between EtOAc (50 mL) and H₂O (50 mL), the organic layer was separated, dried, and concentrated to give a residue. The residue was purified by flash silica gel chromatography to afford Compound 110-4. LCMS: 305.2 [M+H]⁺;

### Step 2: Preparation of the title compound

The title compound was synthesized according to the step 3 in Example 38. LCMS: 635.4 [M+MeCN+H]⁺; H NMR (400 MHz, DMSO-d₆) δ 9.06 (s, 1H), 8.67 (s, 1H), 8.65-8.59 (m, 1H), 8.39 (s, 1H), 8.16 (s, 1H), 7.76 (s, 1H), 7.45 (dd, J=5.2, 1.6 Hz, 1H), 4.89-4.73 (m, 1H), 4.64-4.45 (m, 4H), 3.66 (s, 2H), 2.88-2.54 (m, 7H), 2.08-1.89 (m, 1H), 1.70-1.39 (m, 5H), 1.08 (d, J=5.6 Hz, 3H), 0.90-0.78 (m, 4H).

The following compounds was synthesized according to step 3 in Example 38 with corresponding substrates.

| Ex. | LCMS | ¹H NMR |
|---|---|---|
| 111 | 549.9 | ¹H NMR (400 MHz, Chloroform-d) δ 8.69 (s, 1H), 8.13 (s, 1H), 7.74 (d, J = 8.5 |
| | [M + H]⁺ | Hz, 1H), 7.55-7.33 (m, 1H), 7.04 (s, 1H), 6.92-6.76 (m, 1H), 6.05 (d, J = 51.6 |
| | | Hz, 2H), 4.42-4.21 (m, 2H), 3.93-2.99 (m, 6H), 2.96-2.57 (m, 5H), 2.41- |
| | | 2.27 (m, 1H), 2.20-1.78 (m, 4H), 1.26-1.14 (m, 3H), 1.12-1.00 (m, 1H), |
| | | 0.95 (d, J = 6.1 Hz, 3H). |
| 112 | 532.3 | ¹H NMR (400 MHz, Chloroform-d) δ 8.60 (s, 1H), 8.24 (s, 1H), 8.07 (s, 1H), |
| | [M + H]⁺ | 7.97-7.65 (m, 3H), 6.86 (s, 1H), 6.04 (d, J = 51.9 Hz, 2H), 3.65 (s, 2H), 3.35 (s, |
| | | 3H), 3.06-2.54 (m, 7H), 2.04-1.51 (m, 6H), 1.16 (d, J = 5.8 Hz, 3H), 1.00- |
| | | 0.74 (m, 4H). |
| 115 | 582.1 | ¹H NMR (400 MHz, DMSO-d₆) δ 8.78 (s, 1H), 8.37 (s, 1H), 8.36 (s, 1H), 8.14 |
| | [M + H]⁺ | (s, 1H), 7.35 (s, 1H), 6.92 (s, 1H), 3.91 (s, 3H), 3.65 (s, 2H), 3.26 (s, 3H), 2.94- |
| | | 2.80 (m, 2H), 2.76-2.65 (m, 2H), 2.63-2.53 (m, 3H), 1.94 (t, J = 10.1 Hz, |
| | | 1H), 1.69-1.55 (m, 4H), 1.48 (d, J = 11.8 Hz, 1H), 1.08 (d, J = 5.1 Hz, 3H), |
| | | 0.92-0.84 (m, 1H), 0.82 (d, J = 6.1 Hz, 3H). |
| 117 | 618.4 | ¹H NMR (400 MHz, Chloroform-d) δ 8.64 (s, 1H), 8.43 (s, 1H), 8.16 (s, 1H), |
| | [M + H]⁺ | 8.06 (s, 1H), 7.69 (s, 1H), 7.34 (t, J = 72.2 Hz, 1H), 6.96 (s, 1H), 3.62 (s, 2H), |
| | | 3.43-3.31 (m, 3H), 3.27-2.53 (m, 7H), 2.37-1.89 (m, 2H), 1.74-1.65 (m, |
| | | 4H), 1.19-1.14 (m, 3H), 0.92-0.81 (m, 4H). |

### Example 113

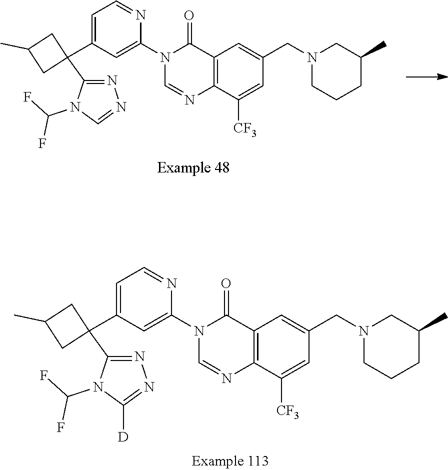

A suspension of Example 48 (110 mg, 0.193 mmol) in D₂O (1.5 mL, 0.193 mmol) was stirred at 120′˜C overnight. The mixture was purified by prep-HPLC to afford Example 113 as a FA salt. LCMS: 589.4 [M+H]⁺; ¹H HNMR (400 MHz, DMSO-d₆) δ 8.73-8.57 (m, 2H), 8.39 (s, 1H), 8.16 (s, 2H), 7.86 (s, 1H), 7.63-7.30 (m, 2H), 3.67 (s, 2H), 2.93-2.43 (m, 7H), 2.04-1.89 (m, 1H), 1.71-1.54 (m, 4H), 1.54-1.39 (m, 1H), 1.09 (d, J=4.6 Hz, 3H), 0.86-0.79 (in, 4H).

### Example 114

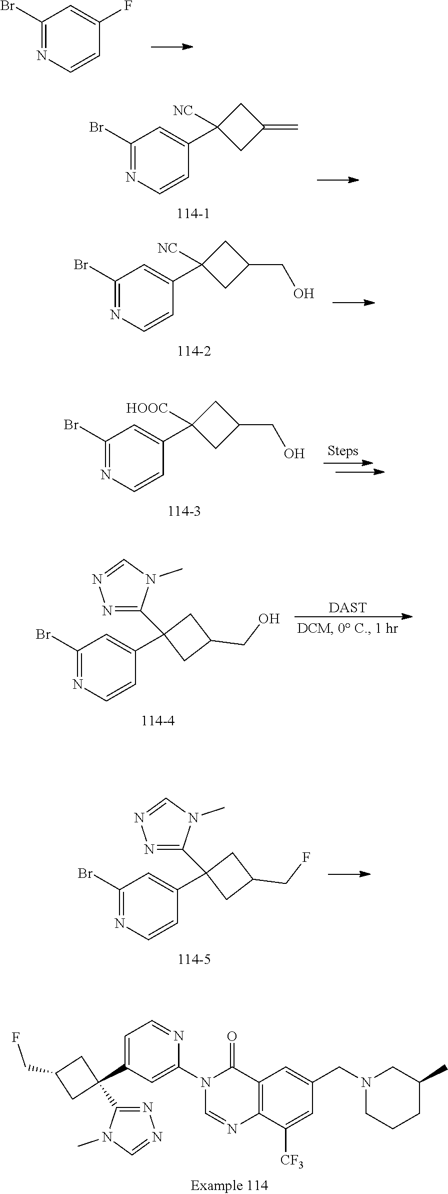

### Step 1: Preparation of Compound 114-1

To a solution of 3-methylidenecyclobutane-1-carbonitrile (5289.8 mg, 56.818 mmol) in THF (60 mL) was added LiHMDS (56.82 mL, 56.818 mmol) at −78° C., then 2-bromo-4-fluoropyridine (10.0 g, 56.818 mmol) was added. The resulting mixture was stirred for 2 hours at −78° C. The reaction was quenched by the addition of saturated NH₄Cl solution and the resulting mixture was extracted with EtOAc (100 mL×2). Then the organic layers were collected and washed with brine (20 mL×2), dried over anhydrous sodium sulfate, filtered, and concentrated under reduced pressure to give a residue. The residue was purified by flash silica gel chromatography to afford Compound 114-1 (6.8 g, 48.0%). LCMS: 248.8/250.8 [M+H]⁺;

### Step 2: Preparation of Compound 114-2

To a solution of Compound 114-1 (6.5 g, 26.094 mmol) in dry THF (20 mL), was added 9-BBN (67.84 mL, 33.922 mmol) at −10° C. The resulting mixture was stirred for overnight at room temperature. The resulting mixture was cooled to 0° C.; NaOH (7.5 mL, 7.500 mmol) and H₂O₂ (3.25 mL, 28.703 mmol) were added in sequence, the reaction mixture was stirred for 30 mins. The mixture was quenched by the addition of aq. Na₂S₂O₃. The reaction mixture was partitioned between water (30 mL) and EtOAc (50 mL). The organic phase was separated, washed with water (20 mL×2), dried over anhydrous sodium sulfate, filtered, and concentrated under reduced pressure to give a residue. The residue was purified by flash silica gel chromatography to afford Compound 114-2 (6 g, 86.1%). LCMS: 266.9/268.9 [M+H]⁺;

### Step 3: Preparation of Compound 114-3

To a solution of Compound 114-2 (5.34 g, 19.993 mmol) in EtOH (16 mL) and H₂O (30 mL) was added NaOH (3.2 g, 79.972 mmol) at room temperature. After addition, the mixture was stirred at 80° C. for overnight. The mixture was adjusted to pH=4 with aq. HCl (1 N). The mixture was concentrated under reduced pressure to give a residue. The residue was purified by Reverse Phase Chromatography to give Compound 114-3 (5 g, 87.4%). LCMS: 286.0/288.0 [M+H]⁺;

### Step 4: Preparation of Compound 114-4

Compound 114-4 was synthesized according to the previous procedures. LCMS: 323.0/325.0 [M+H]⁺;

### Step 5: Preparation of Compound 114-5

To a solution of Compound 114-4 (240 mg, 0.743 mmol) in DCM (2 mL) was added DAST (143.6 mg, 0.891 mmol) at 0° C., the mixture was stirred at room temperature for 2 hrs. The reaction was diluted with DCM (10 mL) and water (5 mL). The organic phase was separated, washed with H₂O (5 mL×2), dried over anhydrous sodium sulfate, filtered, and concentrated under reduced pressure to give a crude Compound 114-5. LCMS: 325.0/327.0 [M+H]³⁰;

### Step 6: Preparation of the title compound

The title compound was synthesized according to the step 3 in Example 38. LCMS: 570.4 [M+H]³⁰;

### Example 116

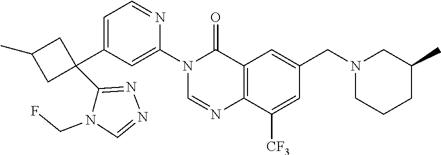

The title compound was synthesized according to the procedure of Example 48. LCMS: 570.2 [M+H]⁺; ¹H NMR (400 MHz, DMSO-d₆) δ 8.76-8.69 (m, 11H), 8.69-8.60 (m, 2H), 8.40 (s, 1H), 8.16 (s, 1H), 7.93 (s, 1H), 7.48 (s, 1H), 5.74 (d, J=50.9 Hz, 2H), 3.66 (s, 2H), 2.93-2.89 (m, 2H), 2.75-2.65 (m, 5H), 2.00-1.90 (m, 11H), 1.72-1.57 (m, 4H), 1.53-1.45 (m, 1H), 1.13-1.06 (m, 3H), 0.89-0.79 (m, 4H).

### Example 118

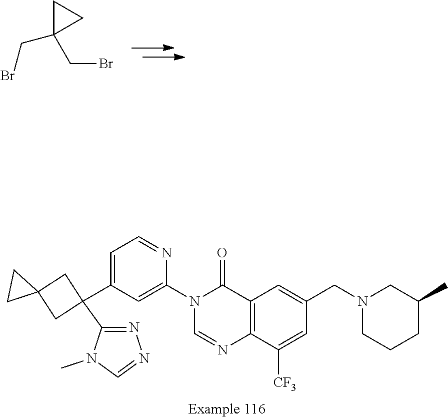 [SMILES: BrCC1(CBr)CC1 ; C[C@H]1CCCN(Cc2cc(C(F)(F)F)c3ncn(-c4cc(C5(c6nncn6C)CC6(CC6)C5)ccn4)c(=O)c3c2)C1]

The title compound was synthesized according to the previous procedures with 1,1-bis(bromomethyl)cyclopropane as the starting material. LCMS: 564.2 [M+H]⁺;

### Example 119

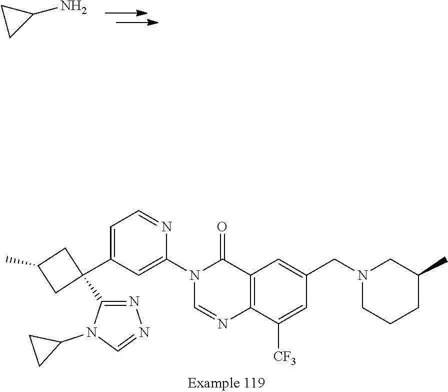

The title compound was synthesized according to Example 110 with cyclopropanamine as the substrate. LCMS: 578.4 [M+H]⁺; ¹H NMR (400 MHz, Chloroform-d) δ 8.74 (s, 1H), 8.56 (d, J=4.9 Hz, 1H), 8.44 (s, 1H), 8.25-7.83 (m, 3H), 7.29 (s, 1H), 3.61 (s, 2H), 3.00-2.88 (m, 2H), 2.79-2.54 (m, 6H), 1.96 (s, 1H), 1.67 (s, 4H), 1.25 (s, 1H), 1.16 (d, J=6.3 Hz, 3H), 1.01-0.66 (m, 8H).

### Example 121

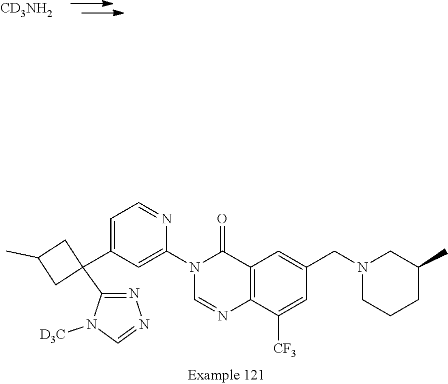

The title compound was synthesized according to Example 110 with CD₃NH₂ as the substrate. LCMS: 555.5 [M+H]⁺; ¹H NMR (400 MHz, DMSO-d₆) δ 8.69 (s, 1H), 8.64 (d, J=5.4 Hz, 1H), 8.40 (s, 1H), 8.37 (s, 1H), 8.17 (s, 1H), 7.83 (s, 1H), 7.47 (d, J=5.4 Hz, 1H), 3.68 (s, 2H), 2.98-2.48 (m, 7H), 2.37-1.97 (m, 1H), 1.77-1.55 (m, 4H), 1.54-1.41 (m, 1H), 1.16-1.00 (m, 3H), 0.94-0.75 (m, 4H).

### Example 124

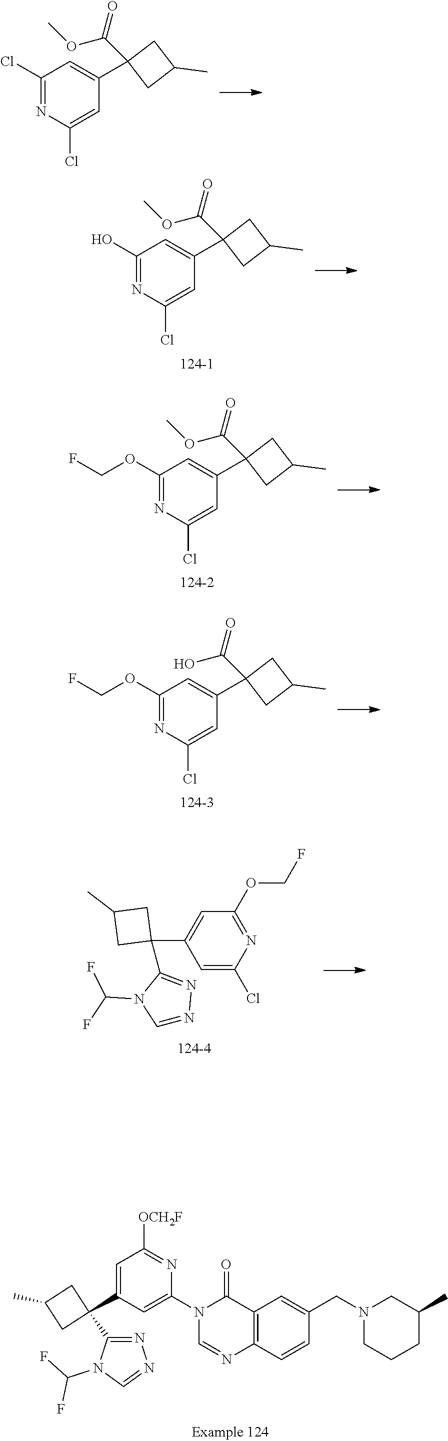

### Step 1: Preparation of Compound 124-1

To a solution of methyl 1-(2,6-dichloropyridin-4-yl)-3-methylcyclobutane-1-carboxylate (15 g, 54.725 mmol) in DMSO (50 mL) was added N-hydroxyacetamide (12.3 g, 164.174 mmol) and K₂CO₃ (22.7 g, 164.174 mmol). The resulting mixture was stirred at 80′C for 12 hrs. The reaction mixture was purified by reverse phase chromatography to afford Compound 124-1 (8.7 g). LCMS: 256.1 [M+H]⁺;

### Step 2: Preparation of Compound 124-2

To a solution of Compound 124-1 (5.8 g, 43.801 mmol) in DMF (29 mL) was added fluoromethyl 4-methylbenzenesulfonate (13.4 g, 65.702 mmol) and Cs₂CO₃ (14.3 g, 43.801 mmol). The resulting mixture was stirred at 80° C. for 12 hrs. The reaction mixture was purified by reverse phase chromatography to afford Compound 124-2. LCMS: 288.0 [M+H]⁺;

### Step 3: Preparation of Compound 124-3

To a solution of Compound 124-2 (4.8 g, 16.684 mmol) in ethanol (50 mL) and H₂O (25 mL) was added NaOH (3.3 g, 83.420 mmol). The resulting mixture was stirred at room temperature for 1 hr.

The mixture was extracted with ethyl acetate. The aqueous phase was adjusted to pH 4 with HCl (1M), then was extracted with ethyl acetate. The combined organic phase was dried over anhydrous Na₂SO₄, concentrated in vacuo to give the crude Compound 124-3. LCMS: 274.0 [M+H]⁺;

### Step 4: Preparation of Compound 124-4

The Compound 124-4 was synthesized according to the previous procedures and Example 48. LCMS: 347.2 [M+H]⁺;

### Step 5: Preparation of Example 124

The title compound was synthesized according to Step 3 in Example 38. LCMS: 568.3 [M+H]⁺; ¹H NMR (400 MHz, DMSO-d₆) δ 8.98 (s, 1H), 8.56 (s, 1H), 8.11 (s, 1H), 7.84-7.78 (m, 1H), 7.70 (d, J=8.3 Hz, 1H), 7.55 (s, 1H), 7.48 (t, J=58.6 Hz, 1H), 7.06 (s, 1H), 6.13 (d, J=52.2 Hz, 2H), 3.58 (s, 2H), 2.96-2.83 (m, 2H), 2.78-2.68 (m, 2H), 2.65-2.55 (m, 3H), 1.90 (t, J=10.3 Hz, 1H), 1.68-1.54 (m, 4H), 1.53-1.40 (m, 1H), 1.08 (d, J=5.0 Hz, 3H), 0.91-0.82 (m, 1H), 0.80 (d, J=5.6 Hz, 3H).

### Example A: Assay

An HTRF based binding assay was applied to assess the affinity of candidate compounds to Cbl-b protein. CBL-B (GST-Tag) was purchased from BPS (Cat #80415). A fluorescence labeled Cbl-b binding probe was made in-house. Anti GST-Tb cryptate monoclonal antibody was from Cisbio (61GSTTLB). In brief, compounds were diluted and transferred to opti-384F black plate (PE, 6007279) by echo. 1× Assay Buffer (HEPES pH 7.5 50 mM, NaCl 50 mM, MgCl₂ 5 mM, TCEP 1 mM, Tween-20 0.01%), 2× Protein mix (10 nM), and 2× Probe mix (120 nM) were prepared. 10 μL 2× Protein mix (or 1× Assay buffer, as no protein control) was added to assay plate. After 30 min incubation 25° C., 10 μl 2× Probe mix was added for another 2 h incubation at 25° C. Data were collected by Envision reading using Ex340/Em495/520.

The results are found in Table 3.

TABLE 3

| EX | IC₅₀ |
|---|---|
| 1 | C |
| 2 | A |
| 3 | NT |
| 4 | A |
| 5 | C |
| 6 | B |
| 7 | A |
| 8 | C |
| 9 | C |
| 10 | E |
| 11 | C |
| 12 | A |
| 13 | D |
| 14 | C |
| 15 | E |
| 16 | A |
| 17 | C |
| 18 | E |
| 19 | C |
| 20 | D |
| 21 | C |
| 22 | C |
| 23 | A |
| 24 | A |
| 25 | B |
| 26 | E |
| 27 | C |
| 28 | A |
| 29 | A |
| 30 | B |
| 31 | C |
| 32 | A |
| 33 | B |
| 34 | B |
| 35 | C |
| 36 | B |
| 37 | C |
| 38 | A |
| 39 | D |
| 40 | C |
| 41 | B |
| 42 | B |
| 43 | C |
| 44 | B |
| 45 | C |
| 46 | C |
| 47 | A |
| 48 | A |
| 49 | A |
| 50 | A |
| 51 | C |
| 52 | A |
| 53 | C |
| 54 | C |
| 55 | D |
| 56 | A |
| 57 | A |
| 58 | B |
| 59 | C |
| 60 | C |
| 61 | B |
| 62 | B |
| 63 | A |
| 64 | A |
| 65 | D |
| 66 | B |
| 67 | C |
| 68 | C |
| 69 | A |
| 70 | A |
| 71 | C |
| 72 | B |
| 73 | E |
| 74 | C |
| 75 | A |
| 76 | C |
| 77 | A |
| 78 | A |
| 79 | B |
| 80 | D |
| 81 | C |
| 82 | A |
| 83 | C |
| 84 | A |
| 85 | C |
| 86 | C |
| 87 | B |
| 88 | A |
| 89 | C |
| 90 | A |
| 91 | B |
| 92 | C |
| 93 | B |
| 94 | A |
| 95 | A |
| 96 | A |
| 97 | C |
| 98 | C |
| 99 | A |
| 100 | A |
| 101 | A |
| 102 | A |
| 103 | A |
| 104 | A |
| 105 | A |
| 106 | A |
| 107 | A |
| 108 | A |
| 109 | A |
| 110 | C |
| 111 | A |
| 112 | A |
| 113 | A |
| 114 | B |
| 115 | A |
| 116 | A |
| 117 | A |
| 118 | B |
| 119 | A |
| 120 | D |
| 121 | A |
| 122 | NT |
| 123 | A |
| 124 | A |

0 nM < A ≤ 20 nM; 20 nM < B ≤ 50 nM; 50 nM < C ≤ 500 nM; 500 nM < D ≤ 1000 nM; E > 1000 nM
NT: not tested

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound of Formula (I), or a pharmaceutically acceptable salt thereof: 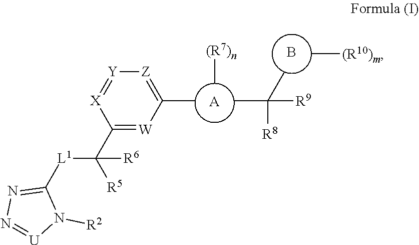
wherein:
U is —N— or —CR¹—;
R¹ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R² is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
L¹ is absent or —CR³R⁴—;
R³ and R⁴ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or R³ and R⁴ are taken together to form a cycloalkyl or heterocycloalkyl; each optionally substituted with one or more R;
or R³ and R⁴ are taken together to form an oxo;
R⁵ and R⁶ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{5a};
or R⁵ and R⁶ are taken together to form a cycloalkyl or heterocycloalkyl; each optionally substituted with one or more R^{5a};
each R^{5a} is independently halogen, —CN, —NO₂, —OH, —OR^{a}, —OC(═O)R^{a}, —OC(═O)OR^{b}, —OC(═O)NR_{c}R^{d}, —SF₅, —SH, —SR^{a}, —S(═O)R^{a}, —S(═O)₂R^{a}, —S(═O)₂NR_{c}R^{d}, —S(═O)(=NR^{b})R^{b}, —NR_{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{5a} on the same atom are taken together to form an oxo;
X is —N— or —CR^{X}—;
R^{X} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Y is —N— or —CR^{Y}—;
R^{Y} is hydrogen, halogen, —CN, —OH, —OR^{a}, —NR^{c}R^{d}, —S(═O)R^{a}, —S(═O)₂R^{a}, —S(═O)₂NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆alkoxy, C₁-C₆haloalkoxy, C₁-C₆hydroxyalkyl, C₁-C₆aminoalkyl, C₁-C₆heteroalkyl, C₂-C₆alkenyl, C₂-C₆haloalkenyl C₂-C₆alkynyl, cycloalkyl optionally substituted with one or more halogens, or heterocycloalkyl optionally substituted with one or more halogens;
Z is —N— or —CR^{Z}—;
R^{Z} is hydrogen, halogen, —CN, —OH, —OR^{a}, —NR_{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
or R^{X} and R^{Y} are taken together to form a cycloalkyl, heterocycloalkyl, aryl, or heteroaryl; each optionally substituted with one or more R;
or R^{Y} and R^{Z} are taken together to form a cycloalkyl, heterocycloalkyl, aryl, or heteroaryl; each optionally substituted with one or more R; W is —N— or —CR^{W}—;
R^{W} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Ring A is cycloalkyl, heterocycloalkyl, aryl, or heteroaryl;
each R⁷ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR_{c}R^{d}, —S(═O)(=NR^{b})R^{b}, —NR_{c}R^{d}, —NR^{b}C(═O)NR_{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R⁷ on the same atom are taken together to form an oxo;
n is 0, 1, 2, 3, 4, 5, or 6;
R⁸ and R⁹ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or R⁸ and R⁹ are taken together to form a cycloalkyl or heterocycloalkyl; each optionally substituted with one or more R;
or R⁸ and R⁹ are taken together to form an oxo;
Ring B is cycloalkyl, heterocycloalkyl, aryl, or heteroaryl;
each RIU is independently halogen, —CN, —NO₂, —OH, —OR^{a}, —OC(═O)R^{a}, —OC(═O)OR^{a}, —OC(═O)NR^{c}R^{d}, —SF₅, —SH, —SR^{a}, —S(═O)R^{a}, —S(═O)₂R^{a}, —S(═O)₂NR_{c}R^{d}, —S(═O)(=NR^{b})R^{a}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or two R¹⁰ on the same atom are taken together to form an oxo;
m is 0, 1, 2, 3, 4, 5, or 6;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(=NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, or C₃-C₆cycloalkyl;
or two R on the same atom form an oxo;
provided that 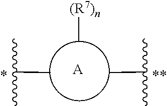
is not 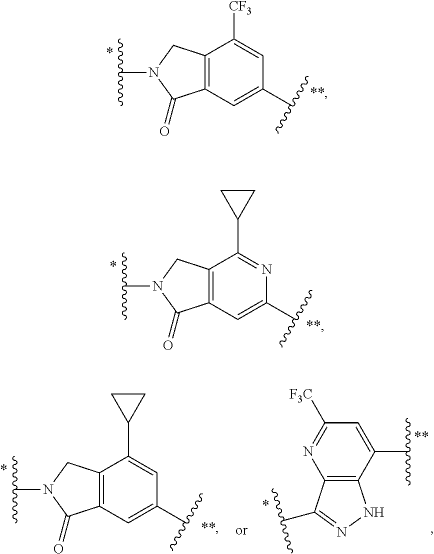 [SMILES: *c1cc2c(c(C(F)(F)F)c1)CN(*)C2=O ; *c1cc2c(c(C3CC3)n1)CN(*)C2=O ; *c1cc2c(c(C3CC3)c1)CN(*)C2=O ; *c1n[nH]c2c(*)cc(C(F)(F)F)nc12]
wherein * represents the attachment point to the ring containing X, Y, Z and W and ** represents the attachment point to —CR⁸R⁹—.

2. The compound of claim 1, or a pharmaceutically acceptable salt thereof, wherein the compound is of Formula (Ia):

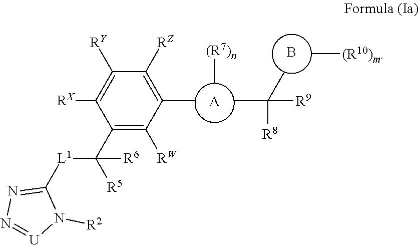

3. The compound of claim 1, or a pharmaceutically acceptable salt thereof, wherein the compound is of Formula (Id):

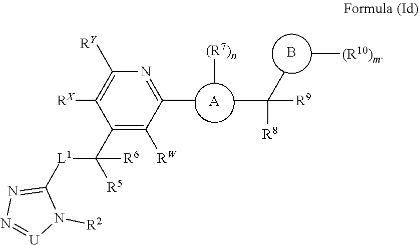

4. The compound of any one of claims 1-3, or a pharmaceutically acceptable salt thereof, wherein R^{X} is hydrogen or C₁-C₆alkyl.

5. The compound of any one of claims 1-4, or a pharmaceutically acceptable salt thereof, R is hydrogen, halogen, —CN, —OR^{a}, —C(═O)NR^{c}R^{d}, C₁-C₆alkyl, C₁-C₆hydroxyalkyl, C₂-C₆alkenyl, C₂-C₆alkynyl, or cycloalkyl optionally substituted with one or more halogens.

6. The compound of any one of claims 1-5, or a pharmaceutically acceptable salt thereof, wherein R^{Y} is hydrogen, C₃-C₆cycloalkyl, or C₁-C₆haloalkoxy.

7. The compound of any one of claims 1-6, or a pharmaceutically acceptable salt thereof, wherein R^{Y} is —OR^{a}.

8. The compound of any one of claims 1-7, or a pharmaceutically acceptable salt thereof, wherein R^{Z} is hydrogen or C₁-C₆alkyl.

9. The compound of any one of claims 1-8, or a pharmaceutically acceptable salt thereof, wherein R^{W} is hydrogen or C₁-C₆alkyl.

10. The compound of any one of claims 1-9, or a pharmaceutically acceptable salt thereof, wherein R⁸ and R⁹ are each independently hydrogen or C₁-C₆alkyl.

11. The compound of any one of claims 1-10, or a pharmaceutically acceptable salt thereof, wherein Ring B is 5- or 6-membered heterocycloalkyl.

12. The compound of any one of claims 1-11, or a pharmaceutically acceptable salt thereof, wherein Ring B is azetidinyl, pyrrolidinyl, piperidinyl, or morpholinyl.

13. The compound of any one of claims 1-12, or a pharmaceutically acceptable salt thereof, wherein each R¹⁰ is independently halogen, —CN, —OH, —OR^{a}, —NR^{c}R^{d}, C₁-C₆alkyl, C₁-C₆haloalkyl, C₂-C₆alkynyl, cycloalkyl, heterocycloalkyl, C₁-C₆ alkylene(cycloalkyl), or C_{G}-C₆alkylene(heterocycloalkyl); wherein each alkyl, alkylene, alkynyl, cycloalkyl, and heterocycloalkyl is independently optionally substituted with one or more R.

14. The compound of any one of claims 1-13, or a pharmaceutically acceptable salt thereof, wherein each R¹⁰ is independently halogen, C₁-C₆alkyl, C₁-C₆haloalkyl, C₂-C₆ alkynyl, C₁-C₆alkylene(cycloalkyl), or C₁-C₆alkylene(heterocycloalkyl); wherein each alkyl, alkylene, alkynyl, cycloalkyl, and heterocycloalkyl is independently optionally substituted with one or more R.

15. The compound of any one of claims 1-14, or a pharmaceutically acceptable salt thereof, wherein each R¹⁰ is independently halogen or C₁-C₆alkyl.

16. The compound of any one of claims 1-15, or a pharmaceutically acceptable salt thereof, wherein m is 0, 1, 2, or 3.

17. The compound of any one of claims 1-16, or a pharmaceutically acceptable salt thereof, wherein

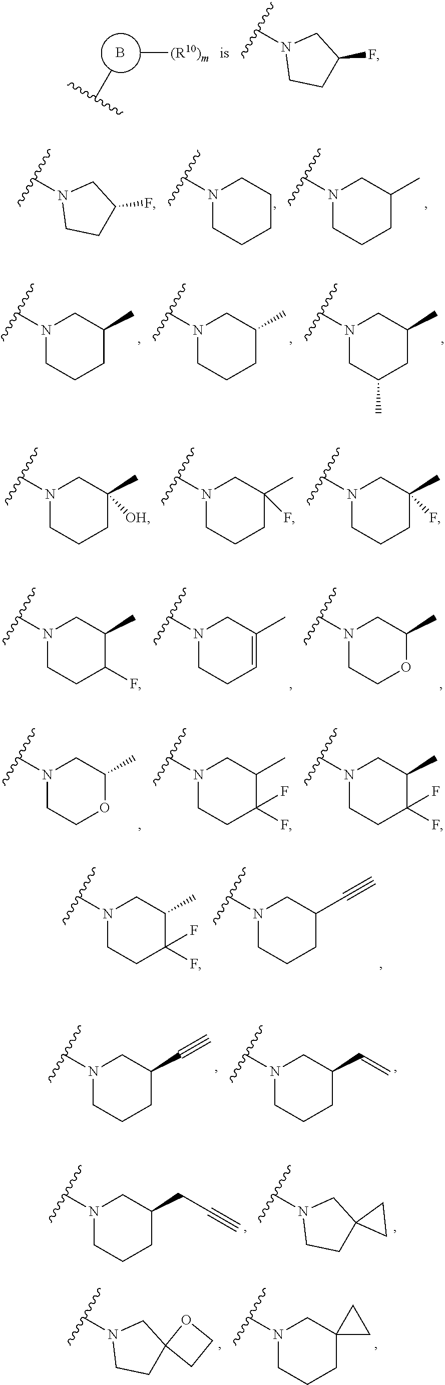

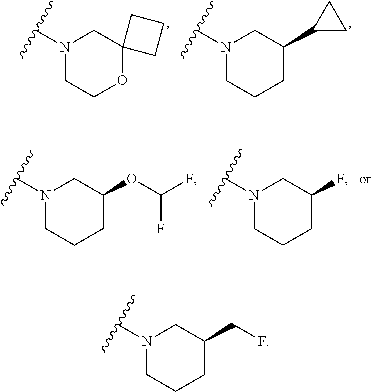

18. The compound of any one of claims 1-17, or a pharmaceutically acceptable salt thereof, wherein 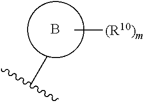
is

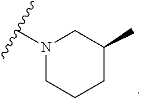 [SMILES: *N1CCC[C@H](C)C1]

19. The compound of any one of claims 1-18, or a pharmaceutically acceptable salt thereof, wherein L¹ is absent.

20. The compound of any one of claims 1-19, or a pharmaceutically acceptable salt thereof, wherein L¹ is —CR³R⁴—.

21. The compound of any one of claims 1-18 or 20, or a pharmaceutically acceptable salt thereof, wherein R³ and R⁴ are each independently hydrogen, halogen, C₁-C₆alkyl, or C₁-C₆haloalkyl.

22. The compound of any one of claims 1-18 or 20 or 21, or a pharmaceutically acceptable salt thereof, wherein R³ and R⁴ are each hydrogen.

23. The compound of any one of claims 1-22, or a pharmaceutically acceptable salt thereof, wherein R⁶ is hydrogen, halogen, —CN, —OH, —OR^{a}, —NR^{c}R^{d}, C₁-C₆alkyl, or C₁-C₆haloalkyl.

24. The compound of any one of claims 1-23, or a pharmaceutically acceptable salt thereof, wherein R⁶ is hydrogen, C₁-C₆alkyl, or C₁-C₆haloalkyl.

25. The compound of any one of claims 1-24, or a pharmaceutically acceptable salt thereof, wherein R⁶ is hydrogen.

26. The compound of any one of claims 1-25, or a pharmaceutically acceptable salt thereof, wherein R⁵ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R⁵.

27. The compound of any one of claims 1-16, or a pharmaceutically acceptable salt thereof, wherein R⁵ is cycloalkyl, heterocycloalkyl, aryl, or heteroaryl; wherein each cycloalkyl, heterocycloalkyl, aryl, and heteroaryl is independently optionally substituted with one or more R⁵.

28. The compound of any one of claims 1-27, or a pharmaceutically acceptable salt thereof, wherein R⁵ is cycloalkyl or heterocycloalkyl; wherein each cycloalkyl and heterocycloalkyl is independently optionally substituted with one or more R^{5a}.

29. The compound of any one of claims 1-22, or a pharmaceutically acceptable salt thereof, wherein R⁵ and R⁶ are taken together to form a cycloalkyl optionally substituted with one or more R^{5a}.

30. The compound of any one of claims 1-22 or 29, or a pharmaceutically acceptable salt thereof, wherein R⁵ and R⁶ are taken together to form a heterocycloalkyl optionally substituted with one or more R^{5a}.

31. The compound of any one of claims 1-22 or 29 or 30, or a pharmaceutically acceptable salt thereof, wherein R⁵ and R⁶ are taken together to form a cyclobutyl optionally substituted with one or more R^{5a}.

32. The compound of any one of claims 1-31, or a pharmaceutically acceptable salt thereof, wherein each R⁵, is independently halogen, —CN, —OH, —OR^{a}, —NR_{c}R^{d}, C₁-C₆alkyl, C₁-C₆haloalkyl, C₂-C₆alkynyl, cycloalkyl, or heterocycloalkyl; wherein each alkyl, alkynyl, cycloalkyl, and heterocycloalkyl is independently optionally substituted with one or more R.

33. The compound of any one of claims 1-32, or a pharmaceutically acceptable salt thereof, wherein each R^{5a} is independently halogen, —CN, C₁-C₆alkyl, C₁-C₆haloalkyl, or C₂-C₆alkynyl; wherein each alkyl and alkynyl is independently optionally substituted with one or more R.

34. The compound of any one of claims 1-22, or a pharmaceutically acceptable salt thereof, wherein 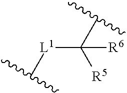
is

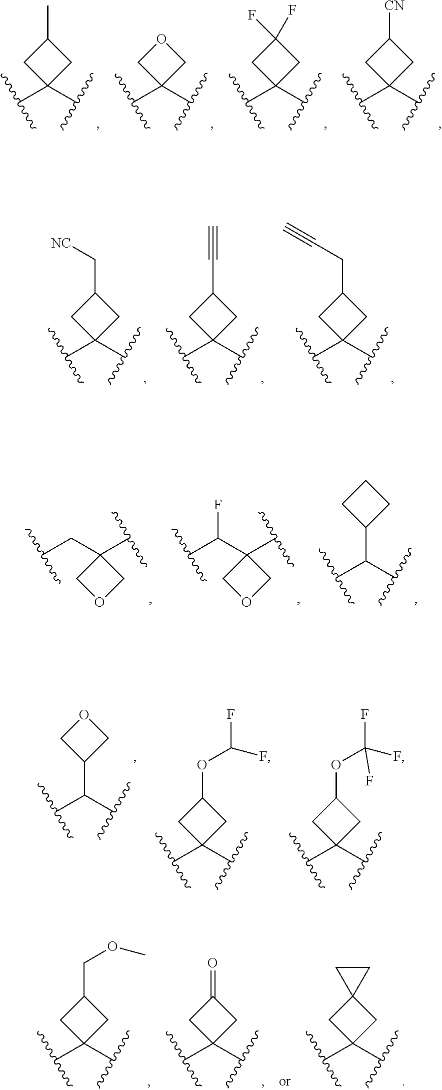

35. The compound of any one of claims 1-22, or a pharmaceutically acceptable salt thereof, wherein 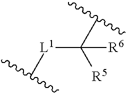
is

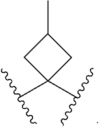 [SMILES: *C1(*)CC(C)C1]

36. The compound of any one of claims 1-35, or a pharmaceutically acceptable salt thereof, wherein R¹ is hydrogen.

37. The compound of any one of claims 1-36, or a pharmaceutically acceptable salt thereof, wherein R² is C₁-C₆alkyl.

38. The compound of any one of claims 1-37, or a pharmaceutically acceptable salt thereof, wherein Ring A is a bicyclic cycloalkyl, bicyclic heterocycloalkyl, bicyclic aryl, or bicyclic heteroaryl.

39. The compound of any one of claims 1-37, or a pharmaceutically acceptable salt thereof, wherein Ring A is a bicyclic cycloalkyl or bicyclic heterocycloalkyl.

40. The compound of any one of claims 1-37, or a pharmaceutically acceptable salt thereof, wherein Ring A is a bicyclic aryl or bicyclic heteroaryl.

41. The compound of any one of claims 1-37, or a pharmaceutically acceptable salt thereof, wherein Ring A is a bicyclic heteroaryl.

42. The compound of any one of claims 1-37, or a pharmaceutically acceptable salt thereof, wherein Ring A is benzothiazolyl, benzimidazolyl, indazolyl, pyrazolopyridinyl, thiazolopyridinyl, quinoxalinyl, or pyridopyrazinyl.

43. The compound of any one of claims 1-37, or a pharmaceutically acceptable salt thereof, wherein Ring A is tetrahydroisoquinolinyl, dihydrophthalazinyl, dihydroisoquinolinyl, dihydroquinazolinyl, dihydropyrroloimidazolyl, dihydroimidazopyridinyl, dihydrothienopyridazinyl, dihydroindazolyl, tetrahydropyrrolopyridinyl, or dihydropyrrolopyridine.

44. The compound of any one of claims 1-43, or a pharmaceutically acceptable salt thereof, wherein each R⁷ is independently halogen, —CN, —OH, —OR^{a}, —NR_{c}R^{d}, C₁-C₆alkyl, C₁-C₆haloalkyl, C₁-C₆hydroxyalkyl, C₁-C₆aminoalkyl, or C₁-C₆heteroalkyl; wherein each alkyl is independently optionally substituted with one or more R; or two R⁷ on the same atom are taken together to form an oxo.

45. The compound of any one of claims 1-44, or a pharmaceutically acceptable salt thereof, wherein each R⁷ is independently —OH, C₁-C₆alkyl, or C₁-C₆haloalkyl; or two R⁷ on the same atom are taken together to form an oxo.

46. The compound of any one of claims 1-45, or a pharmaceutically acceptable salt thereof, wherein n is 0, 1, 2, 3, or 4.

47. The compound of any one of claims 1-46, or a pharmaceutically acceptable salt thereof, wherein n is 1, 2, or 3.

48. The compound of any one of claims 1-47, or a pharmaceutically acceptable salt thereof, wherein 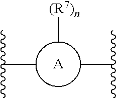
is

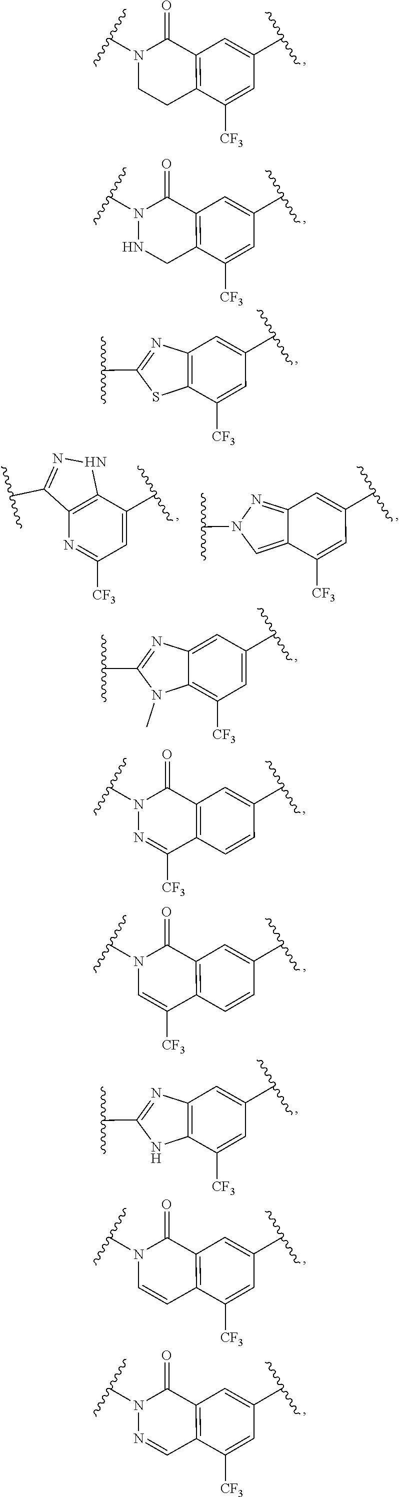

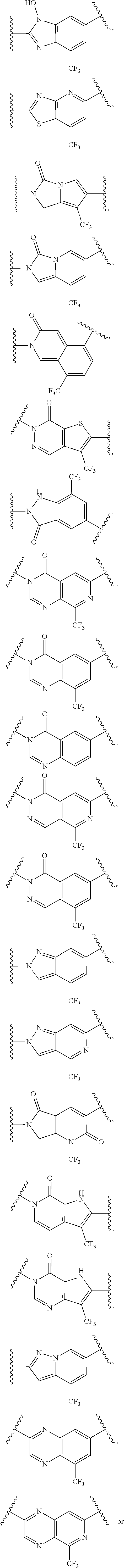

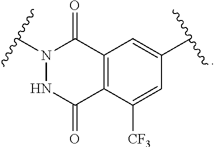 [SMILES: *c1cc(C(F)(F)F)c2c(=O)[nH]n(*)c(=O)c2c1]

49. A compound of Formula (III), or a pharmaceutically acceptable salt thereof: 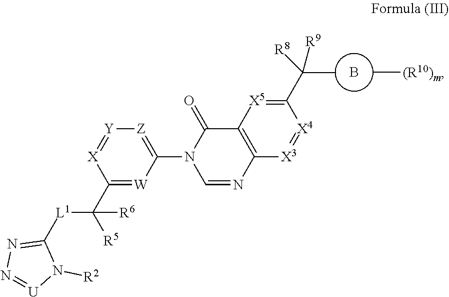
wherein:
U is —N— or —CR¹—;
R¹ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
R² is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
L¹ is absent or —CR³R⁴—;
R³ and R⁴ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or R³ and R⁴ are taken together to form a cycloalkyl or heterocycloalkyl; each optionally substituted with one or more R;
or R³ and R⁴ are taken together to form an oxo;
R⁵ and R⁶ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{5a};
or R⁵ and R⁶ are taken together to form a cycloalkyl or heterocycloalkyl; each optionally substituted with one or more R^{a};
each R^{5a} is independently halogen, —CN, —NO₂, —OH, —OR^{a}, —OC(═O)R^{a}, —OC(═O)OR^{b}, —OC(═O)NR_{c}R^{d}, —SF₅, —SH, —SR^{a}, —S(═O)R^{a}, —S(═O)₂R^{a}, —S(═O)₂NR^{c}R^{d}, —S(═O)(=NR^{b})R^{b}, —NR^{c}R^{d}, —NR^{b}C(═O)NR_{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{5a} on the same atom are taken together to form an oxo;
X is —N— or —CR^{X}—;
R^{X} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
Y is —N— or —CR^{Y}—;
R^{Y} is hydrogen, halogen, —CN, —OH, —OR^{a}, —NR^{c}R^{d}, —S(═O)R^{a}, —S(═O)₂R^{a}, —S(═O)₂NR^{c}R^{d}, —NR^{b}C(═O)NR^{c}R^{d}, —NR^{b}C(═O)R^{a}, —NR^{b}C(═O)OR^{b}, —NRS(═O)₂R^{a}, —C(═O)R^{a}, —C(═O)OR^{a}, —C(═O)NR^{c}R^{d}, C₁-C₆alkyl, C₁-C₆haloalkyl, C₁-C₆alkoxy, C₁-C₆haloalkoxy, C₁-C₆hydroxyalkyl, C₁-C₆aminoalkyl, C₁-C₆heteroalkyl, C₂-C₆alkenyl, C₂-C₆haloalkenyl C₂-C₆alkynyl, cycloalkyl optionally substituted with one or more halogens, or heterocycloalkyl optionally substituted with one or more halogens;
Z is —N— or —CR^{Z}—;
R^{Z} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
or R^{X} and R^{Y} are taken together to form a cycloalkyl, heterocycloalkyl, aryl, or heteroaryl; each optionally substituted with one or more R;
or R^{Y} and R^{Z} are taken together to form a cycloalkyl, heterocycloalkyl, aryl, or heteroaryl; each optionally substituted with one or more R; W is —N— or —CR^{W}—;
R^{W} is hydrogen, halogen, —CN, —OH, —OR^{a}, —NR_{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl;
X³, X⁴, and X⁵ are independently N or CR^{7′};
each R^{7′} is independently hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR_{c}R^{d}, —S(═O)(=NR^{b})R^{b}, —NR_{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR_{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
R⁸ and R⁹ are each independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or R⁸ and R⁹ are taken together to form a cycloalkyl or heterocycloalkyl; each optionally substituted with one or more R;
or R⁸ and R⁹ are taken together to form an oxo;
Ring B is cycloalkyl, heterocycloalkyl, aryl, or heteroaryl;
each R¹⁰ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(=NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or two R¹⁰ on the same atom are taken together to form an oxo;
m is 0, 1, 2, 3, 4, 5, or 6;
each R is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(=NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, or C₃-C₆cycloalkyl;
or two R on the same atom form an oxo.

50. The compound of claim 49, or a pharmaceutically acceptable salt thereof, wherein X is —N—.

51. The compound of claim 49, or a pharmaceutically acceptable salt thereof, wherein X is —CR^{X}—.

52. The compound of any one of claims 49-51, or a pharmaceutically acceptable salt thereof, wherein Y is —N—.

53. The compound of any one of claims 49-51, or a pharmaceutically acceptable salt thereof, wherein Y is —CR¹—.

54. The compound of any one of claims 49-53, or a pharmaceutically acceptable salt thereof, wherein Z is —N—.

55. The compound of any one of claims 49-54, or a pharmaceutically acceptable salt thereof, wherein Z is —CR^{Z}—.

56. The compound of any one of claims 49-55, or a pharmaceutically acceptable salt thereof, wherein W is —N—.

57. The compound of any one of claims 49-55, or a pharmaceutically acceptable salt thereof, wherein W is —CR^{W}—.

58. The compound of claim 49, or a pharmaceutically acceptable salt thereof, wherein the compound is of Formula (IIId):

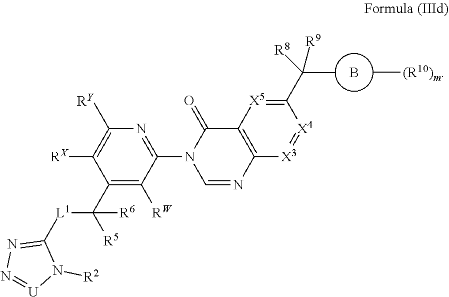

59. The compound of any one of claims 49-58, or a pharmaceutically acceptable salt thereof, wherein R^{X} is hydrogen or C₁-C₆alkyl.

60. The compound of any one of claims 49-59, or a pharmaceutically acceptable salt thereof, wherein R^{X} is hydrogen.

61. The compound of any one of claims 49-60, or a pharmaceutically acceptable salt thereof, wherein R^{Y} is hydrogen, halogen, —CN, —OR^{a}, —C(═O)NR^{c}R^{d}, C₁-C₆alkyl, C₁-C₆hydroxyalkyl, C₂-C₆alkenyl, C₂-C₆alkynyl, or cycloalkyl optionally substituted with one or more halogens.

62. The compound of any one of claims 49-61, or a pharmaceutically acceptable salt thereof, wherein R^{Y} is hydrogen, —OR^{a}, or cycloalkyl optionally substituted with one or more halogens.

63. The compound of any one of claims 49-62, or a pharmaceutically acceptable salt thereof, wherein R^{Y} is hydrogen, —OR^{a}, or cycloalkyl.

64. The compound of any one of claims 49-63, or a pharmaceutically acceptable salt thereof, wherein R^{Y} is —OR^{a}.

65. The compound of any one of claims 49-64, or a pharmaceutically acceptable salt thereof, wherein R^{Y} is —OCH₂F.

66. The compound of any one of claims 49-63, or a pharmaceutically acceptable salt thereof, wherein R^{Y} is hydrogen.

67. The compound of any one of claims 49-66, or a pharmaceutically acceptable salt thereof, wherein R^{Z} is hydrogen or C₁-C₆alkyl.

68. The compound of any one of claims 49-67, or a pharmaceutically acceptable salt thereof, wherein R^{Z} is hydrogen.

69. The compound of any one of claims 49-68, or a pharmaceutically acceptable salt thereof, wherein R^{W} is hydrogen or C₁-C₆alkyl.

70. The compound of any one of claims 49-69, or a pharmaceutically acceptable salt thereof, wherein R^{W} is hydrogen.

71. The compound of any one of claims 49-70, or a pharmaceutically acceptable salt thereof, wherein R⁸ and R⁹ are each independently hydrogen or C₁-C₆alkyl.

72. The compound of any one of claims 49-71, or a pharmaceutically acceptable salt thereof, wherein R⁸ and R⁹ are each hydrogen.

73. The compound of any one of claims 49-72, or a pharmaceutically acceptable salt thereof, wherein Ring B is 5- or 6-membered heterocycloalkyl.

74. The compound of any one of claims 49-73, or a pharmaceutically acceptable salt thereof, wherein Ring B is azetidinyl, pyrrolidinyl, piperidinyl, or morpholinyl.

75. The compound of any one of claims 49-74, or a pharmaceutically acceptable salt thereof, wherein Ring B is piperidinyl.

76. The compound of any one of claims 49-75, or a pharmaceutically acceptable salt thereof, wherein each R¹⁰ is independently halogen, C₁-C₆alkyl, C₁-C₆haloalkyl, C₂-C₆alkynyl, C₁-C₆alkylene(cycloalkyl), or C₁-C₆alkylene(heterocycloalkyl); wherein each alkyl, alkylene, alkynyl, cycloalkyl, and heterocycloalkyl is independently optionally substituted with one or more R.

77. The compound of any one of claims 49-76, or a pharmaceutically acceptable salt thereof, wherein each R¹⁰ is independently halogen or C₁-C₆alkyl.

78. The compound of any one of claims 49-77, or a pharmaceutically acceptable salt thereof, wherein each R¹⁰ is independently C₁-C₆alkyl.

79. The compound of any one of claims 49-78, or a pharmaceutically acceptable salt thereof, wherein m is 1 or 2.

80. The compound of any one of claims 49-71, or a pharmaceutically acceptable salt thereof, wherein 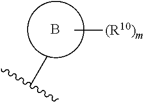
is

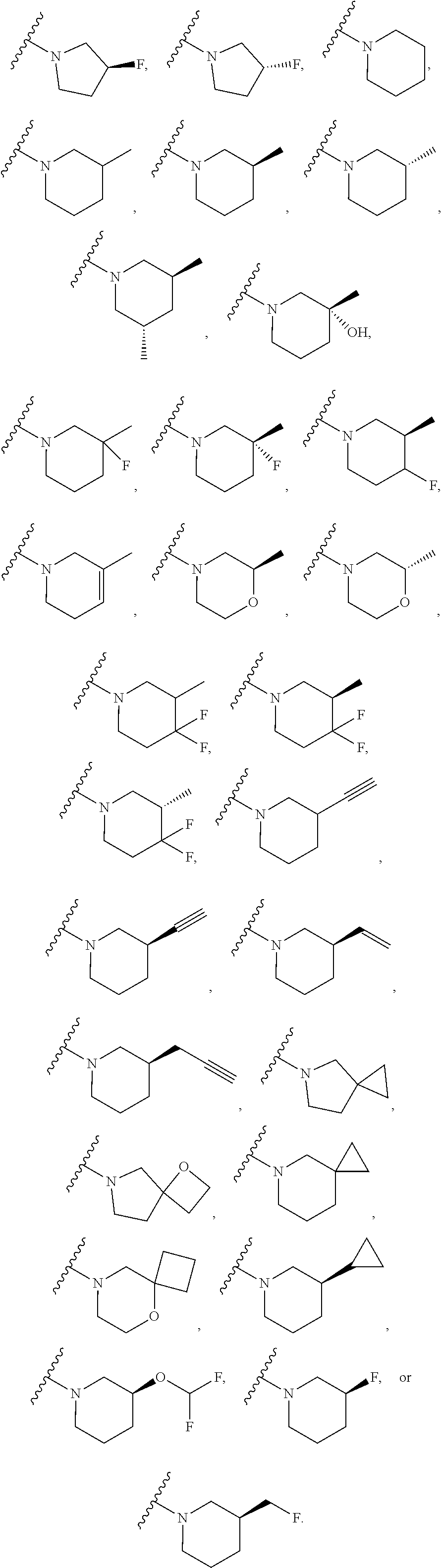

81. The compound of any one of claims 49-71, or a pharmaceutically acceptable salt thereof, wherein 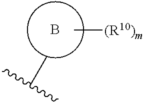
is

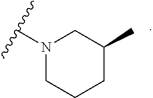 [SMILES: *N1CCC[C@H](C)C1]

82. The compound of any one of claims 49-81, or a pharmaceutically acceptable salt thereof, wherein L¹ is absent.

83. The compound of any one of claims 49-81, or a pharmaceutically acceptable salt thereof, wherein L¹ is —CR³R⁴—.

84. The compound of any one of claims 49-81 or 82, or a pharmaceutically acceptable salt thereof, wherein R³ and R⁴ are each hydrogen.

85. The compound of any one of claims 49-84, or a pharmaceutically acceptable salt thereof, wherein R⁶ is hydrogen, C₁-C₆alkyl, or C₁-C₆haloalkyl.

86. The compound of any one of claims 49-85, or a pharmaceutically acceptable salt thereof, wherein R⁶ is hydrogen.

87. The compound of any one of claims 49-86, or a pharmaceutically acceptable salt thereof, wherein R⁵ is cycloalkyl or heterocycloalkyl; wherein each cycloalkyl and heterocycloalkyl is independently optionally substituted with one or more R^{5a}.

88. The compound of any one of claims 49-84, or a pharmaceutically acceptable salt thereof, wherein R⁵ and R⁶ are taken together to form a cycloalkyl optionally substituted with one or more R^{5a}.

89. The compound of any one of claims 49-84 or 88, or a pharmaceutically acceptable salt thereof, wherein R⁵ and R⁶ are taken together to form a heterocycloalkyl optionally substituted with one or more R⁵.

90. The compound of any one of claims 49-84 or 88, or a pharmaceutically acceptable salt thereof, wherein R⁵ and R⁶ are taken together to form a cyclobutyl optionally substituted with one or more R⁵.

91. The compound of any one of claims 49-80, or a pharmaceutically acceptable salt thereof, wherein each R^{5a} is independently halogen, —CN, —OH, —OR^{a}, —NR_{c}R^{d}, C₁-C₆alkyl, C₁-C₆haloalkyl, C₁-C₆heteroalkyl, C₂-C₅alkynyl, cycloalkyl, or heterocycloalkyl; wherein each alkyl, alkynyl, cycloalkyl, and heterocycloalkyl is independently optionally substituted with one or more R.

92. The compound of any one of claims 49-91, or a pharmaceutically acceptable salt thereof, wherein each R^{5a} is independently halogen, —CN, C₁-C₆alkyl, C₁-C₆haloalkyl, or C₂-C₆alkynyl; wherein each alkyl and alkynyl is independently optionally substituted with one or more R.

93. The compound of any one of claims 49-81, or a pharmaceutically acceptable salt thereof, wherein 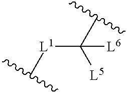
is

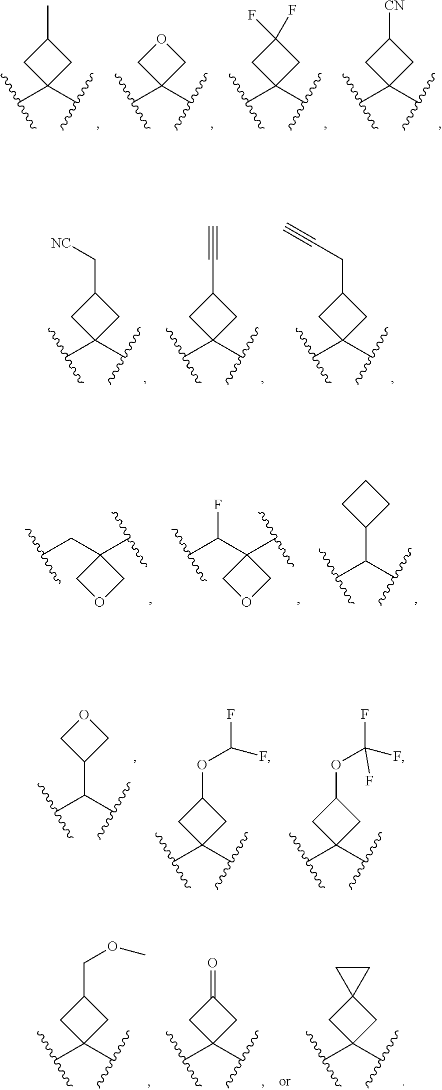

94. The compound of any one of claims 49-81, or a pharmaceutically acceptable salt thereof, wherein 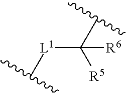
is

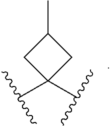 [SMILES: *C1(*)CC(C)C1]

95. The compound of any one of claims 49-81, or a pharmaceutically acceptable salt thereof, wherein 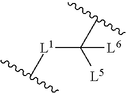
is

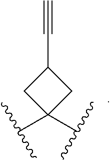 [SMILES: *C1(*)CC(C#C)C1]

96. The compound of any one of claims 49-95, or a pharmaceutically acceptable salt thereof, wherein 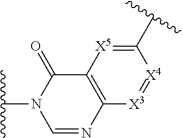
is

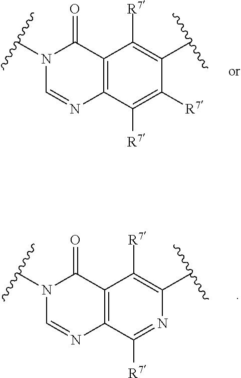

97. The compound of any one of claims 49-95, or a pharmaceutically acceptable salt thereof, wherein 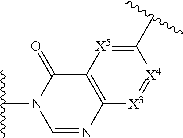
is

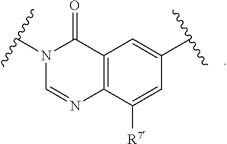

98. The compound of any one of claims 49-95, or a pharmaceutically acceptable salt thereof, wherein 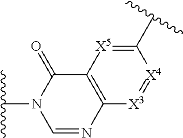
is

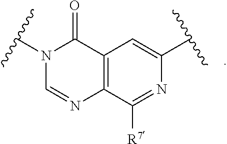

99. The compound of any one of claims 49-98, or a pharmaceutically acceptable salt thereof, wherein each RT is independently hydrogen or C₁-C₆haloalkyl.

100. The compound of any one of claims 49-99, or a pharmaceutically acceptable salt thereof, wherein each R^{7′} is independently hydrogen or —CF₃.

101. The compound of any one of claims 49-95, or a pharmaceutically acceptable salt thereof, wherein 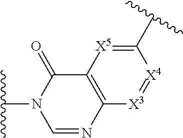
is

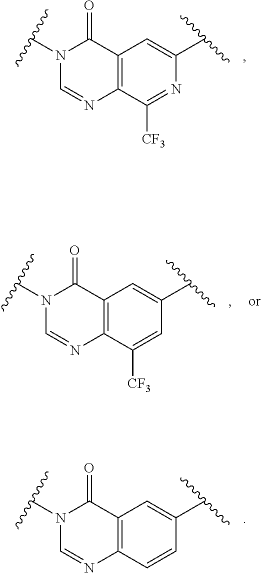 [SMILES: *c1cc2c(=O)n(*)cnc2c(C(F)(F)F)n1 ; *c1cc(C(F)(F)F)c2ncn(*)c(=O)c2c1 ; *c1ccc2ncn(*)c(=O)c2c1]

102. The compound of claim 1 or 49, or a pharmaceutically acceptable salt thereof, selected from a compound in table 1 or table 2.

103. A pharmaceutical composition comprising a compound of any one of claims 1-102, or a pharmaceutically acceptable salt thereof, and a pharmaceutically acceptable excipient.

104. A method of modulating activity of an immune cell, the method comprising contacting the immune cell with an effective amount of a compound of any one of claims 1-102, or a pharmaceutically acceptable salt thereof, to the subject in need thereof.

105. A method of treating a cancer, the method comprising administering an effective amount of a compound of any one of claims 1-102, or a pharmaceutically acceptable salt thereof, to the subject in need thereof.

106. A method of treating a cancer responsive to inhibition of Cbl-b activity, the method comprising administering an effective amount of a compound of any one of claims 1-102, or a pharmaceutically acceptable salt thereof, to the subject in need thereof.

107. The method of claim 105 or 106, wherein the cancer is a hematologic cancer.

108. The method of claim 105 or 106, wherein the cancer is a lymphoma, a leukemia, or a myeloma.

109. The method of claim 105 or 106, wherein the cancer is a non-hematologic cancer.

110. The method of claim 105 or 106, wherein the cancer is a sarcoma, a carcinoma, or a melanoma.

111. The method of claim 105 or 106, wherein the cancer is solid tumor cancer.

112. A method of inhibiting abnormal cell proliferation, the method comprising administering an effective amount of a compound of any one of claims 1-102, or a pharmaceutically acceptable salt thereof, to the subject in need thereof.

113. A method of modulating the immune response, the method comprising administering an effective amount of a compound of any one of claims 1-102, or a pharmaceutically acceptable salt thereof, to the subject in need thereof.

114. A method of inhibiting Cbl-b activity, the method comprising administering an effective amount of a compound of any one of claims 1-102, or a pharmaceutically acceptable salt thereof, to the subject in need thereof.

115. A method for treating a disease or condition associated with Cbl-b activity, the method comprising administering an effective amount of a compound of any one of claims 1-102, or a pharmaceutically acceptable salt thereof, to the subject in need thereof.
